(19) United States
(12) Patent Application Publication
(10) Pub. No.: US 2024/0287006 A1
(43) Pub. Date: Aug. 29, 2024

(54) E3 Ligase Inhibitors and Methods of Use Thereof

(71) Applicant: The Regents of the University of California, Oakland, CA (US)

(72) Inventors: Michael Rape, El Cerrito, CA (US); Julia Sabine Schaletzky, El Cerrito, CA (US); Edward Eric Wehri, Berkeley, CA (US); Andrew Garrett Manford, Berkeley, CA (US)

(21) Appl. No.: 18/570,555

(22) PCT Filed: Jun. 16, 2022
(86) PCT No.: PCT/US22/33793

§ 371 Date: Dec. 14, 2023

Related U.S. Application Data
(60) Provisional application No. 63/321,034, filed on Mar. 17, 2022.
(60) Provisional application No. 63/211,868, filed on Jun. 17, 2021.

(51) Int. Cl. C07D 263/58; A61K 31/422; A61K 31/423; A61K 31/437; A61K 45/06; C07D 215/26; C07D 413/04; C07D 471/04

(52) U.S. Cl. CPC C07D 263/58; A61K 31/422; A61K 31/423; A61K 31/437; A61K 45/06; C07D 215/26; C07D 413/04; C07D 471/04

(57) ABSTRACT

The present disclosure provides pharmaceutical compositions and drug delivery devices comprising a compound of formula I, II, III, or IV. Methods are provided for inhibiting activity of an E3 ligase, involving contacting the E3 ligase with a compound of formula I, II, III, or IV. The present disclosure provides various treatment methods involving administration of such compounds.

### CROSS-REFERENCE

This application claims the benefit of U.S. Provisional Patent Application No. 63/211,868, filed Jun. 17, 2021, and U.S. Provisional Patent Application No. 63/321,034, filed Mar. 17, 2022, which applications are incorporated herein by reference in their entirety.

### INCORPORATION BY REFERENCE OF SEQUENCE LISTING PROVIDED AS A TEXT FILE

A Sequence Listing is provided herewith as a text file, “BERK-447WO_SEQ_LIST_ST25.txt” created on Jun. 7, 2022, and having a size of 12 KB. The contents of the text file are incorporated by reference herein in their entirety.

### INTRODUCTION

E3 ubiquitin ligases (of which over 600 are known in humans) confer substrate specificity for ubiquitination and are more attractive therapeutic targets than general proteasome inhibitors due to their specificity for certain protein substrates. Although the development of ligands of E3 ligases has proven challenging, recent developments have provided specific compounds that can bind a small number of E3 ligases. For most E3 ligases, however, substrate binding sites appear shallow and are thus notoriously difficult to target using small molecules. Conversely, most small molecule drugs bind enzymes or receptors in tight and well-defined pockets. Work in the field has discovered small molecules that interact with E3 ligases covalently, leading to irreversible interactions. These strong interactions can often lead to undesirable effects, particularly when used in the treatment of disease. There is a need in the art for small molecule inhibitors of E3 ligases that are both safe and reversible SUMMARY

The present disclosure provides pharmaceutical compositions and drug delivery devices comprising a compound of formula I, II, III, or IV. Methods are provided for inhibiting activity of an E3 ligase, involving contacting the E3 ligase with a compound of formula I, II, III, or IV. The present disclosure provides various treatment methods involving administration of such compounds.

### BRIEF DESCRIPTION OF THE DRAWINGS

FIG. 1 provides the structures of exemplary compounds and effective concentration for 50% inhibition (EC50) of such compounds in inhibiting Fem1b.

FIG. 2A-2N depict the results of a secondary compound screen.

FIG. 3 provides the structures of exemplary compounds and EC50 values for the compounds.

FIG. 4 provides the structures of exemplary compounds and the EC50 values for inhibition of Fem1b.

### DEFINITIONS

As used herein, the term “linker” or “linkage” refers to a linking moiety that connects two groups and has a backbone of 100 atoms or less in length. A linker or linkage may be a covalent bond that connects two groups or a chain of between 1 and 100 atoms in length, for example of 1, 2, 3, 4, 5, 6, 8, 10, 12, 14, 16, 18 or 20 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol); ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3, or 4 atoms, of the cyclic group are included in the backbone. A linker may be cleavable or non-cleavable.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, humans, non-human primates, mammalian farm animals, mammalian sport animals, and mammalian pets.

The term “binding” refers to a direct association between two molecules, due to, for example, covalent, electrostatic, hydrophobic, and ionic, and/or hydrogen-bond interactions, including interactions such as salt bridges and water bridges.

The term “inhibitor” refers to a compound having the ability to inhibit a biological function of a target protein or a target biological process. When inhibiting a target protein, an inhibitor can inhibit the activity of the target protein.

The terms “simultaneous” or “simultaneously” as applied to administering compounds to a subject refer to administering one or more compounds at the same time, or at two different time points that are separated by no more than 1 hour.

The term “sequentially” refers to administering more than one compounds at two different time points that are separated by more than 1 hour, e.g., about 2 hours, about 5 hours, 8 hours, 1 day, 2 days, 3 days, 4 days, 5 days, 6 days, 7 days, or even longer.

Certain chemical groups named herein may be preceded by a shorthand notation indicating the total number of carbon atoms that are to be found in the indicated chemical group. For example; C7-C12 alkyl describes an alkyl group, as defined below, having a total of 7 to 12 carbon atoms, and C4-C12 cycloalkylalkyl describes a cycloalkylalkyl group, as defined below, having a total of 4 to 12 carbon atoms. The total number of carbons in the shorthand notation does not include carbons that may exist in substituents of the group described.

In addition to the foregoing, unless specified to the contrary, the following terms have the meaning indicated: “Amino” refers to the —NH₂ radical. “Cyano” refers to the —CN radical. “Hydroxy” refers to the —OH radical. “Imino” refers to the ═NH substituent. “Nitro” refers to the —NO₂ radical. “Oxo” refers to the ═O substituent. “Thioxo” refers to the ═S substituent. “Trifluoromethyl” refers to the —CF₃ radical.

“Alkyl” refers to a straight or branched hydrocarbon chain radical consisting solely of carbon and hydrogen atoms, containing no unsaturation, having from one to twelve carbon atoms, e.g., one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group may be optionally substituted by one of the following groups: alkyl, thiol, acyl alkenyl, halo, haloalkenyl, cyano, nitro, aryl, cycloalkyl, heterocyclyl, heteroaryl, oxo, trimethylsilanyl, —OR14, —OC(O)—R14, —N(R14)2, —C(O)R14, —C(O)OR14, —C(O)N(R14)2, —N(R14)C(O)OR16, —N(R14)C(O)R16, —N(R14)S(O)tR16 (where t is 1 to 2), —S(O)tOR16 (where t is 1 to 2), —S(O)pR16 (where p is 0 to 2), and —S(O)tN(R14)2 (where t is 1 to 2) where each R14 is independently hydrogen, alkyl, haloalkyl, cycloalkyl, cycloalkylalkyl, aryl, aralkyl, heterocyclyl, heterocyclylalkyl, heteroaryl or heteroarylalkyl; and each R16 is alkyl, haloalkyl, cycloalkyl, cycloalkylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain may be optionally substituted by one of the following groups: alkyl, alkenyl, halo, haloalkenyl, cyano, nitro, aryl, cycloalkyl, heterocyclyl, heteroaryl, oxo, trimethylsilanyl, —OR14, —OC(O)—R14, —N(R14)2, —C(O)R14, —C(O)OR14, —C(O)N(R14)2, —N(R14)C(O)OR16, —N(R14)C(O)R16, —N(R14)S(O)tR16 (where t is 1 to 2), —S(O)tOR16 (where t is 1 to 2), —S(O)pR16 (where p is 0 to 2), and —S(O)tN(R14)2 (where t is 1 to 2) where each R14 is independently hydrogen, alkyl, haloalkyl, cycloalkyl, cycloalkylalkyl, aryl, aralkyl, heterocyclyl, heterocyclylalkyl, heteroaryl or heteroarylalkyl; and each R16 is alkyl, haloalkyl, cycloalkyl, cycloalkylalkyl, aryl, aralkyl, heterocyclyl, heterocyclylalkyl, heteroaryl or heteroarylalkyl.

“Aryl” refers to a hydrocarbon ring system radical comprising hydrogen, 6 to 18 carbon atoms and at least one aromatic ring. For purposes of this disclosure, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals optionally substituted by one or more substituents independently selected from the group consisting of alkyl, thiol, acyl akenyl, halo, haloalkyl, haloalkenyl, cyano, nitro, aryl, aralkyl, heteroaryl, heteroarylalkyl, —R15-OR14, —R15-OC(O)—R14, —R15-N(R14)2, —R15-C(O)R14, —R15-C(O)OR14, —R15-C(O)N(R14)2, —R15-N(R14)C(O)OR16, —R15-N(R14)C(O)R16, —R15-N(R4)S(O)tR16 (where t is 1 to 2), —R15-N═C(OR14)R14, —R15-S(O)tOR16 (where t is 1 to 2), —R15-S(O)pR16 (where p is 0 to 2), and —R15-S(O)tN(R14)2 (where t is 1 to 2) where each R14 is independently hydrogen, alkyl, haloalkyl, cycloalkyl, cycloalkylalkyl, aryl, aralkyl, heterocyclyl, heterocyclylalkyl, heteroaryl or heteroarylalkyl; each R15 is independently a direct bond or a straight or branched alkylene or alkenylene chain; and each R16 is alkyl, haloalkyl, cycloalkyl, cycloalkylalkyl, aryl, aralkyl, heterocyclyl, heterocyclylalkyl, heteroaryl or heteroarylalkyl.

“Aralkyl” refers to a radical of the formula —Rb-Rc where Rb is an alkylene chain as defined above and Rc is one or more aryl radicals as defined above, for example, benzyl, diphenylmethyl and the like. The alkylene chain part of the aralkyl radical may be optionally substituted as described above for an alkylene chain. The aryl part of the aralkyl radical may be optionally substituted as described above for an aryl group.

“Cycloalkyl” refers to a stable non-aromatic monocyclic or polycyclic hydrocarbon radical consisting solely of carbon and hydrogen atoms, which may include fused or bridged ring systems, having from three to fifteen carbon atoms, or having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptly, and cyclooctyl. Polycyclic radicals include, for example, adamantyl, norbornyl, decalinyl, and the like. Unless otherwise stated specifically in the specification, the term “cycloalkyl” is meant to include cycloalkyl radicals which are optionally substituted by one or more substituents independently selected from the group consisting of alkyl, acyl, thiol alkenyl, halo, haloalkyl, haloalkenyl, cyano, nitro, oxo, aryl, aralkyl, cycloalkyl, cycloalkylalkyl, heterocyclyl, heterocyclylalkyl, heteroaryl, heteroarylalkyl, —R15-OR14, —R15-OC(O)—R14, —R15-N(R14)2, —R15-C(O)R14, —R15-C(O)OR14, —R15-C(O)N(R14)2, —R15-N(R14)C(O)OR16, —R15-N(R14)C(O)R16, —R15-N(R14)S(O)tR16 (where t is 1 to 2), —R15-N═C(OR14)R14, —R15-S(O)tOR16 (where t is 1 to 2), —R15-S(O)pR16 (where p is 0 to 2), and —R15-S(O)tN(R14)2 (where t is 1 to 2) where each R14 is independently hydrogen, alkyl, haloalkyl, cycloalkyl, cycloalkylalkyl, aryl, aralkyl, heterocyclyl, heterocyclylalkyl, heteroaryl or heteroarylalkyl; each R15 is independently a direct bond or a straight or branched alkylene or alkenylene chain; and each R16 is alkyl, haloalkyl, cycloalkyl, cycloalkylalkyl, aryl, aralkyl, heterocyclyl, heterocyclylalkyl, heteroaryl or heteroarylalkyl.

“Halo” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may be optionally substituted as defined above for an alkyl group.

“Heterocyclyl” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxo-1,3-dioxol-4yl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above which are optionally substituted by one or more substituents selected from the group consisting of alkyl, acyl, thiol, alkenyl, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R15-OR14, —R15-OC(O)—R14, —R15-N(R14)2, —R15-C(O)OR14, —R5-C(O)OR14, —R5-C(O)N(R4)2, —R15-N(R4)C(O)OR16, —R15-N(R14)C(O)R16, —R15-N(R14)S(O)tR16 (where t is 1 to 2), —R15-N═C(OR14)R14, —R15-(S(O)tOR16 (where t is 1 to 2), —R15-S(O)pR16 (where p is 0 to 2), and —R15-S(O)tN(R14)2 (where t is 1 to 2) where each R14 is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R15 is independently a direct bond or a straight or branched alkylene or alkenylene chain; and each R16 is alkyl, alkenyl, haloalkyl, cycloalkyl, cycloalkylalkyl, aryl, aralkyl, heterocyclyl, heterocyclylalkyl, heteroaryl or heteroarylalkyl.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical may be optionally substituted as described above for heterocyclyl radicals.

“Heterocyclylalkyl” refers to a radical of the formula —RbRh where Rb is an alkylene chain as defined above and Rh is a heterocyclyl radical as defined above, and if the heterocyclyl is a nitrogen-containing heterocyclyl, the heterocyclyl may be attached to the alkylene chain at the nitrogen atom. The alkylene chain of the heterocyclylalkyl radical may be optionally substituted as defined above for an alkylene chain. The heterocyclyl part of the heterocyclylalkyl radical may be optionally substituted as defined above for a heterocyclyl group.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrazole, pyridine, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, thiophene, benxothiophene triazolyl, tetrazolyl, triazinyl, and thiophenyl (i.e. thienyl). Unless stated otherwise specifically in the specification, the term “heteroaryl” is meant to include heteroaryl radicals as defined above which are optionally substituted by one or more substituents selected from the group consisting of alkyl, acyl, thiol, alkenyl, alkoxy, ethoxy, halo, haloalkyl, haloalkenyl, cyano, oxo, thioxo, nitro, aryl, aralkyl, cycloalkyl, cycloalkylalkyl, heterocyclyl, heterocyclylalkyl, heteroaryl, heteroarylalkyl, —R15-OR14, —R15-OC(O)—R14, —R15-N(R14)2, —R15-C(O)R14, —R15-C(O)OR14, —R15-C(O)N(R14)2, —R15-N(R14)C(O)OR16, —R15-N(R14)C(O)R16, —R15-N(R14)S(O)tR16 (where t is 1 to 2), —R15-N═C(OR14)R14,-R15-S(O)tOR16 (where t is 1 to 2),-R15-S(O)pR16 (where p is 0 to 2), and —R15-S(O)tN(R14)2 (where t is 1 to 2) where each R14 is independently hydrogen, alkyl, alkenyl, haloalkyl, cycloalkyl, cycloalkylalkyl, aryl, aralkyl, heterocyclyl, heterocyclylalkyl, heteroaryl or heteroarylalkyl; each R15 is independently a direct bond or a straight or branched alkylene or alkenylene chain; and each R16 is alkyl, alkenyl, haloalkyl, cycloalkyl, cycloalkylalkyl, aryl, aralkyl, heterocyclyl, heterocyclylalkyl, heteroaryl or heteroarylalkyl.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical may be optionally substituted as described above for heteroaryl radicals.

“Heteroarylalkyl” refers to a radical of the formula —RbRi where Rb is an alkylene chain as defined above and Ri is a heteroaryl radical as defined above. The heteroaryl part of the heteroarylalkyl radical may be optionally substituted as defined above for a heteroaryl group. The alkylene chain part of the heteroarylalkyl radical may be optionally substituted as defined above for an alkylene chain.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. When a functional group is described as “optionally substituted,” and in turn, substituents on the functional group are also “optionally substituted” and so on, for the purposes of this disclosure, such iterations are limited to five, and in some cases, such iterations are limited to two.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Examples of suitable inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Examples of suitable organic bases are isopropylamine, diethylamine, ethanolamine, trimethylamine, dicyclohexylamine, choline, and caffeine.

A “pharmaceutical composition” refers to a formulation of a compound and a medium generally accepted in the art for the delivery of the biologically active compound to a mammal, e.g., humans. Such a medium can include a pharmaceutically acceptable carrier, diluent, or excipient.

“Therapeutically effective amount” refers to that amount of a compound which, when administered to a mammal, e.g., a human, is sufficient to effect treatment of the disease or condition of interest in a mammal, e.g., a human, having the disease or condition. The amount of a compound which constitutes a “therapeutically effective amount” will vary depending on the compound, the disease or condition and its severity, the manner of administration, and the age of the mammal to be treated, but can be determined routinely by one of ordinary skill in the art having regard to his own knowledge and to this disclosure.

A compound as disclosed herein, or a pharmaceutically acceptable salt thereof, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. A compound as disclosed herein is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposeable mirror images of one another.

The use of parentheses in substituent groups is used herein to conserve space. Accordingly, the use of parenthesis in a substituent group indicates that the group enclosed within the parentheses is attached directly to the atom preceding the parenthesis.

The term “drug delivery device” shall be understood to encompass any type of device, system or apparatus designed to immediately dispense a drug to a human or non-human body. By “immediately dispense” is meant an absence of any necessary intermediate manipulation of the drug by a user between discharge of the drug from the drug delivery device and administration to the human or non-human body. Without limitation, typical examples of drug delivery devices may be found in injection devices, inhalers, and stomach tube feeding systems. Again without limitation, exemplary injection devices may include, e.g., syringes, autoinjectors, injection pen devices and spinal injection systems.

A “ubiquitin ligase binding moiety” refers to a portion of a bifunctional compound that binds to a ubiquitin ligase (e.g., an E3 ligase).

A “protein targeting moiety” refers to a portion of a bifunctional compound that binds to a protein of interest. The protein targeting moiety, when in the presence of a ubiquitin ligase binding moiety, brings a protein of interest in close proximity of a ubiquitin ligase thereby leading to the ubiquitination and degradation of said protein of interest.

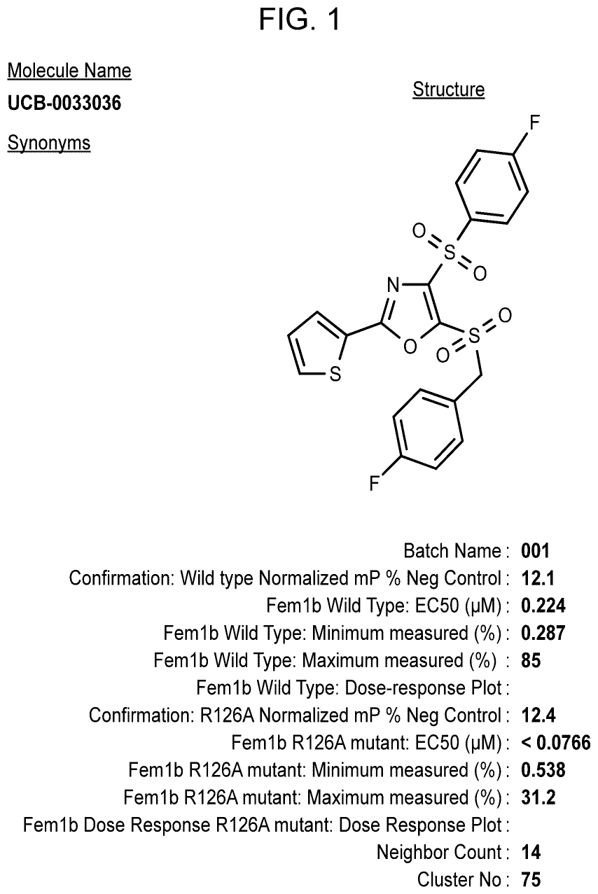

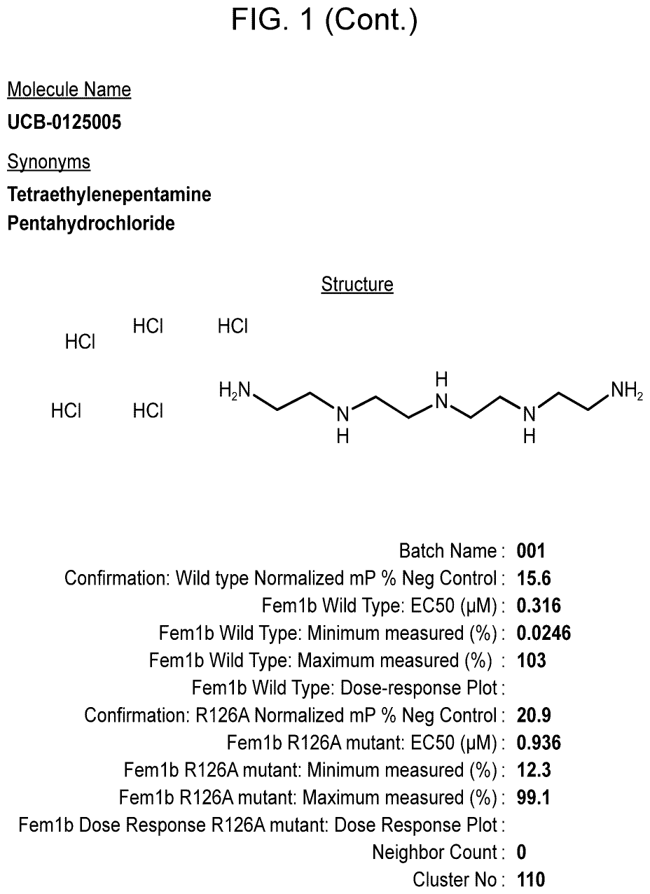

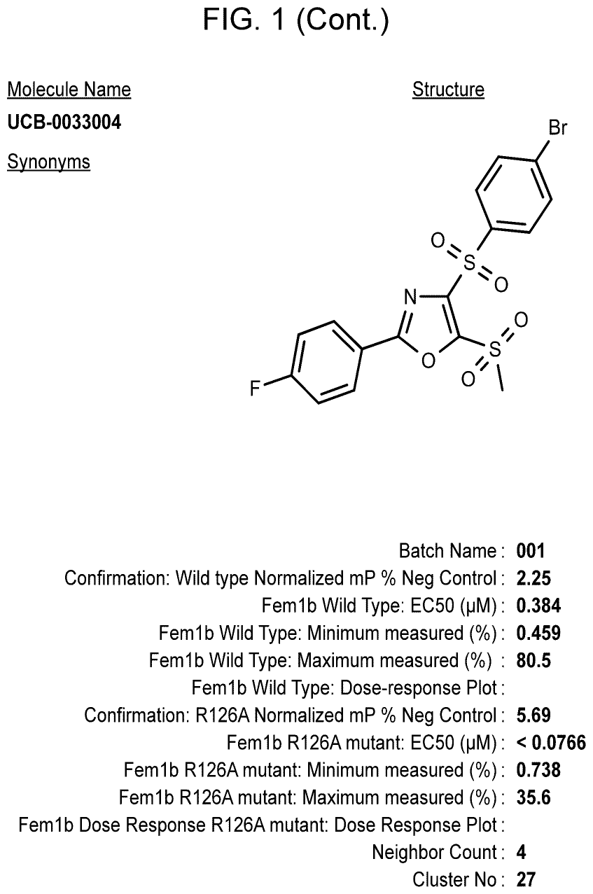

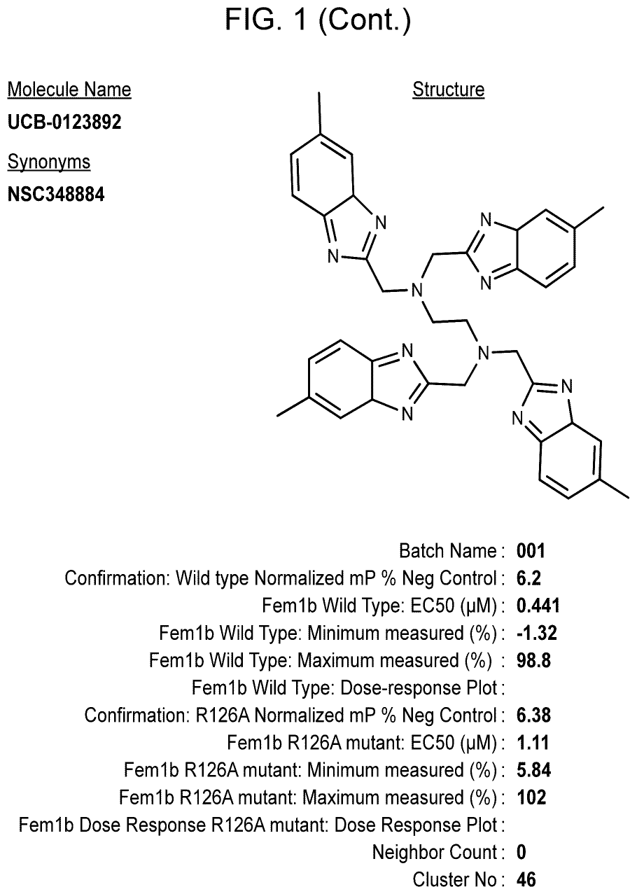

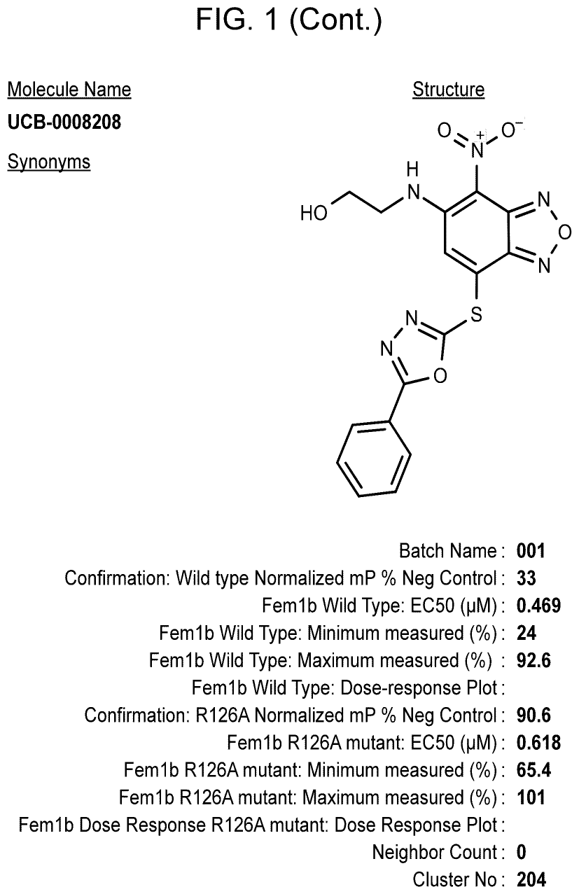

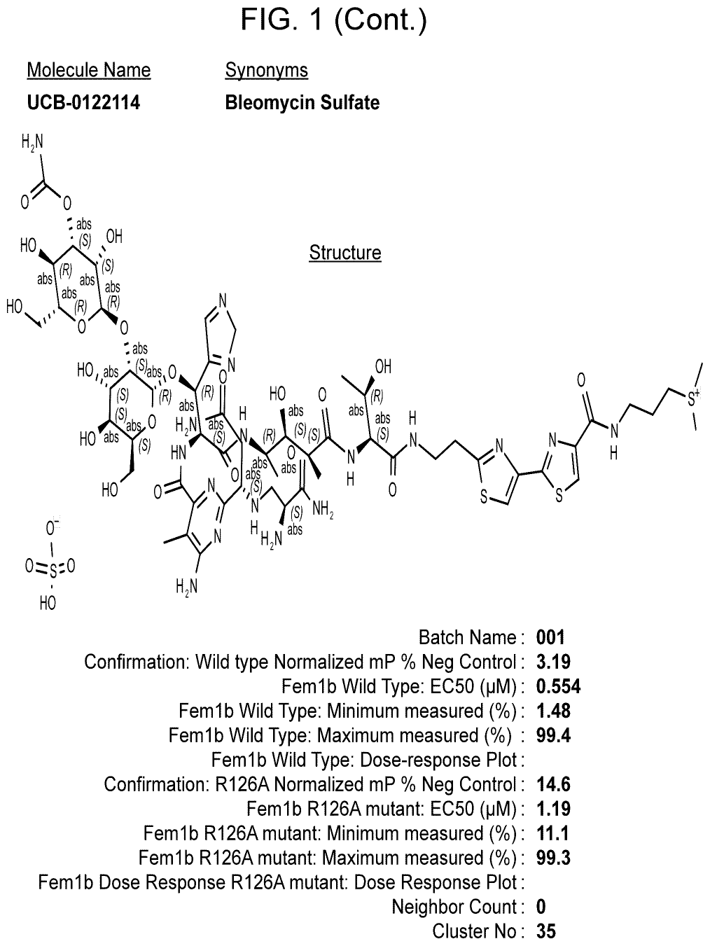

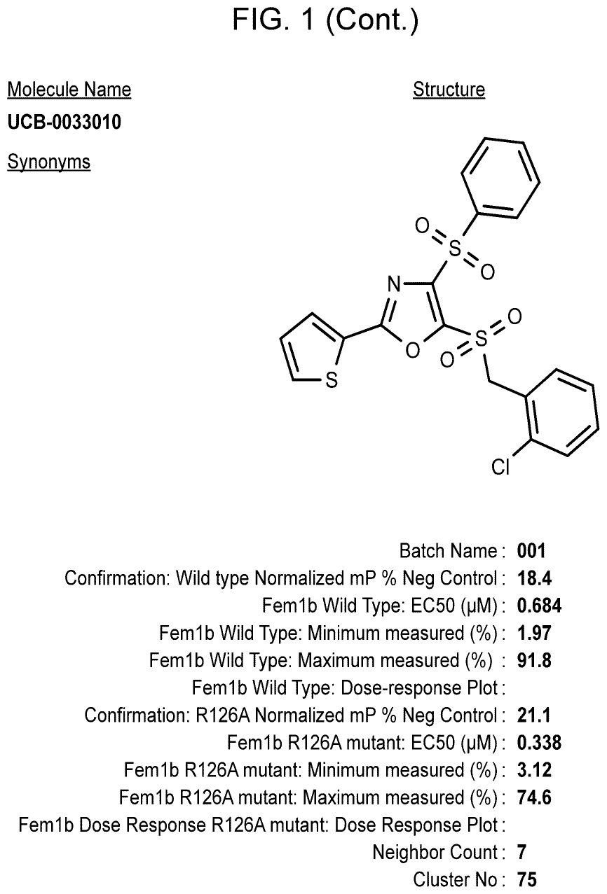

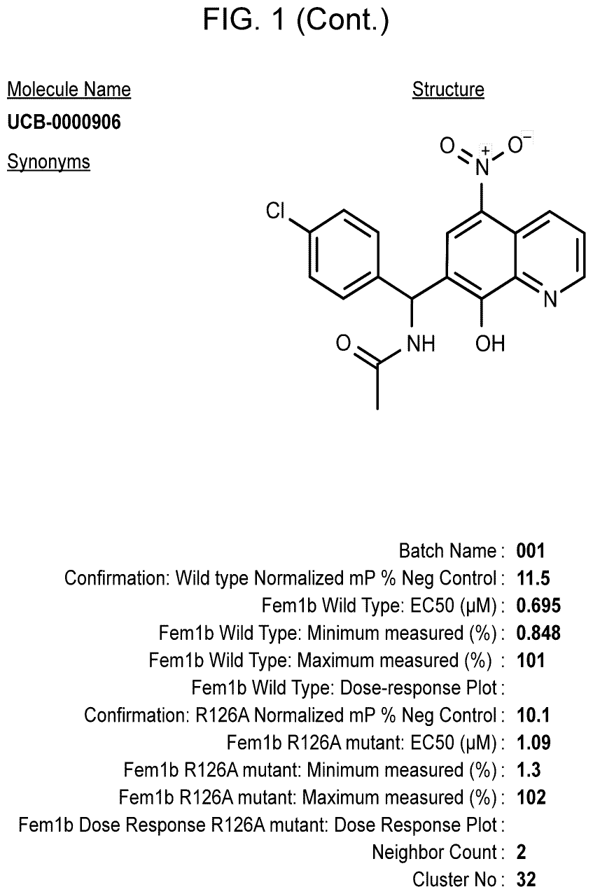

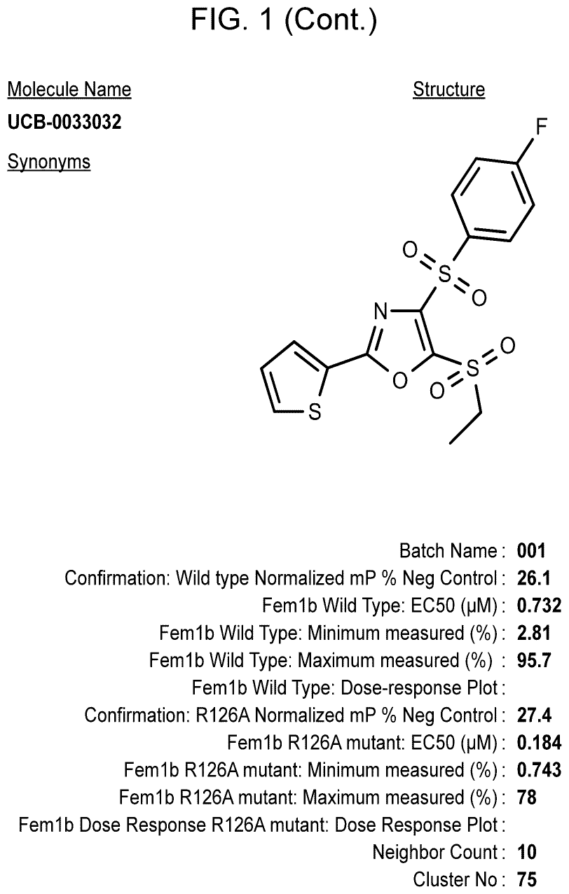

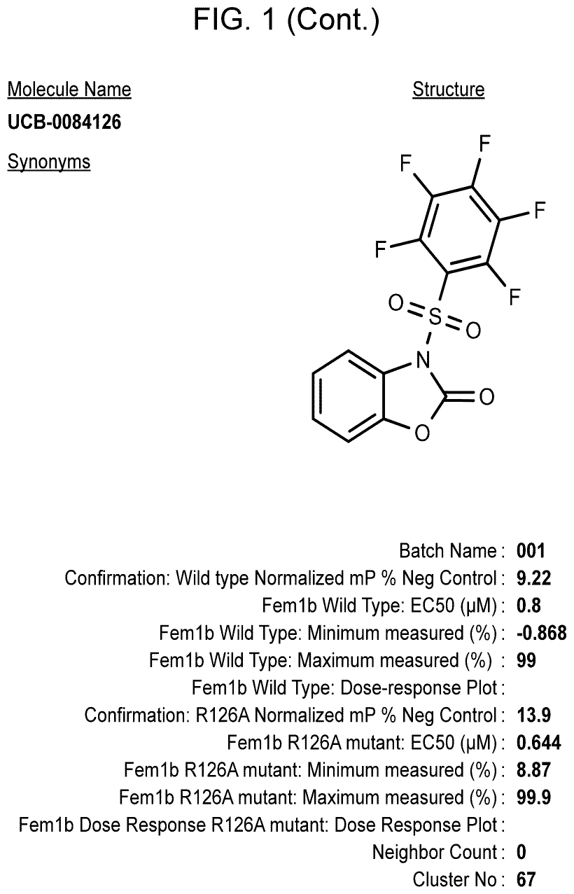

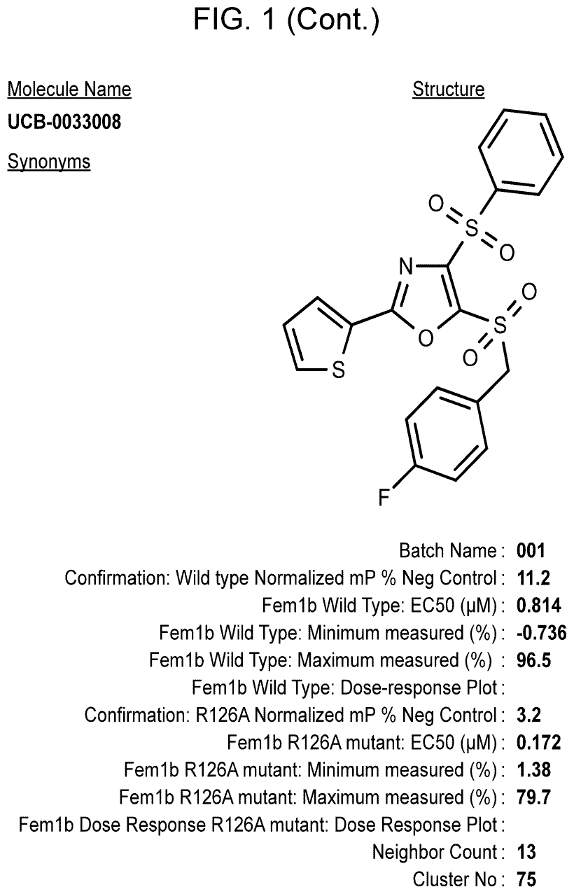

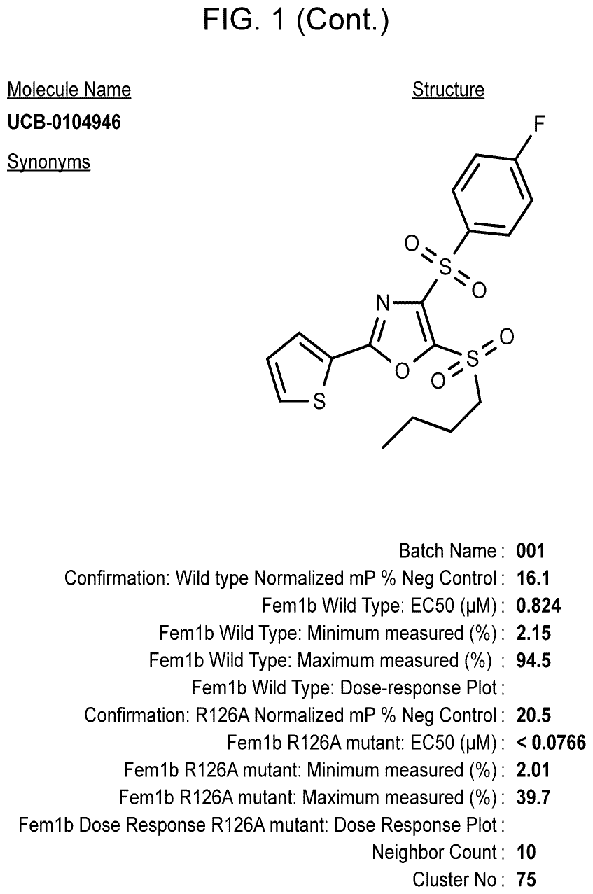

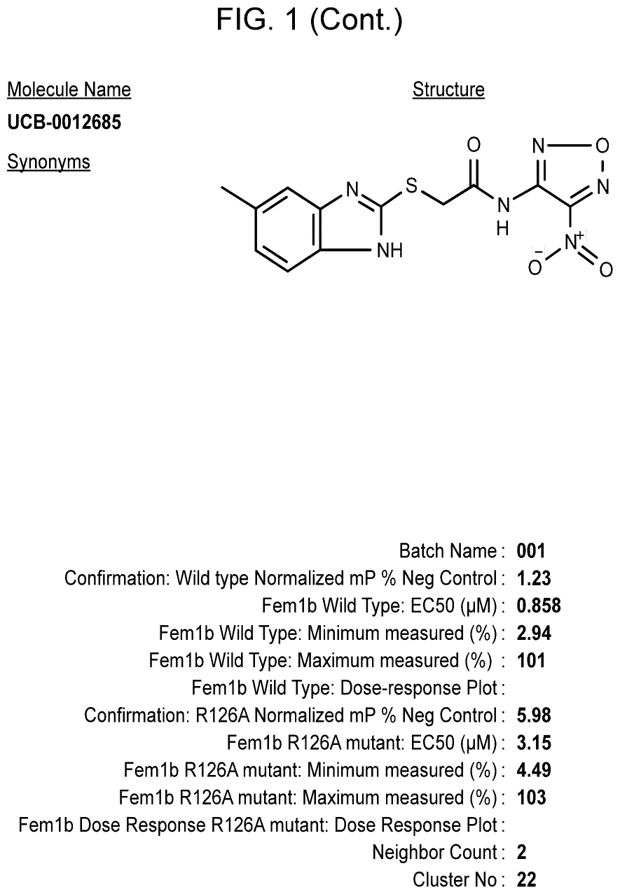

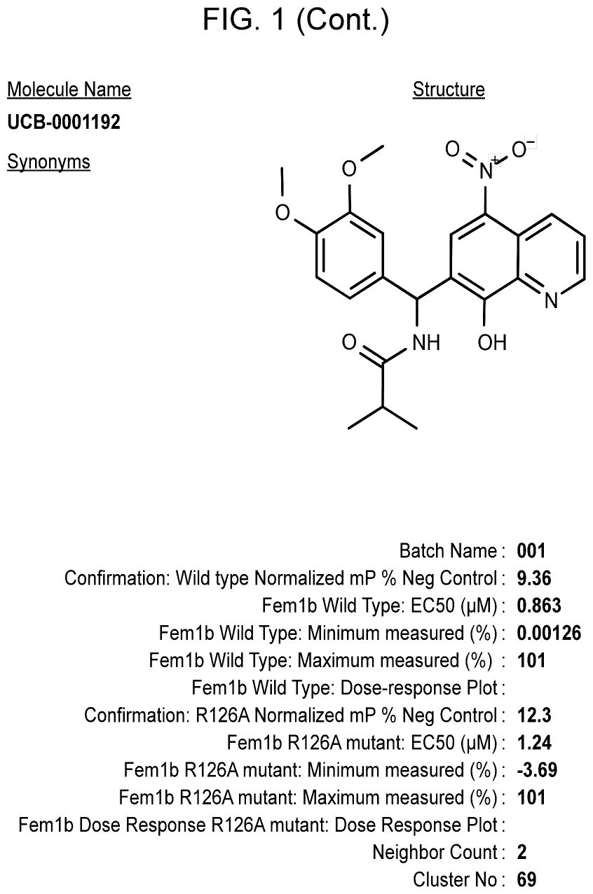

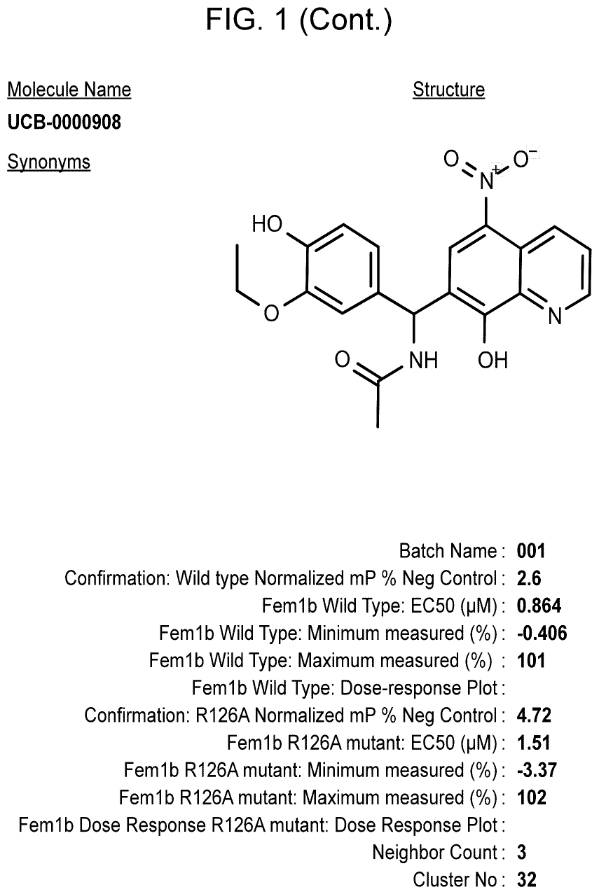

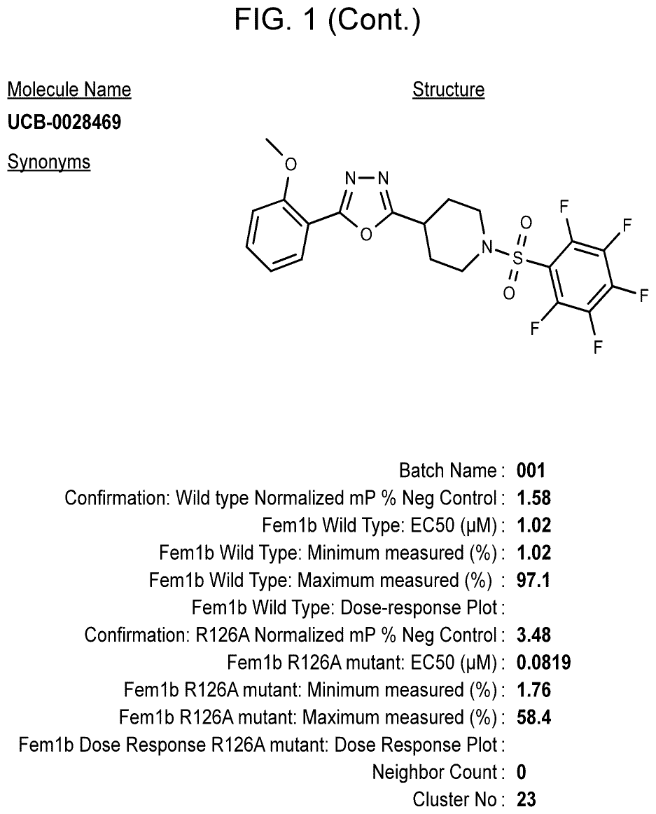

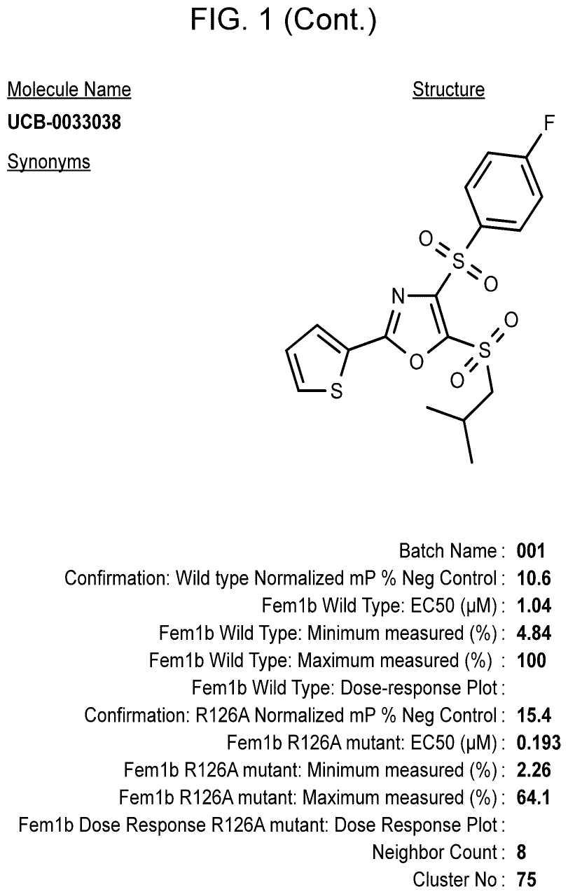

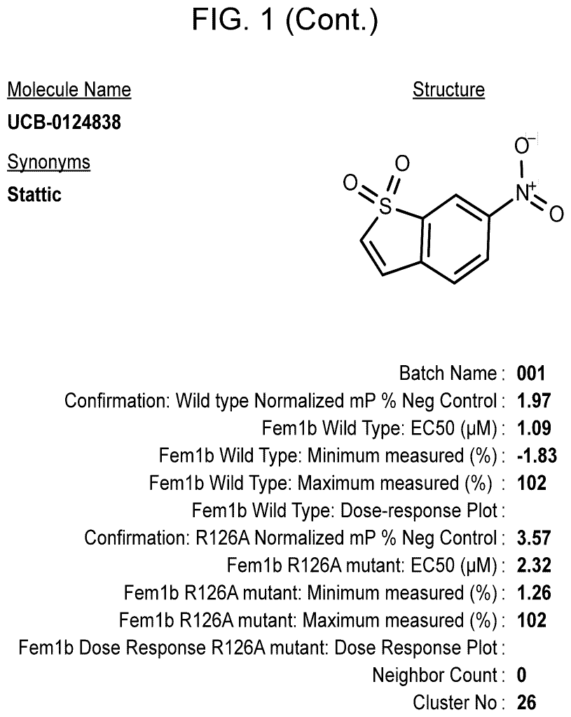

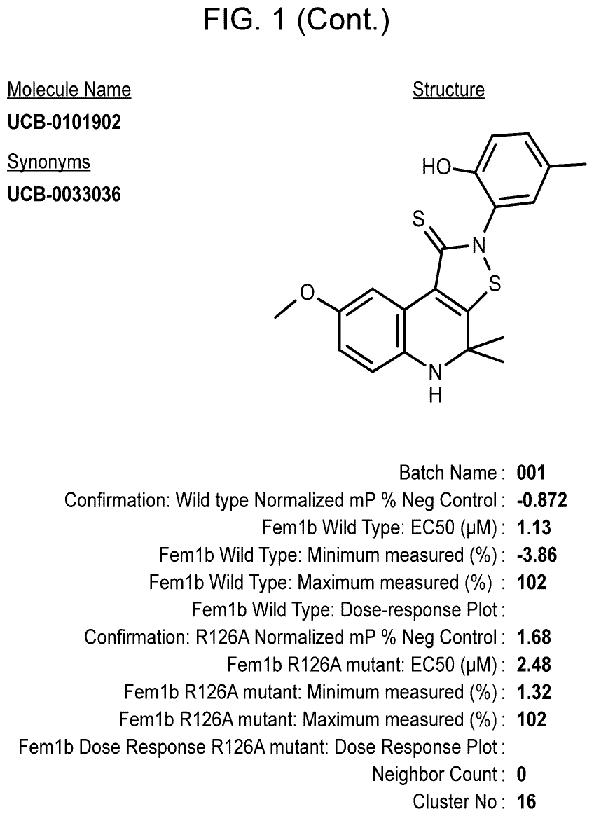

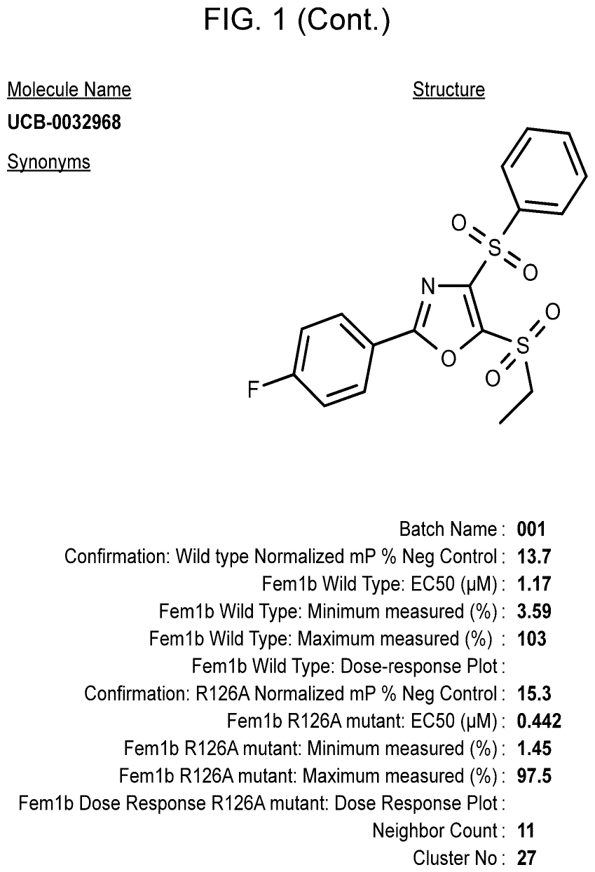

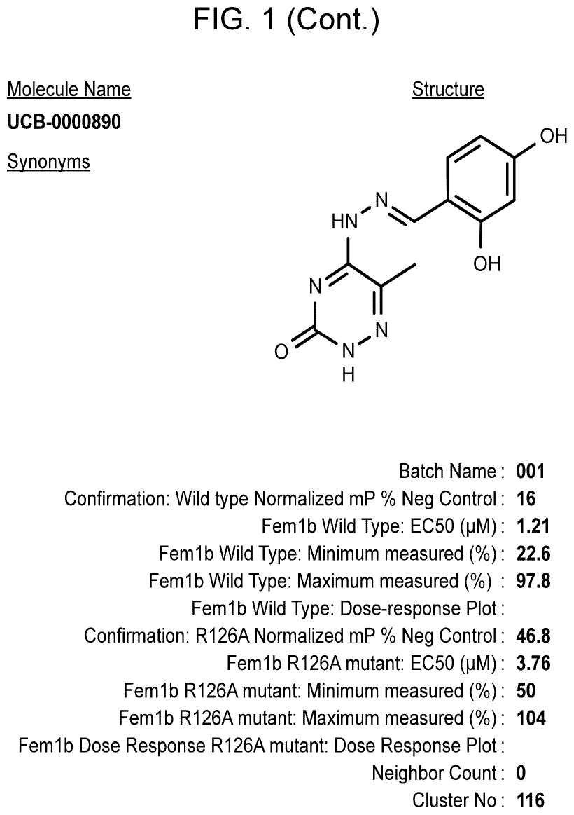

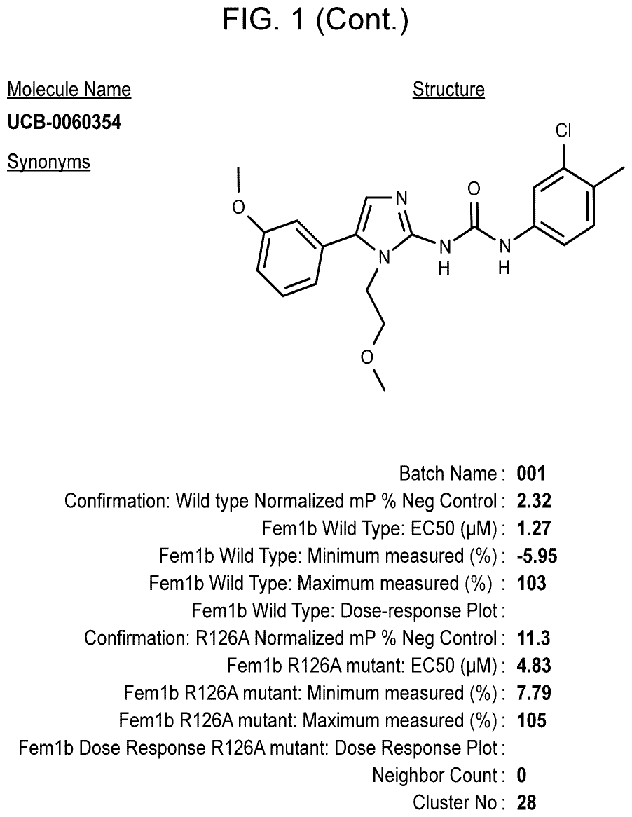

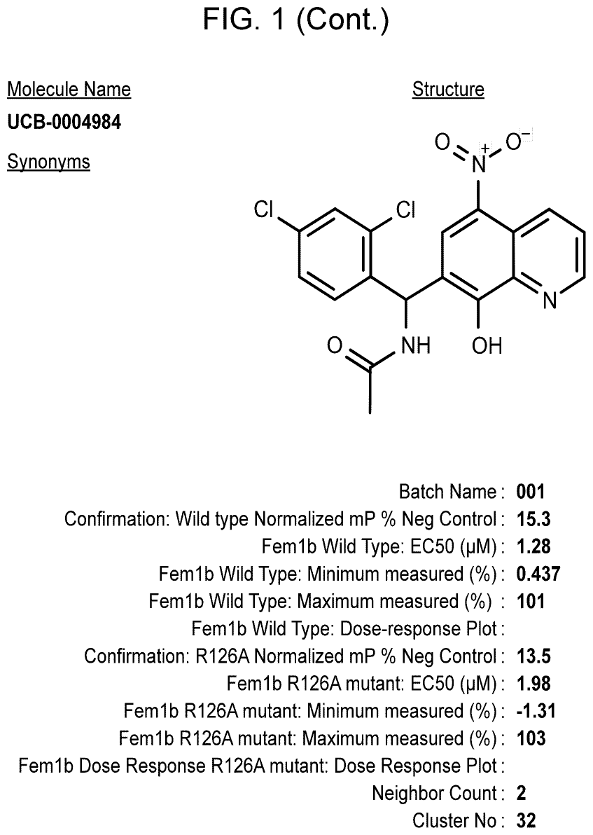

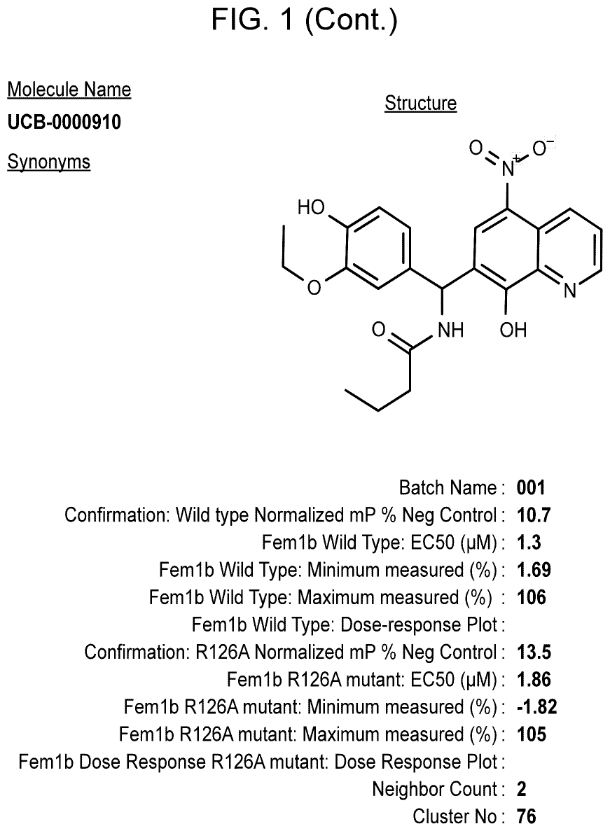

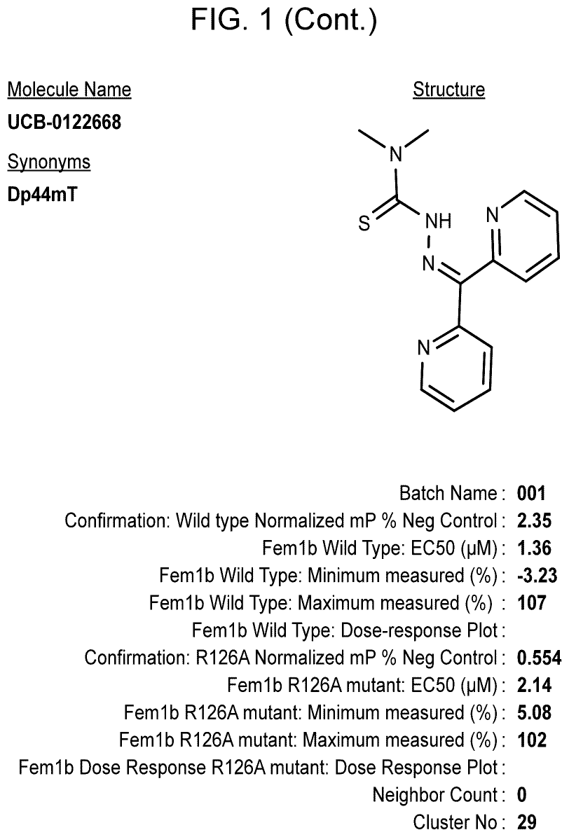

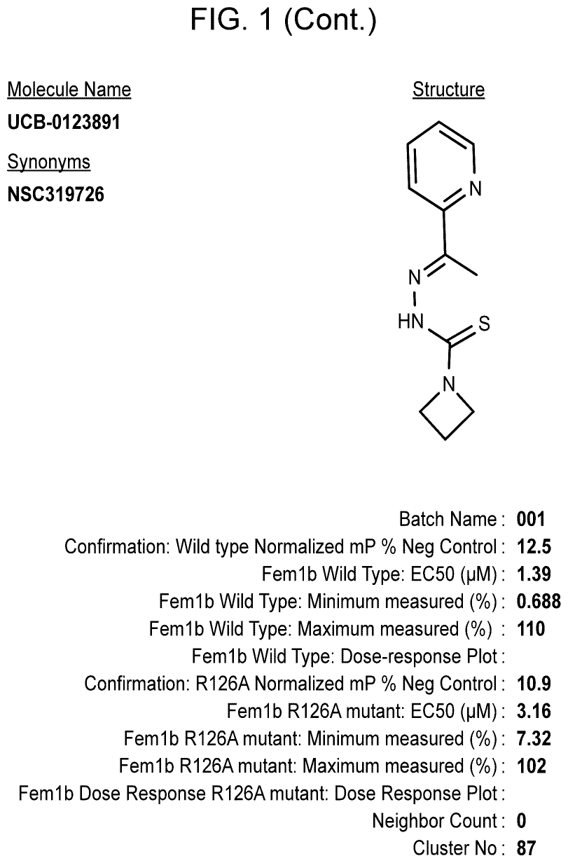

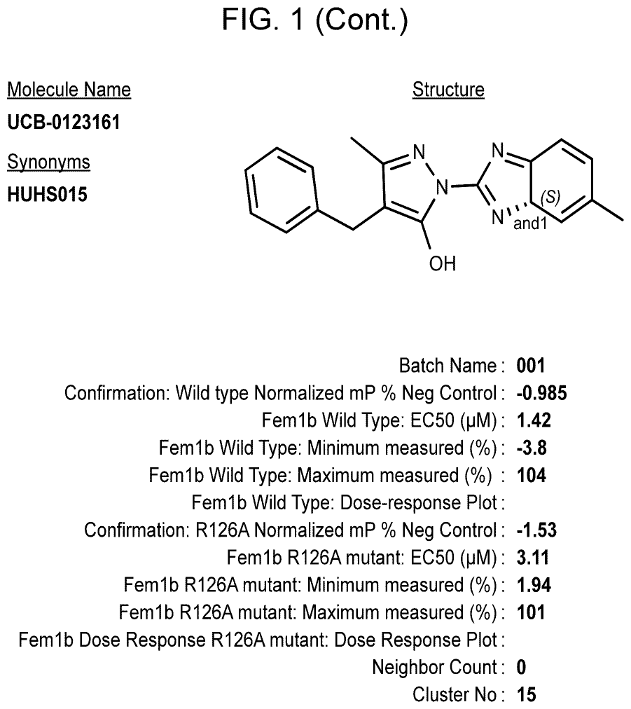

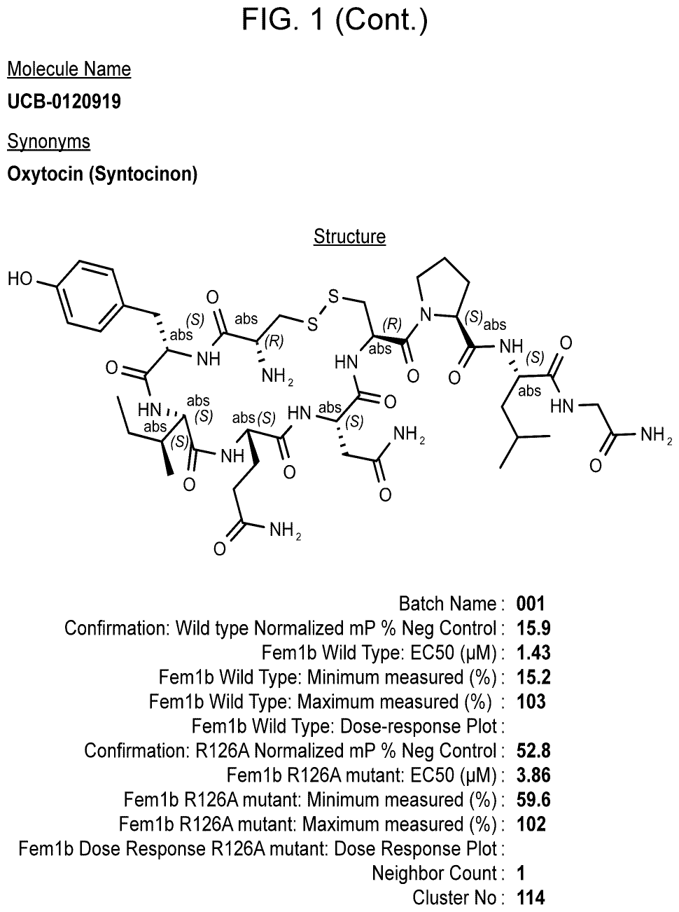

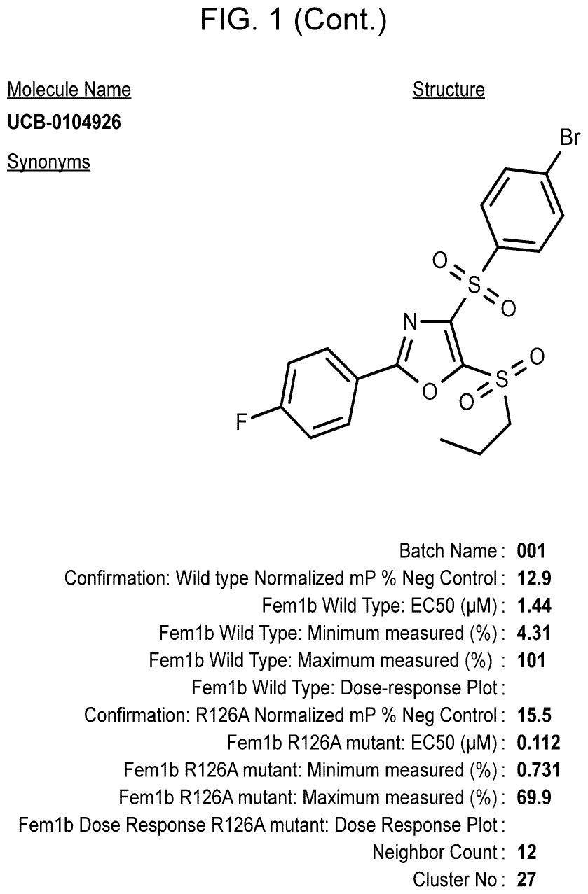

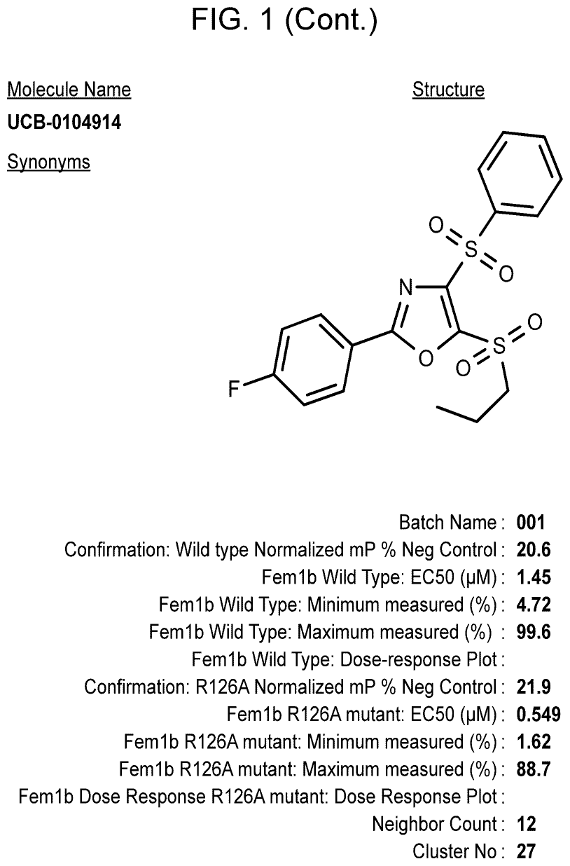

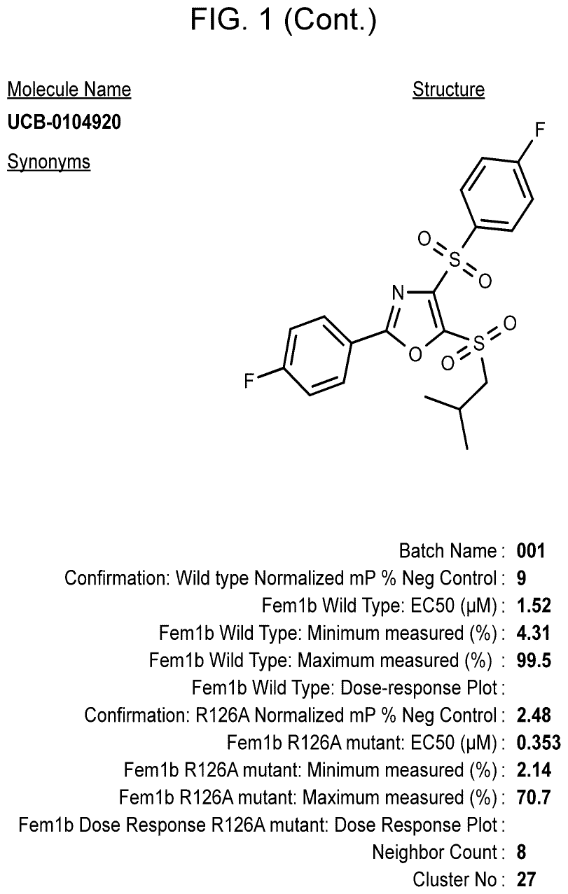

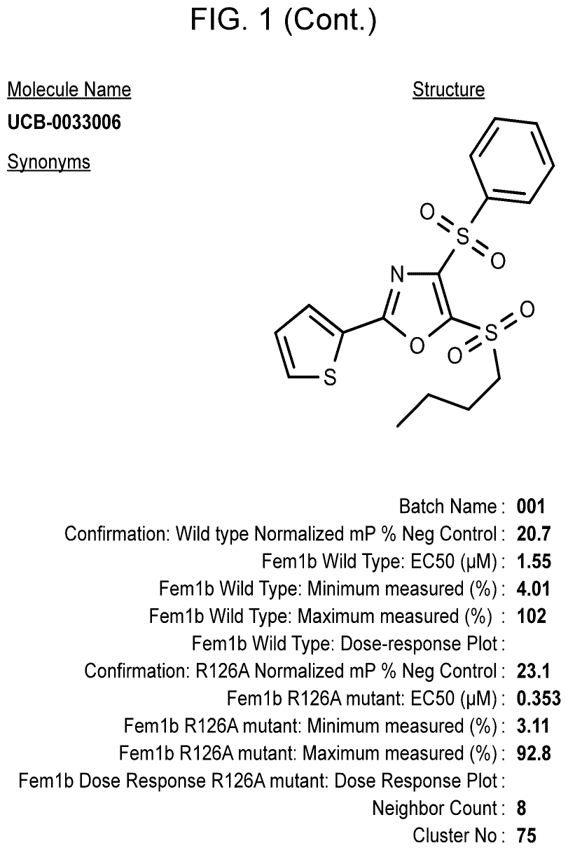

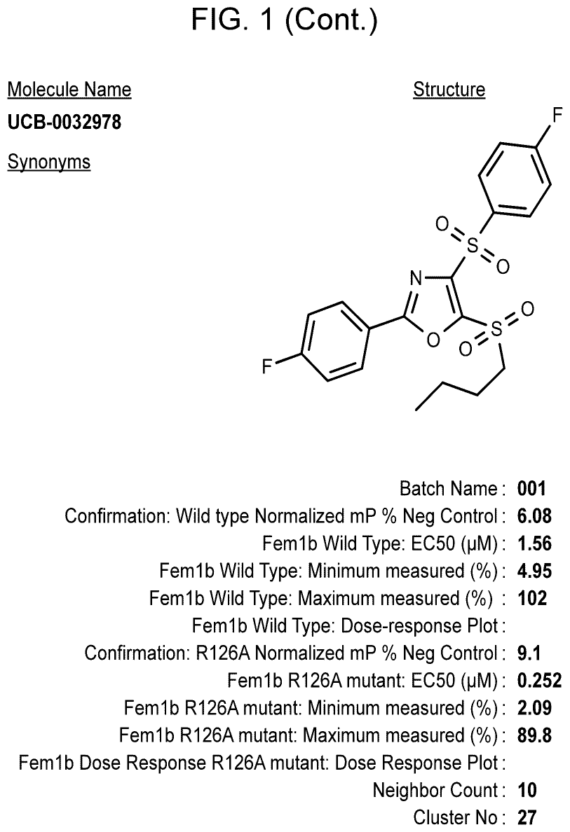

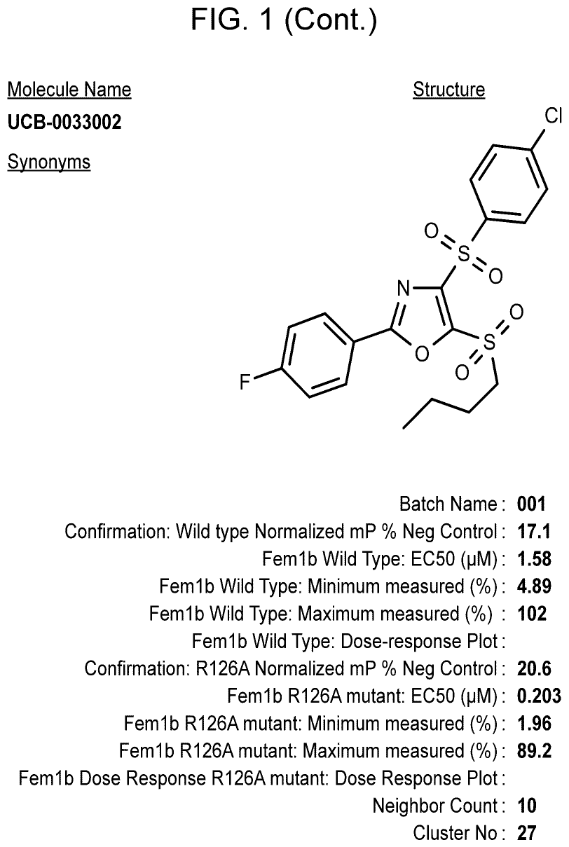

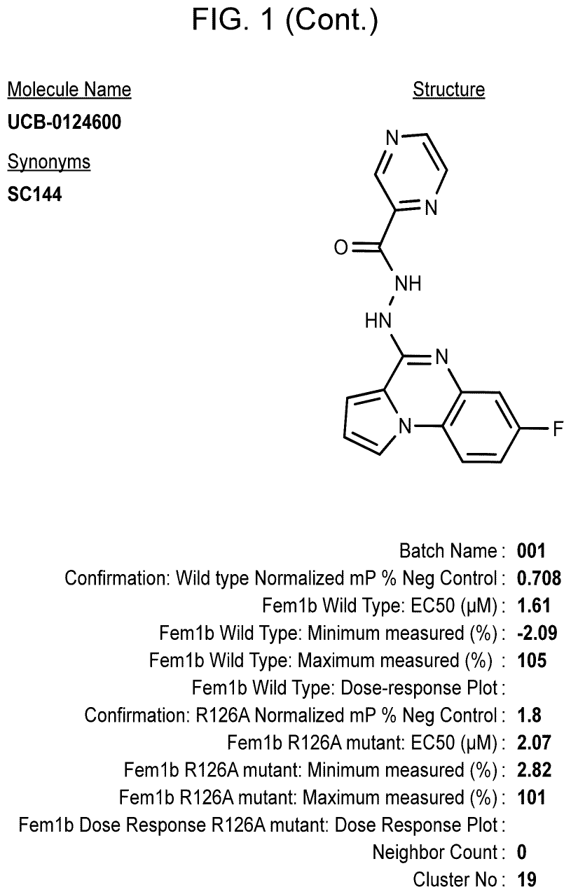

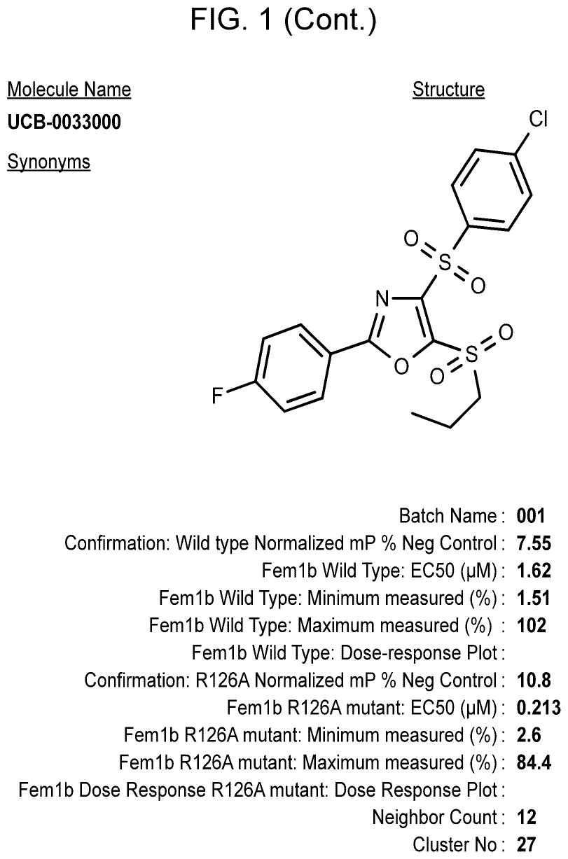

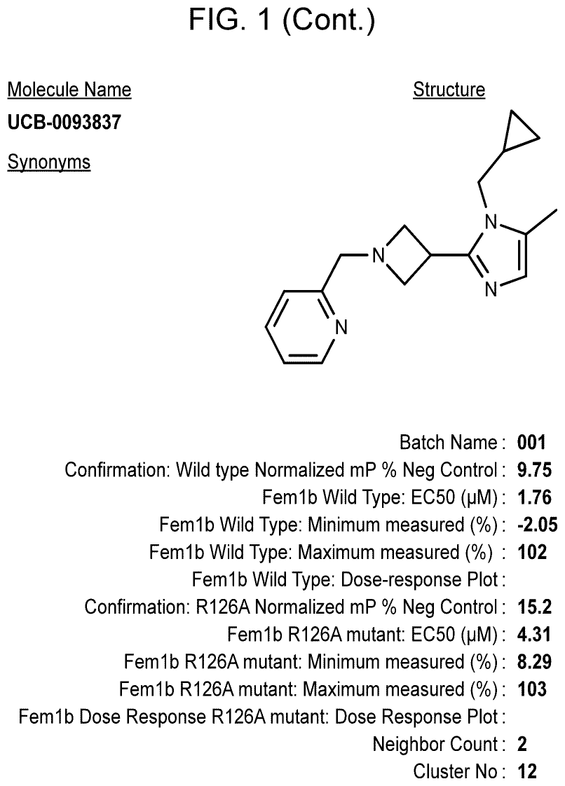

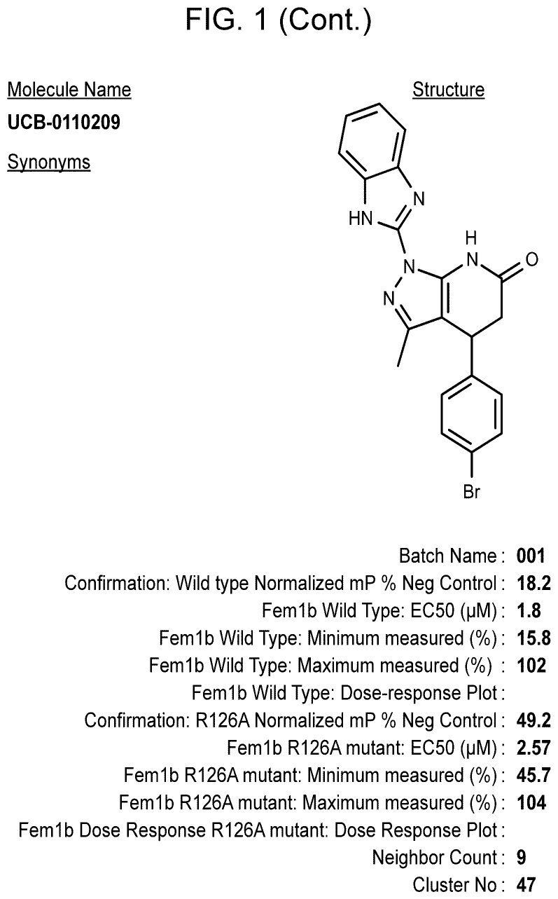

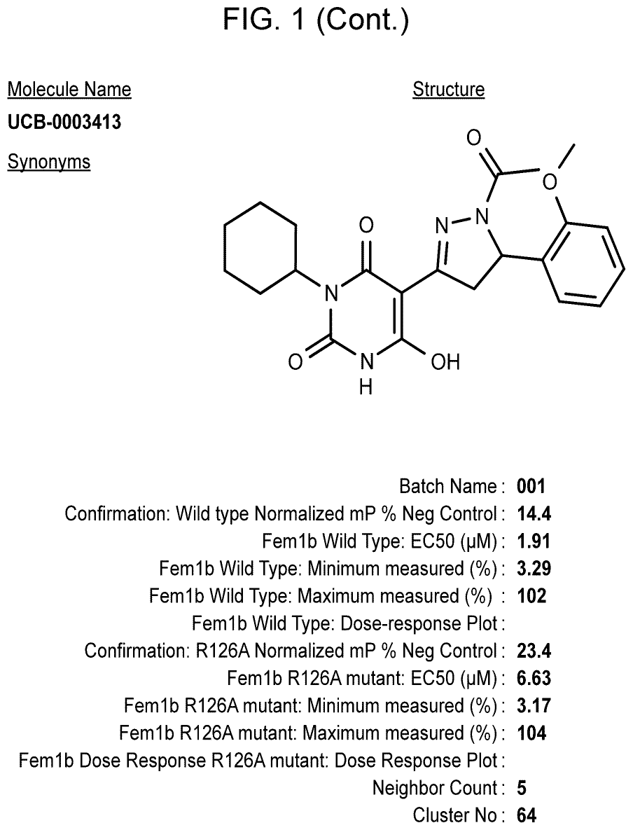

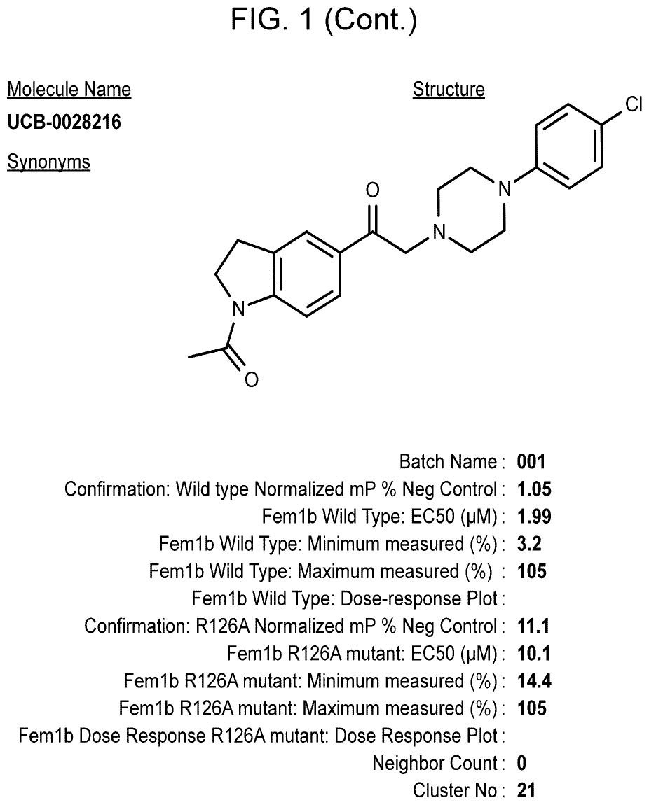

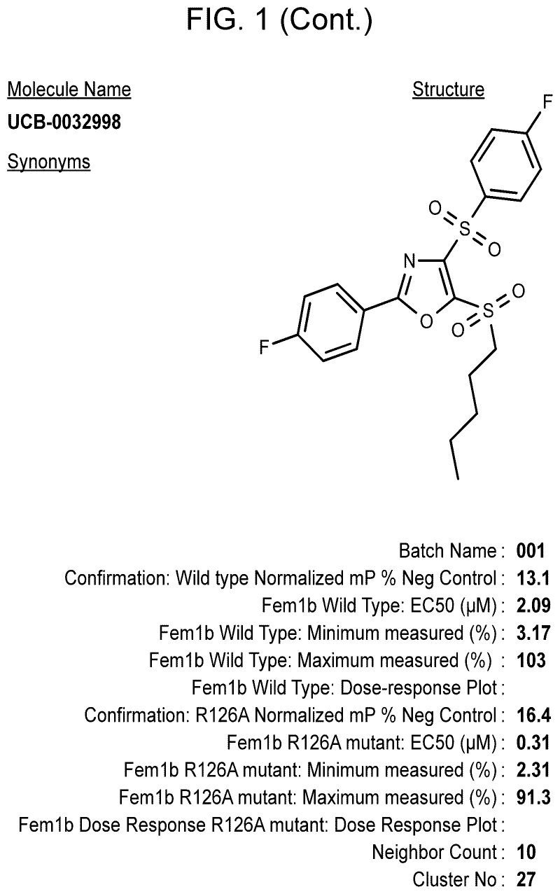

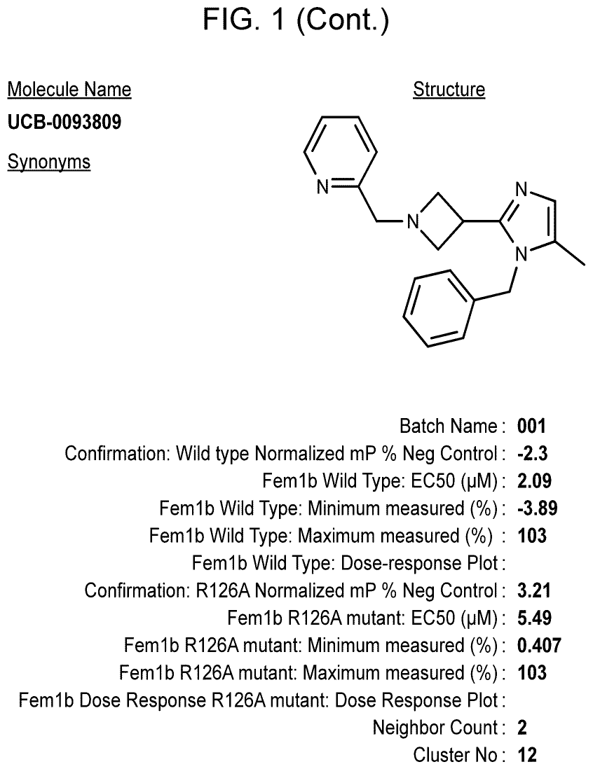

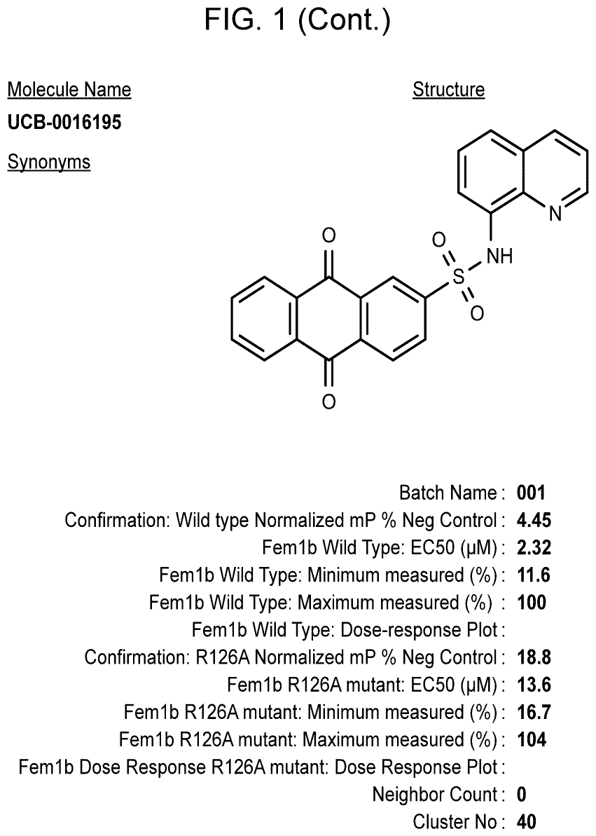

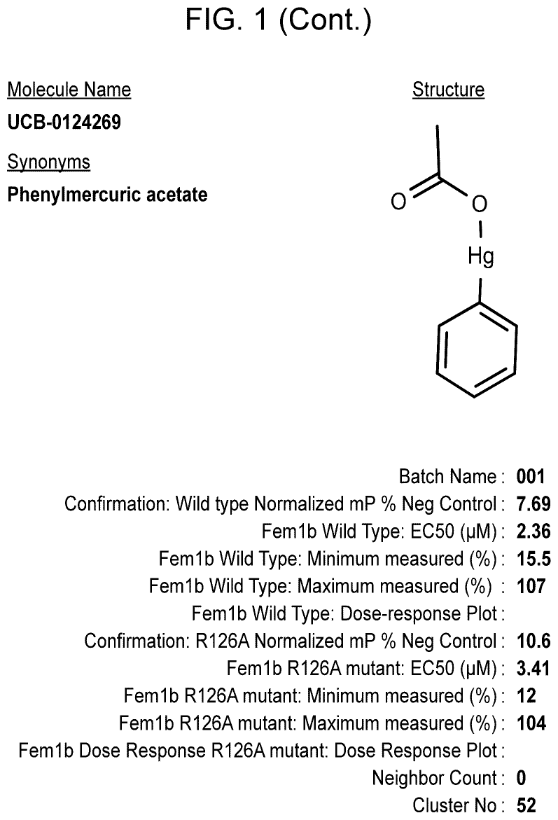

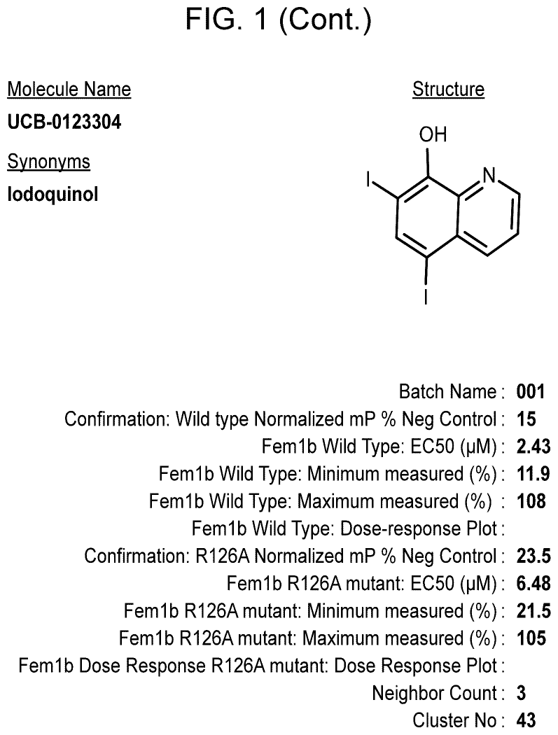

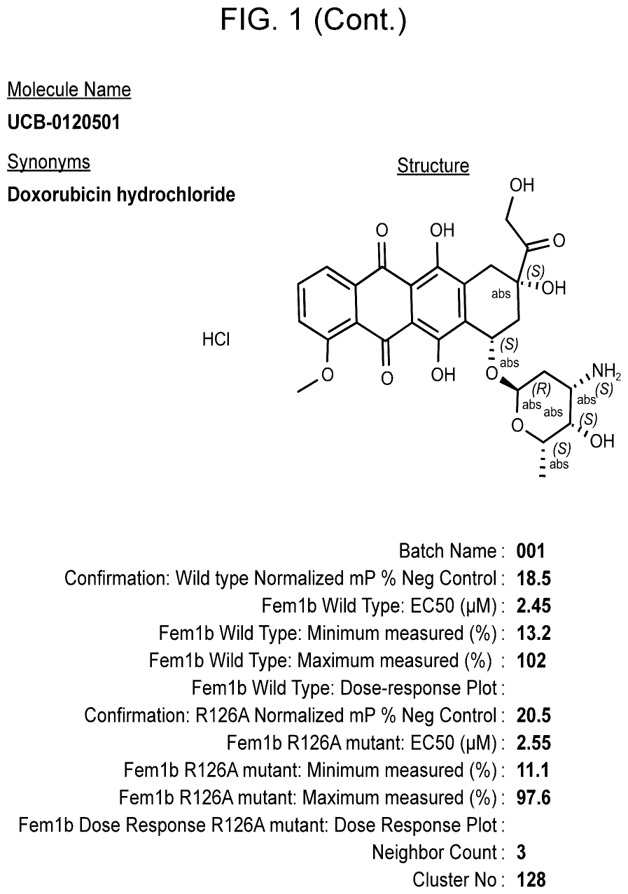

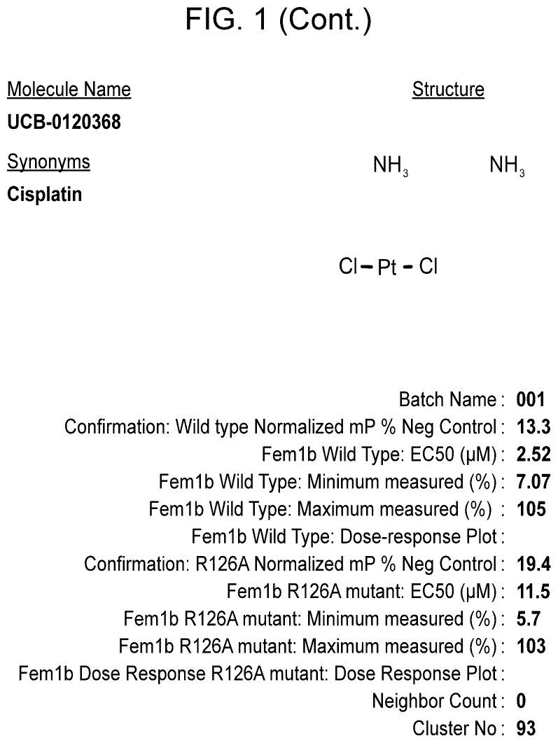

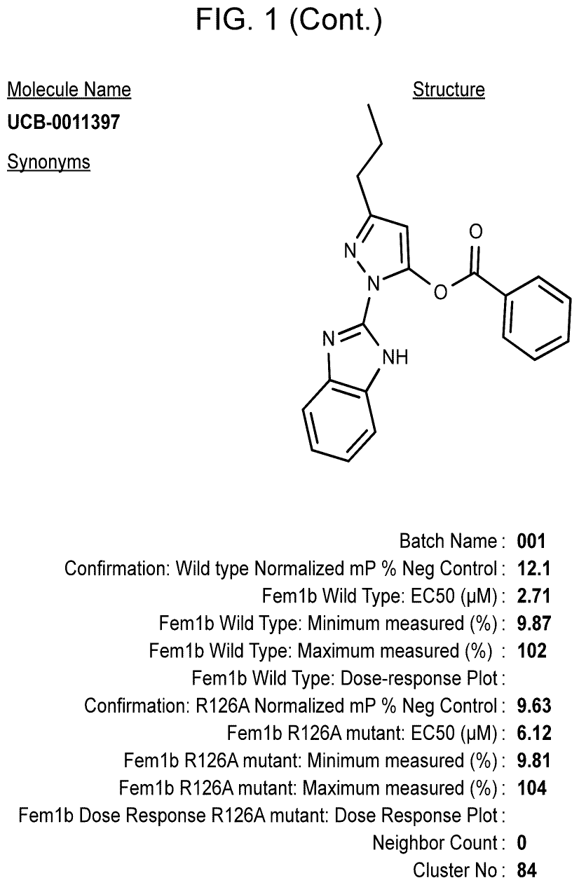

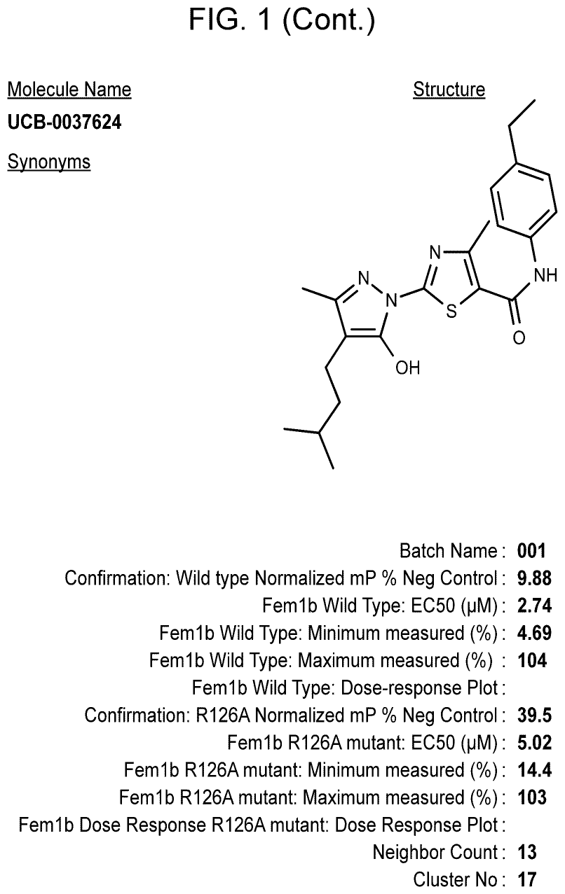

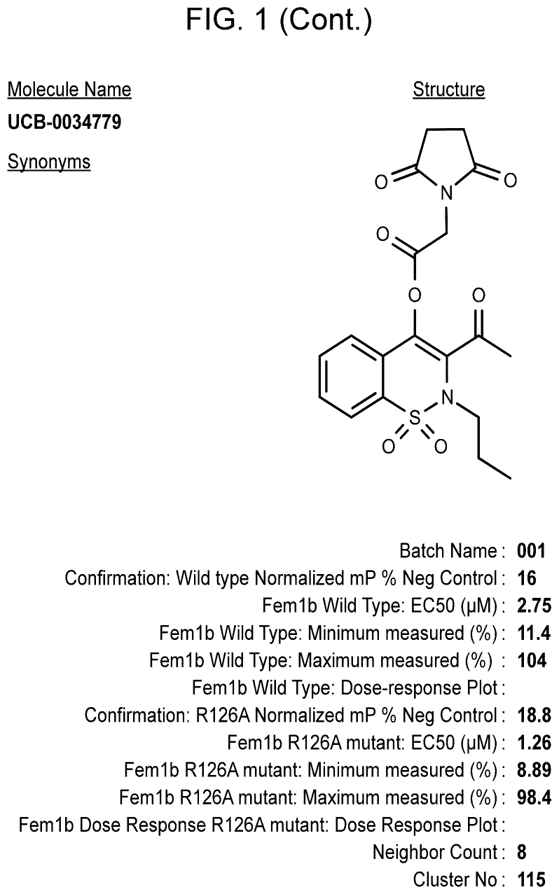

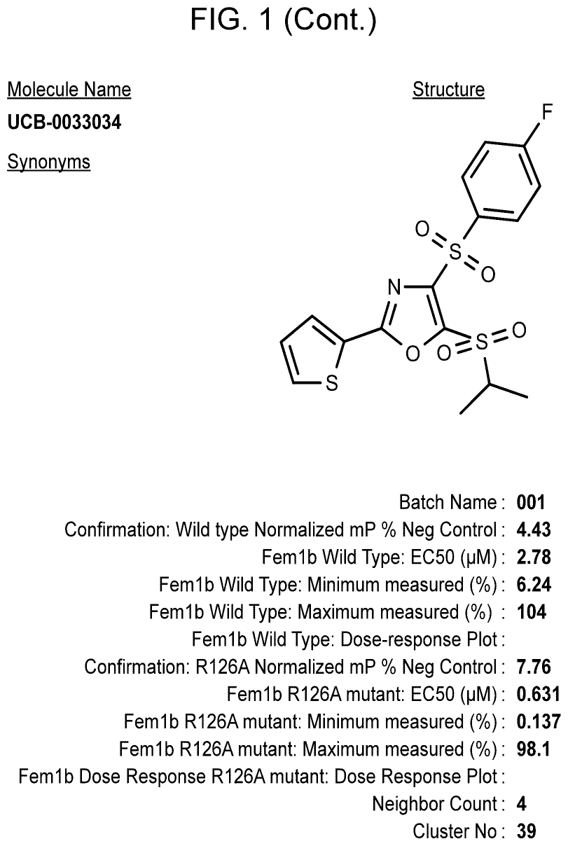

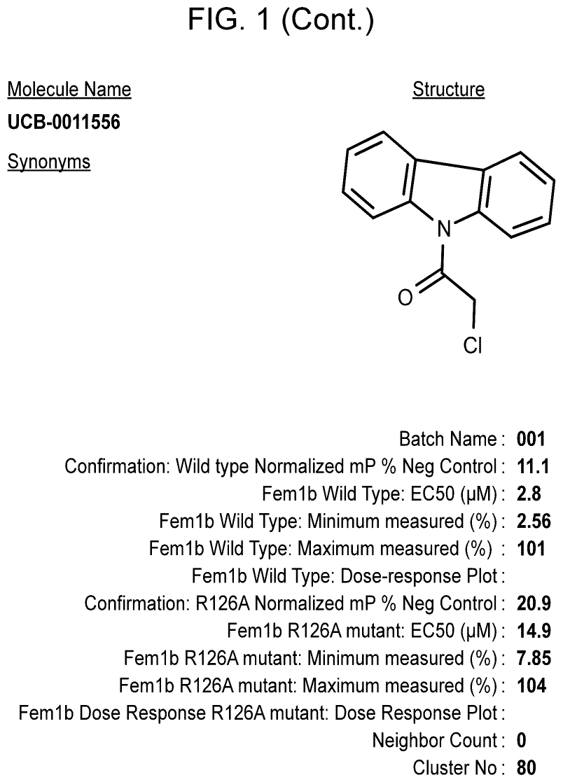

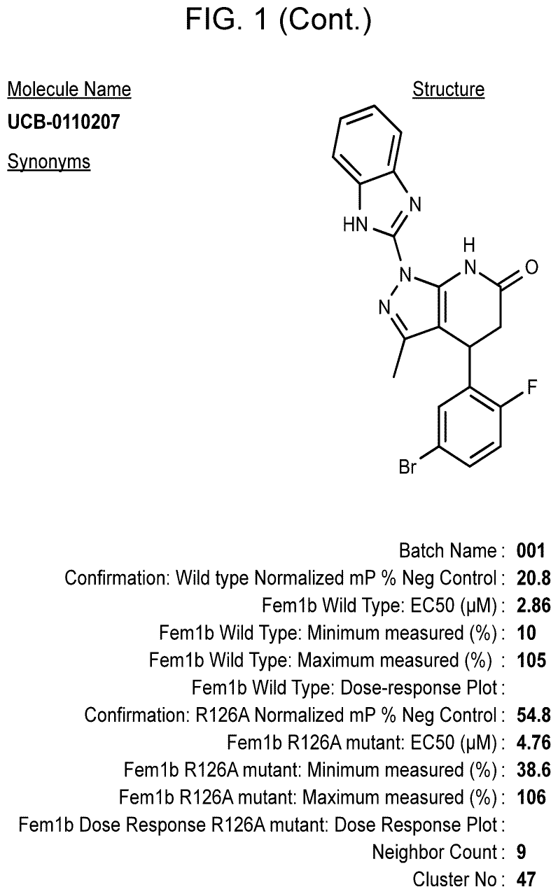

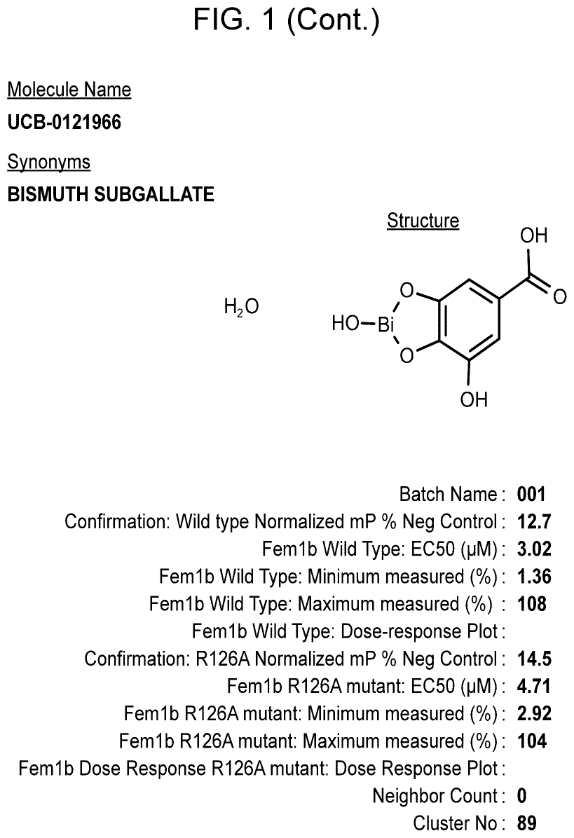

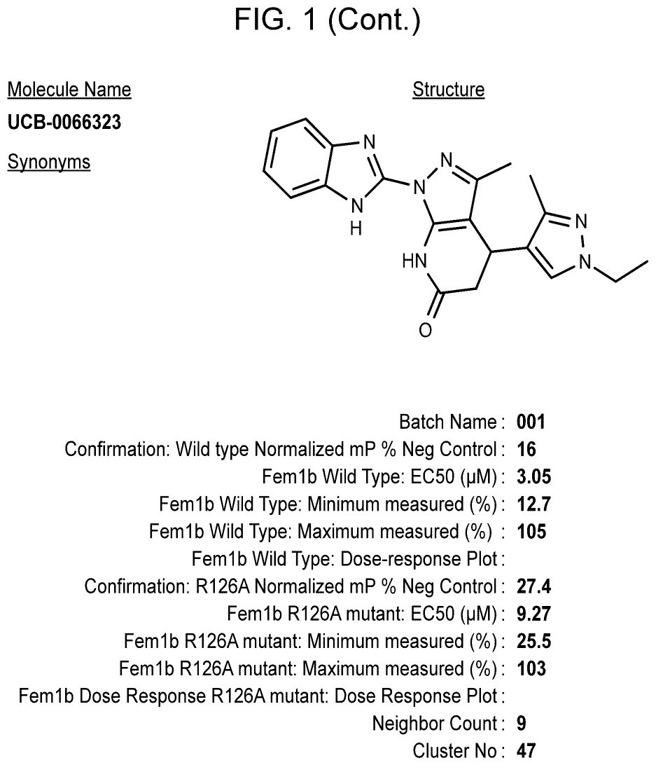

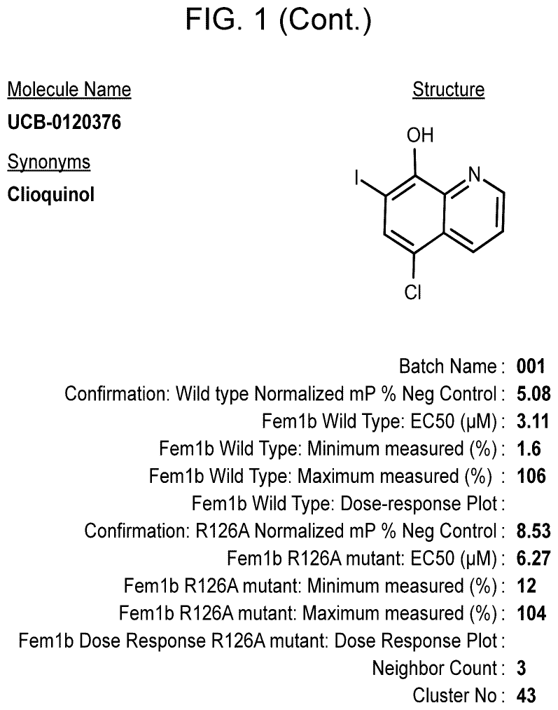

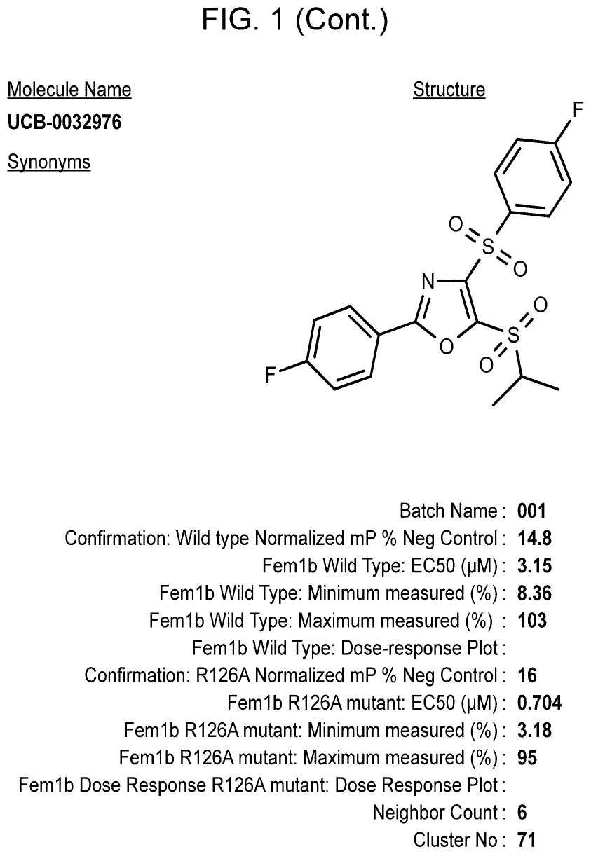

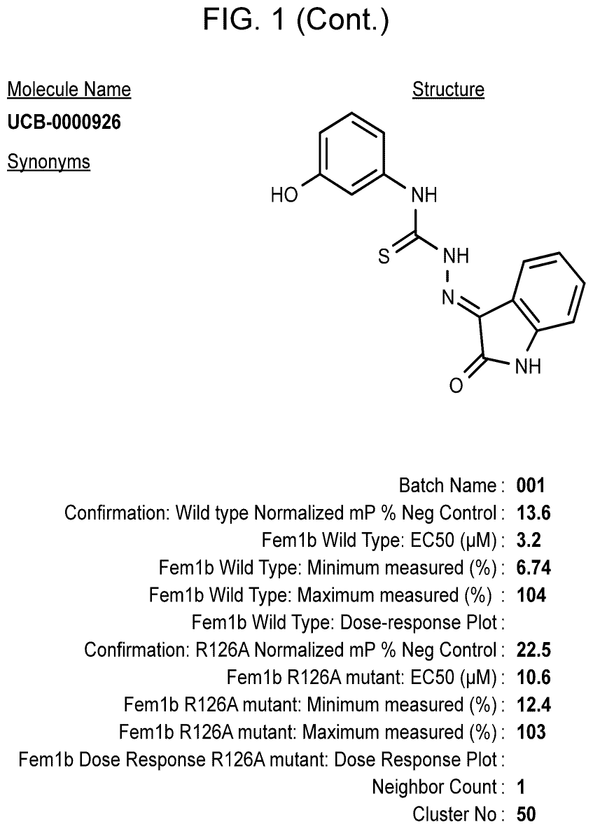

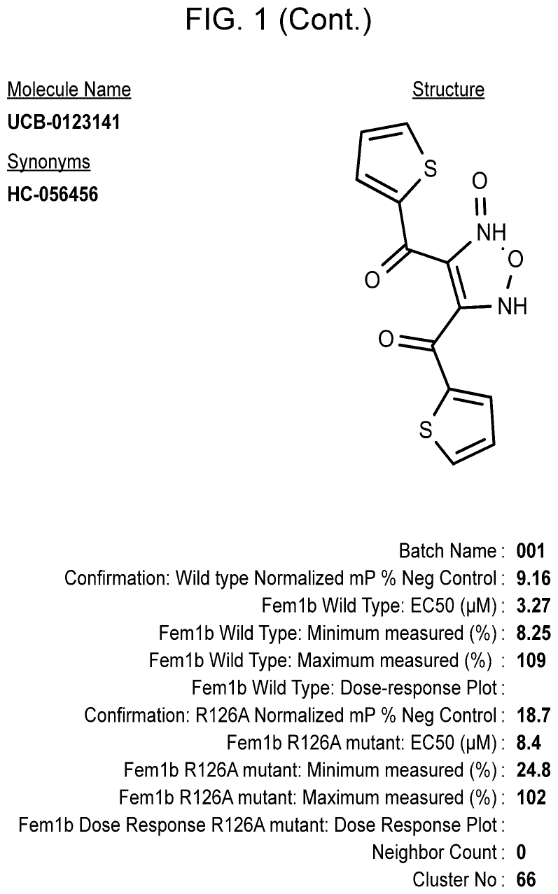

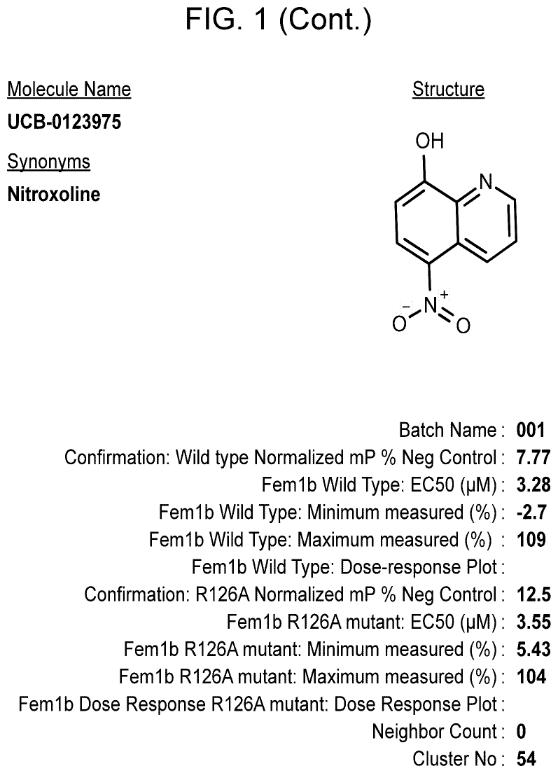

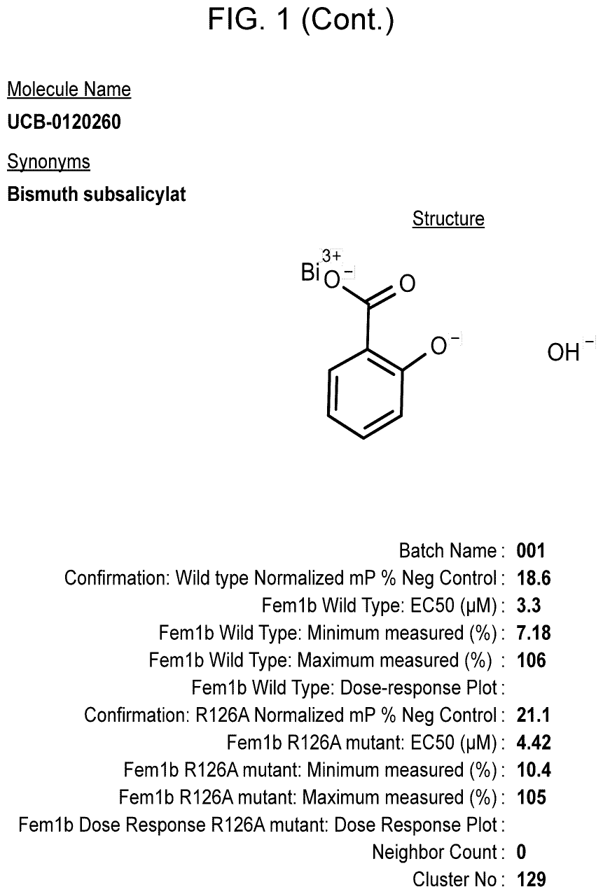

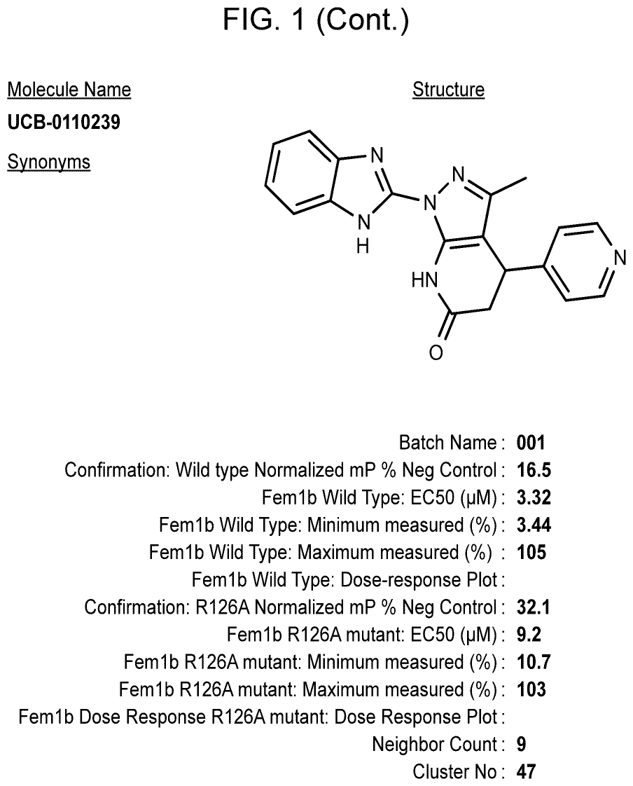

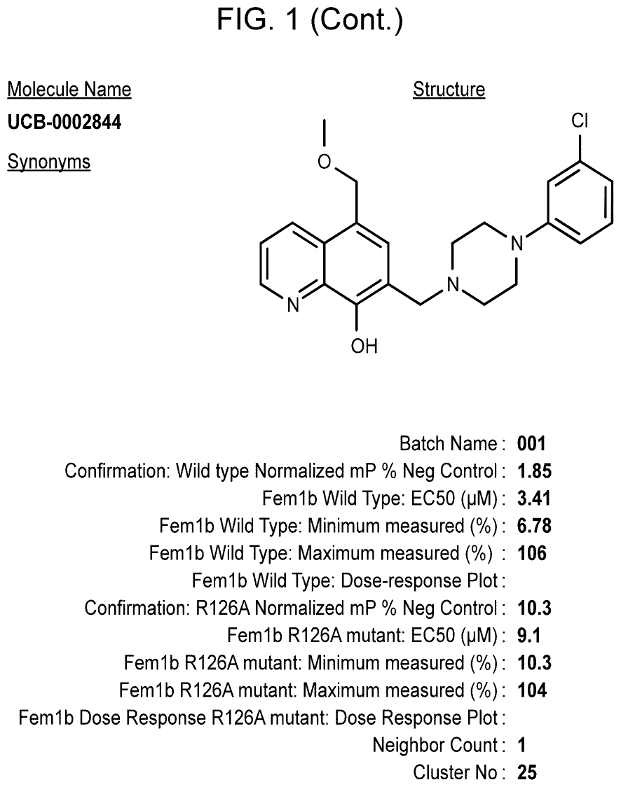

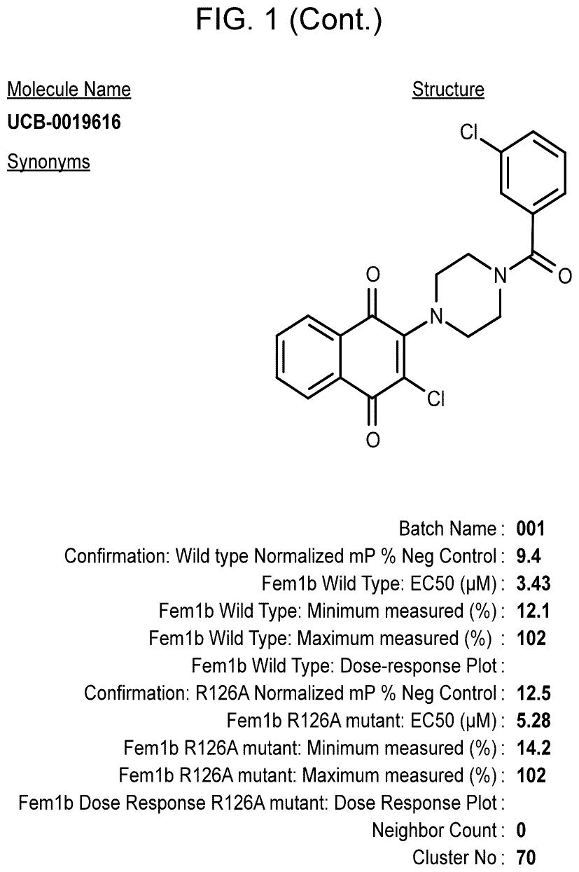

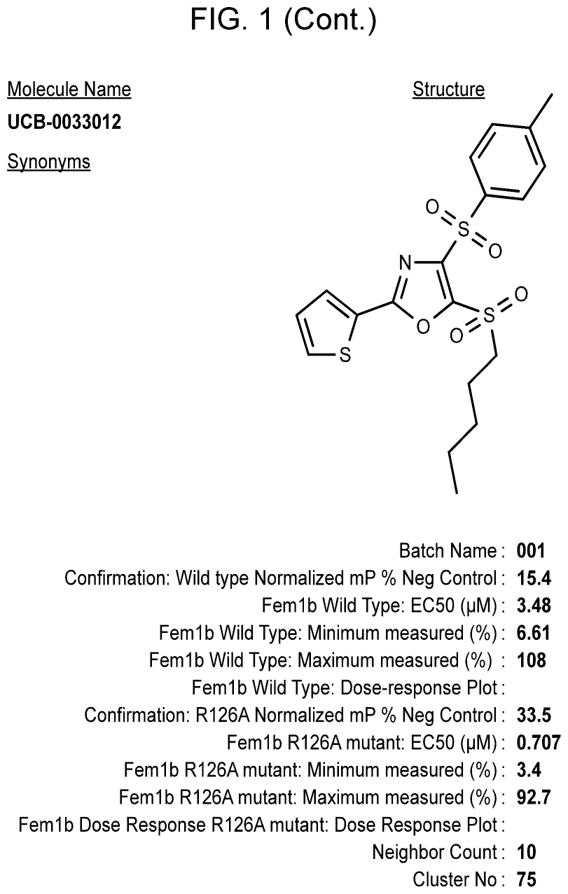

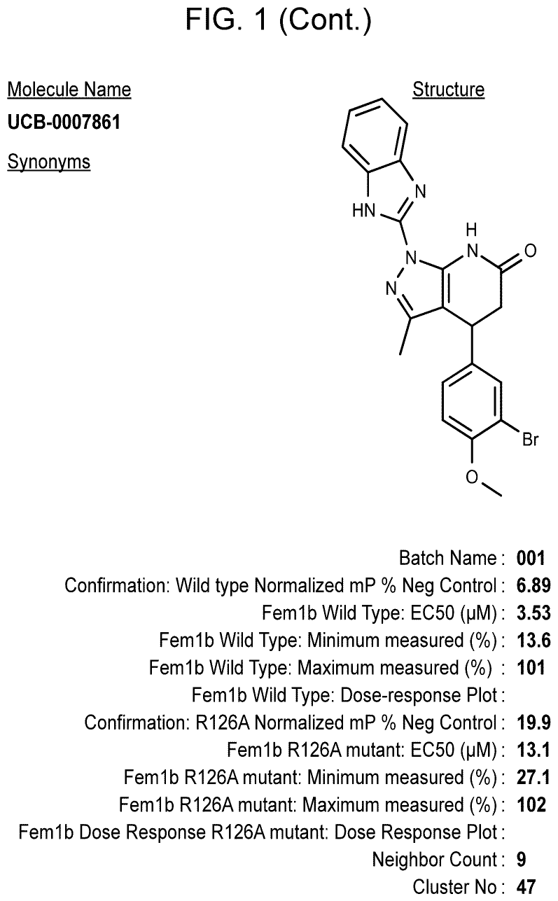

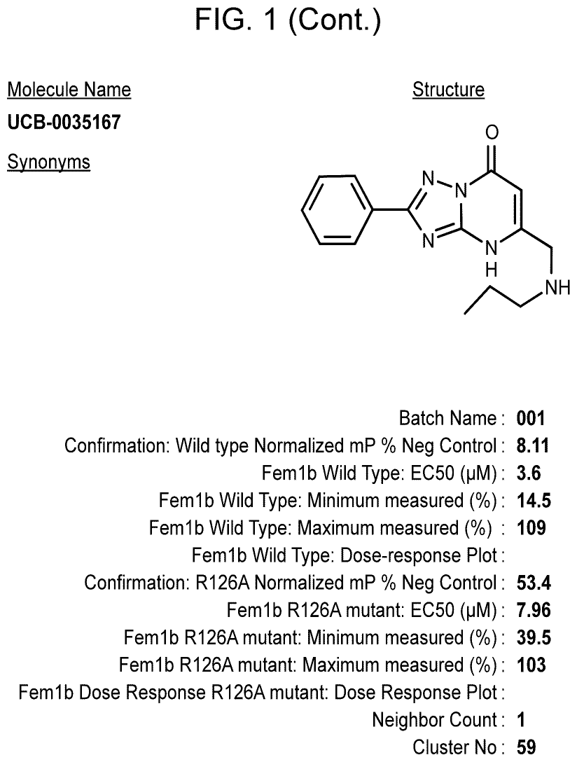

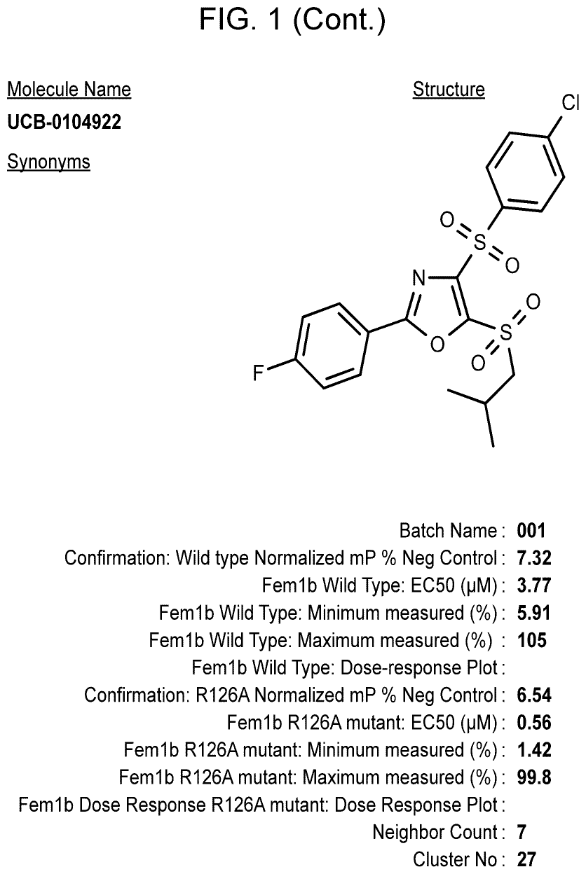

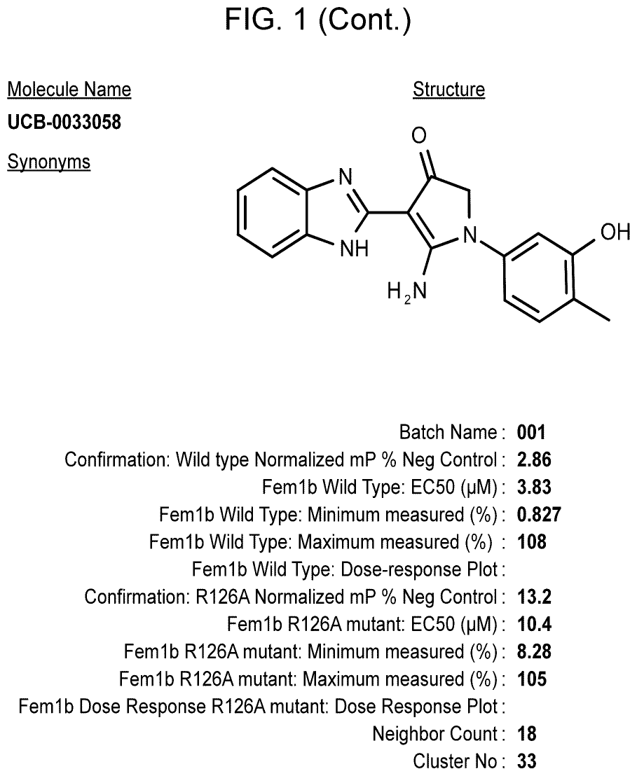

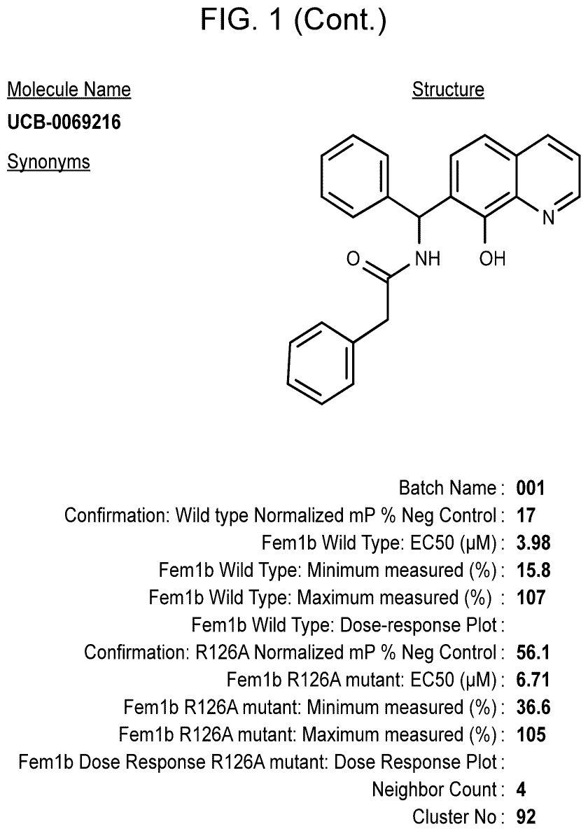

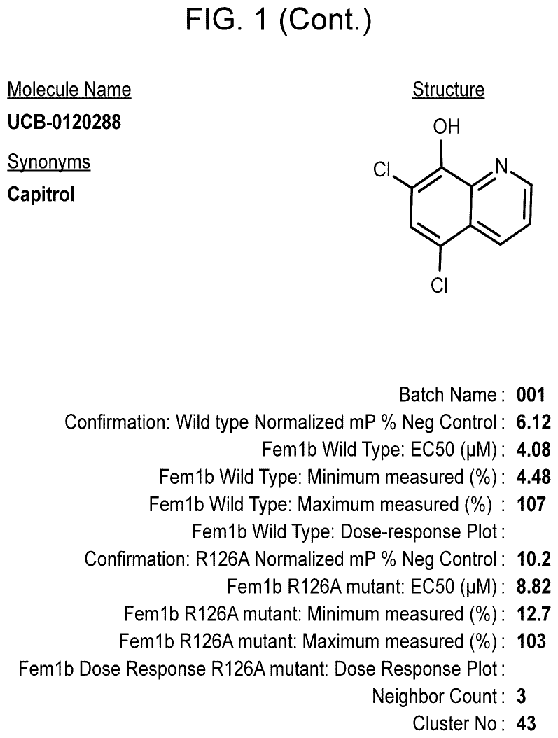

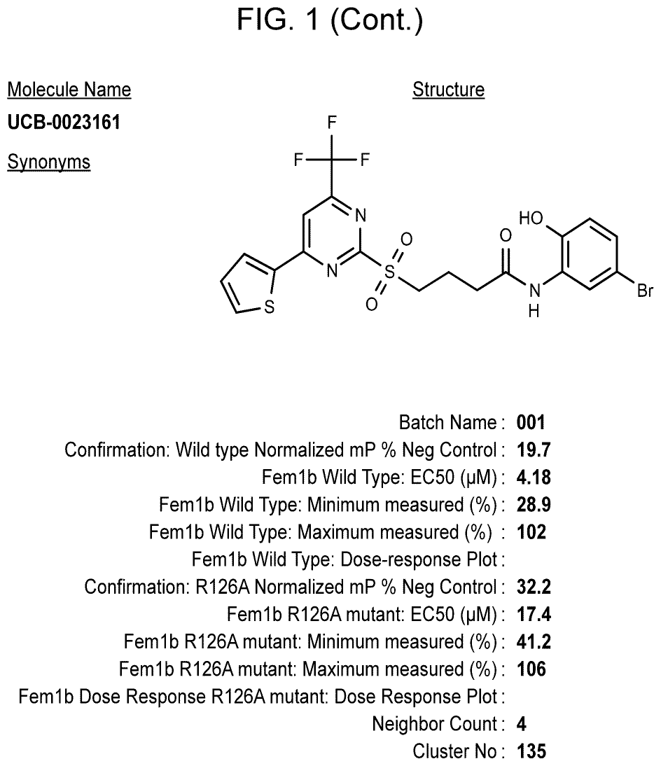

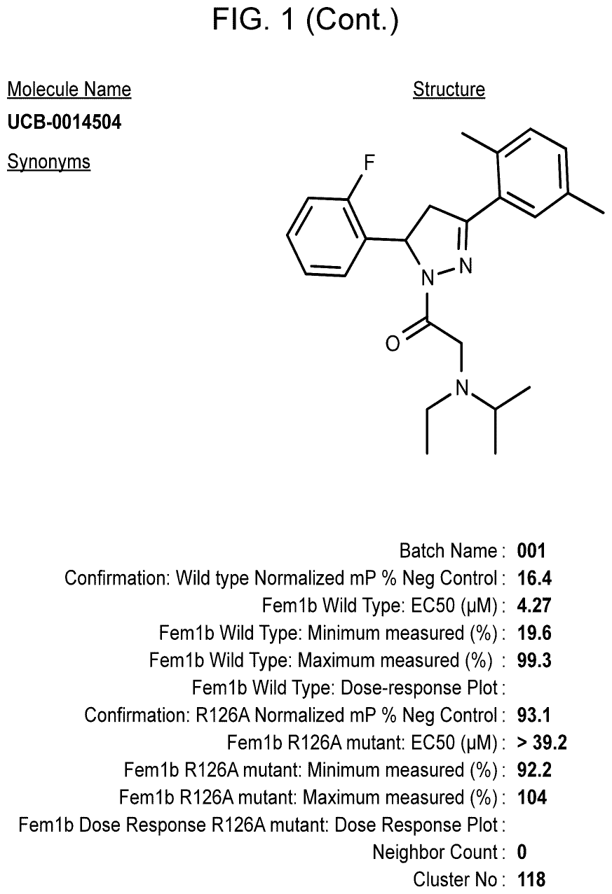

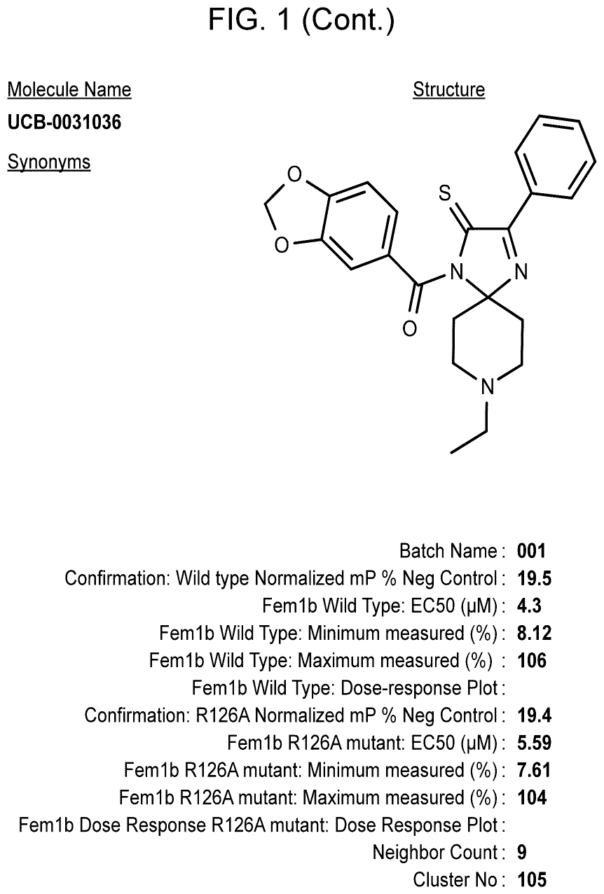

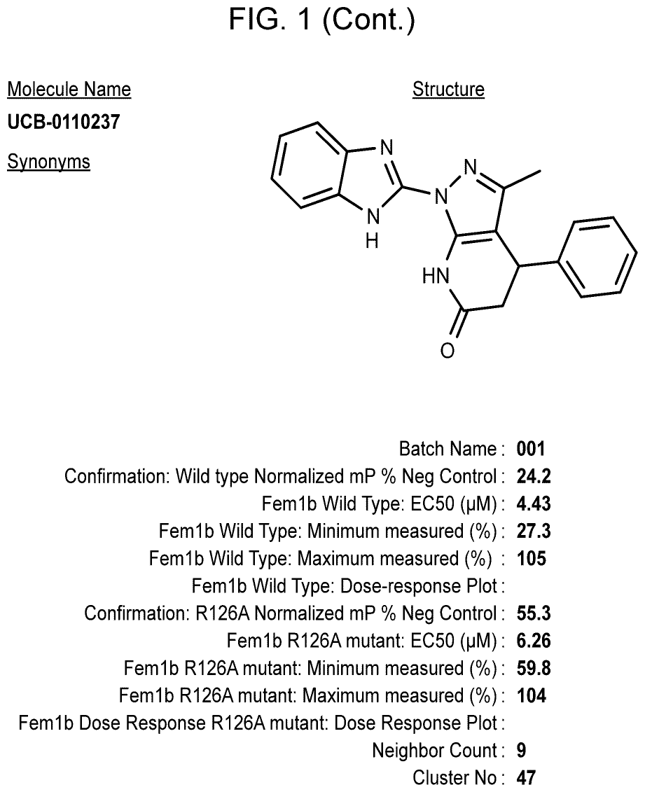

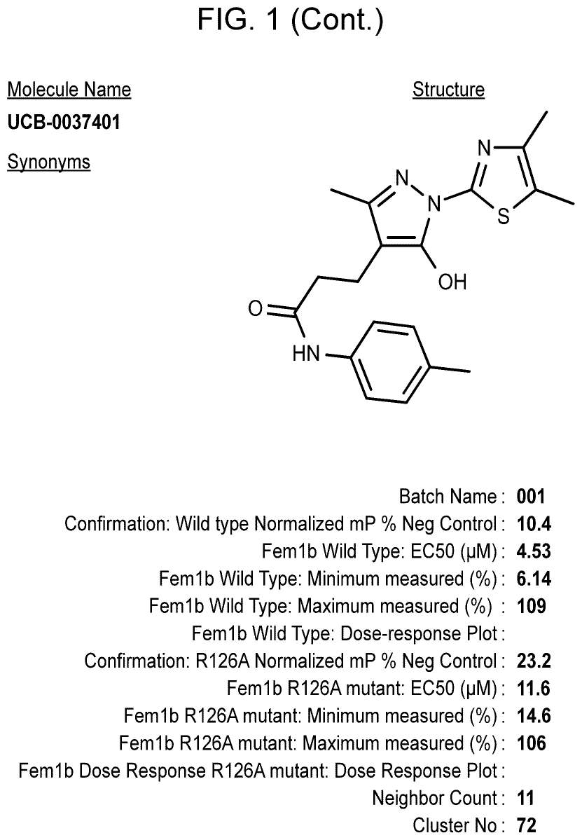

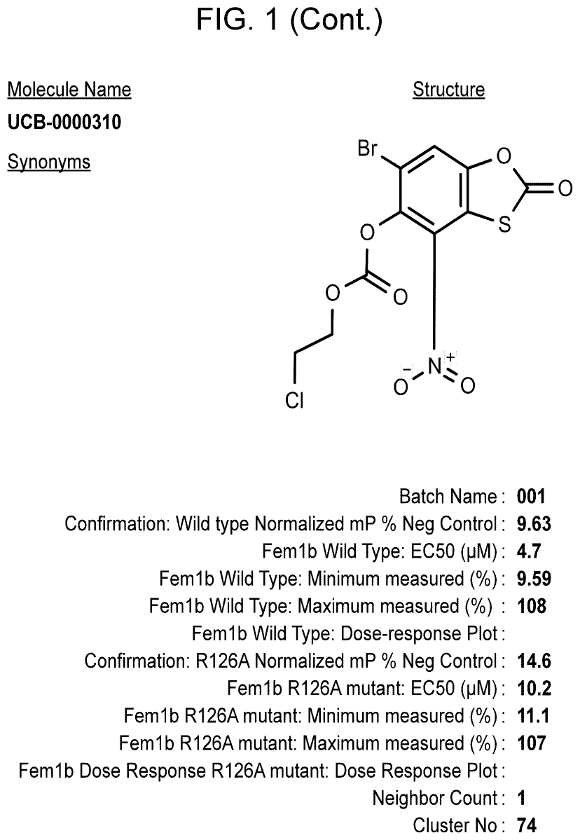

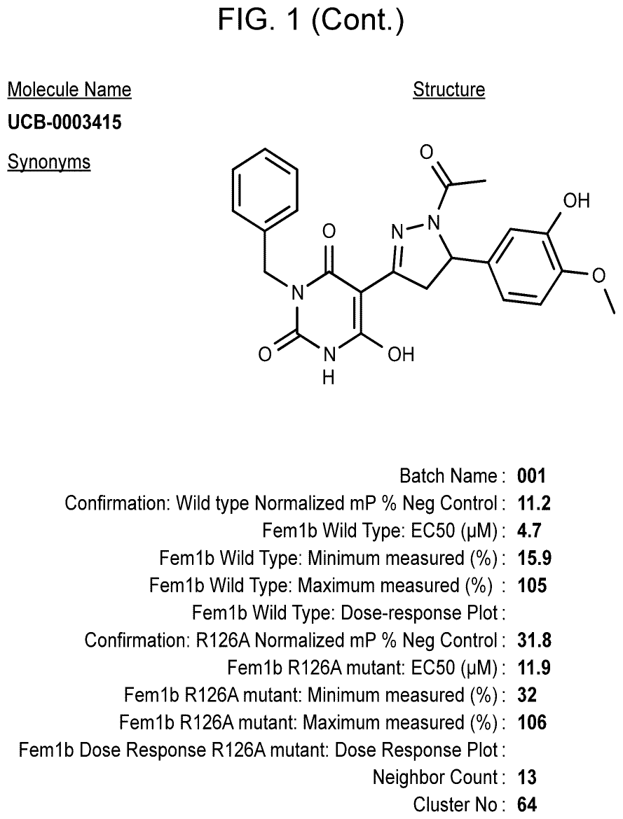

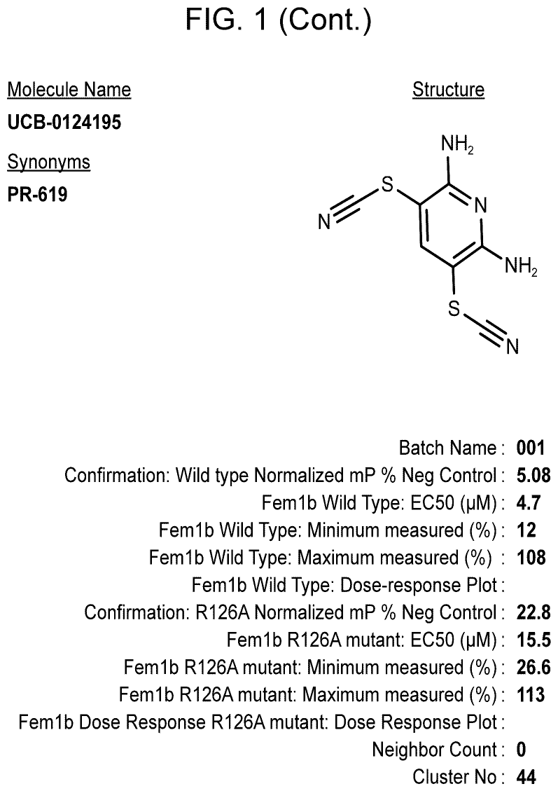

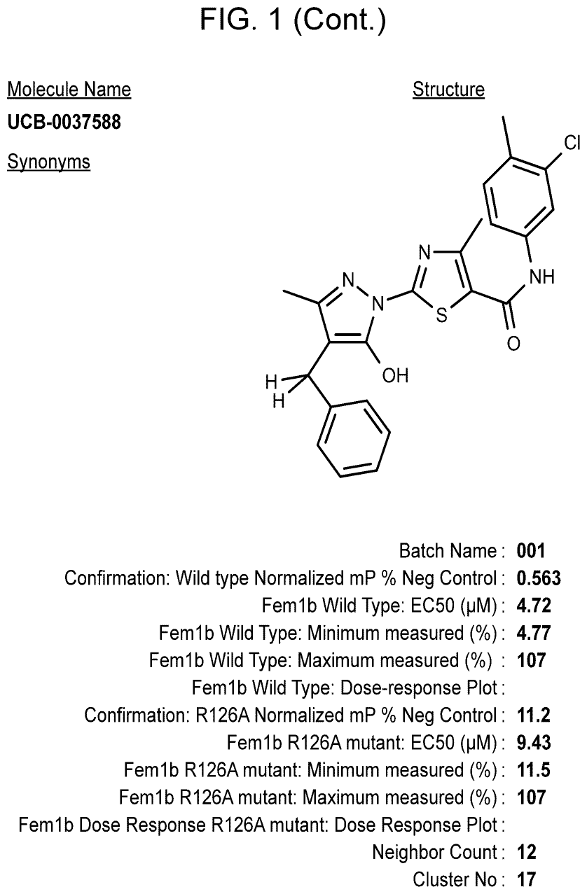

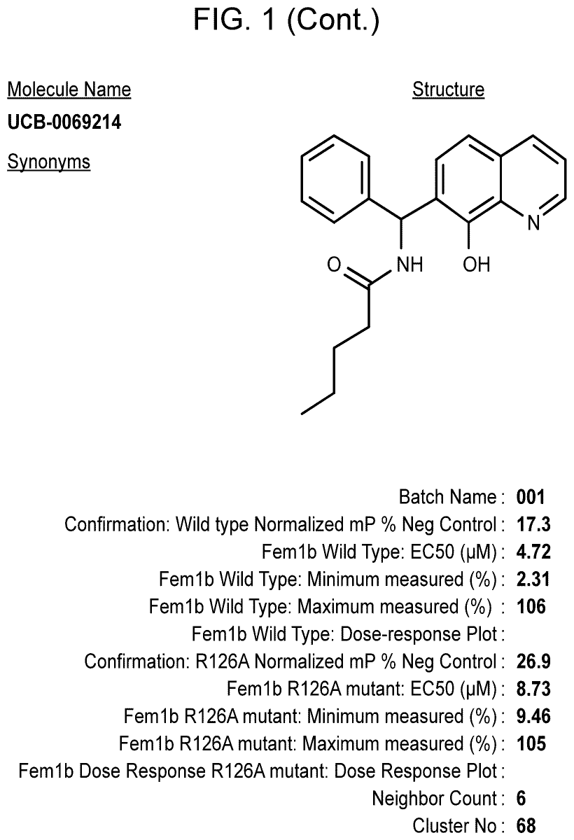

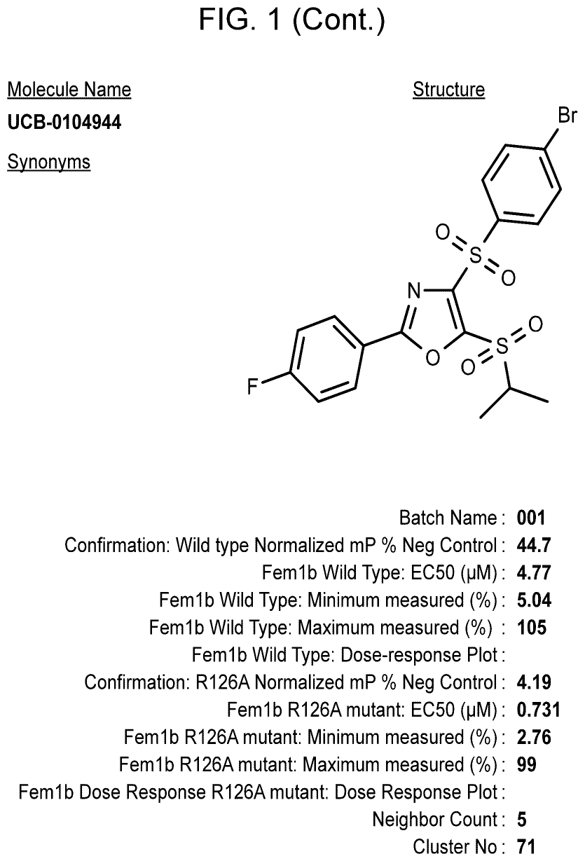

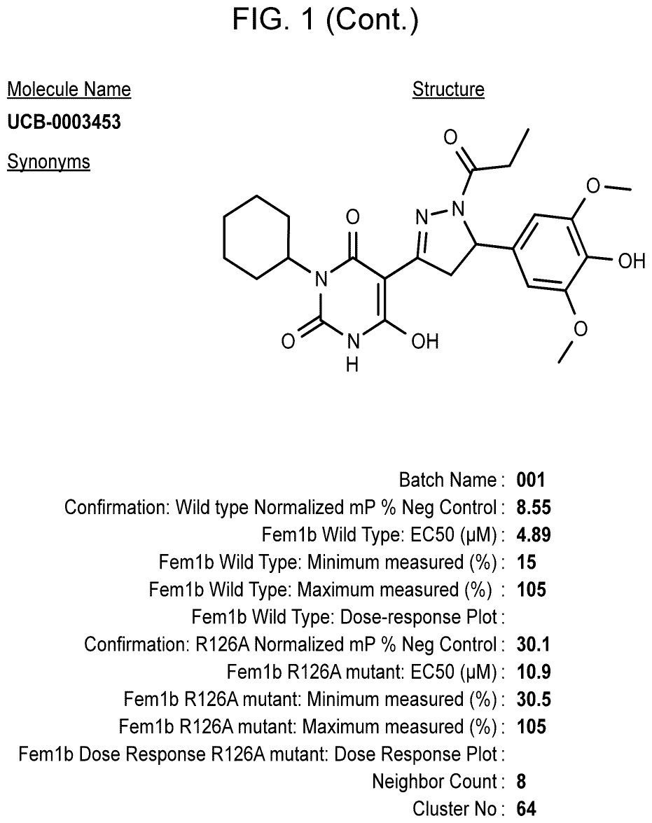

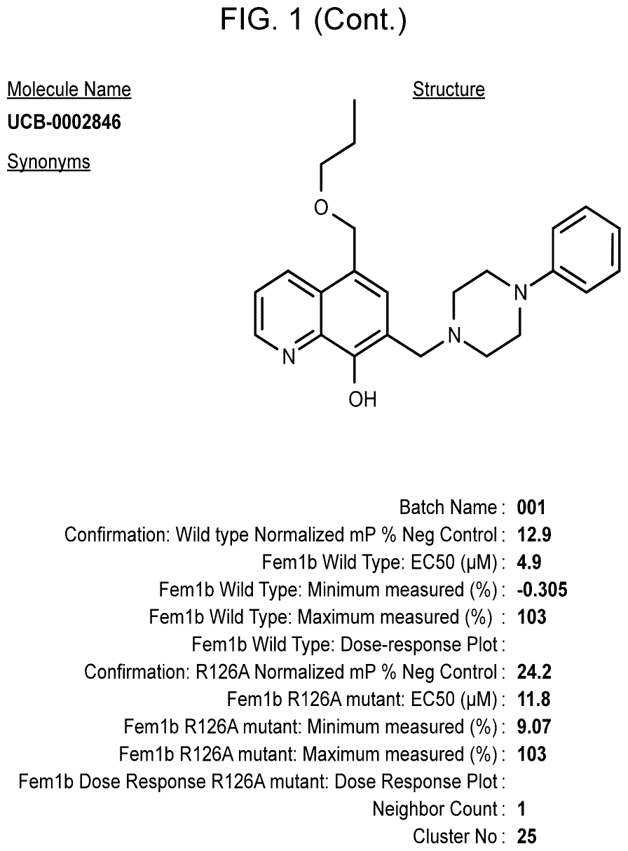

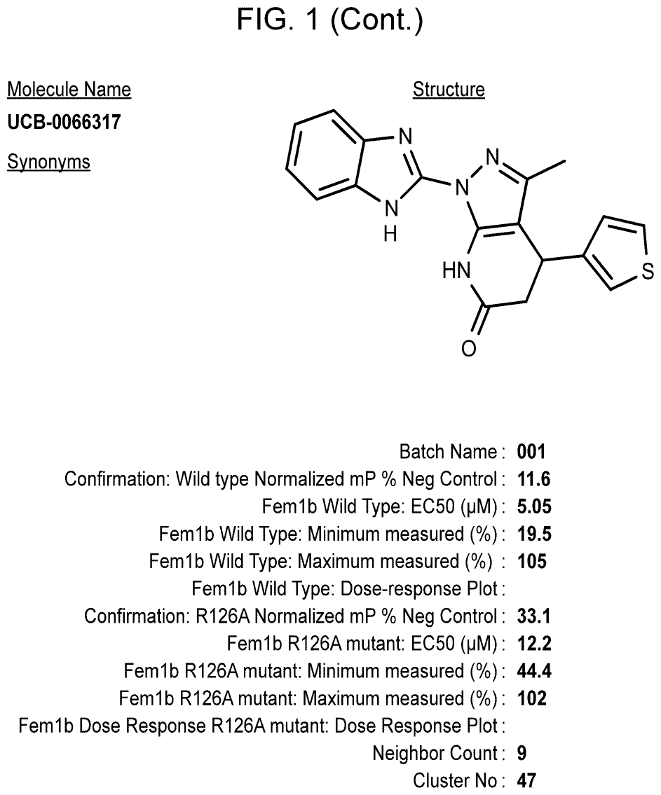

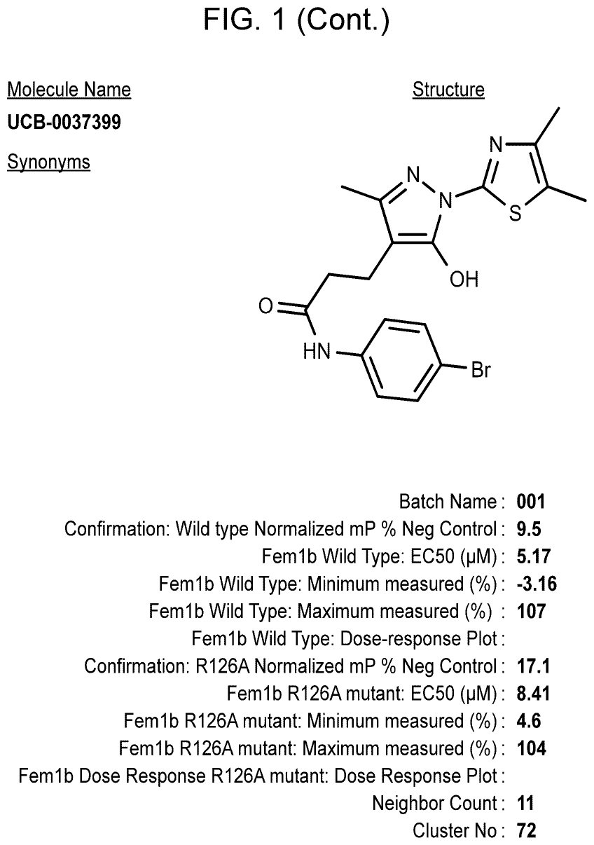

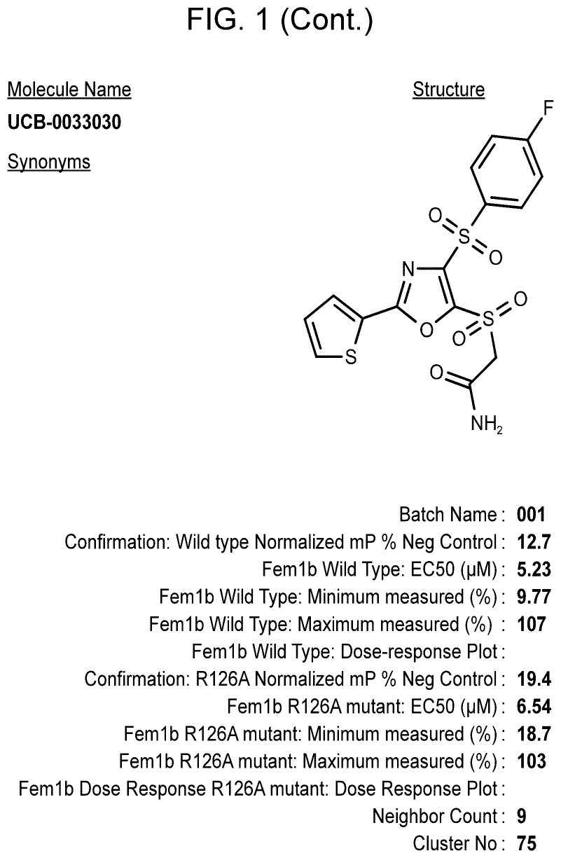

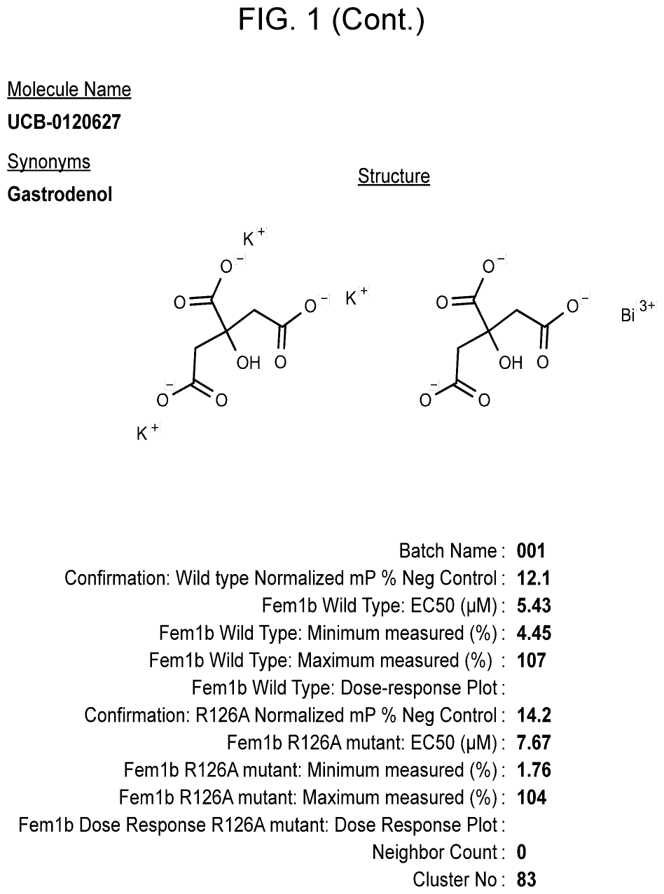

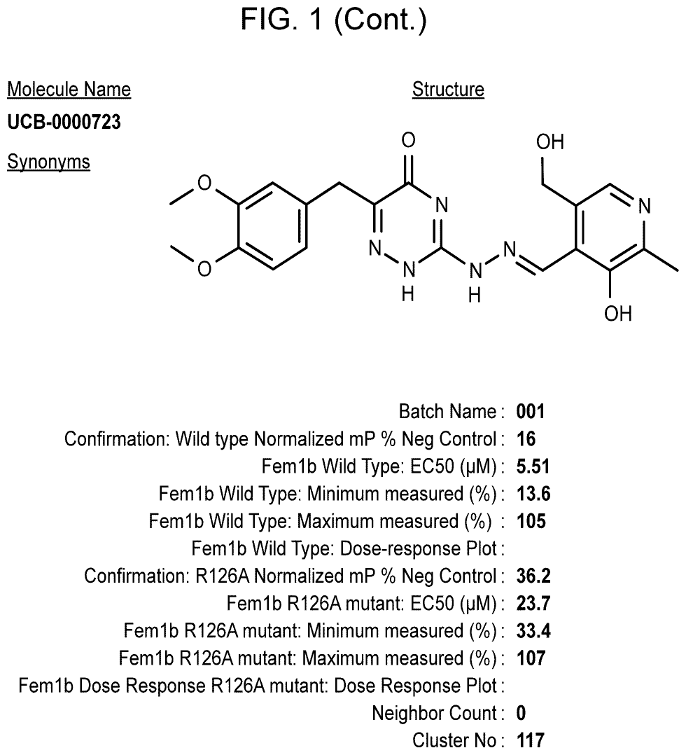

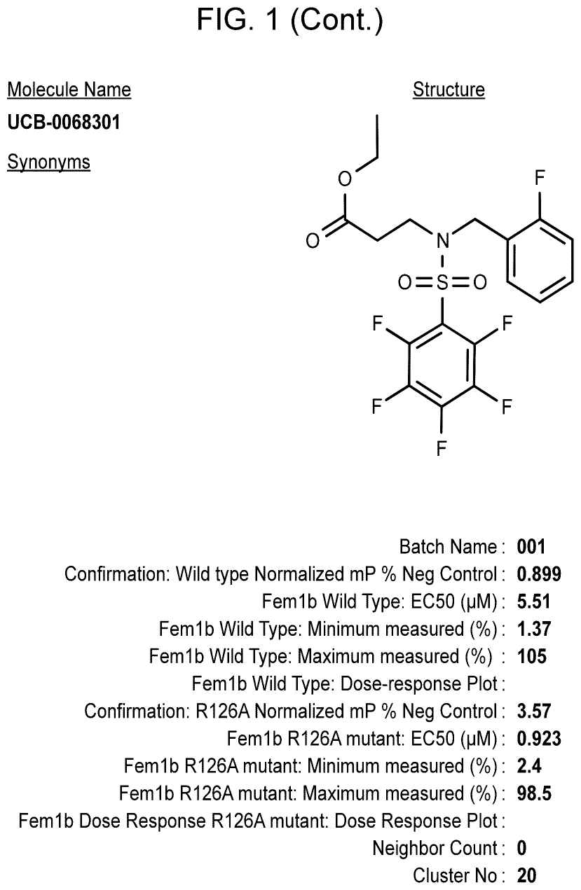

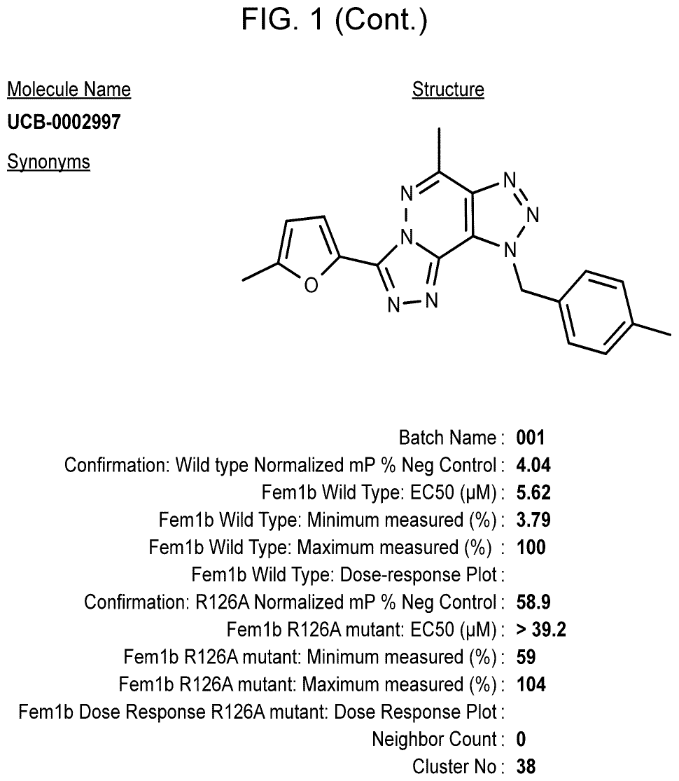

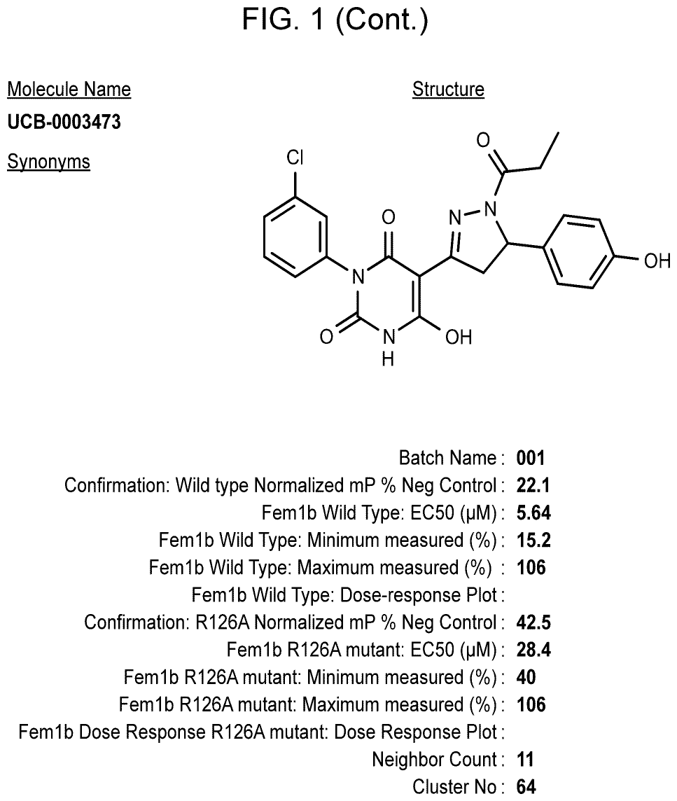

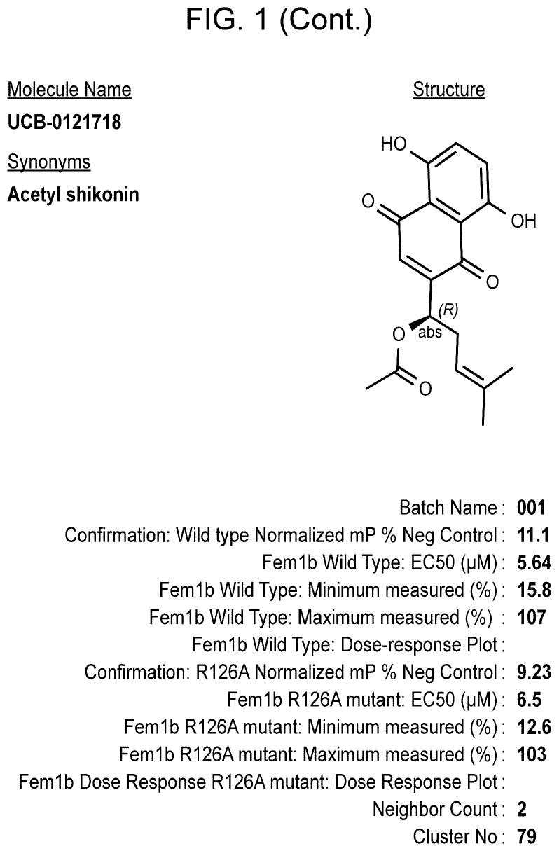

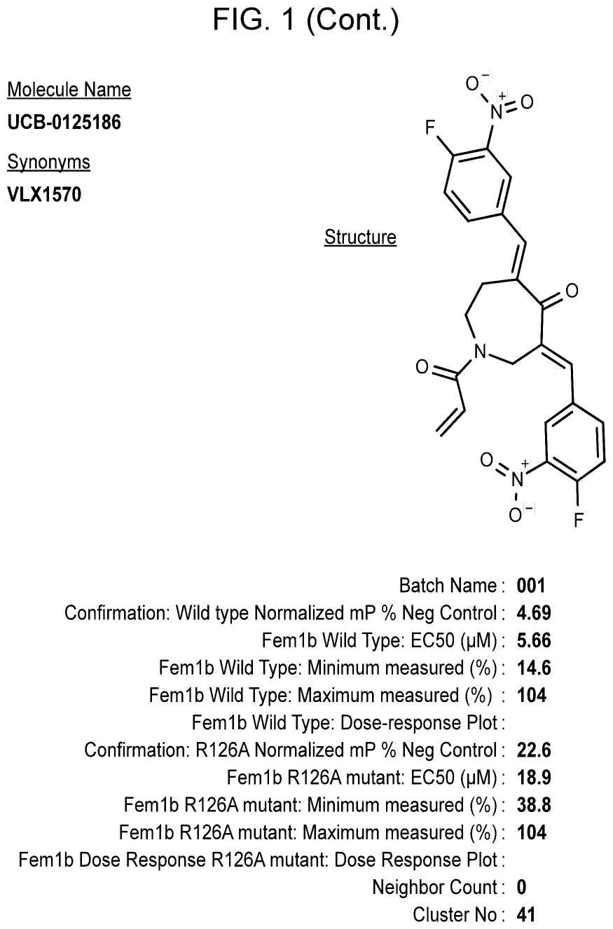

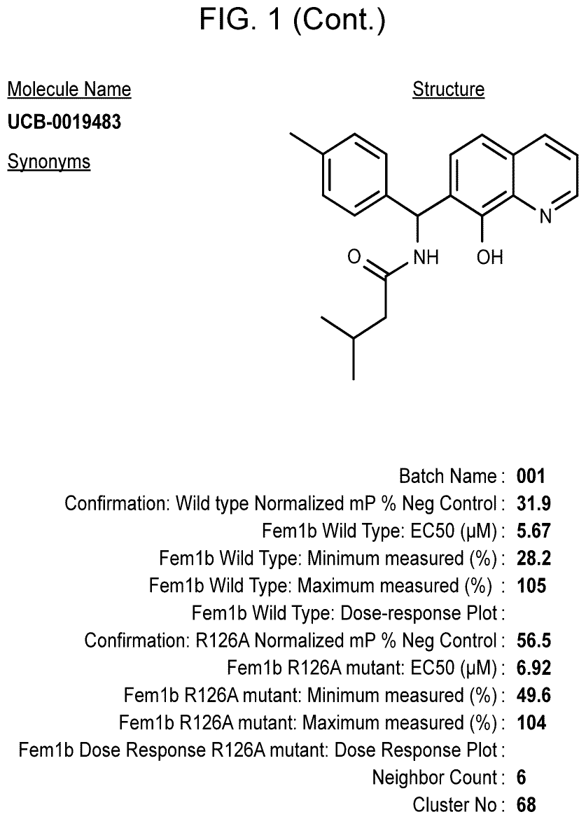

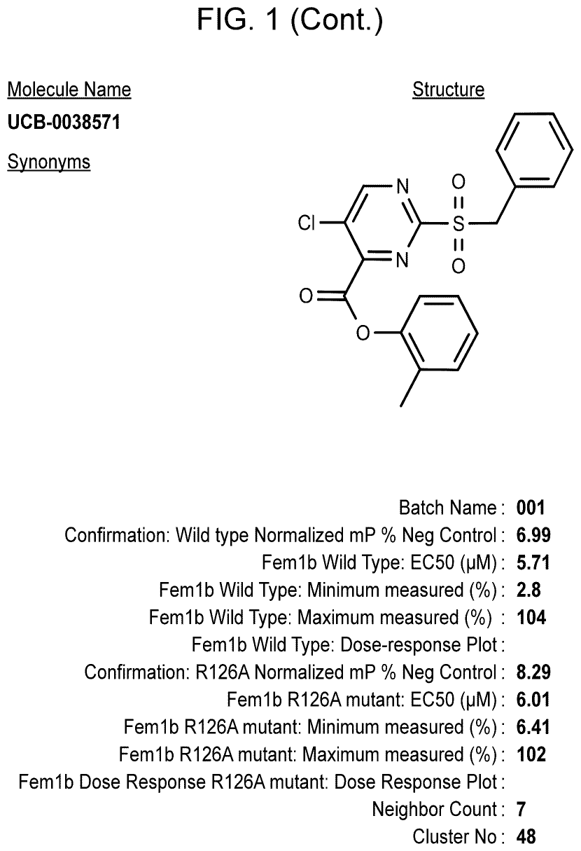

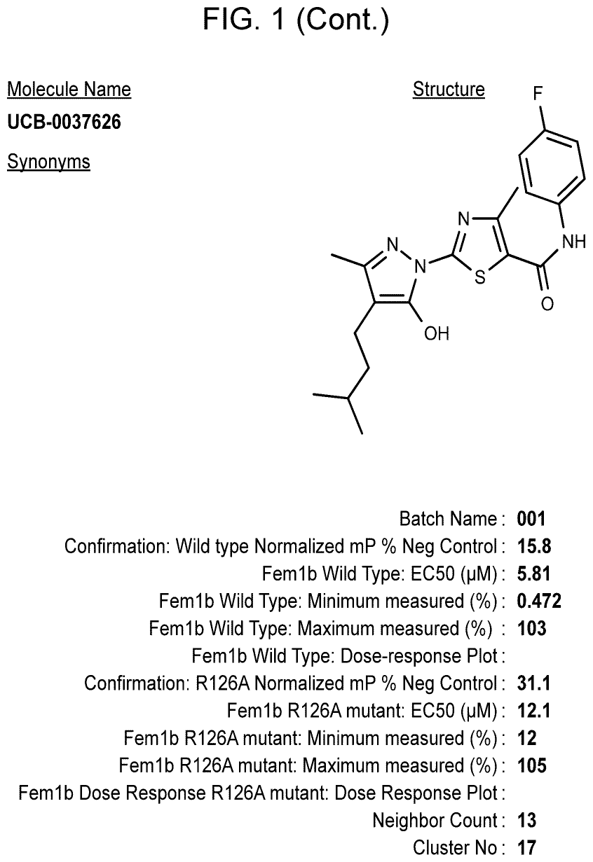

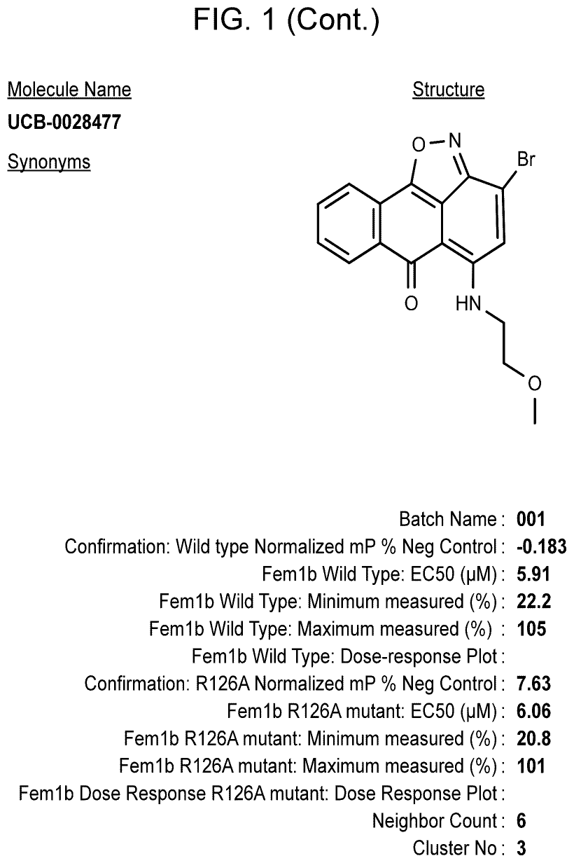

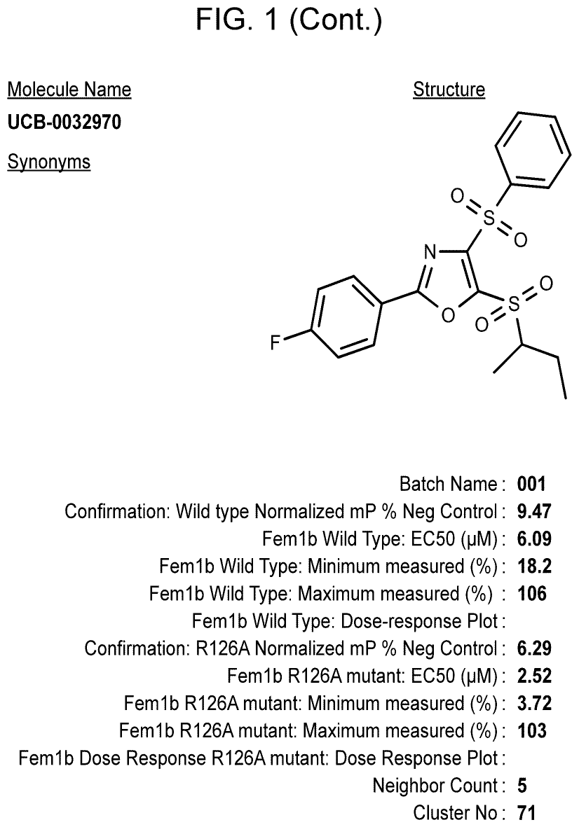

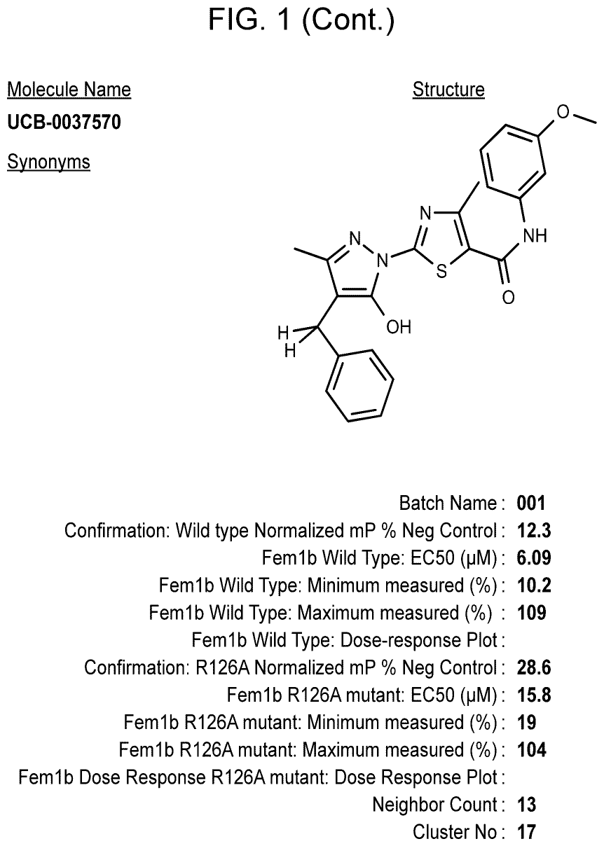

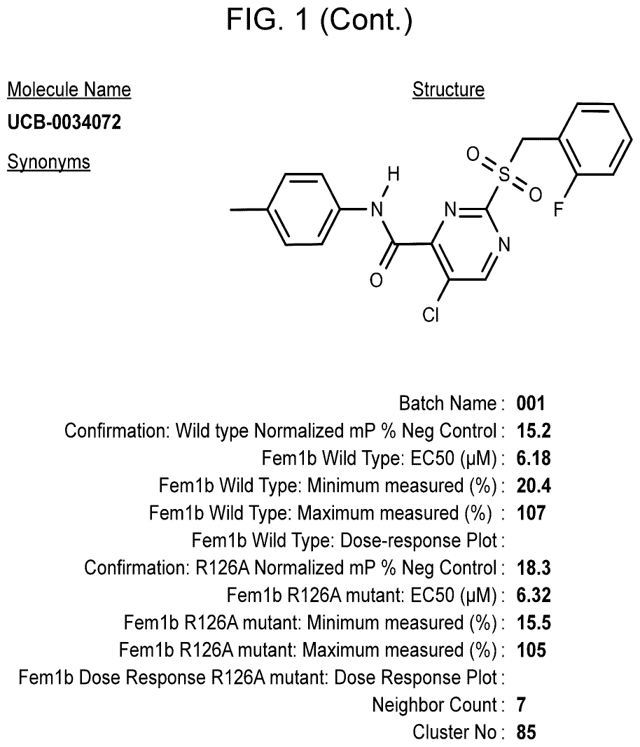

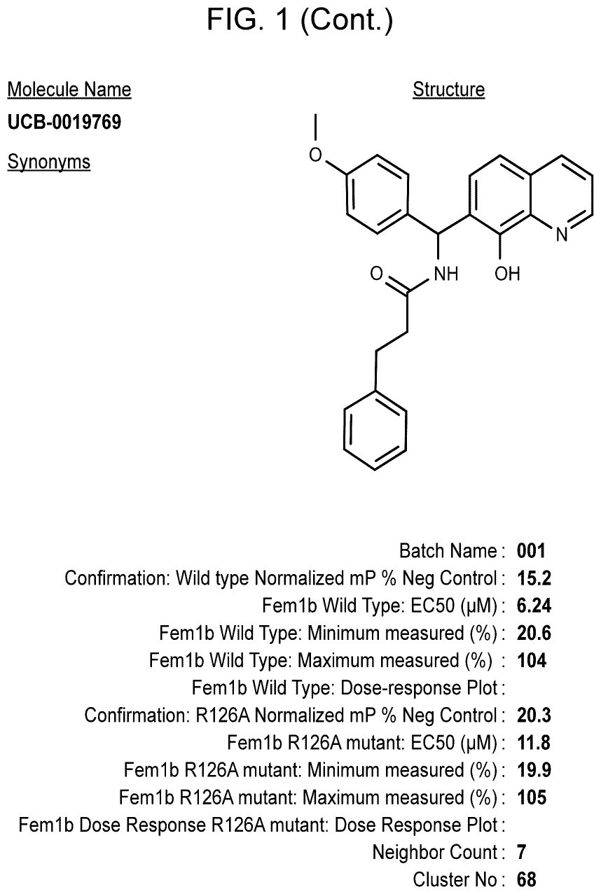

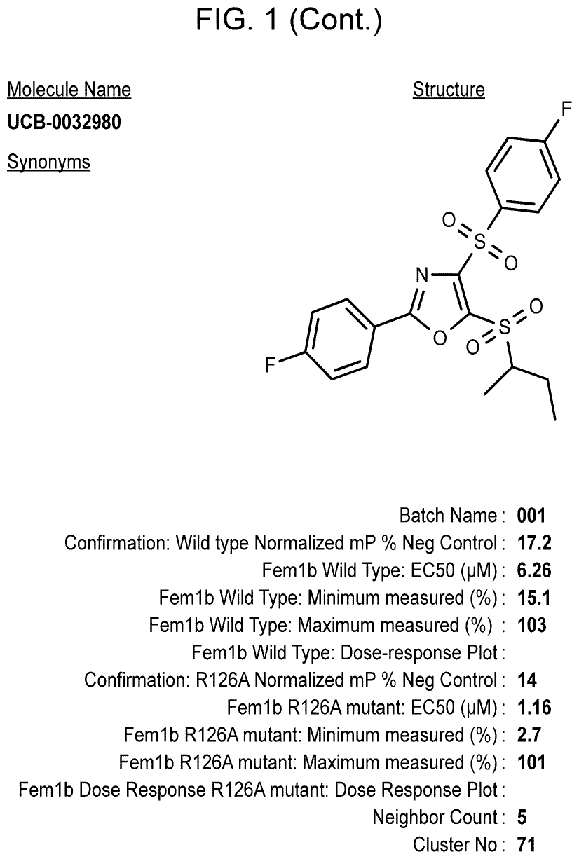

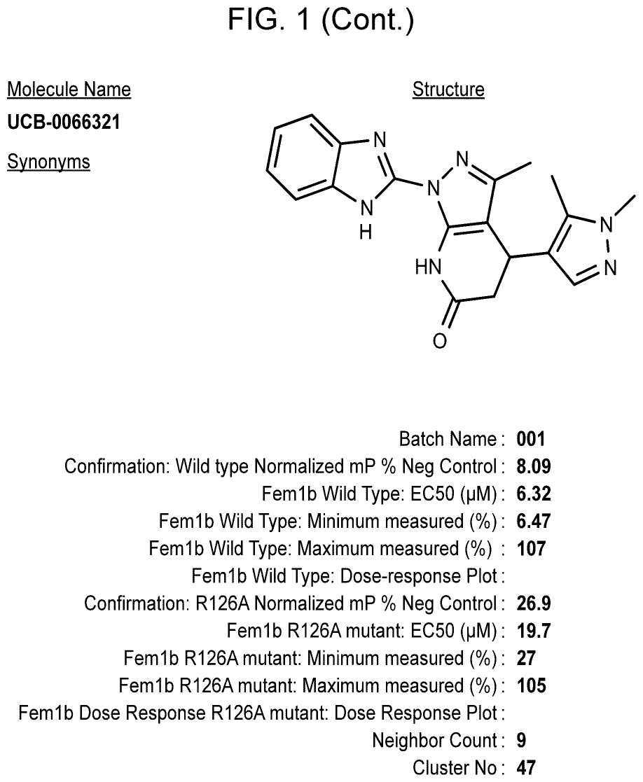

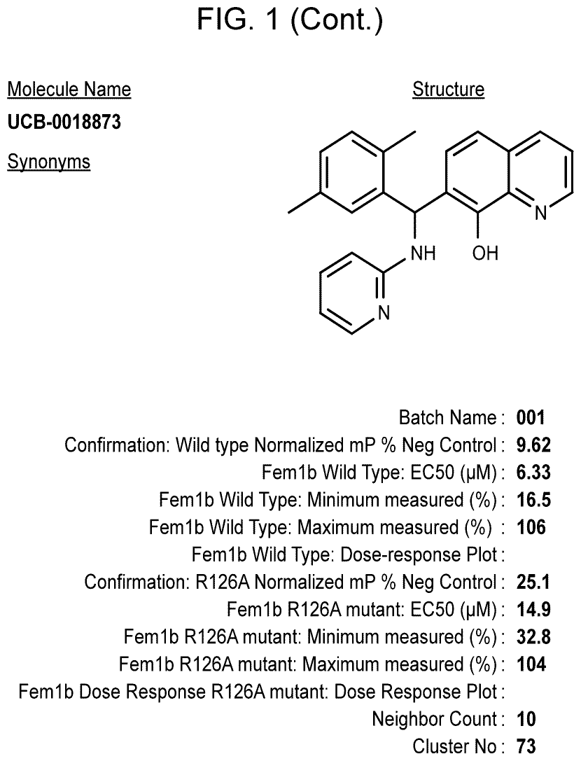

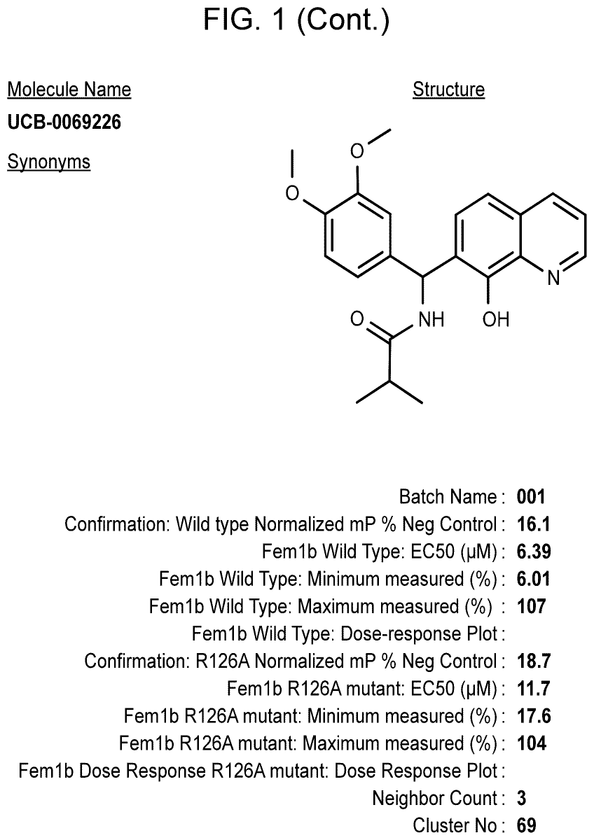

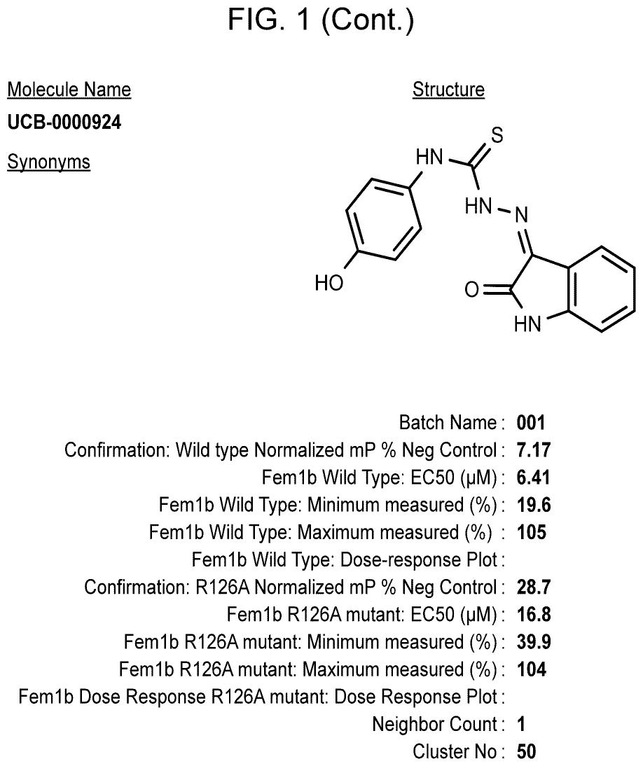

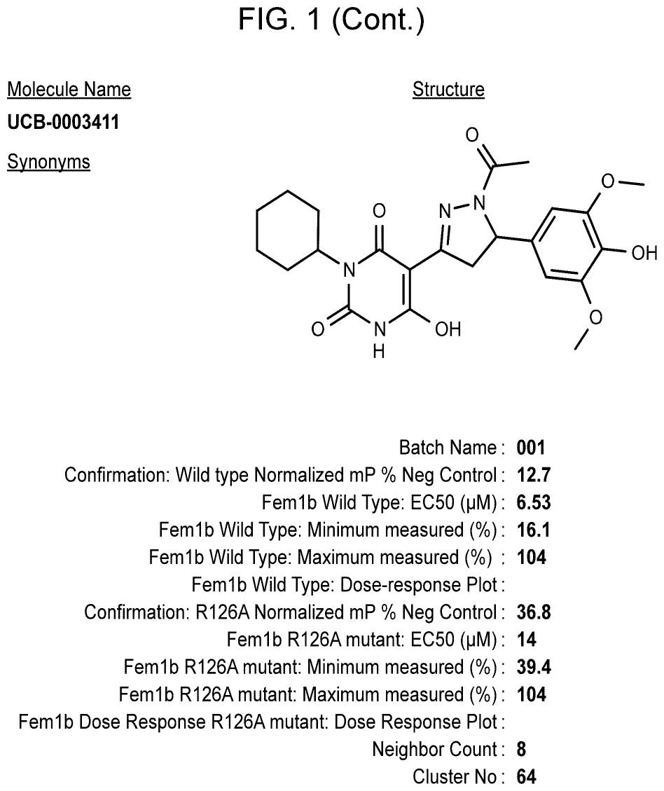

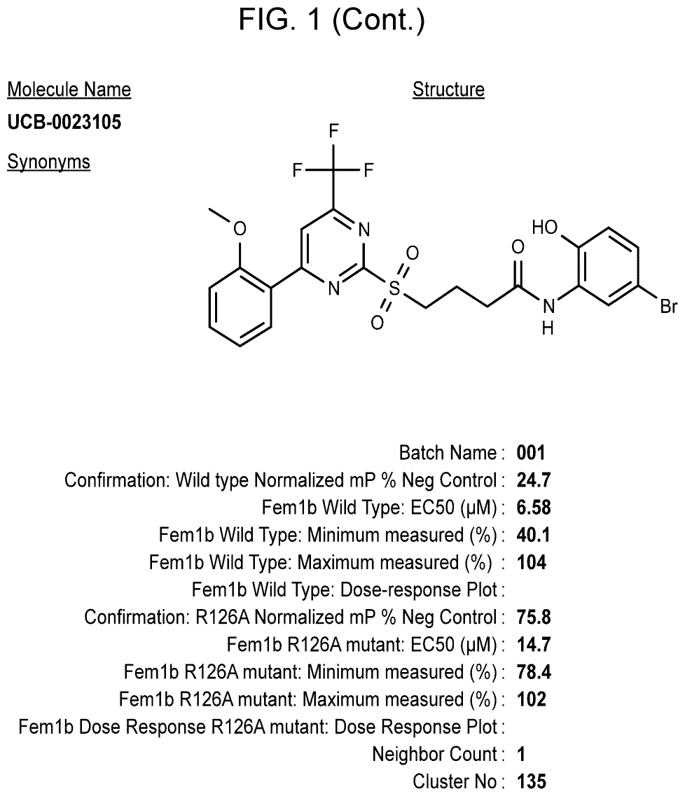

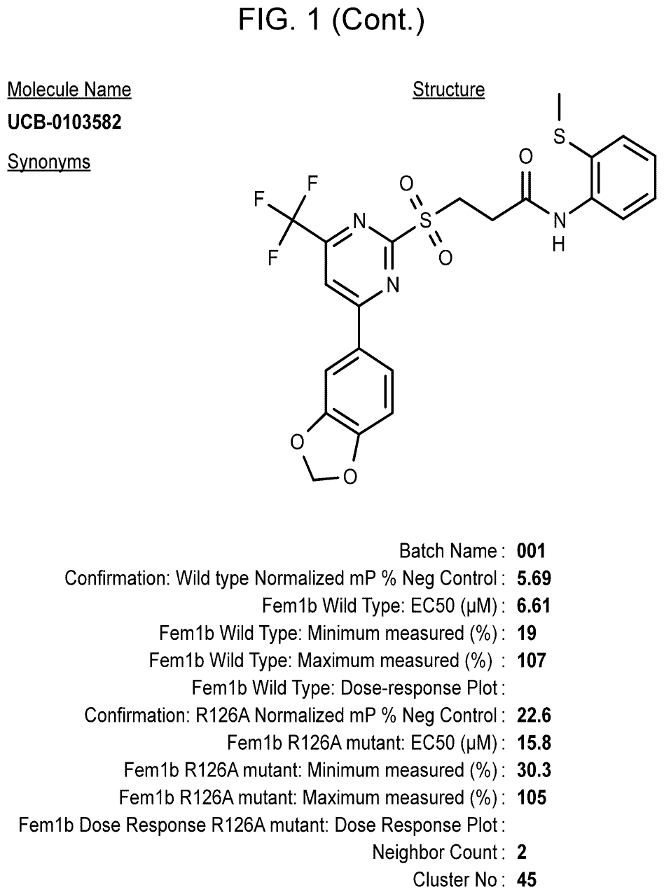

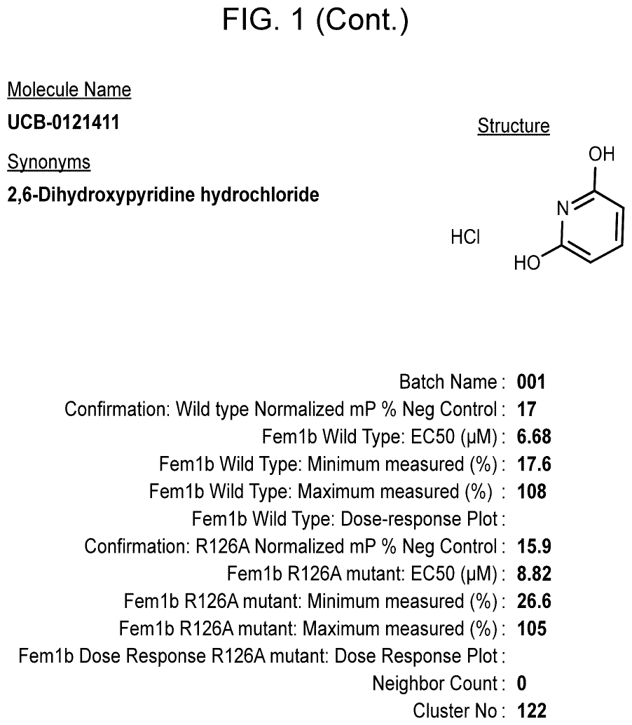

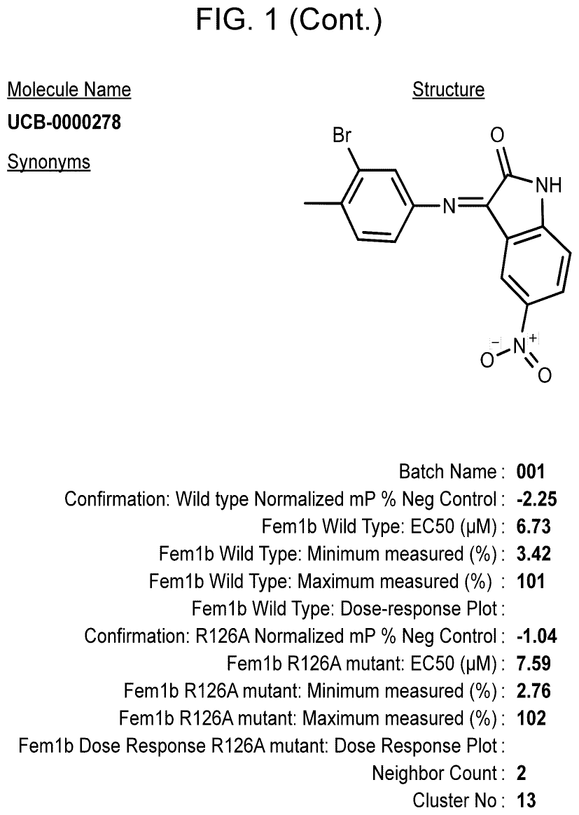

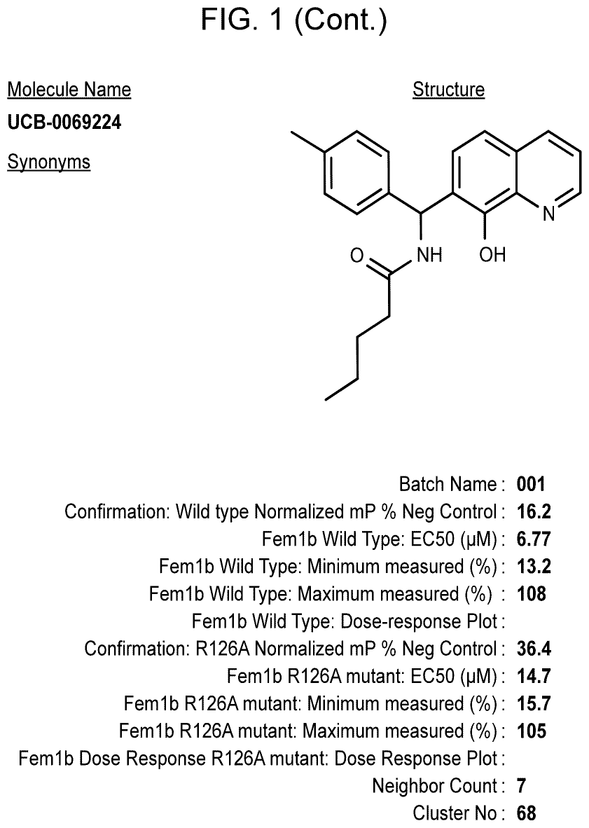

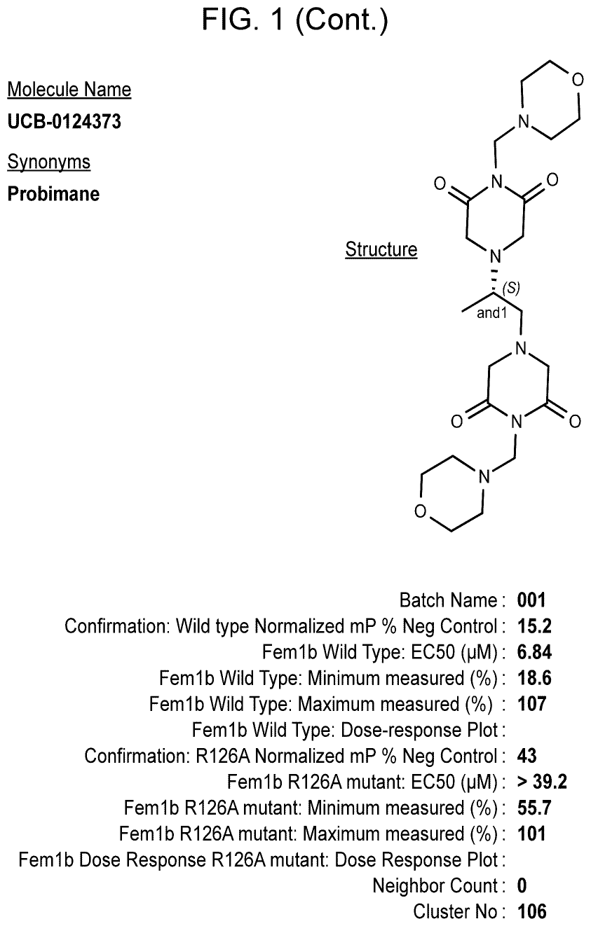

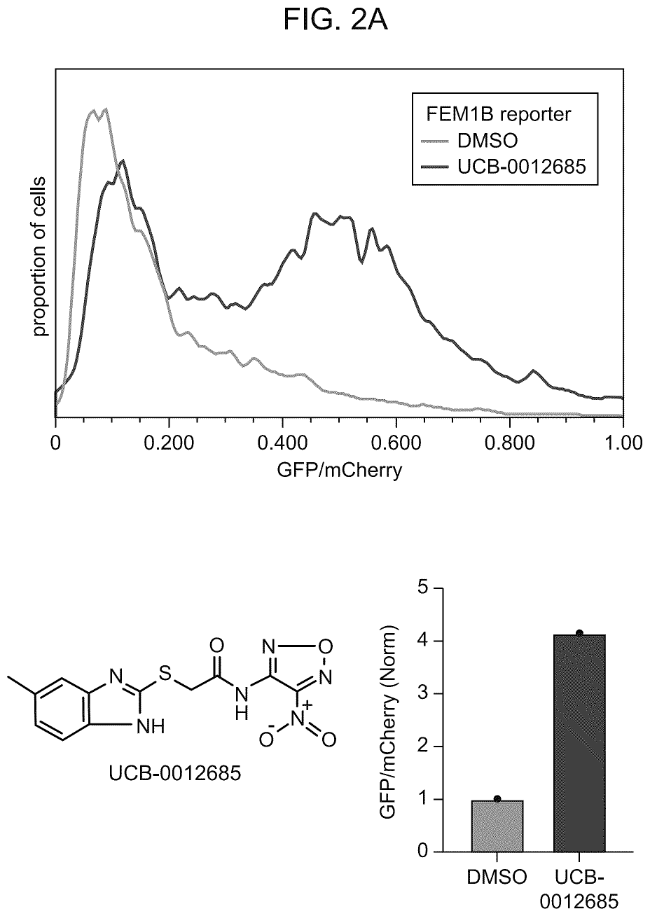

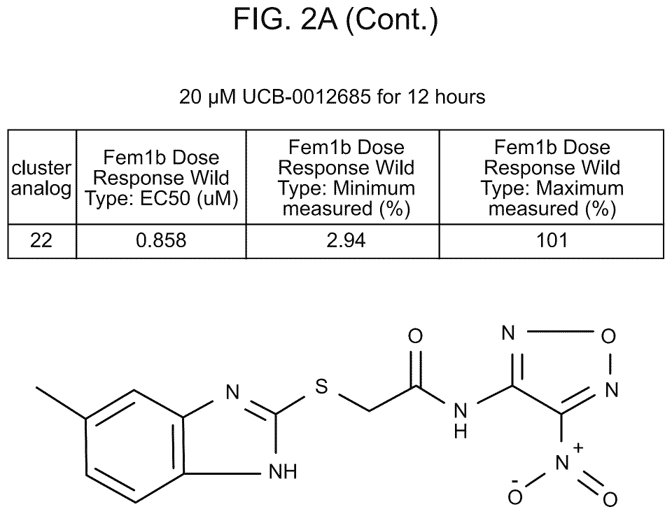

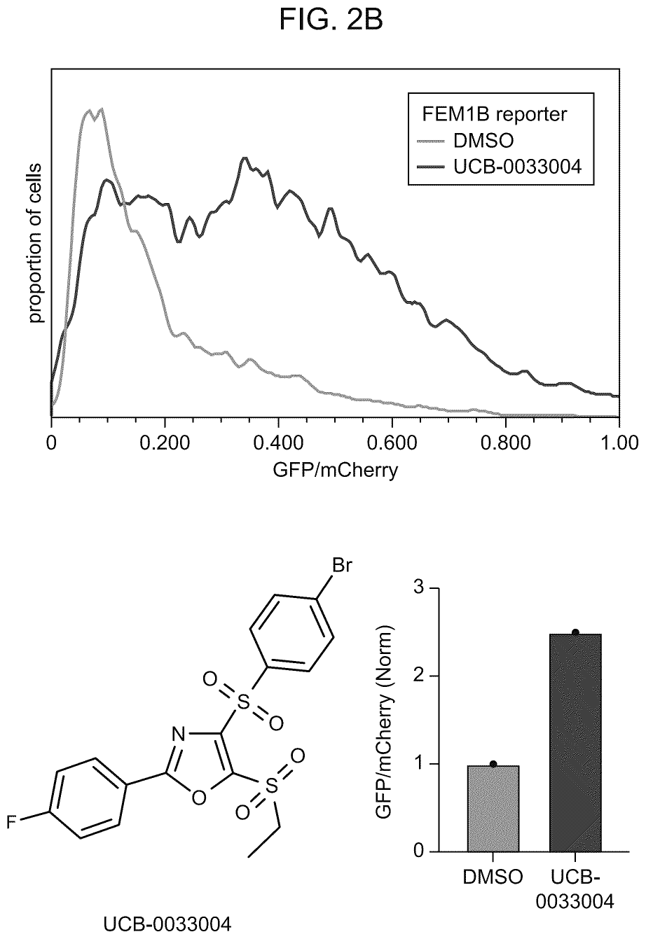

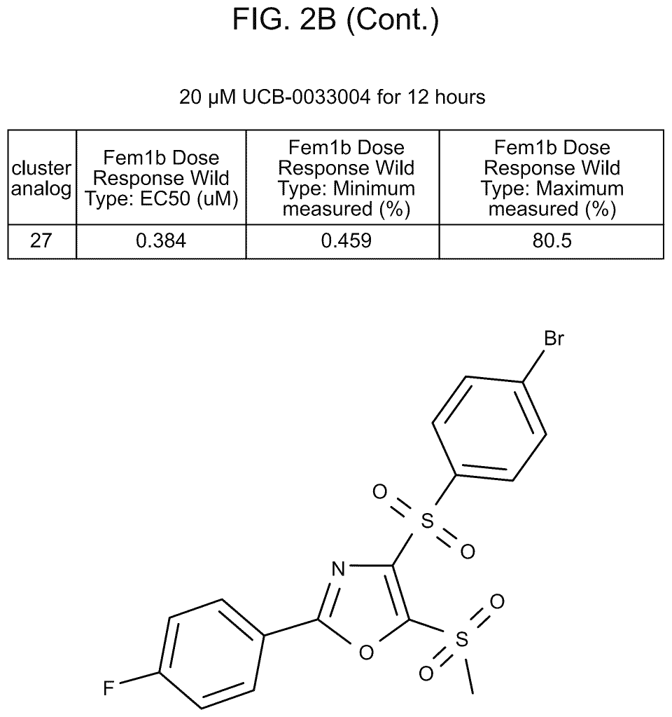

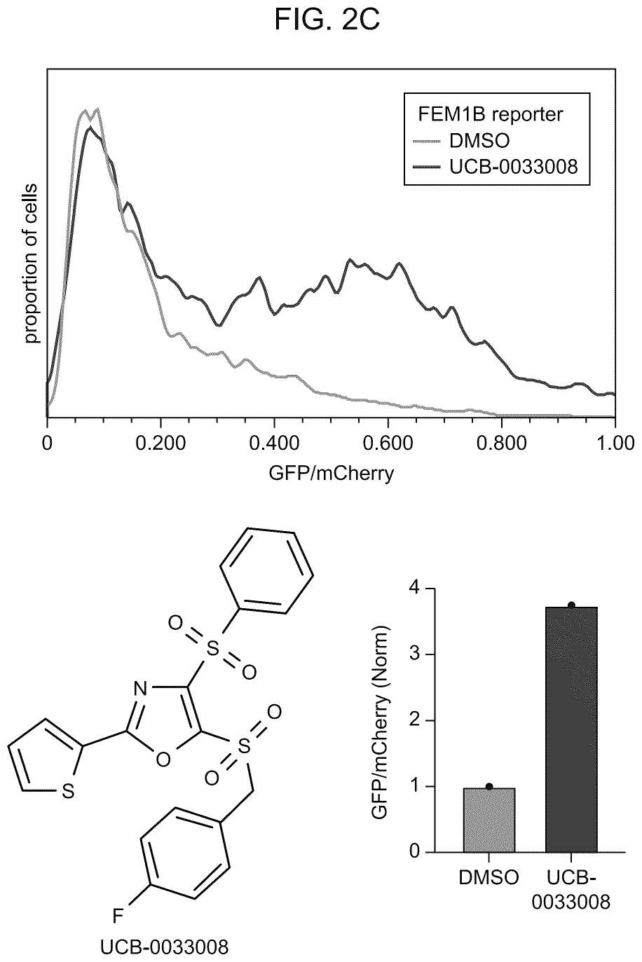

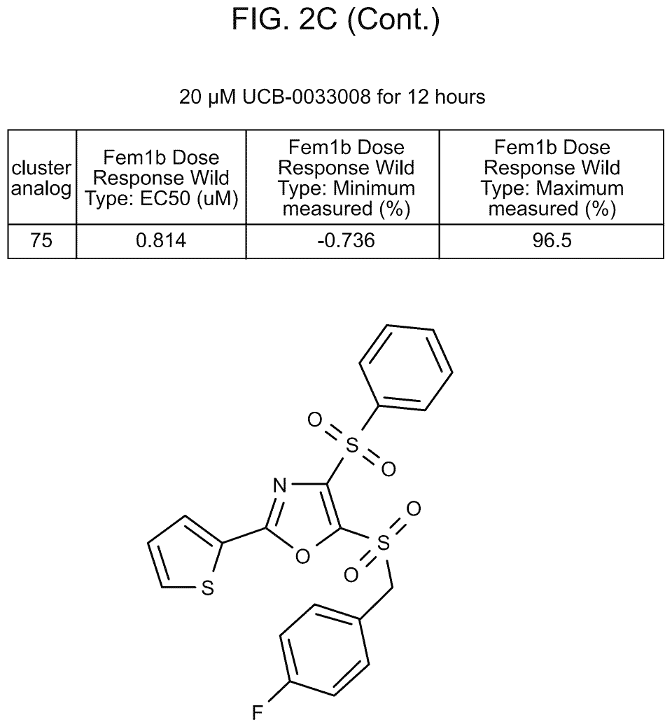

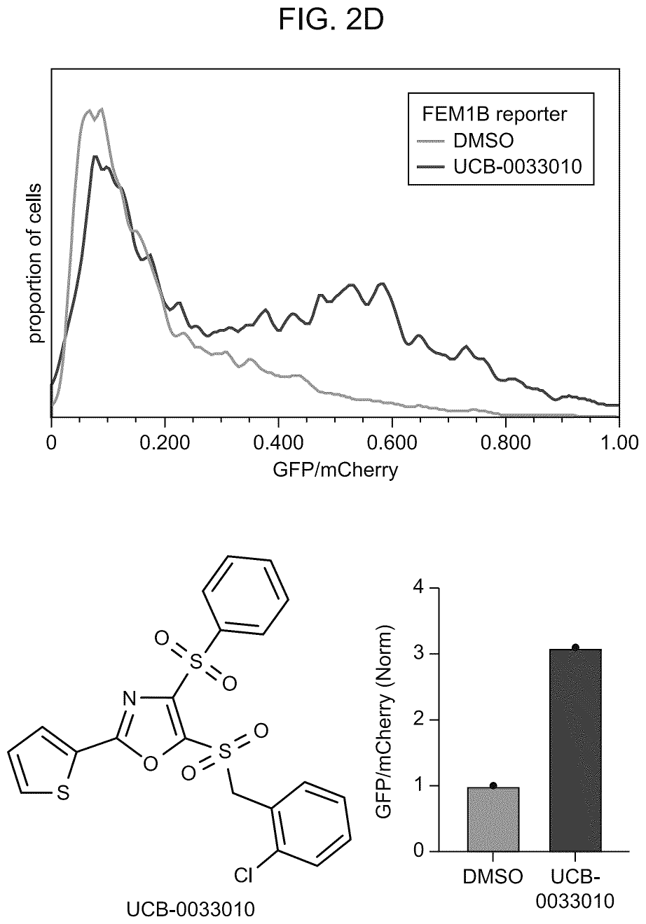

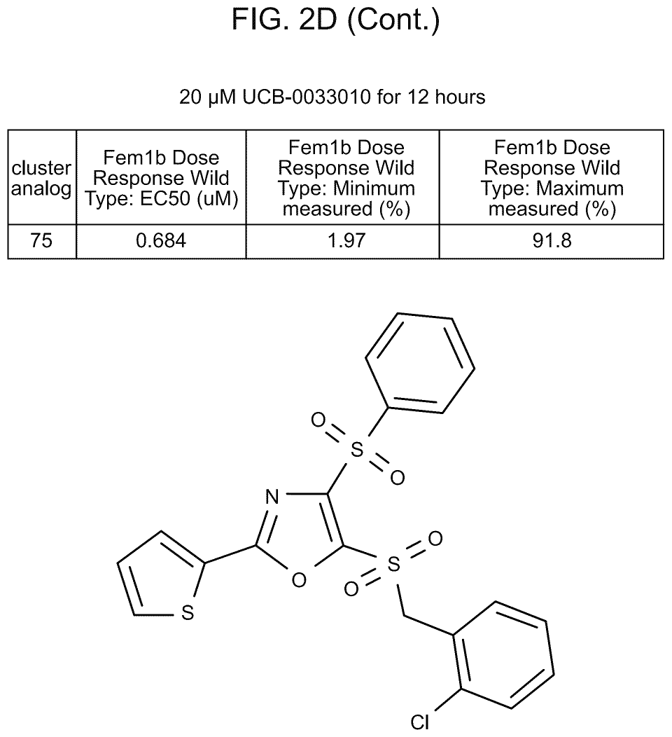

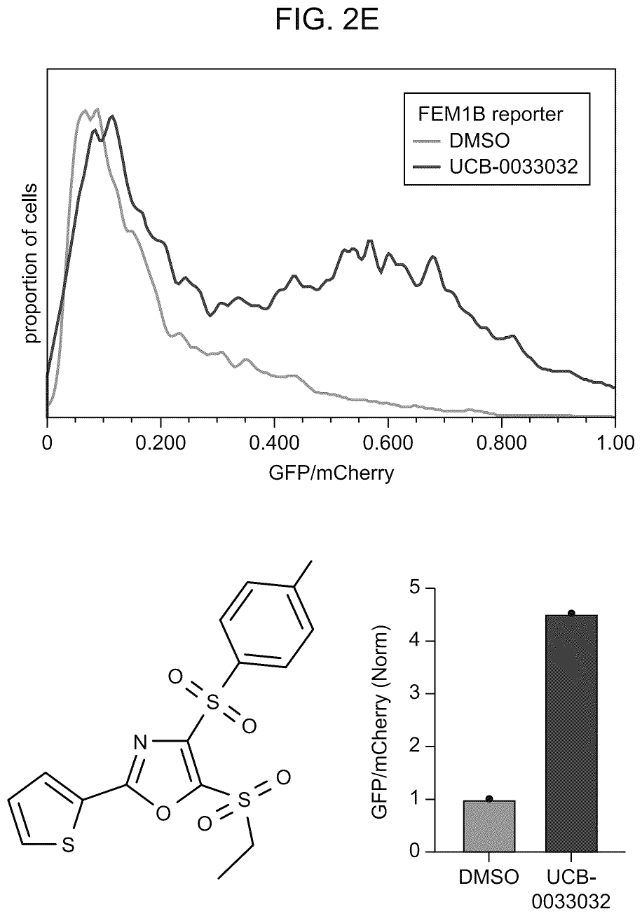

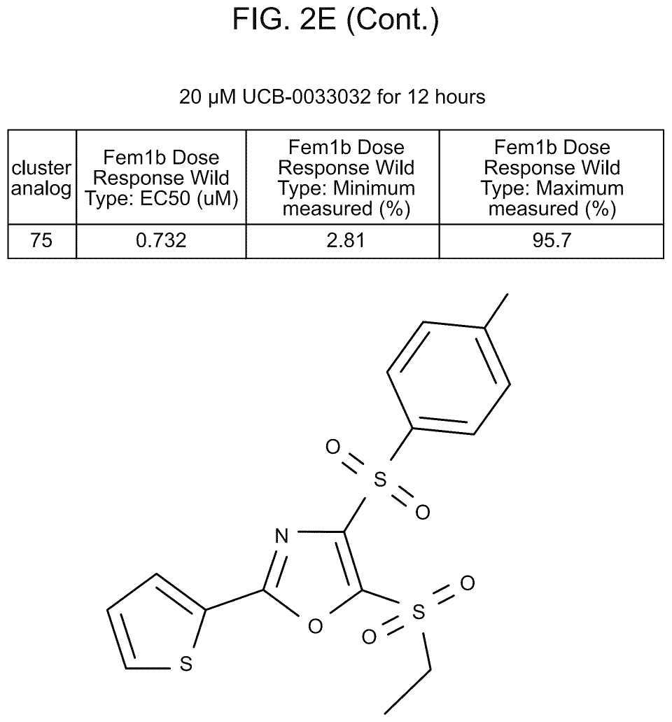

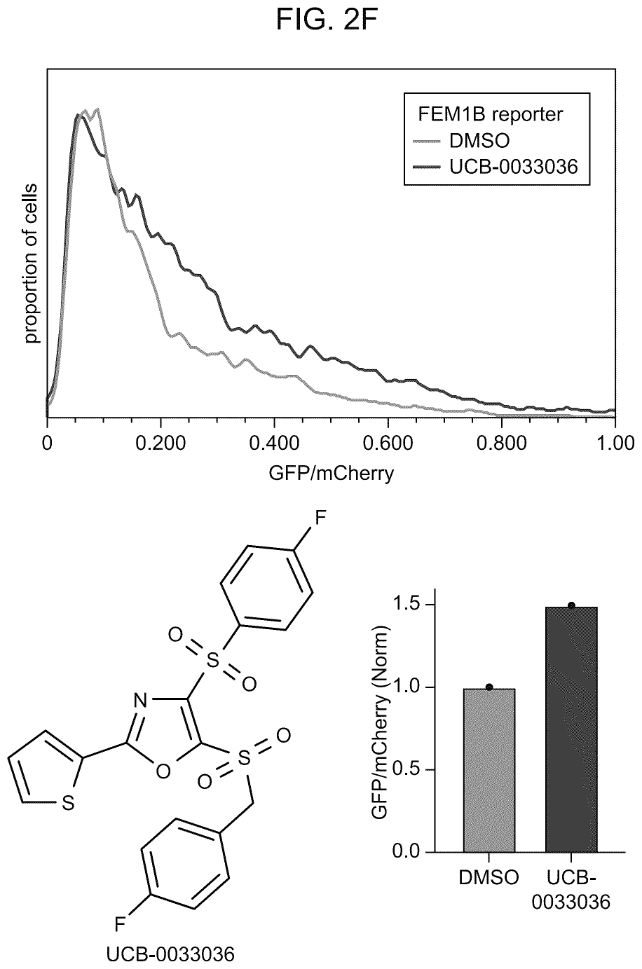

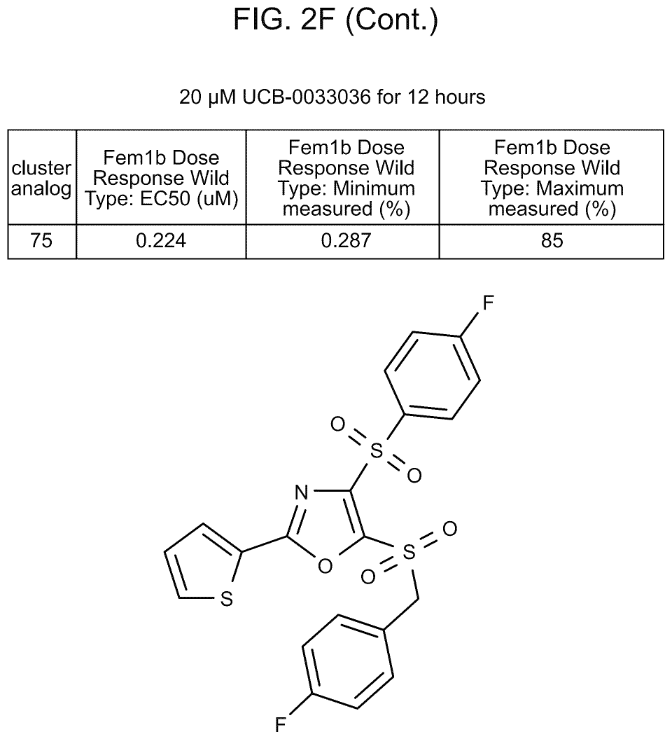

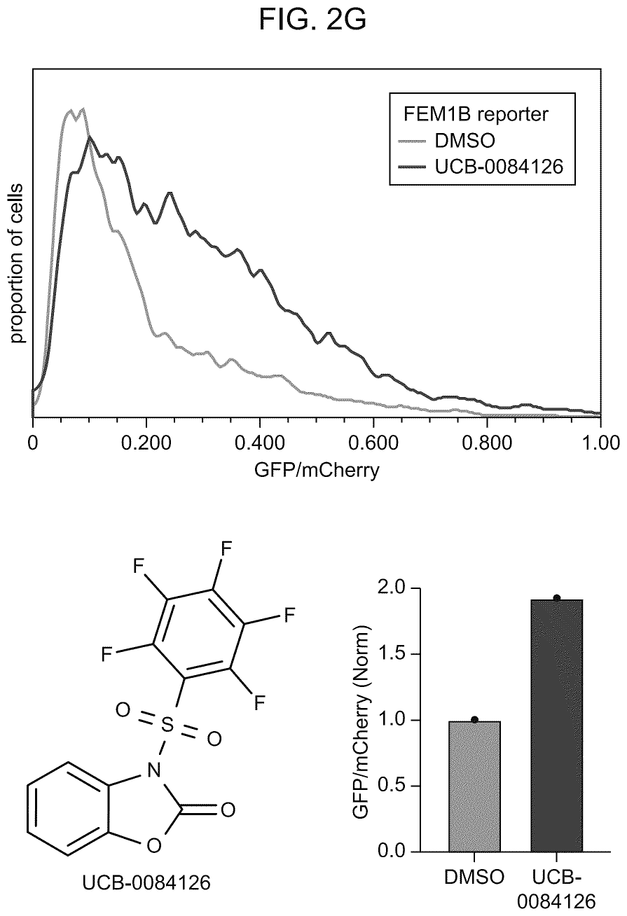

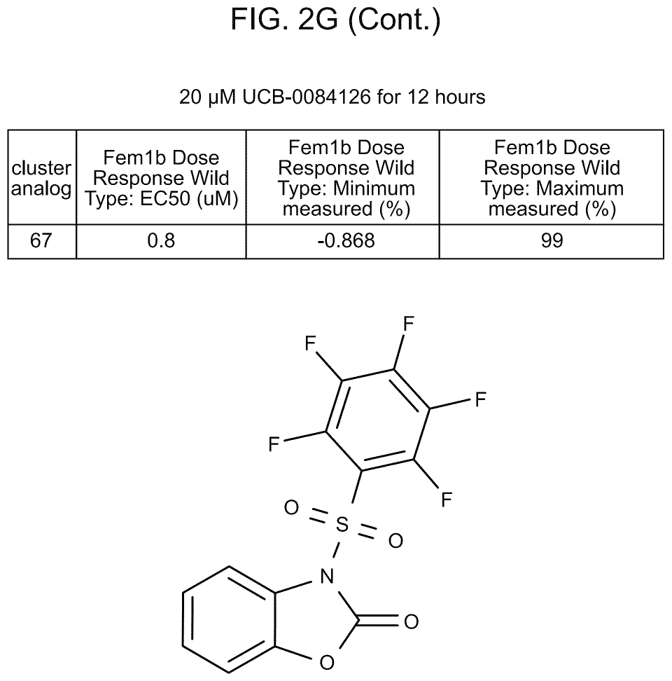

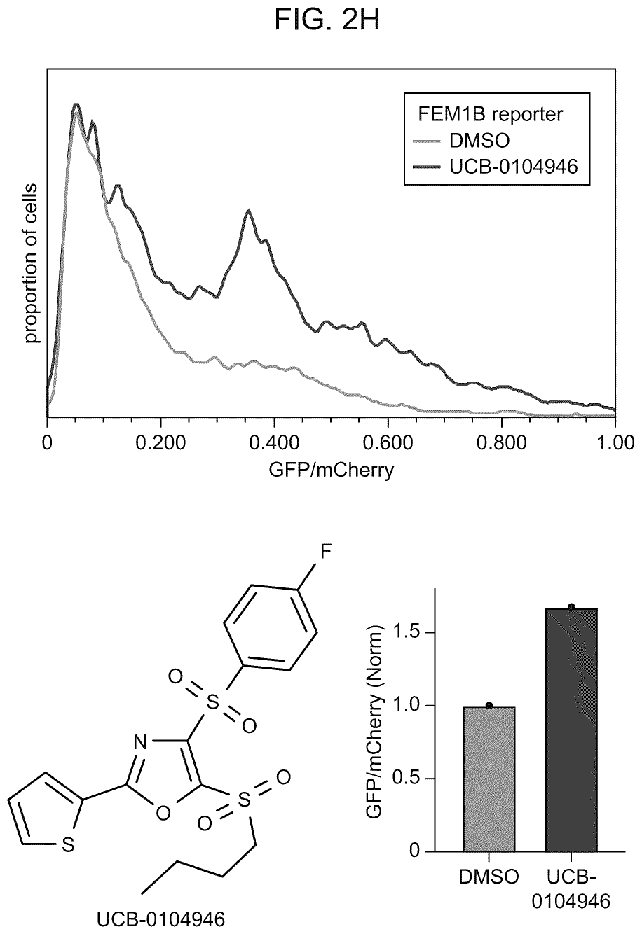

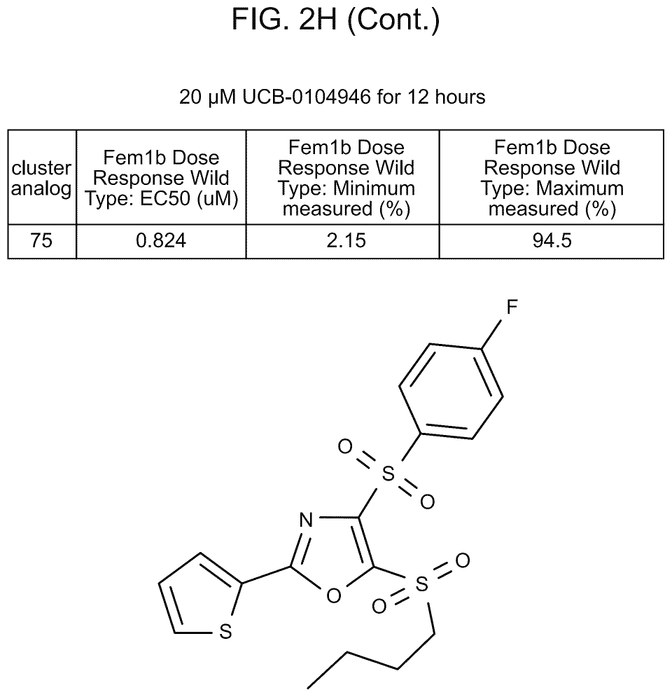

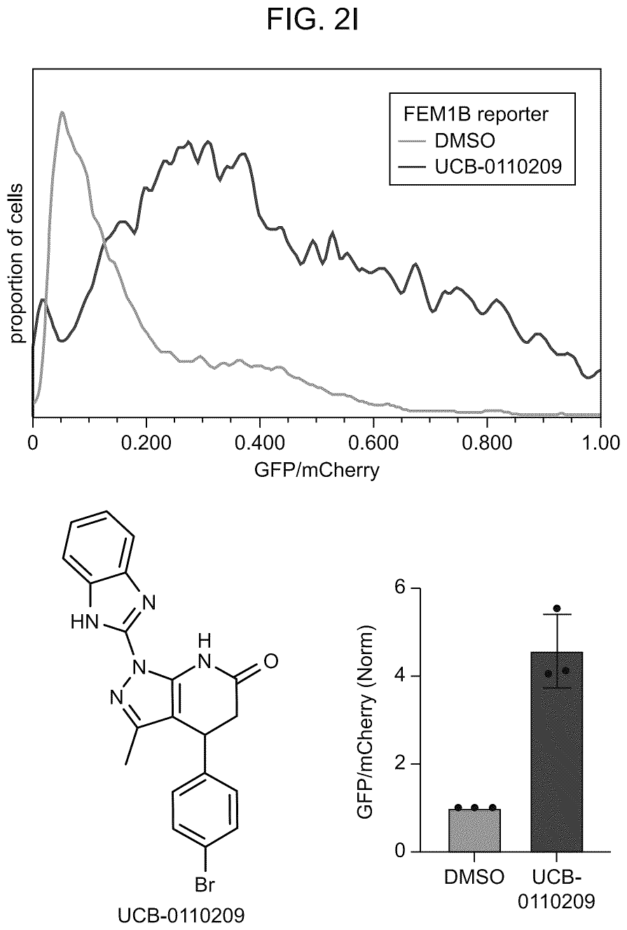

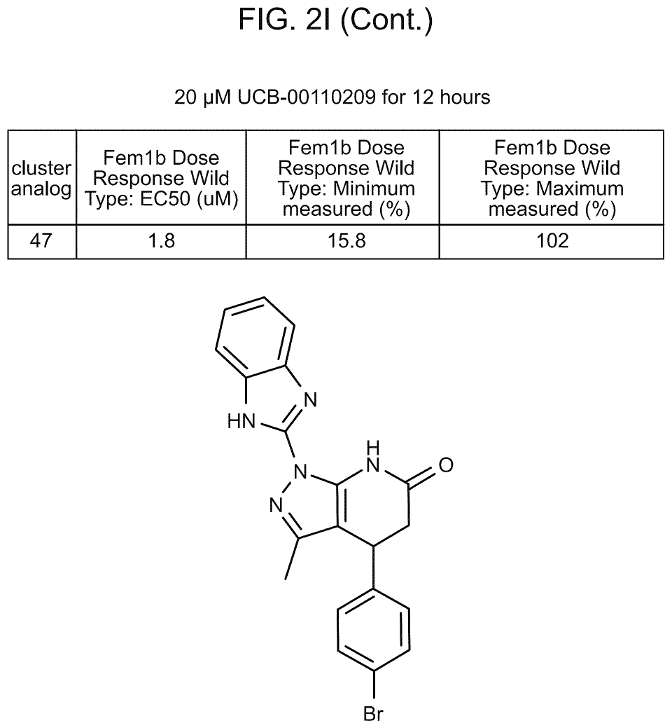

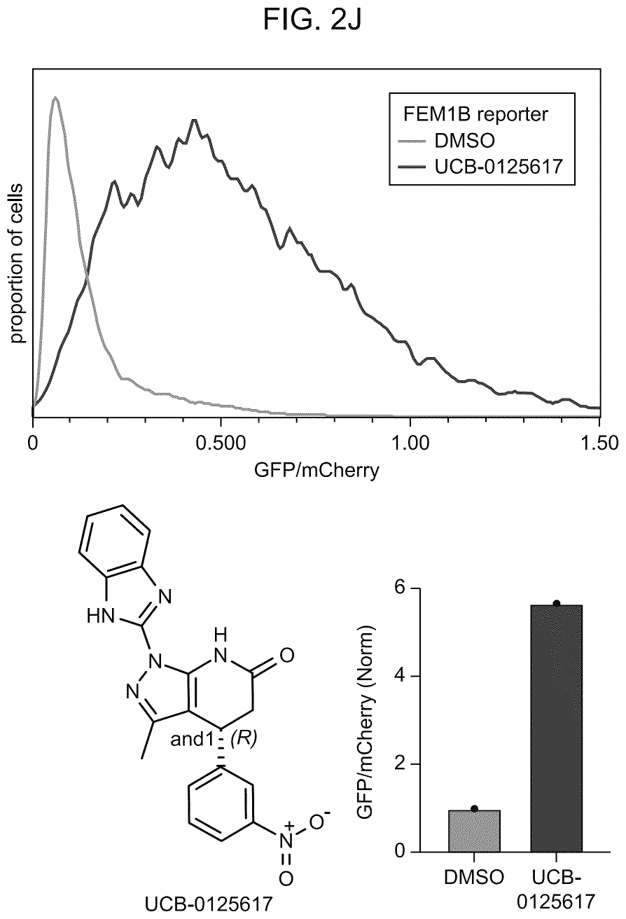

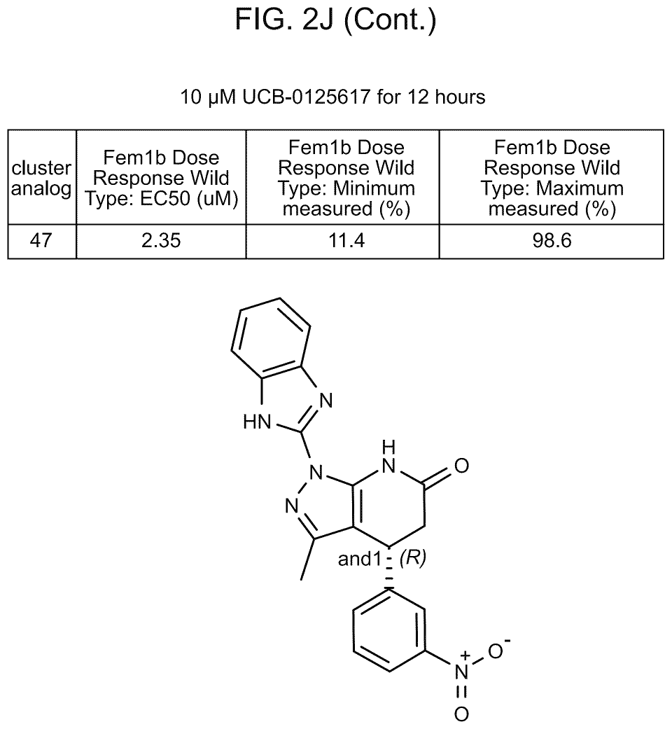

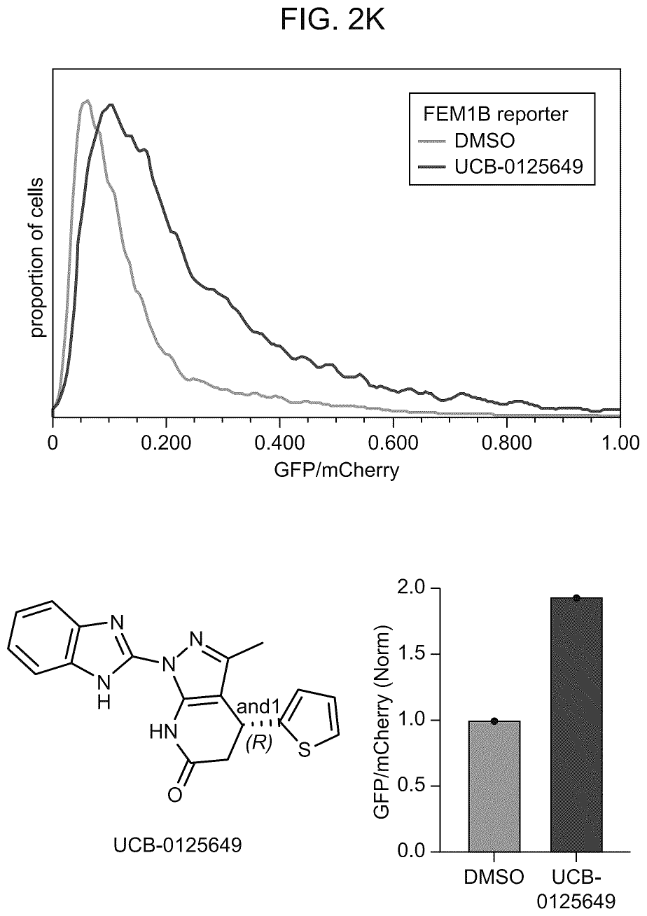

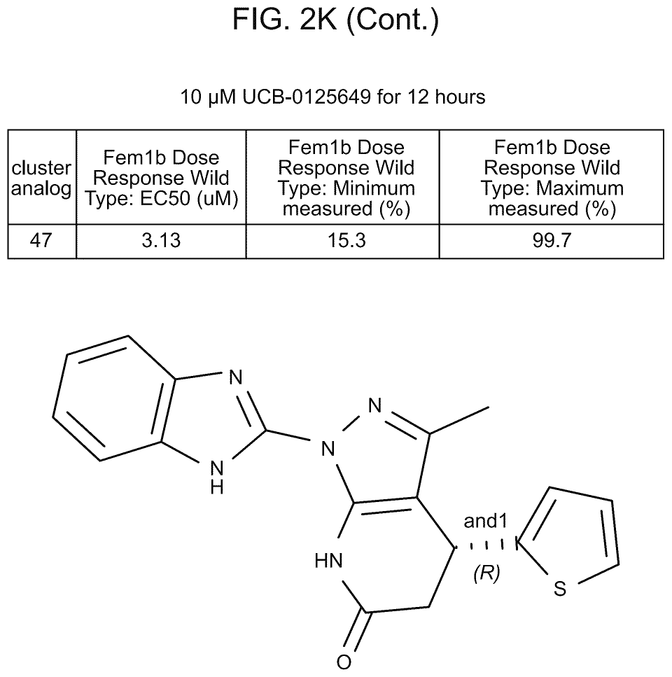

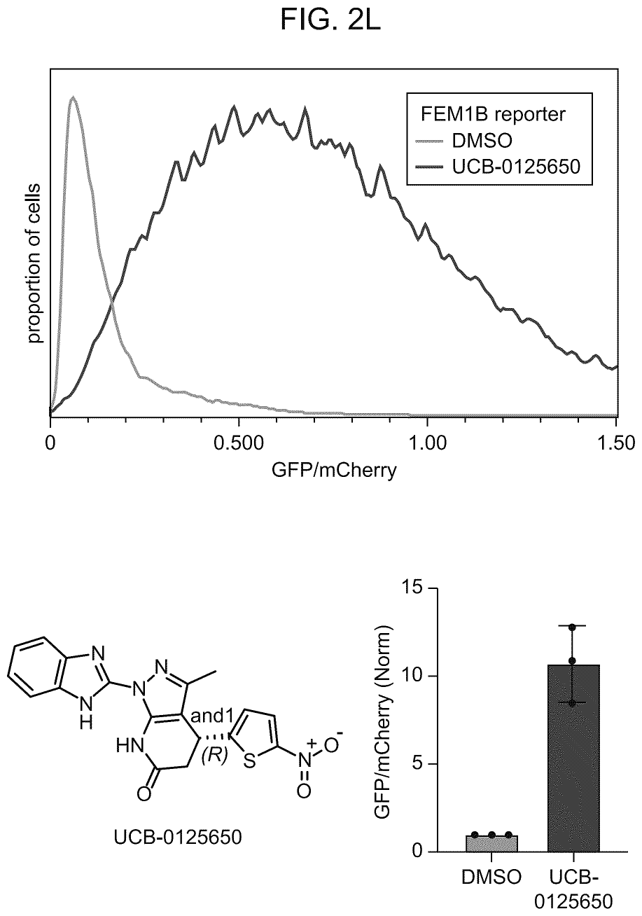

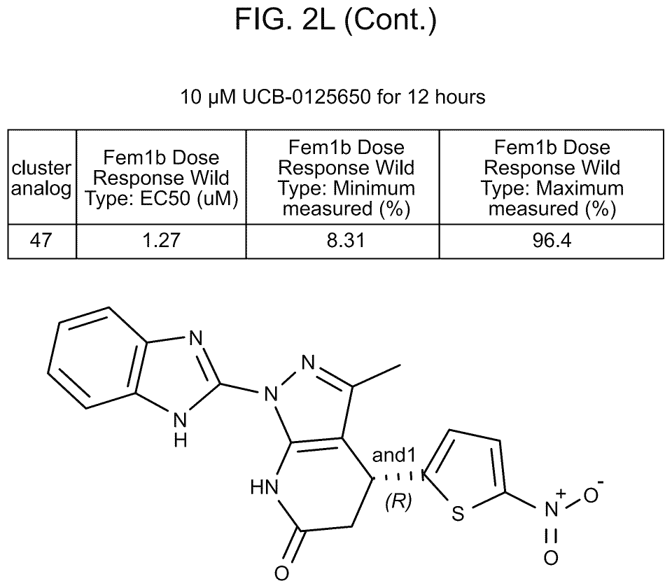

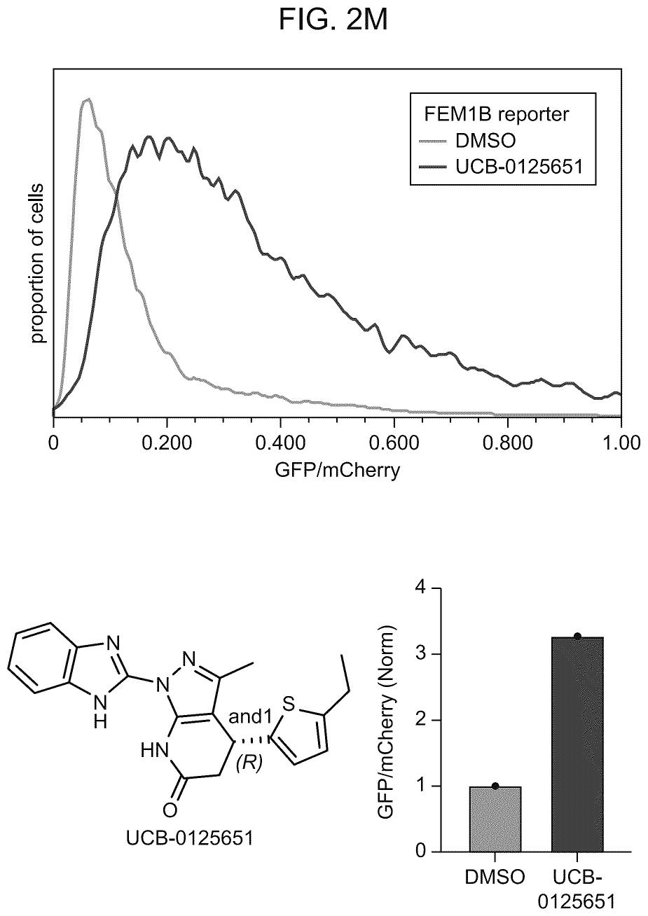

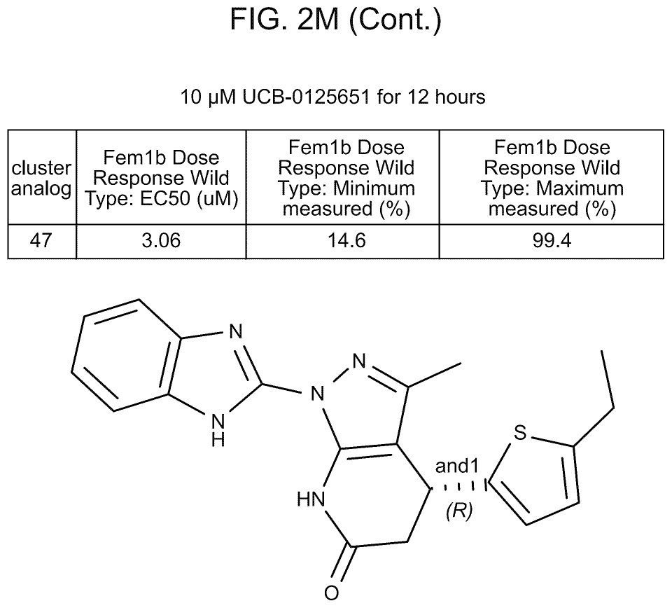

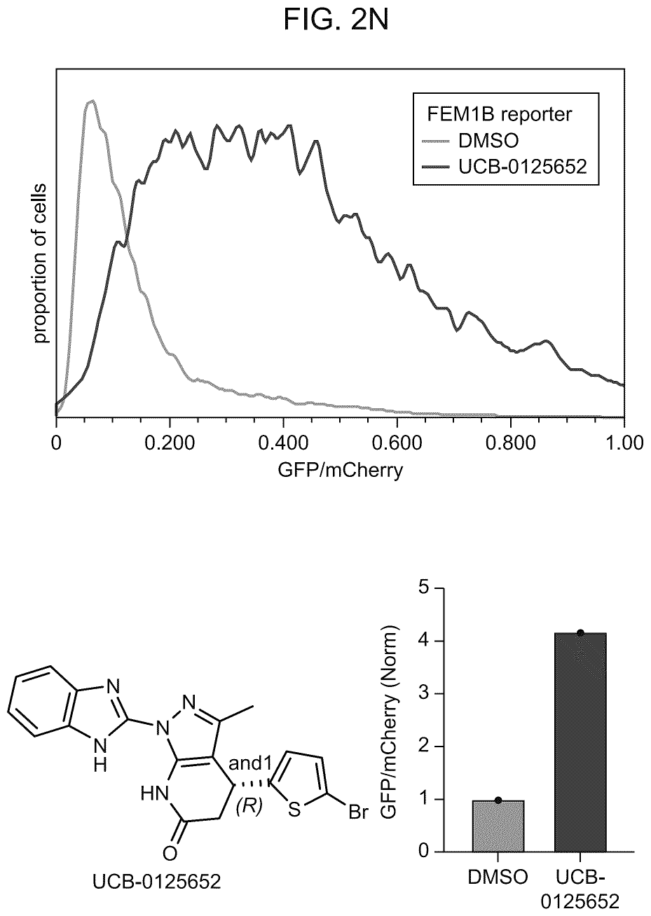

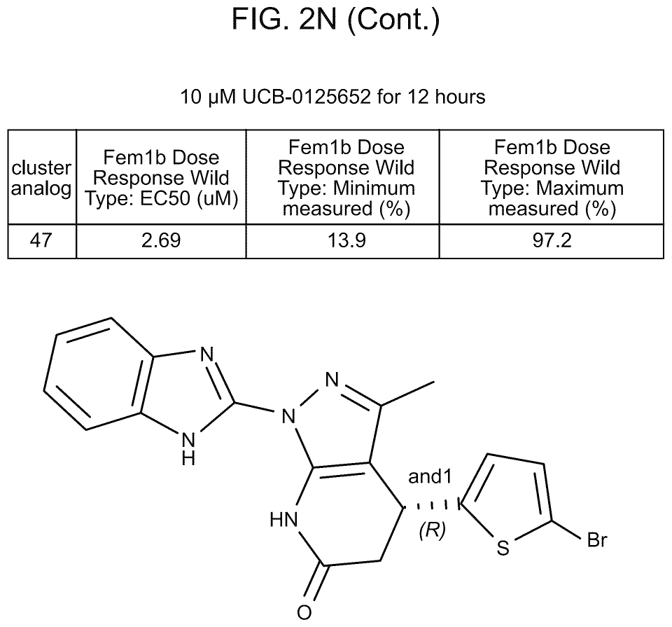

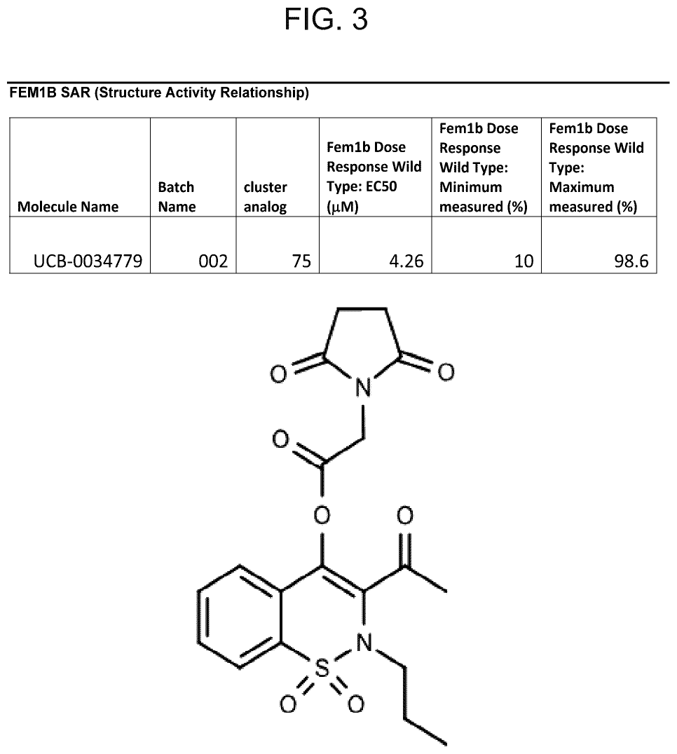

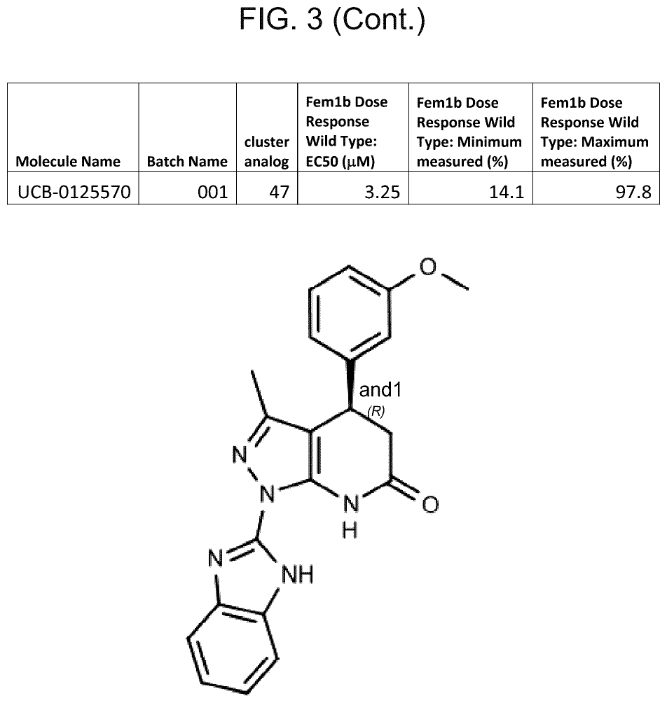

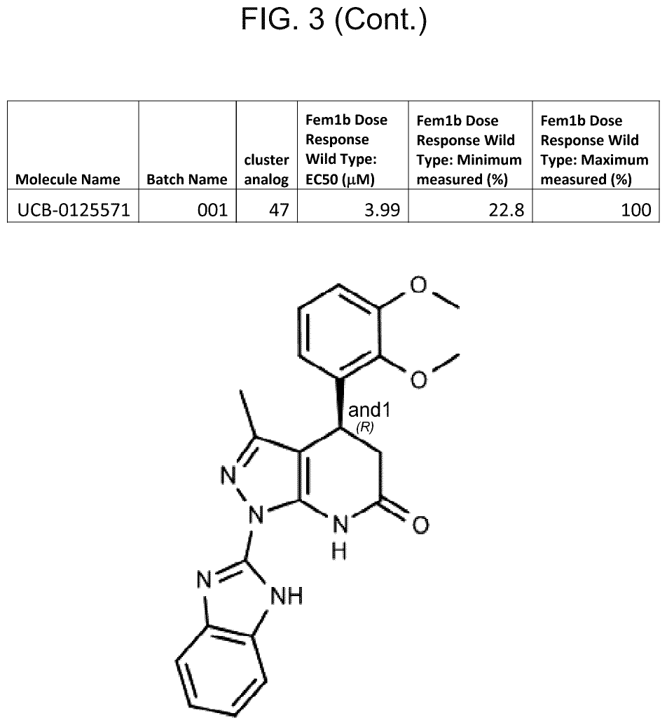

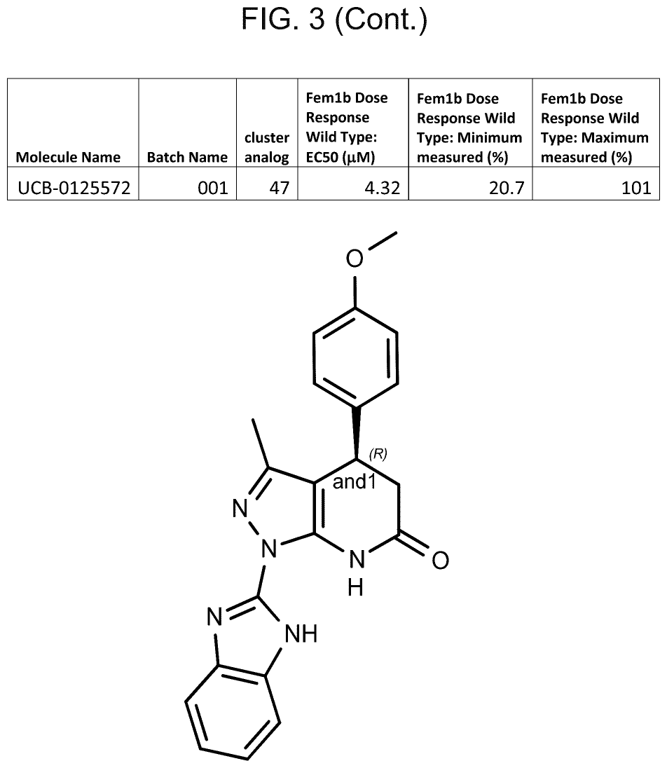

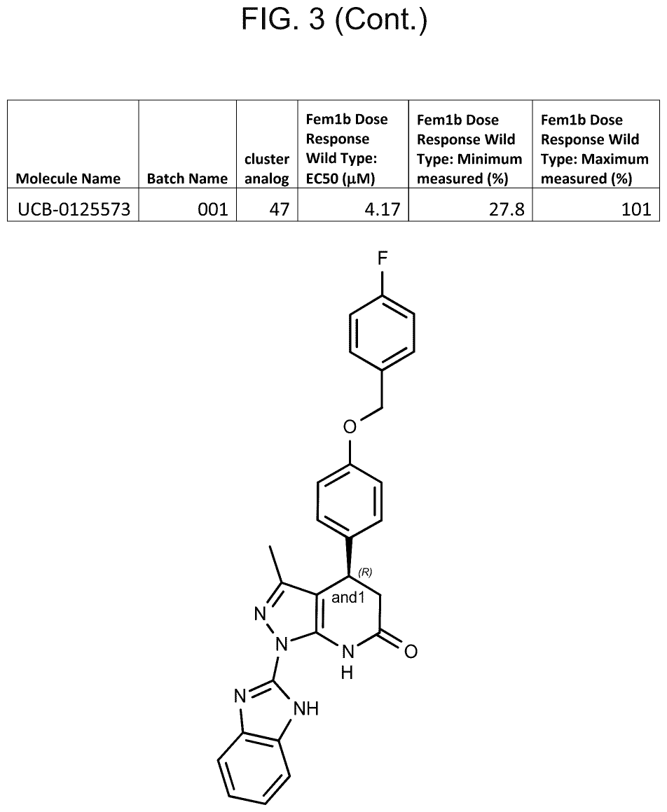

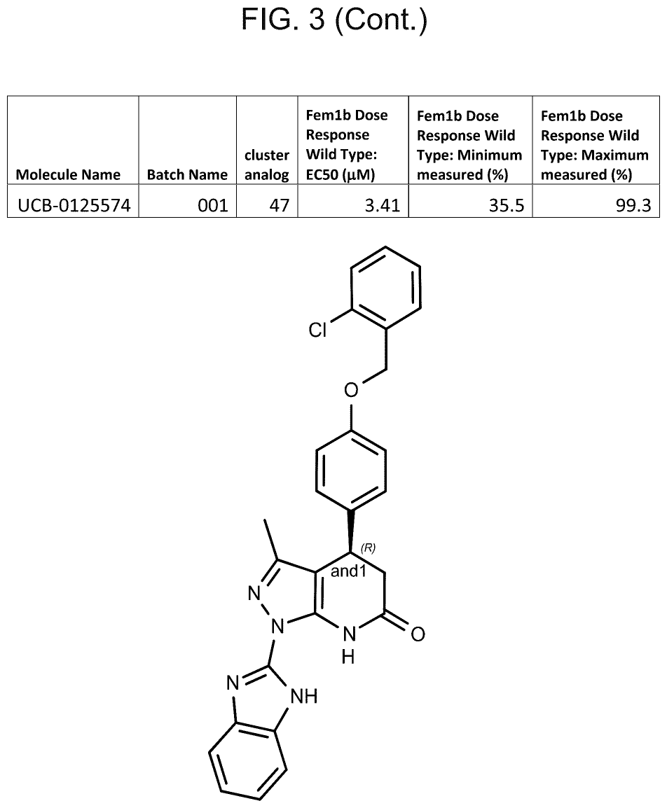

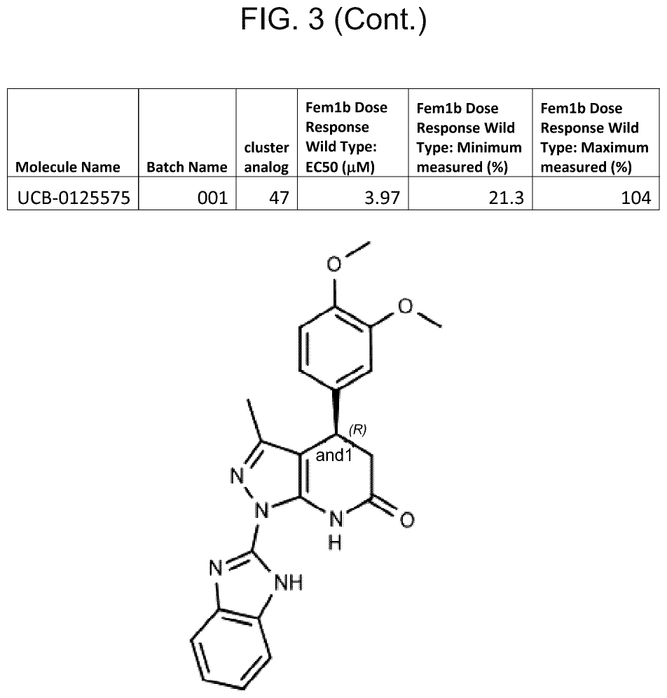

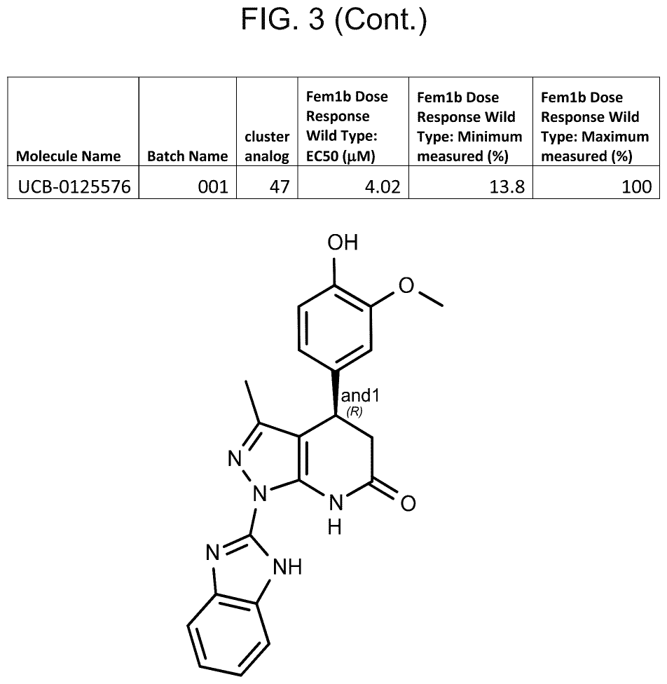

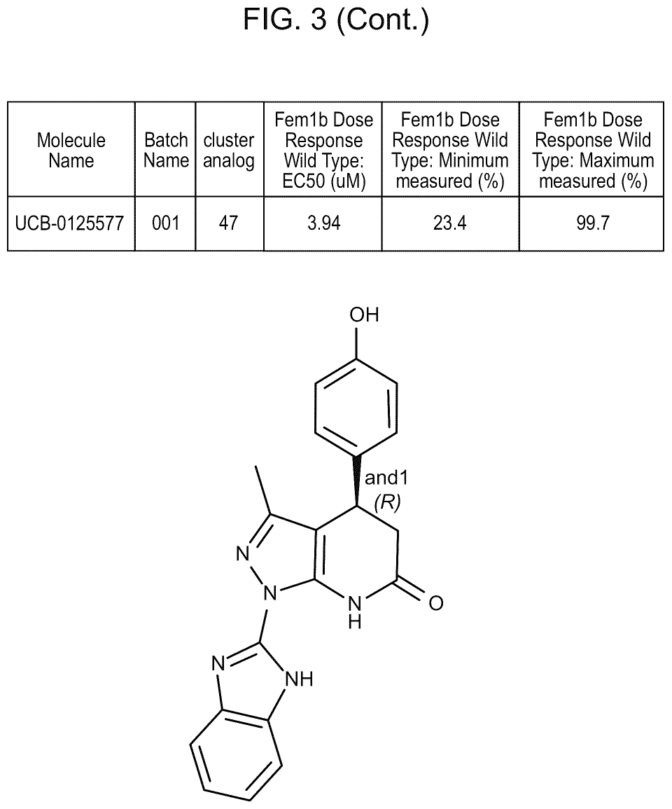

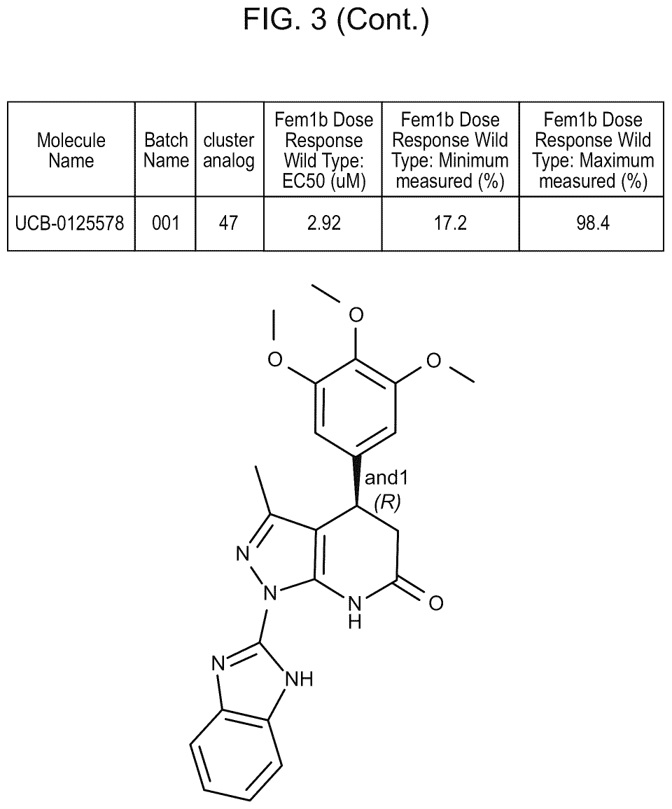

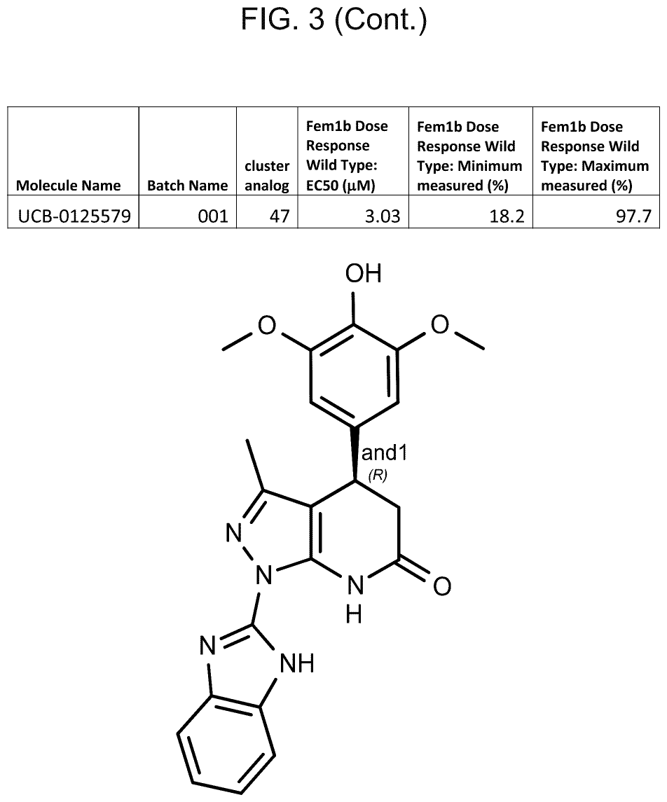

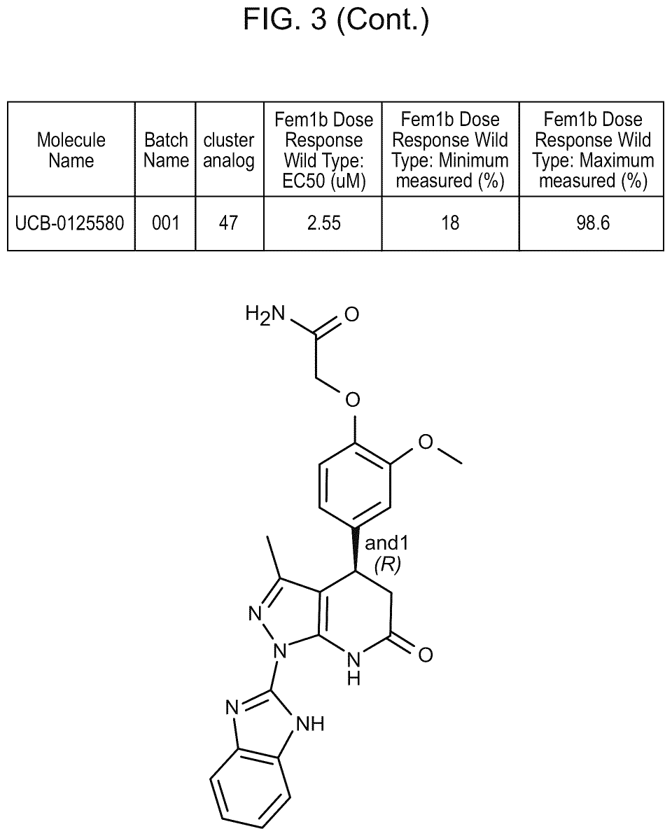

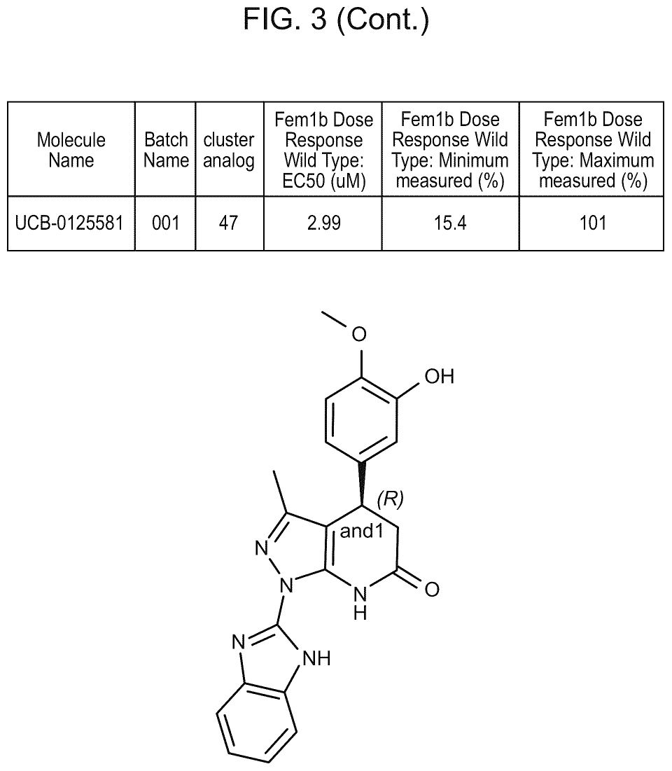

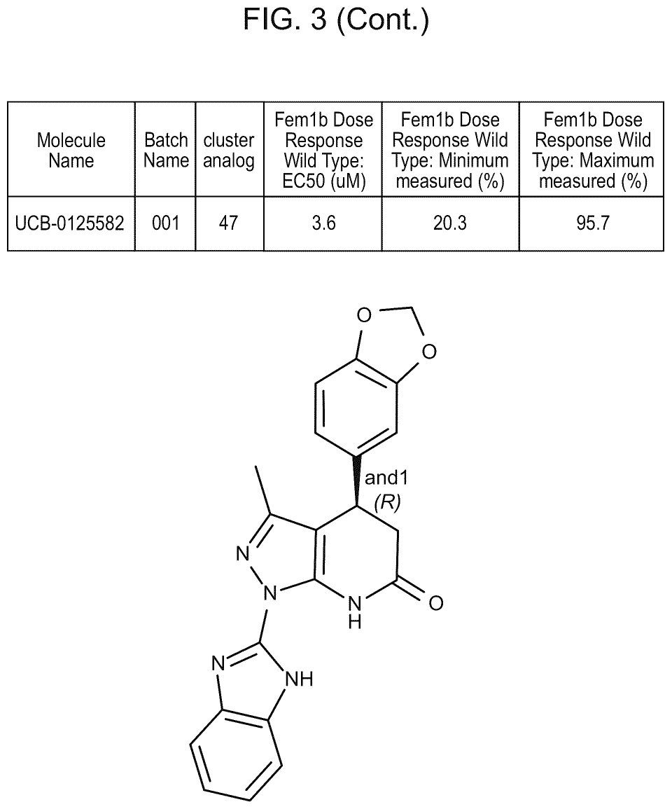

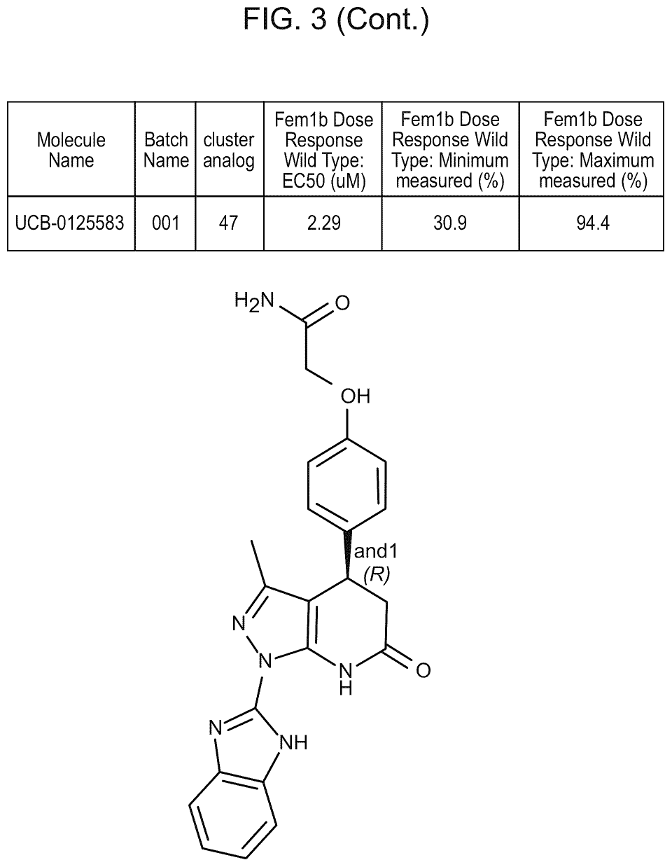

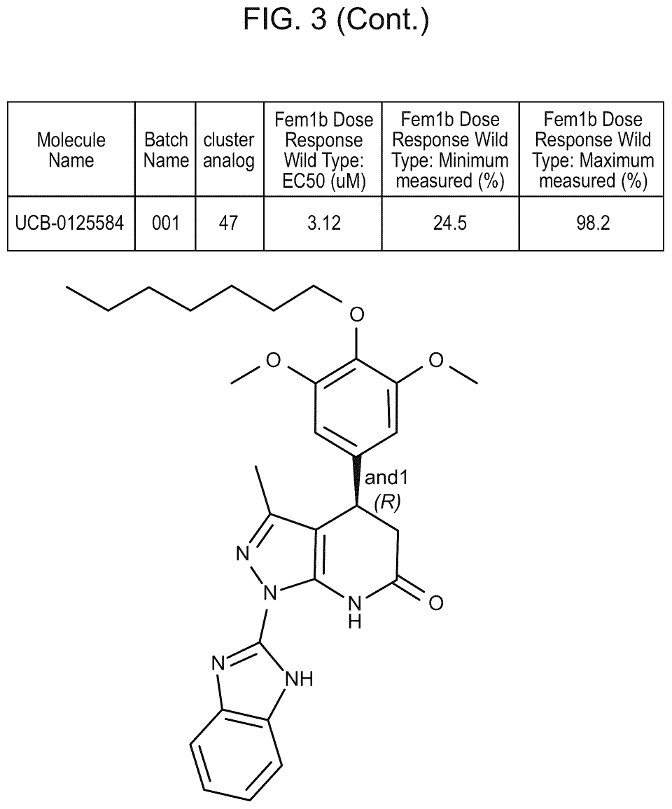

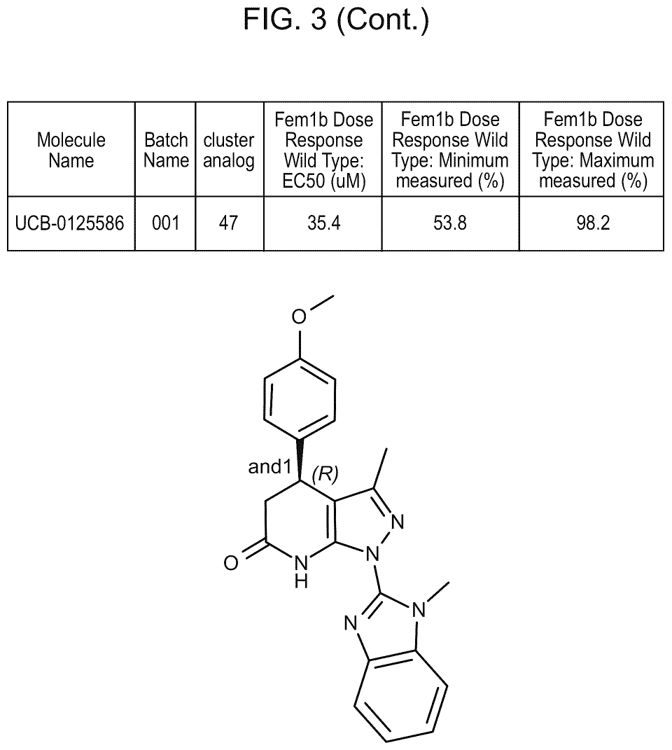

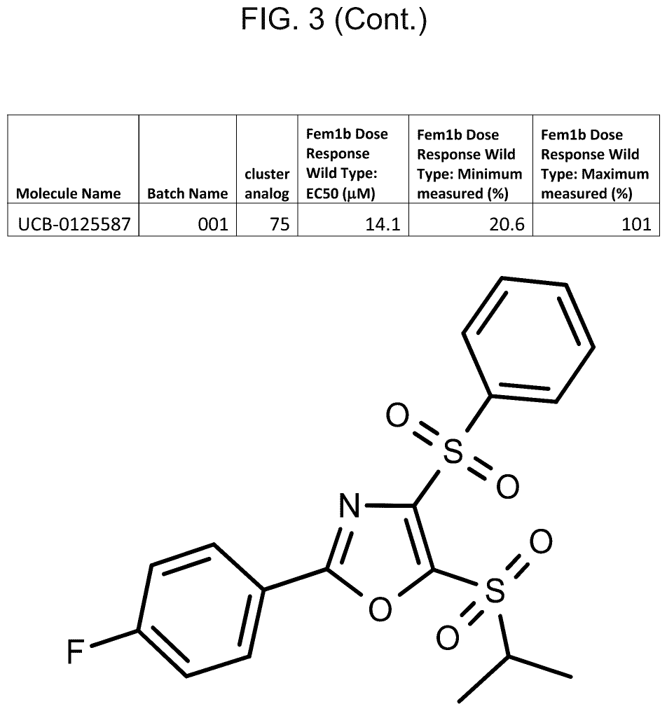

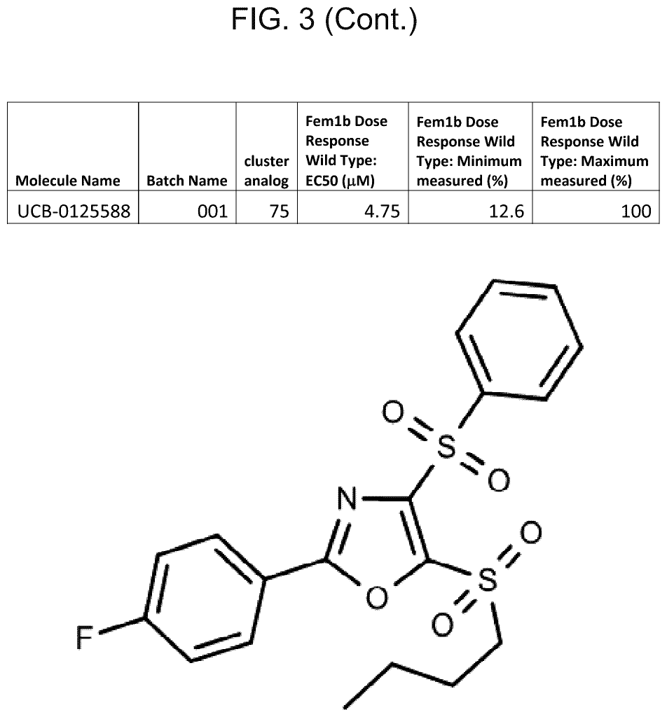

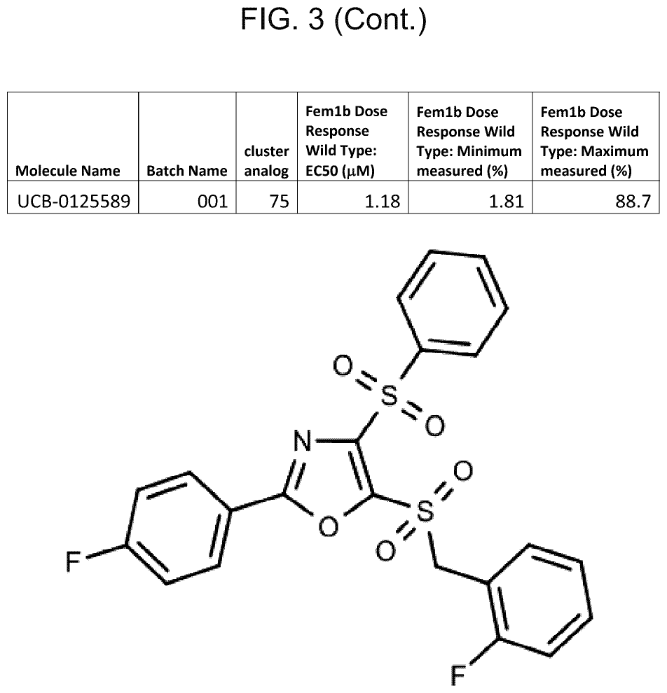

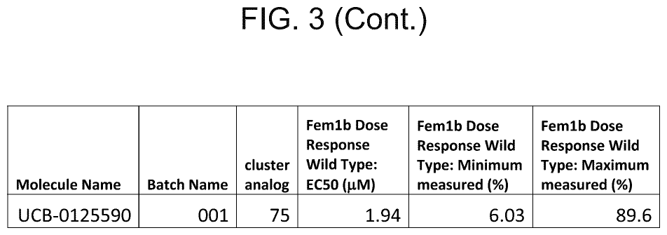

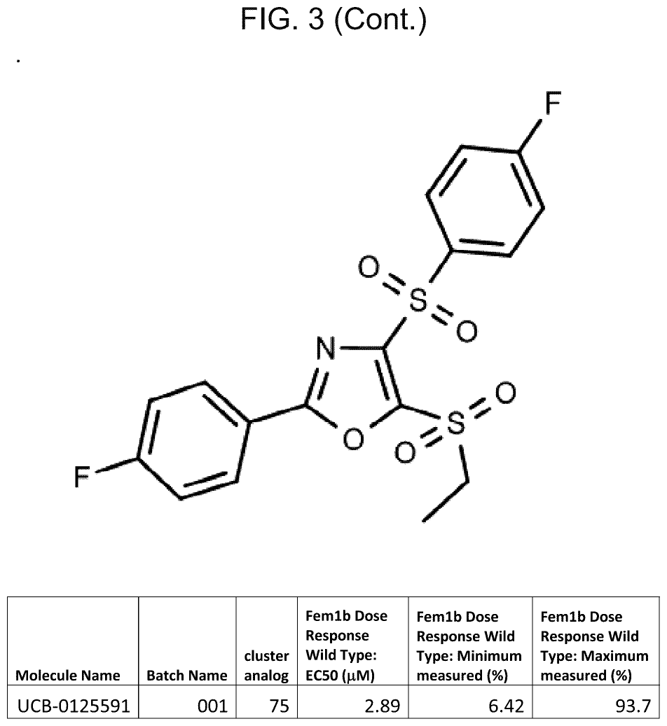

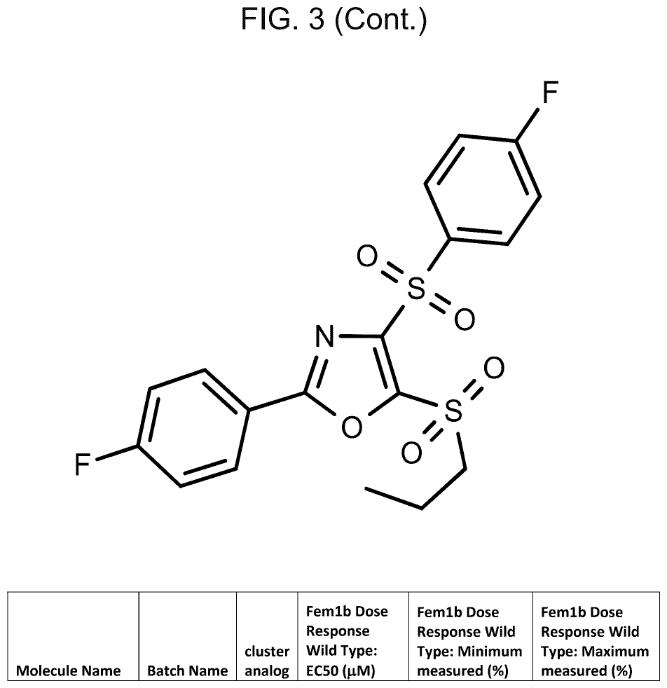

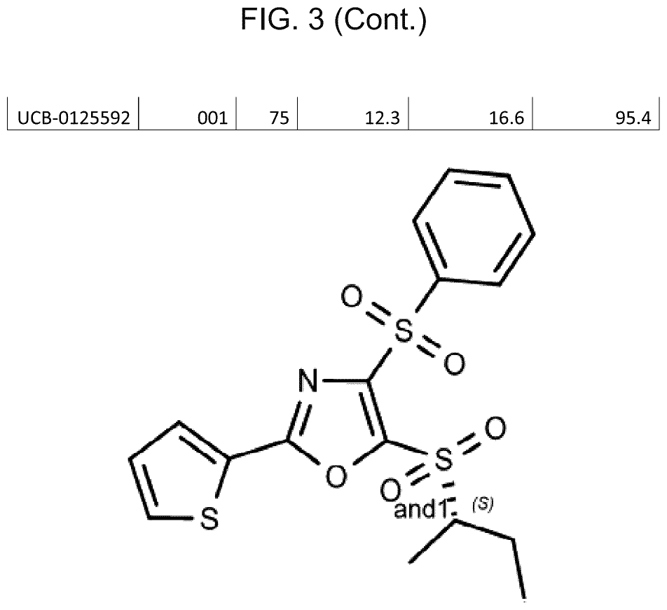

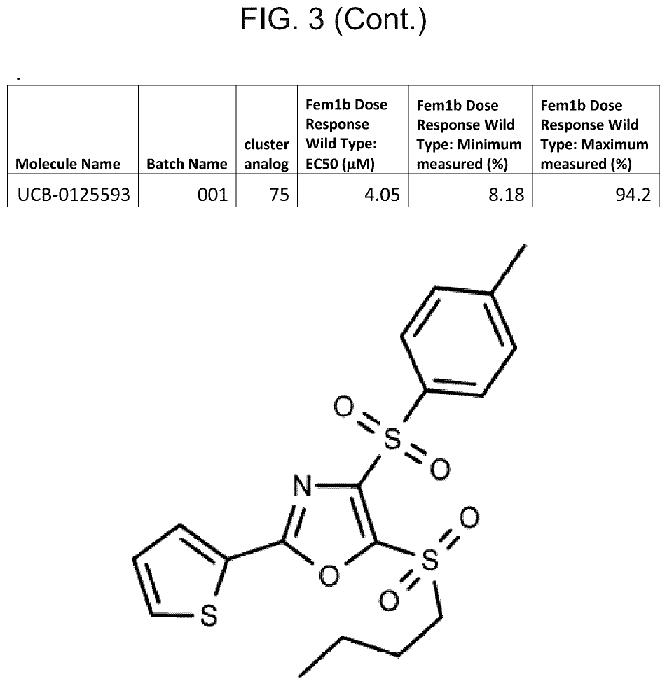

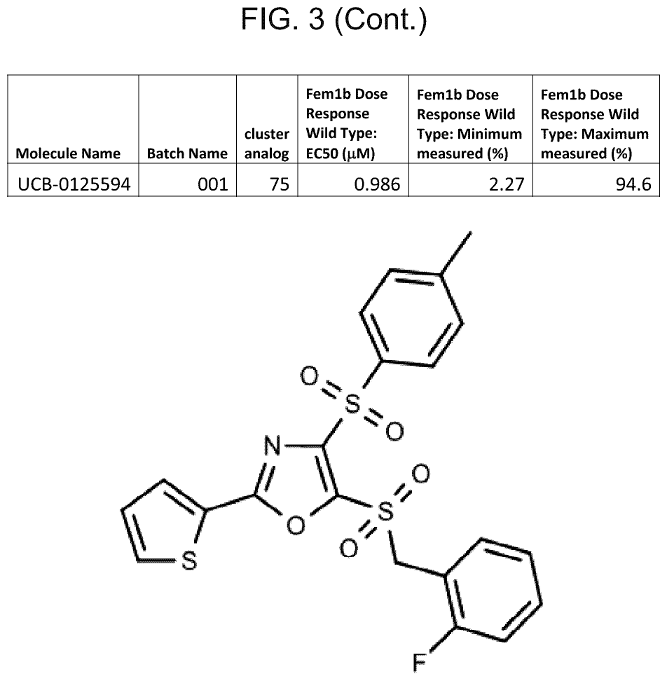

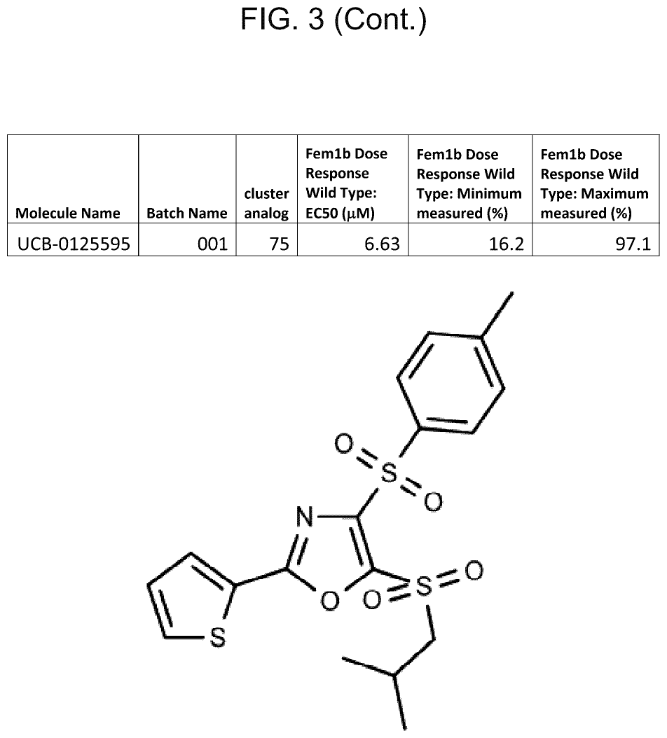

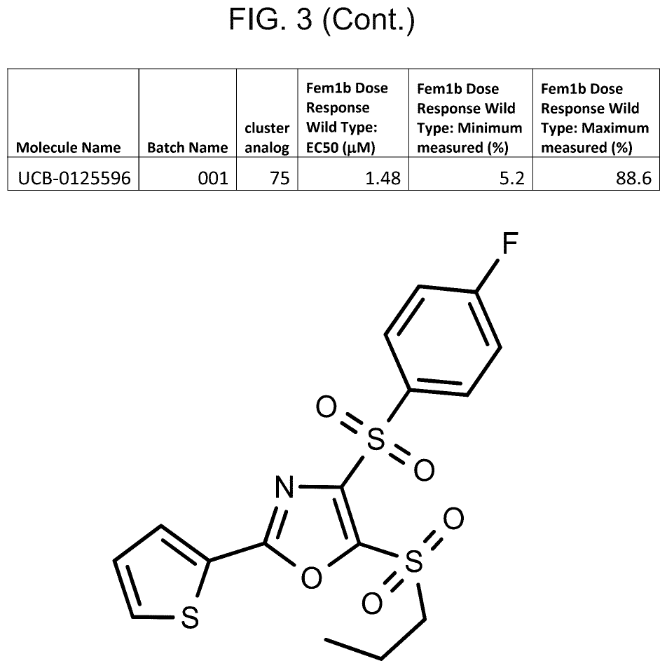

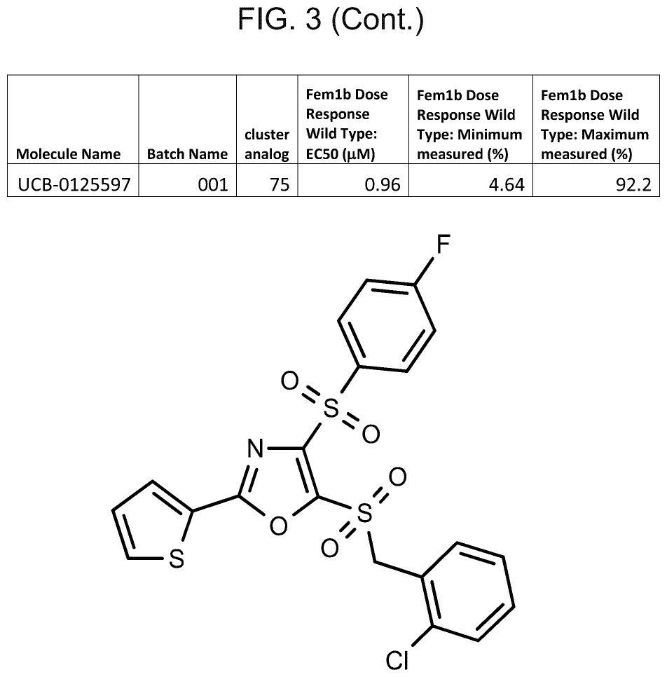

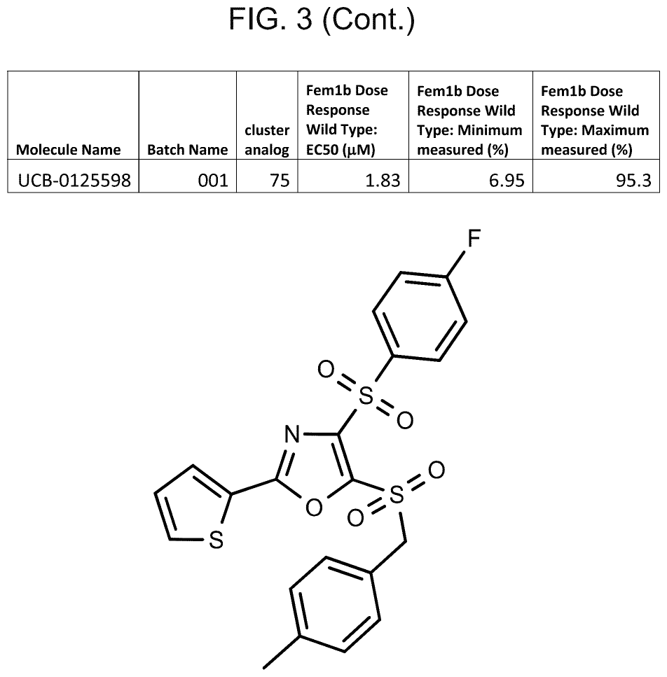

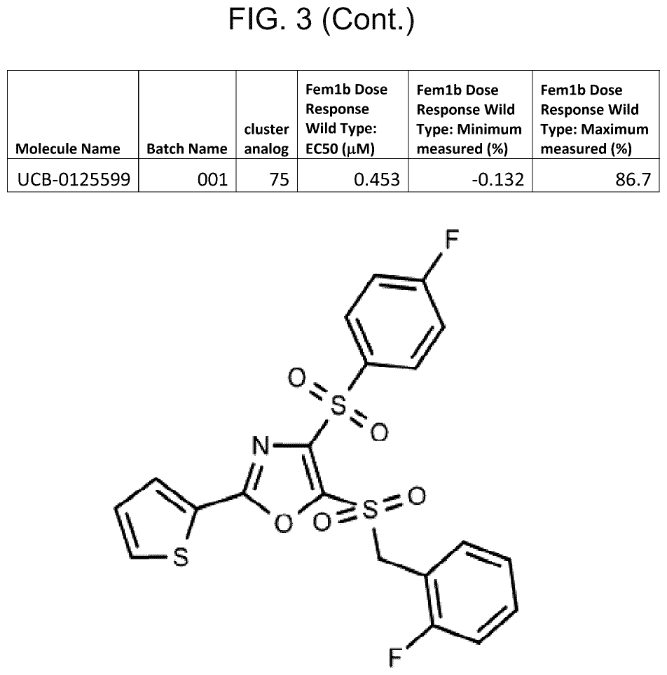

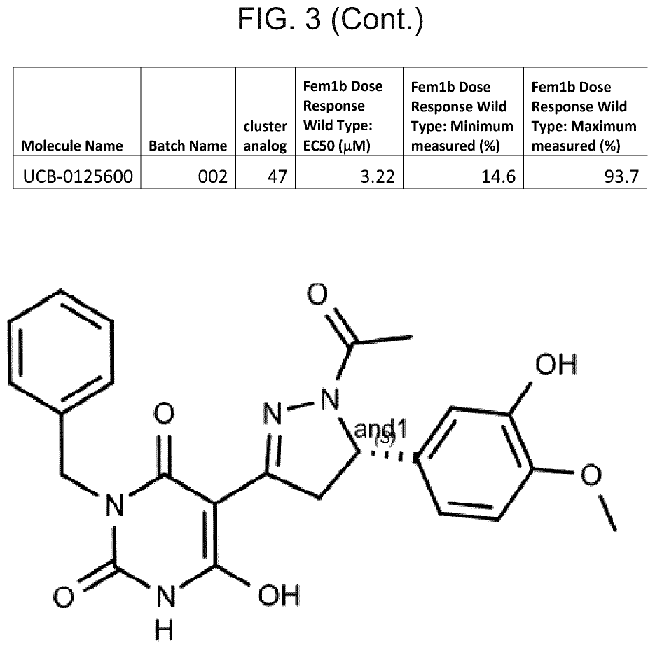

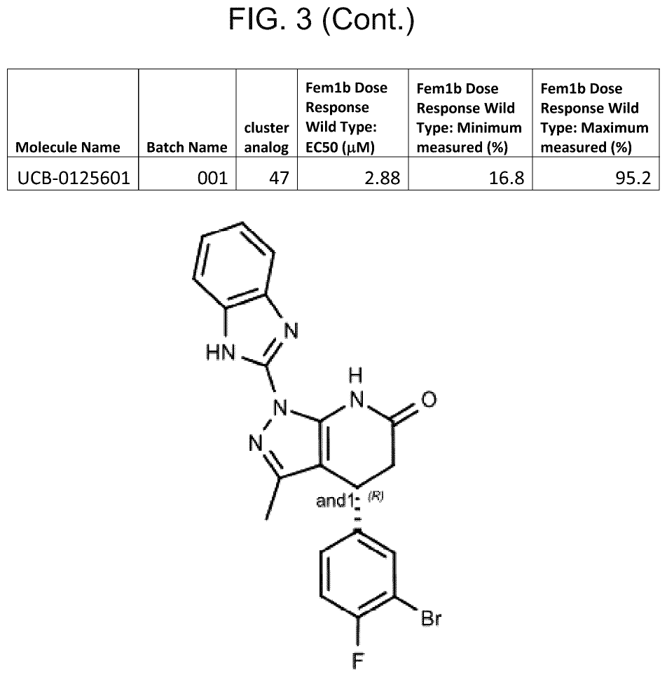

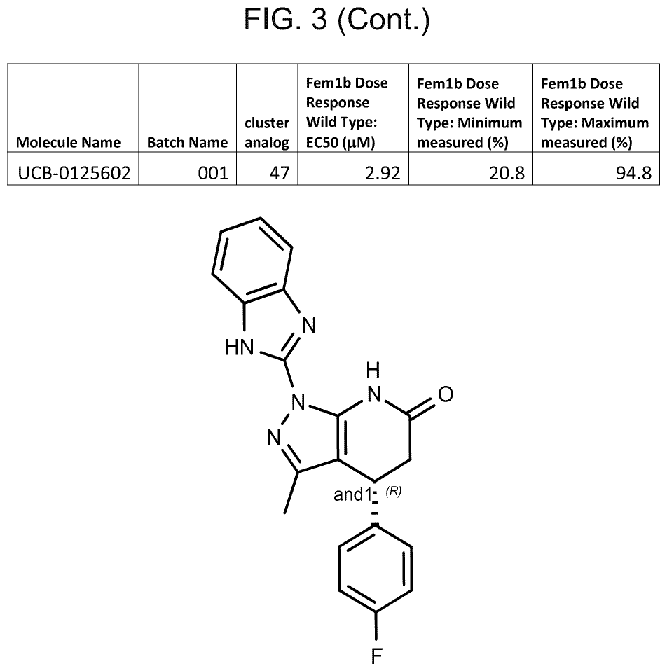

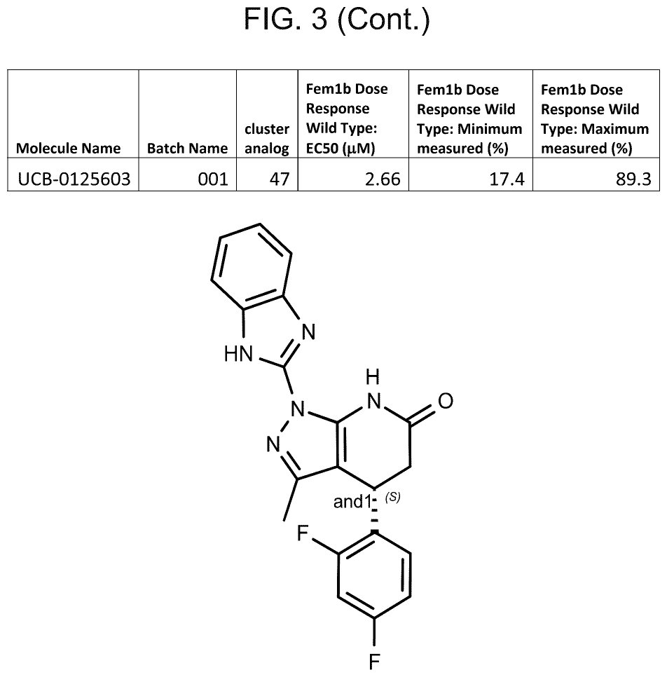

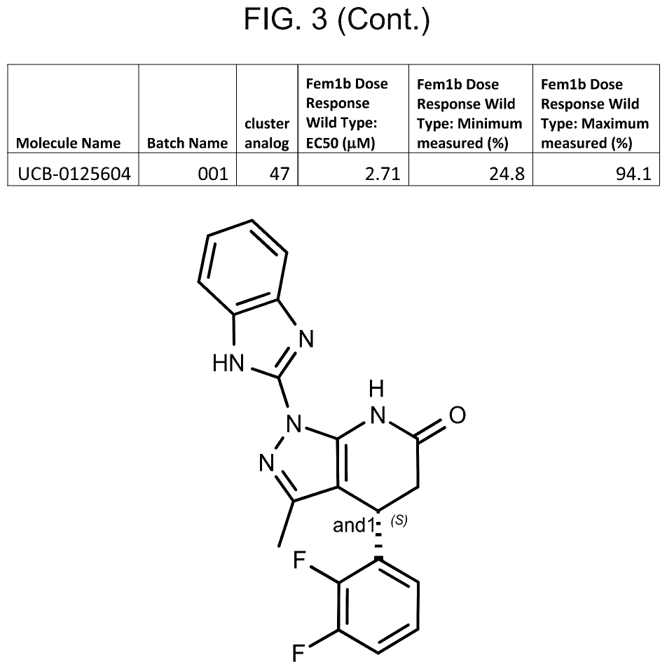

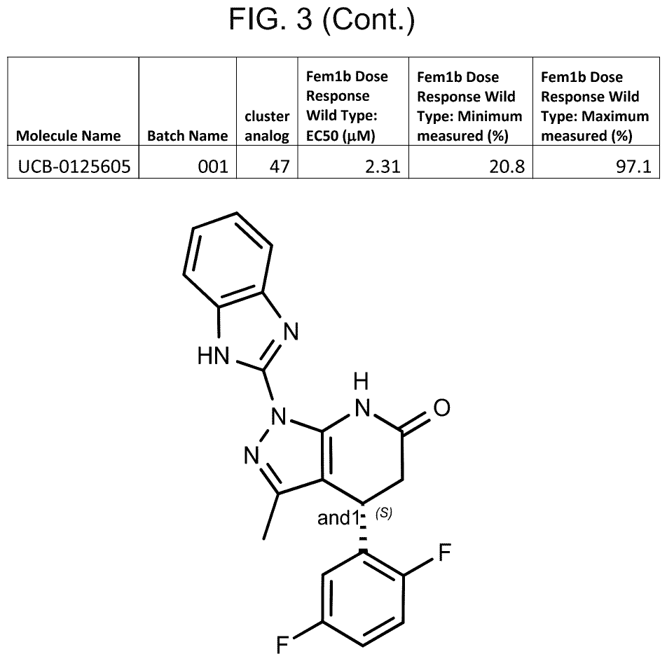

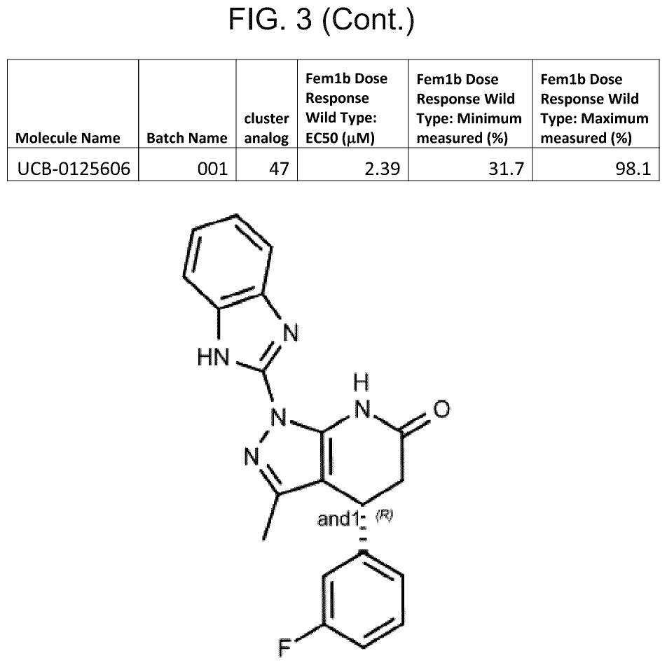

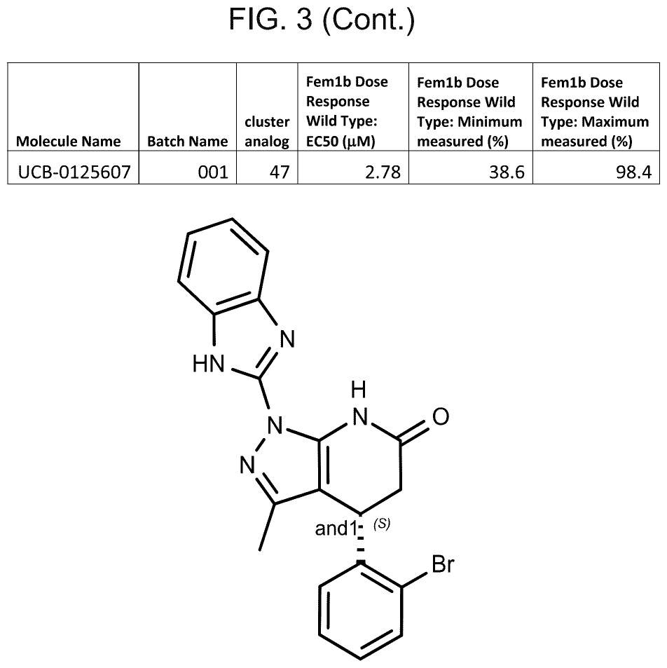

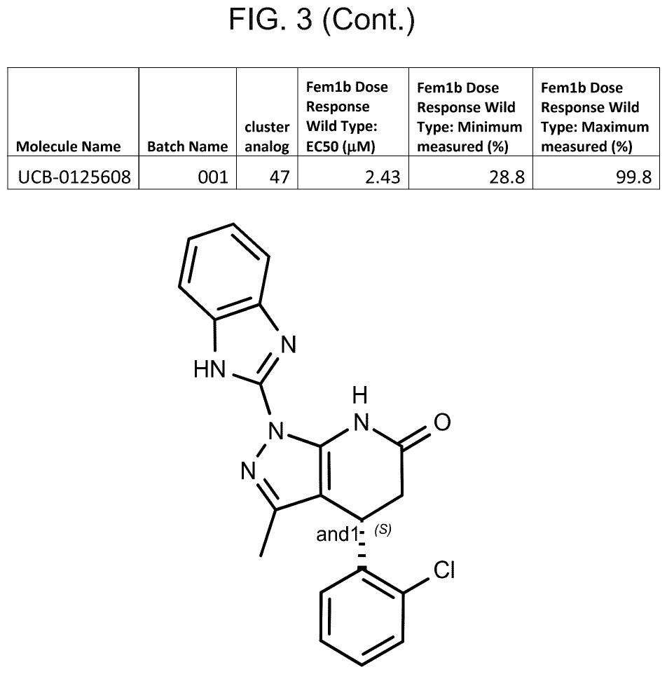

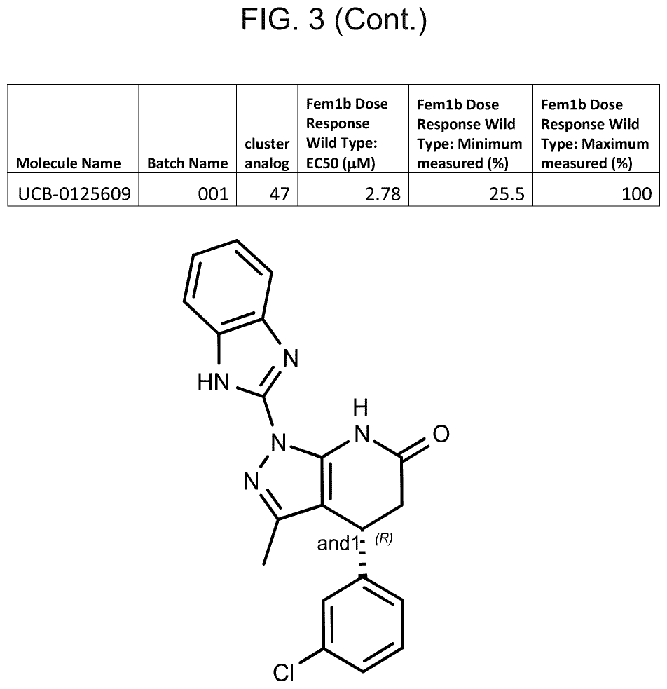

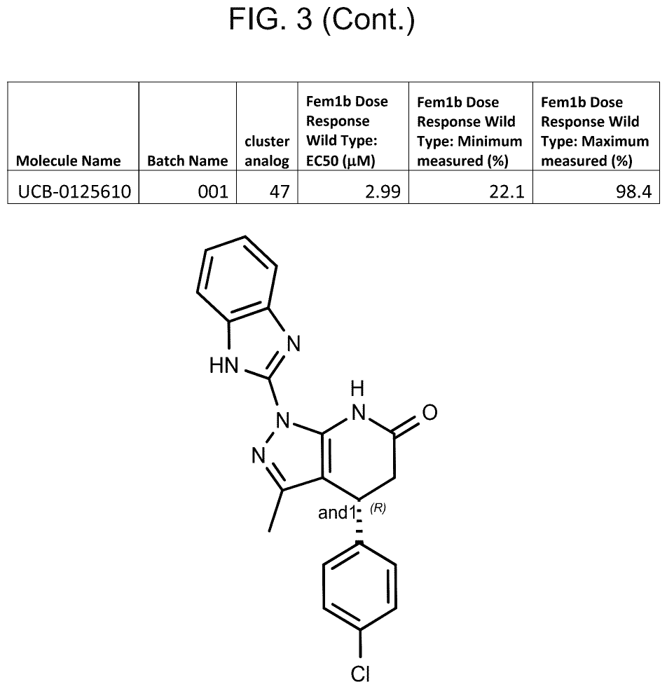

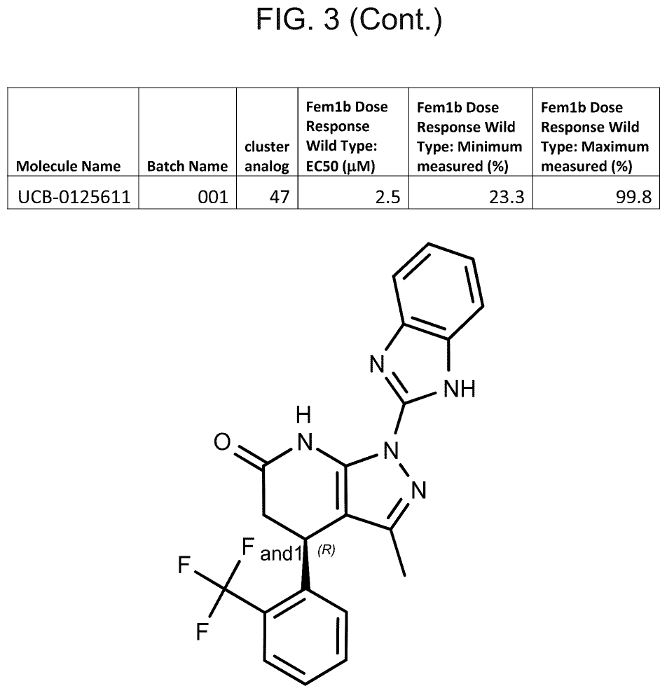

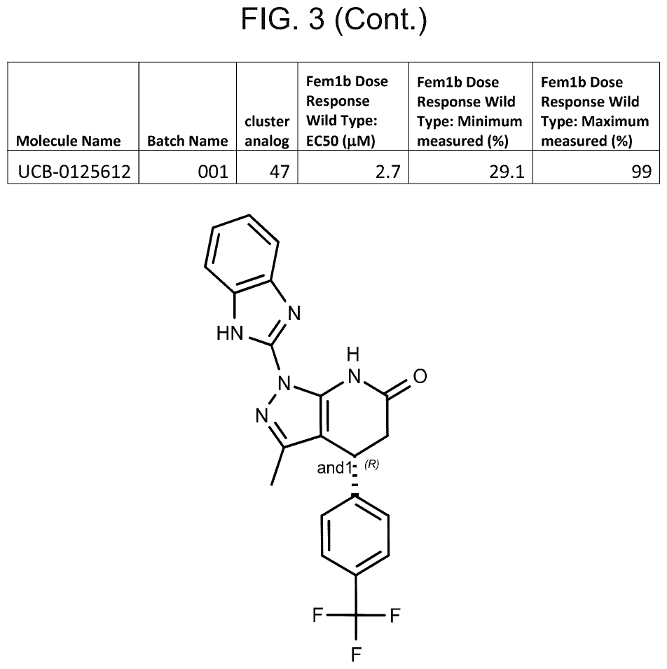

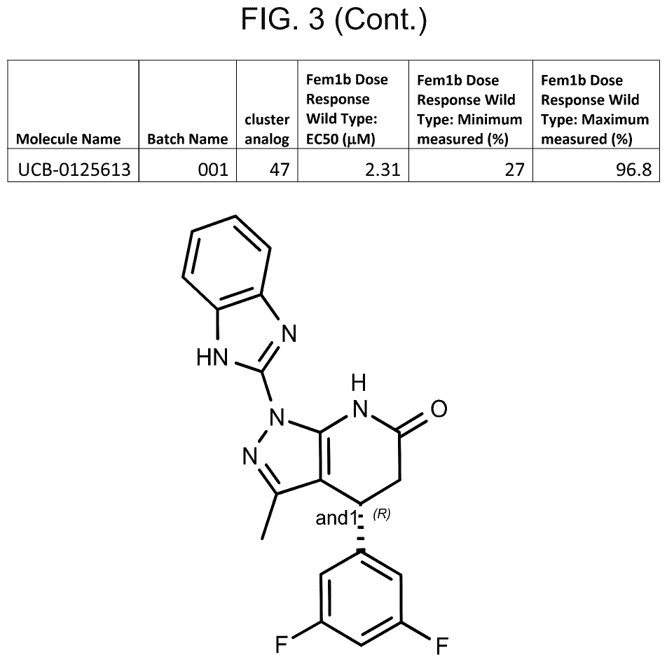

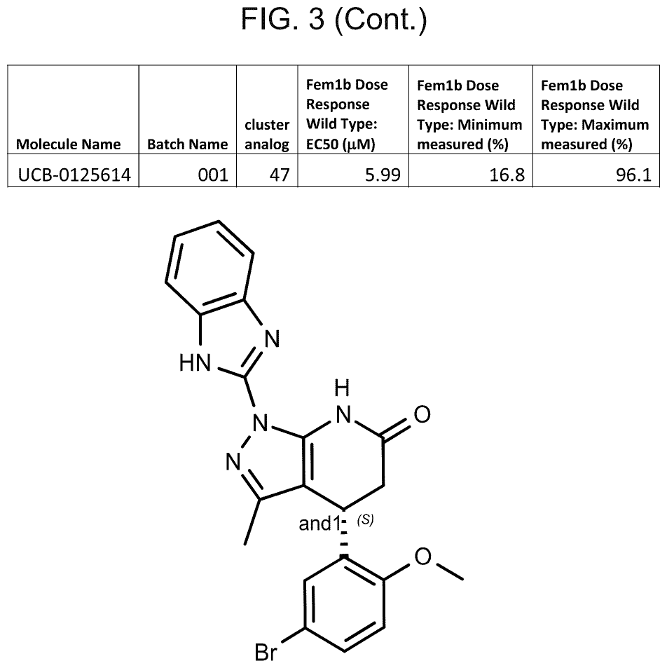

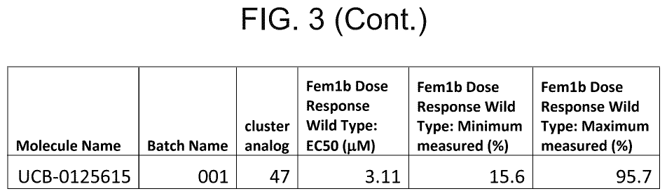

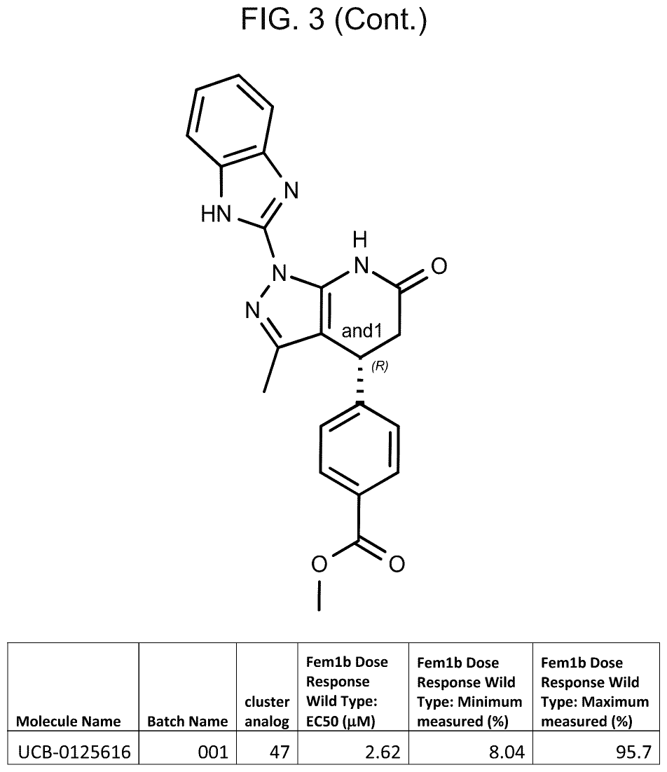

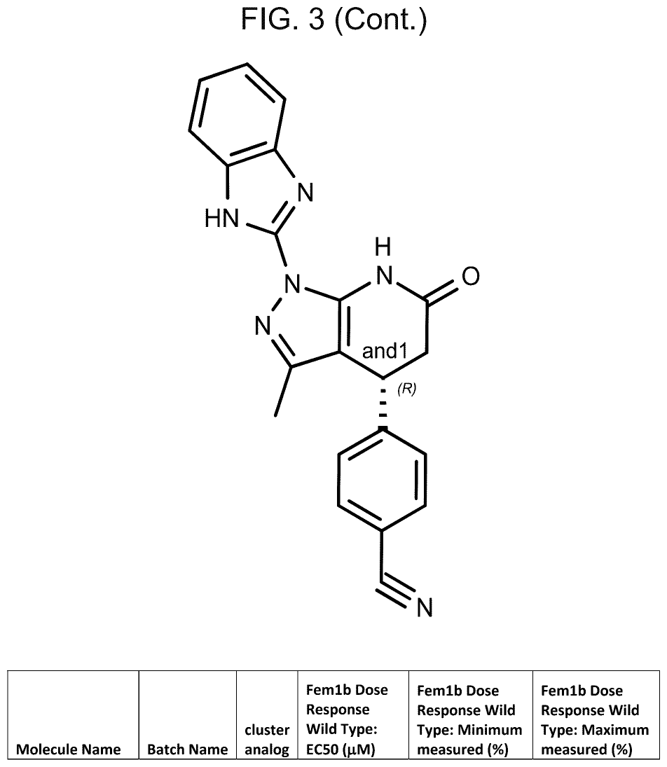

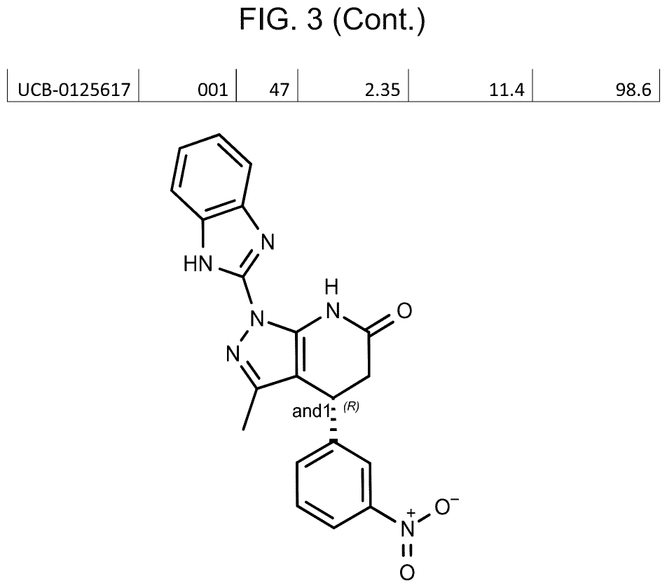

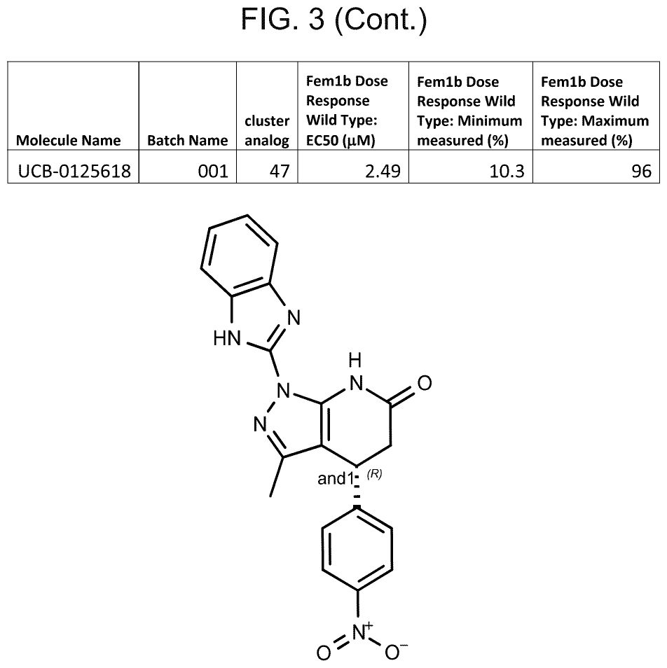

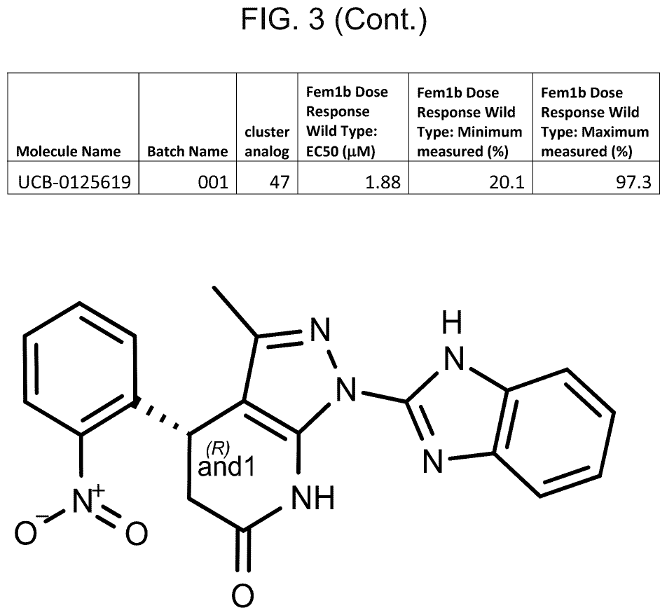

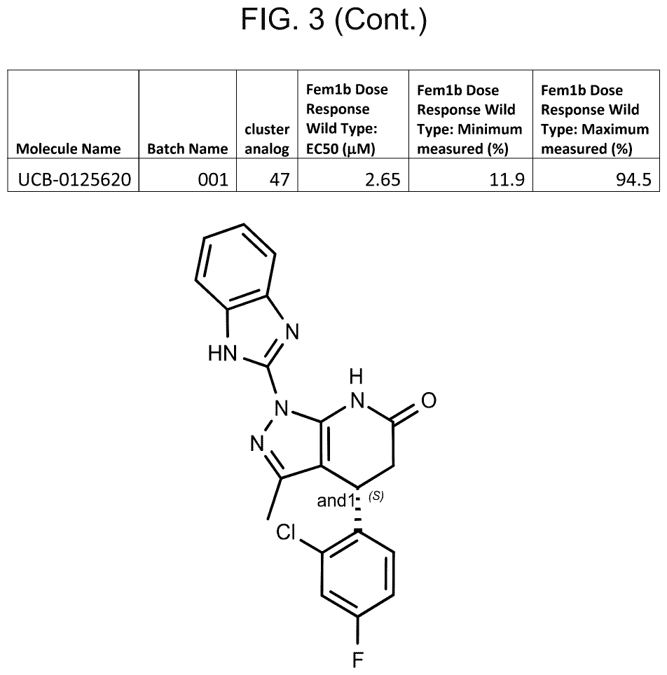

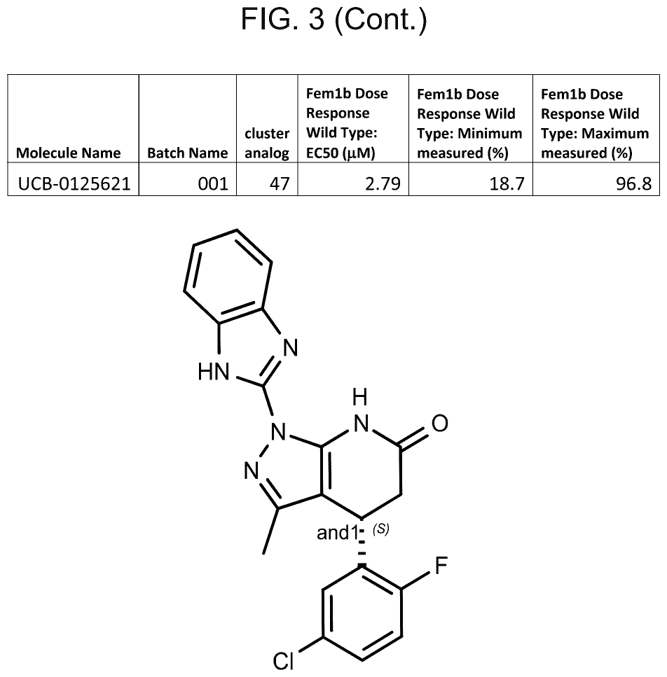

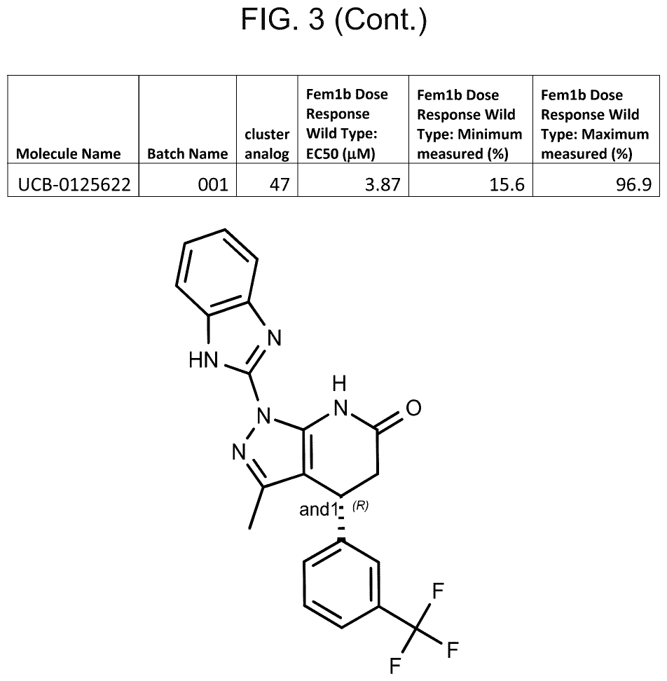

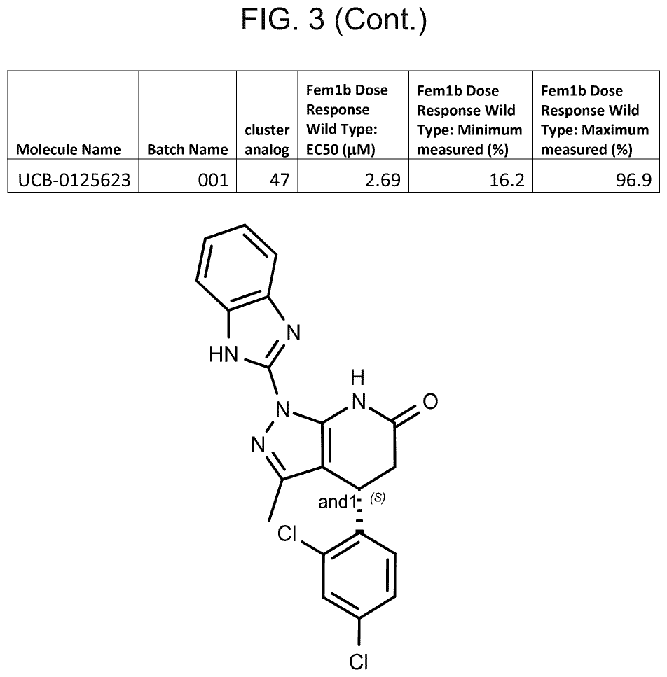

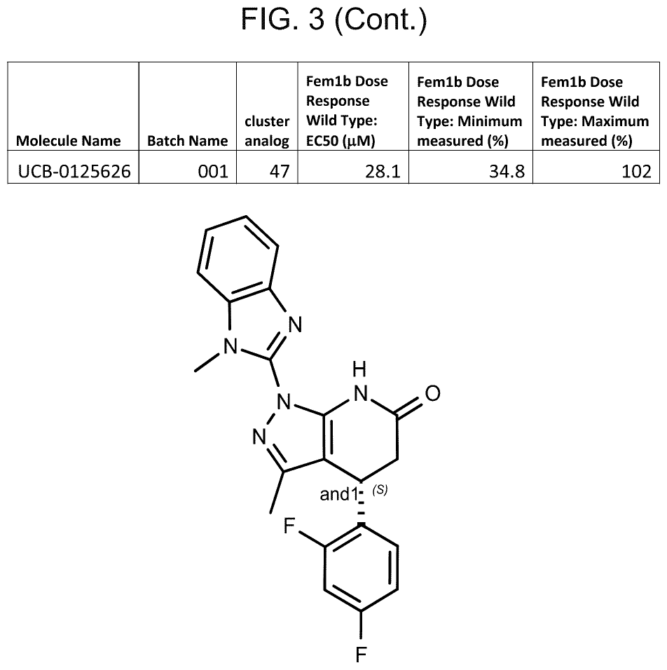

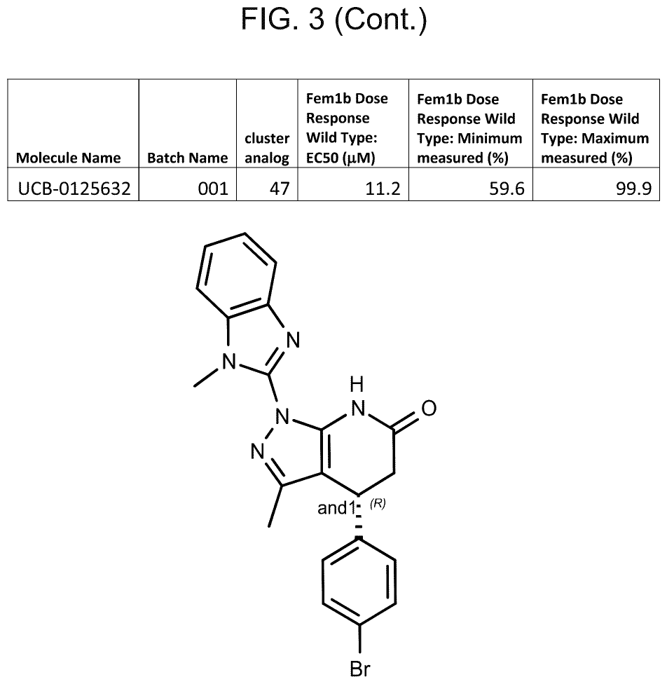

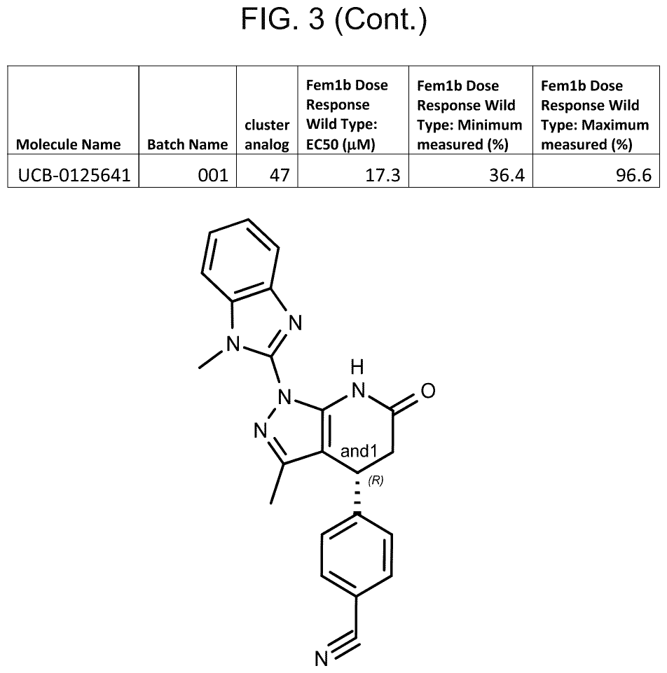

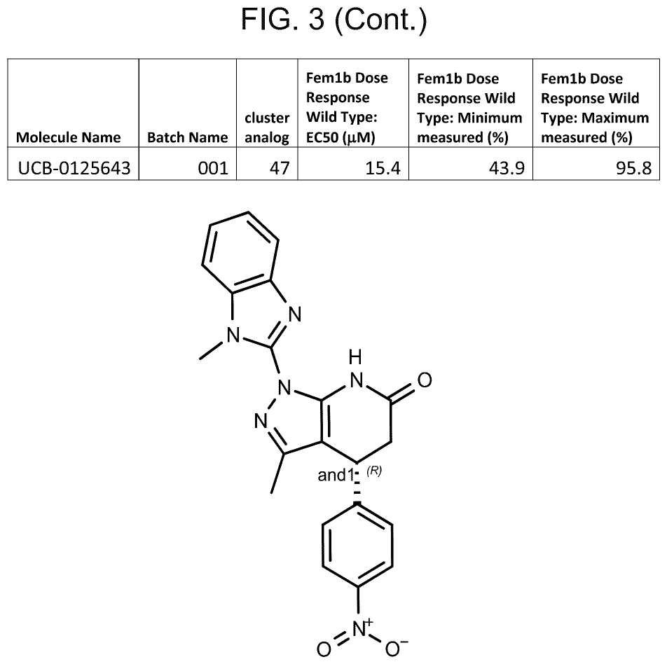

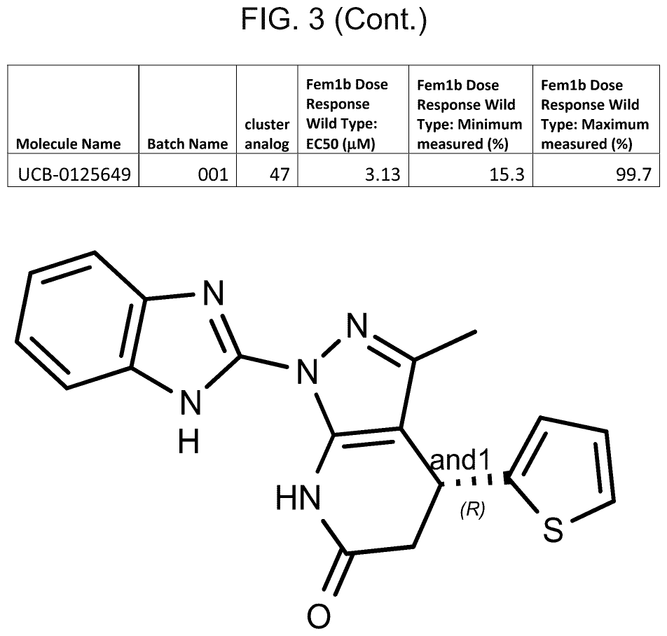

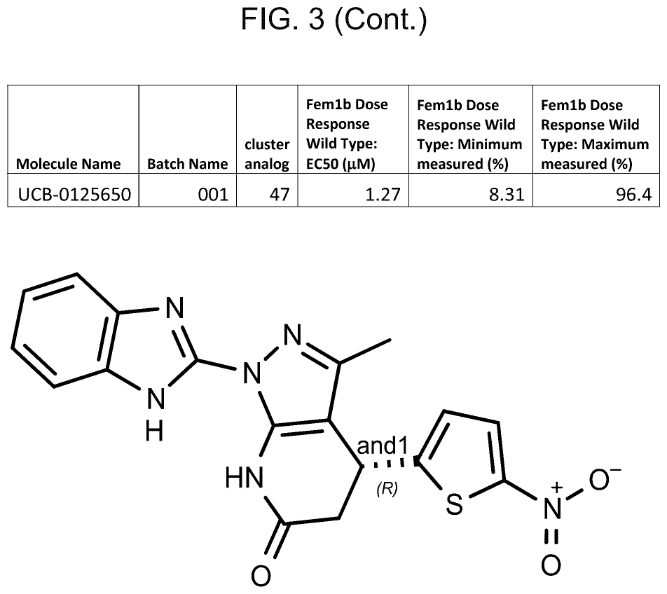

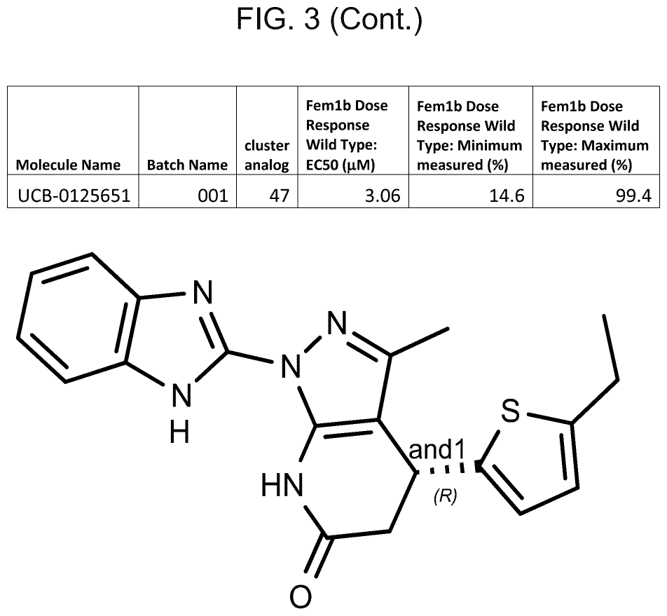

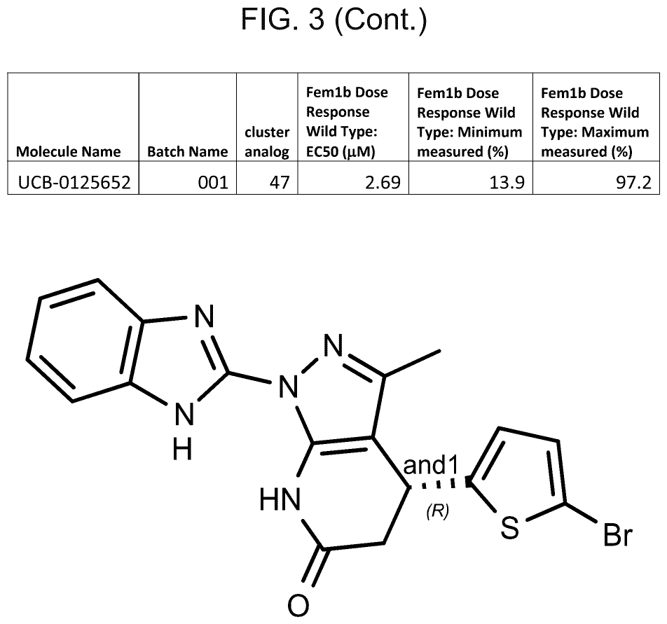

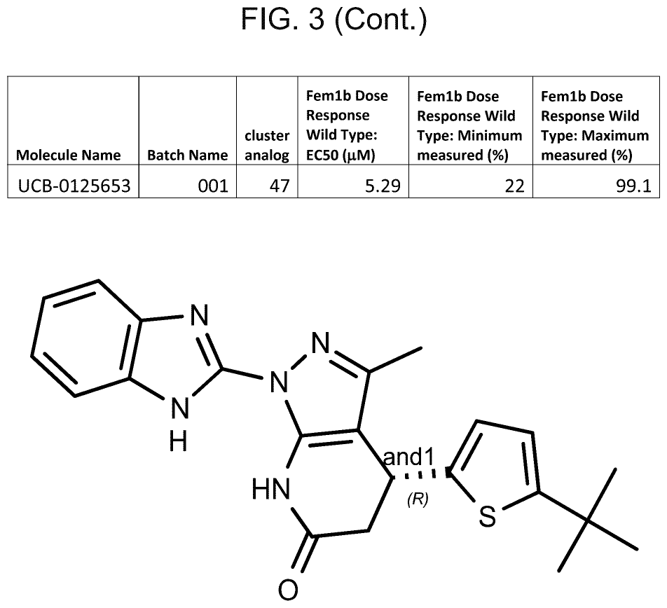

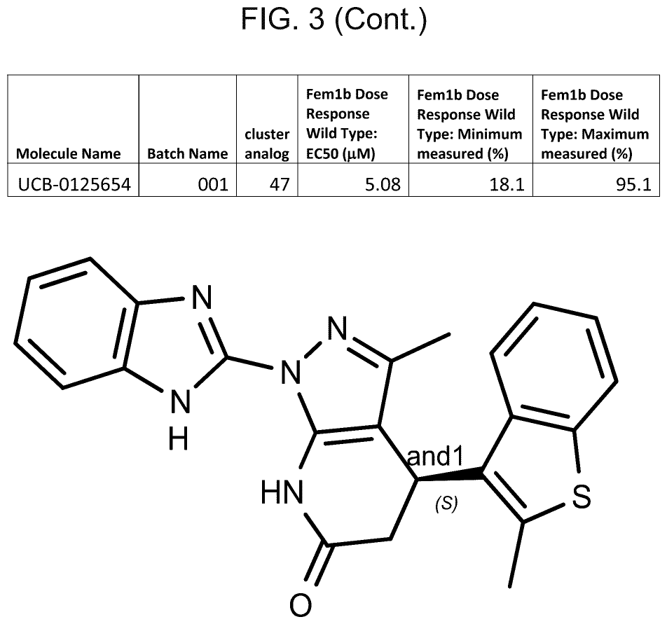

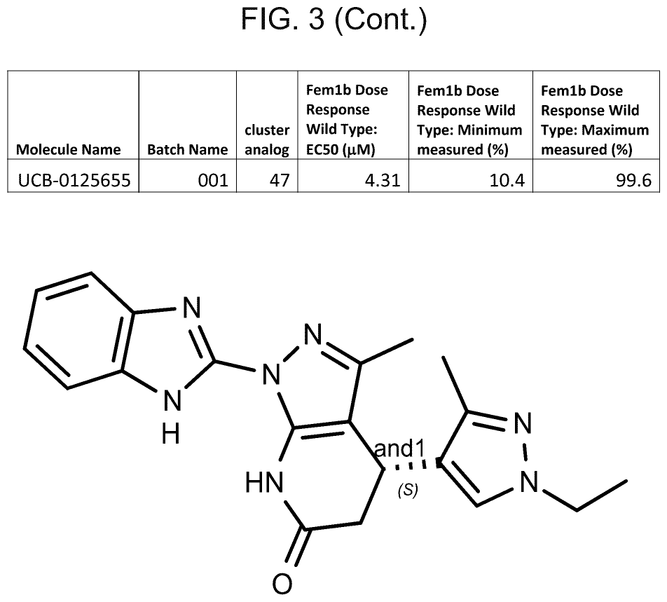

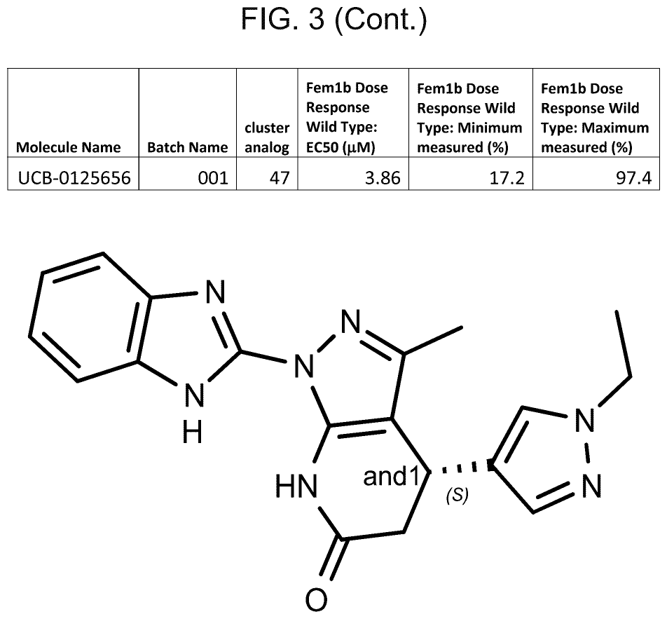

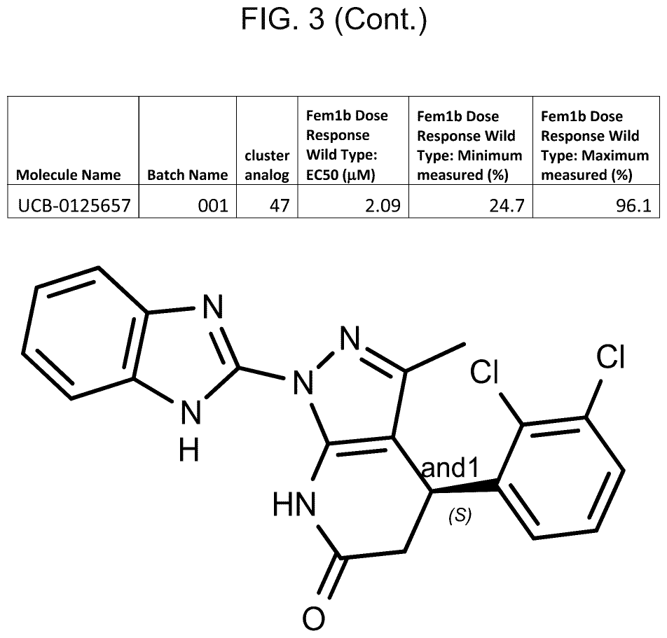

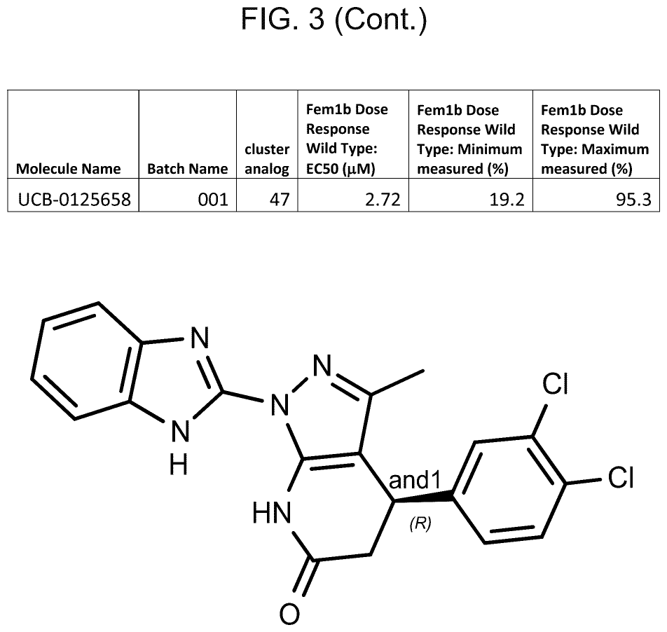

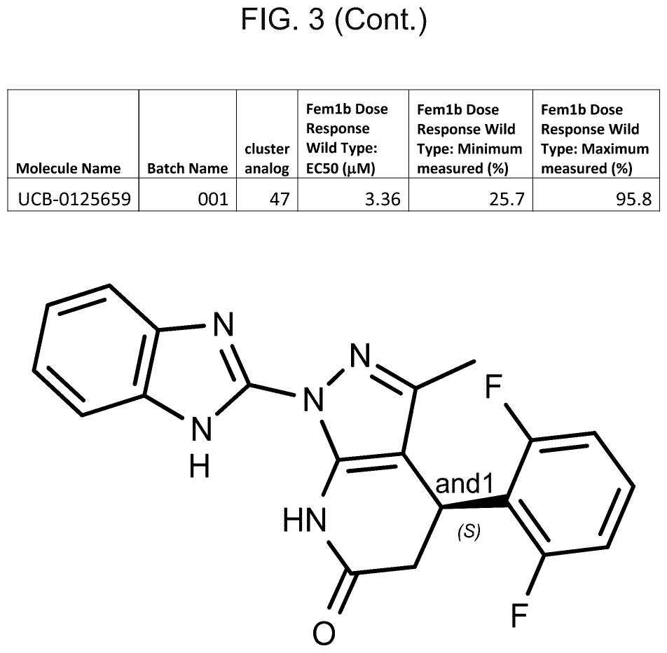

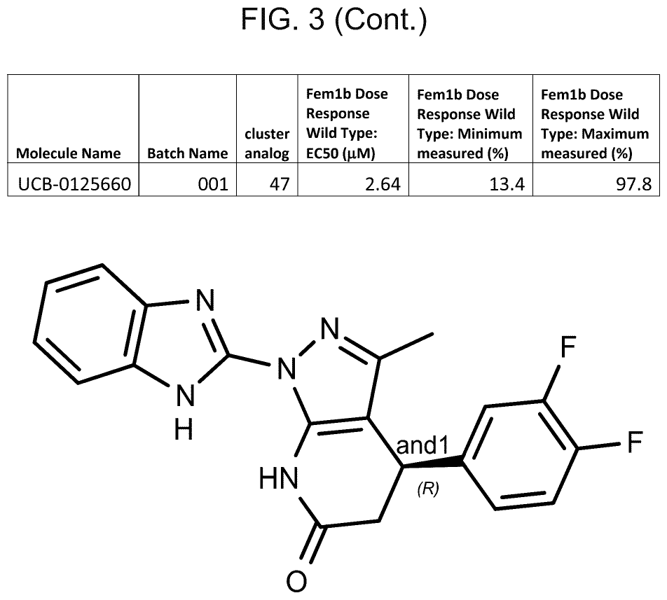

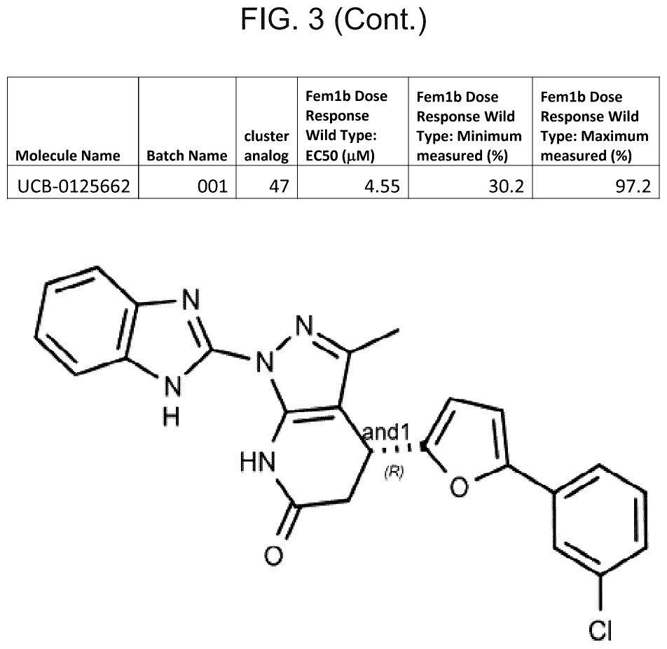

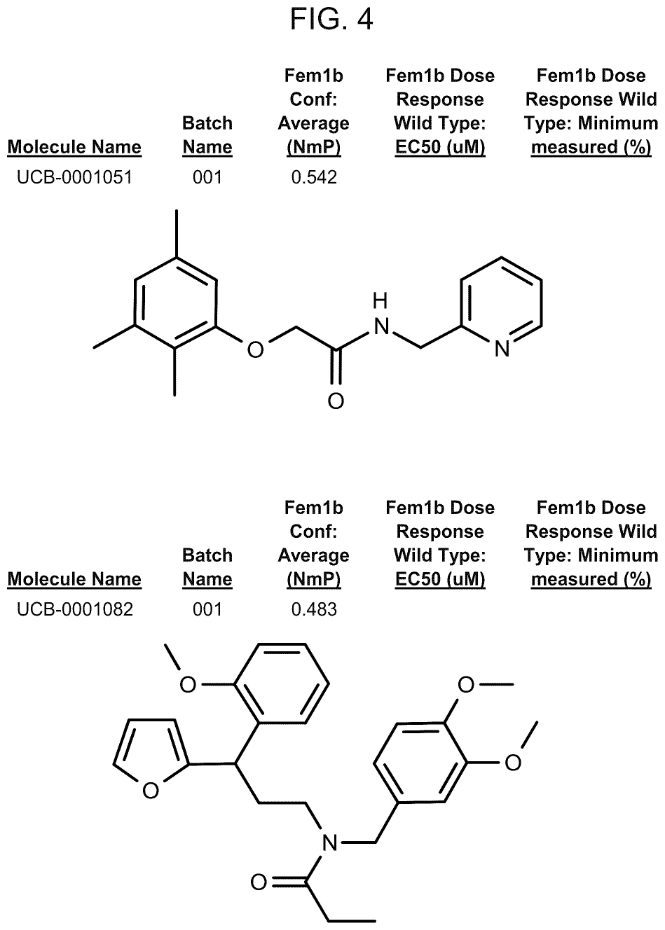

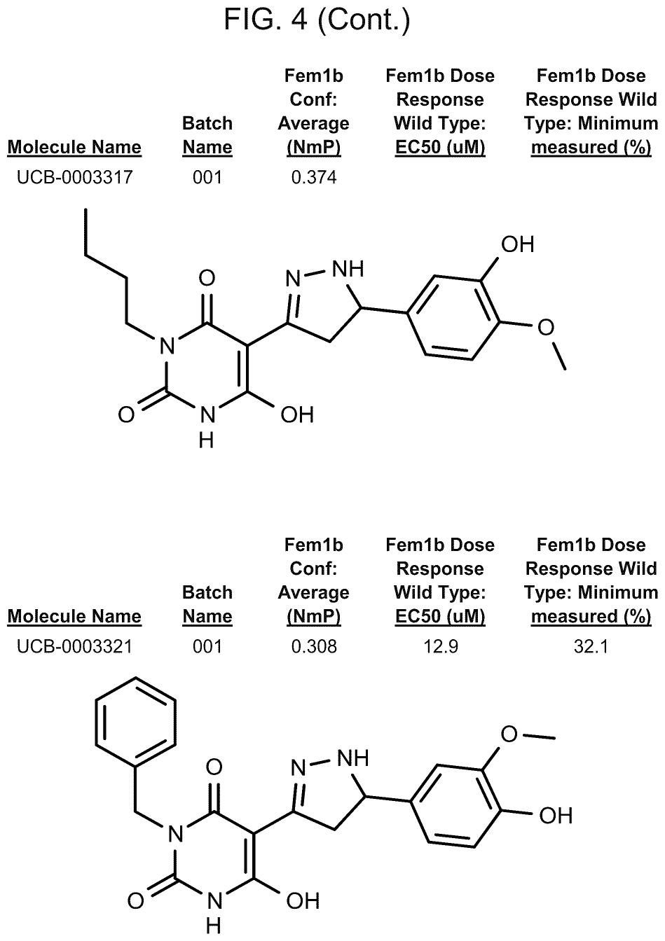

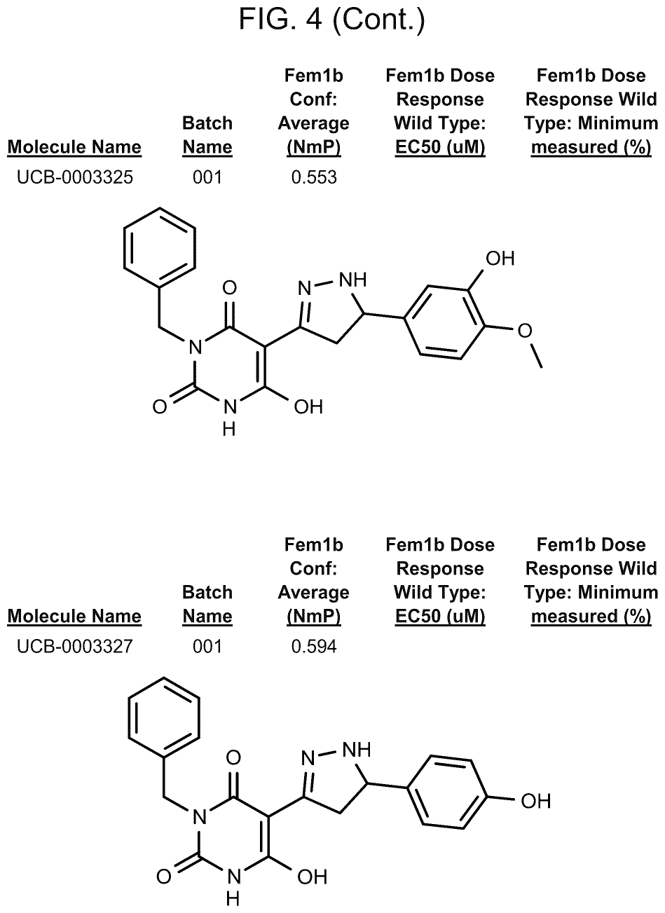

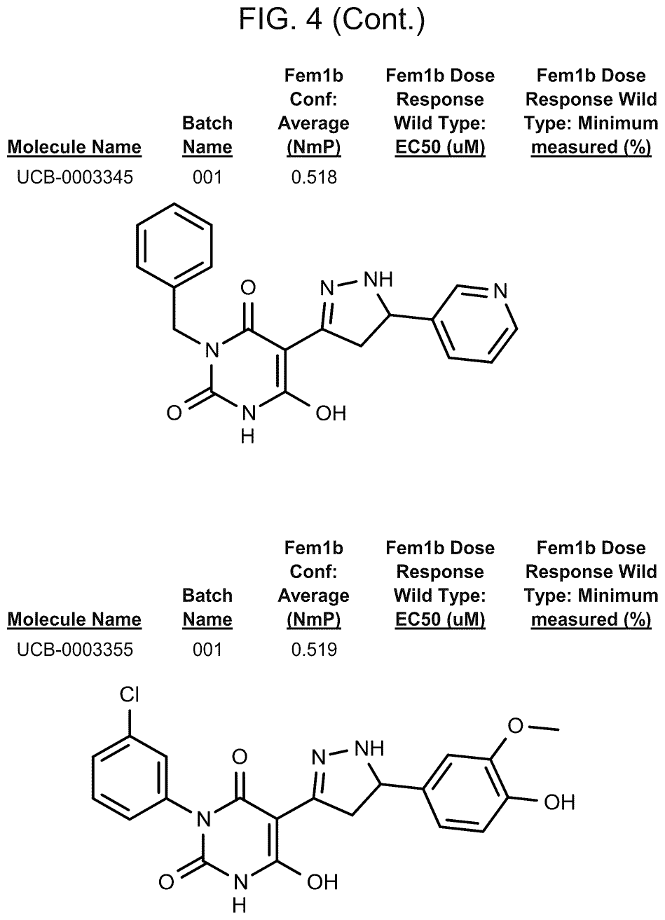

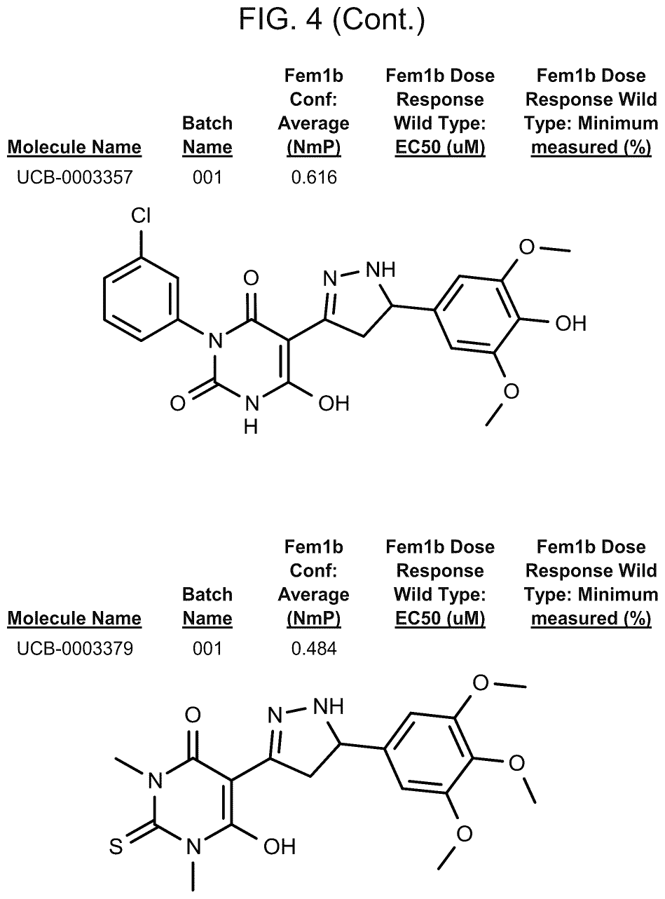

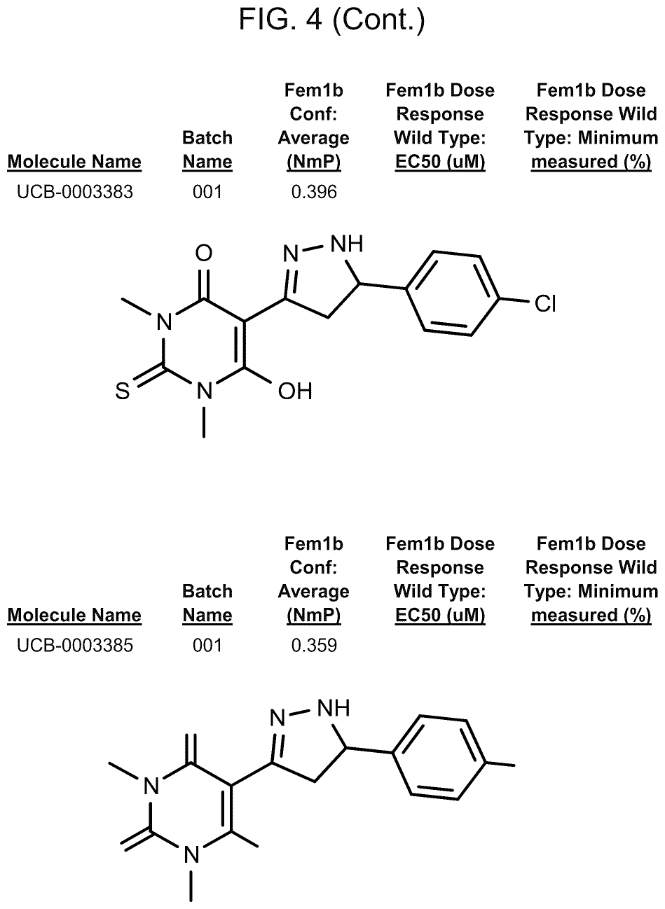

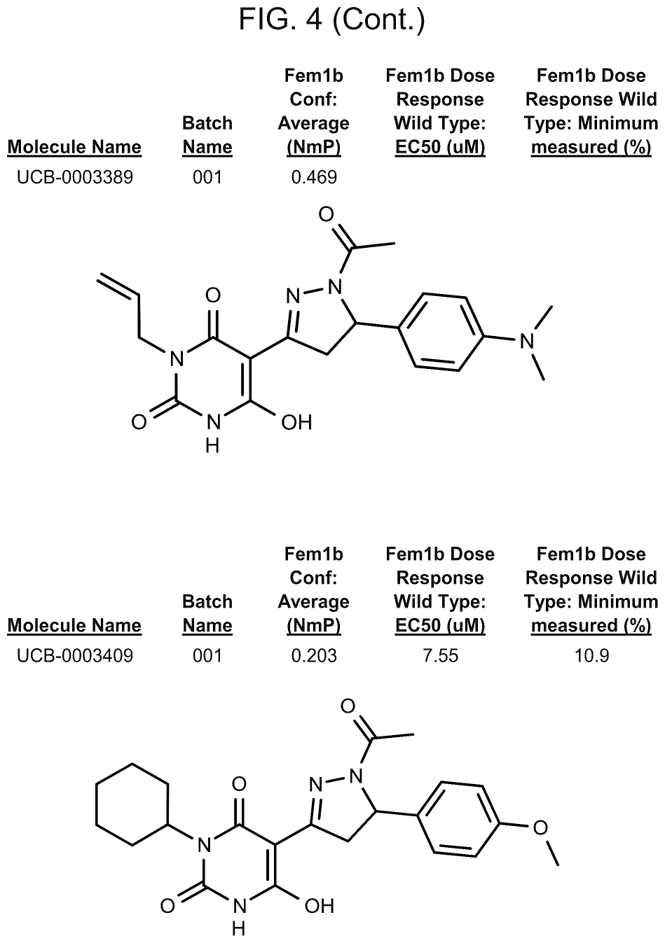

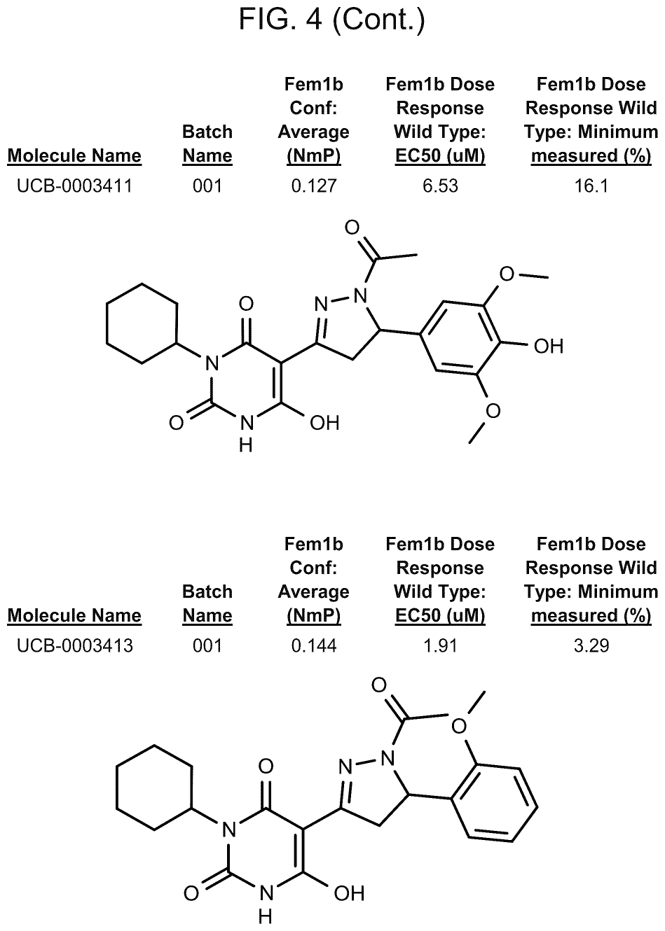

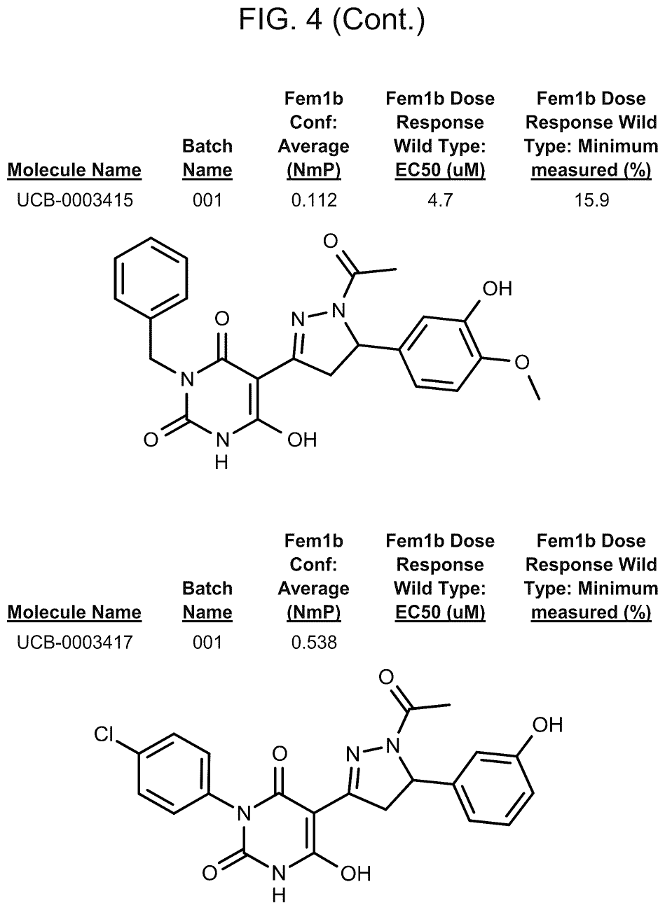

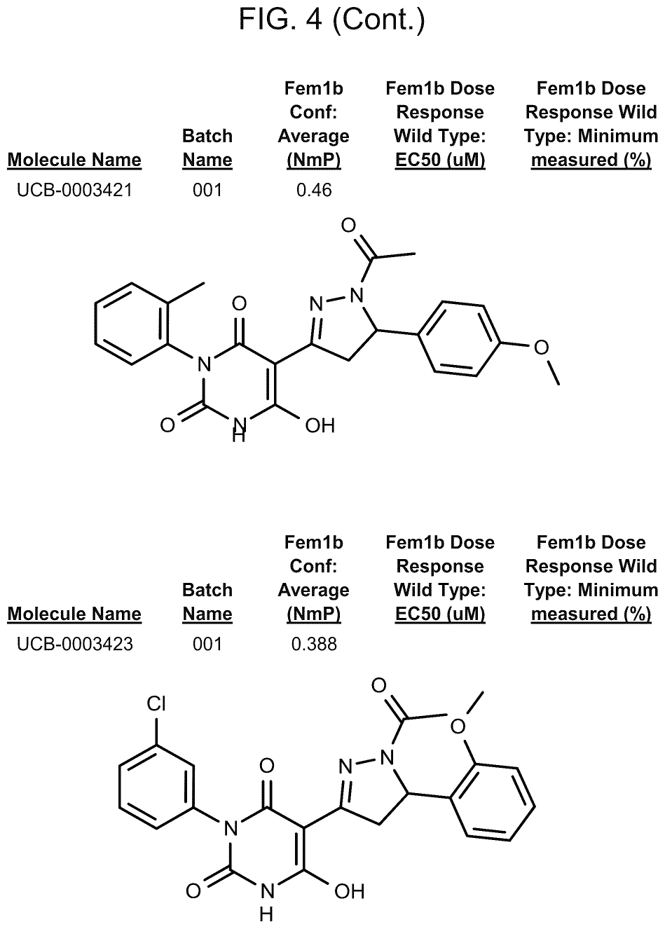

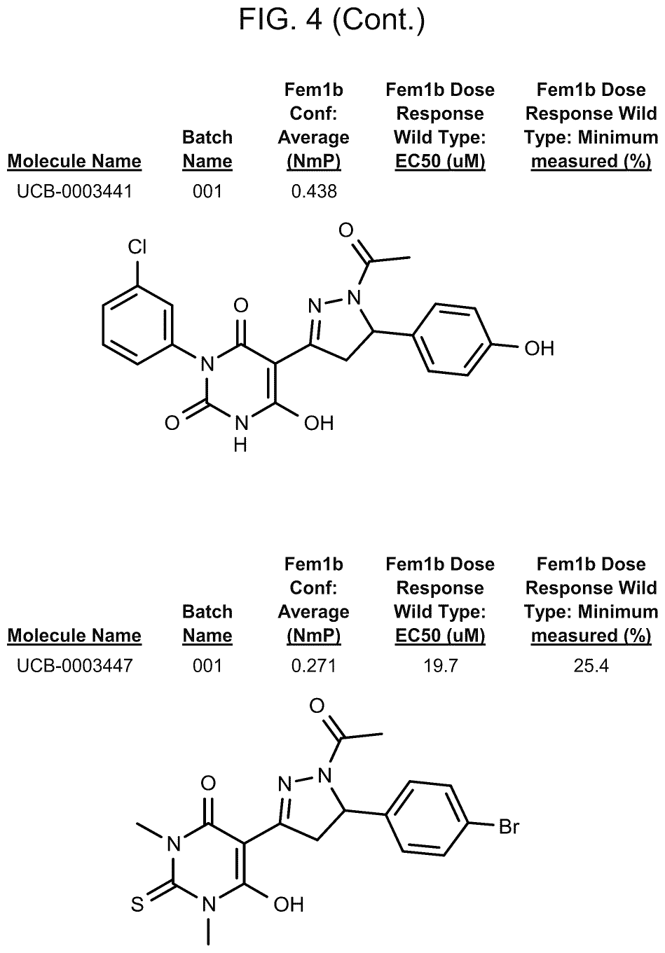

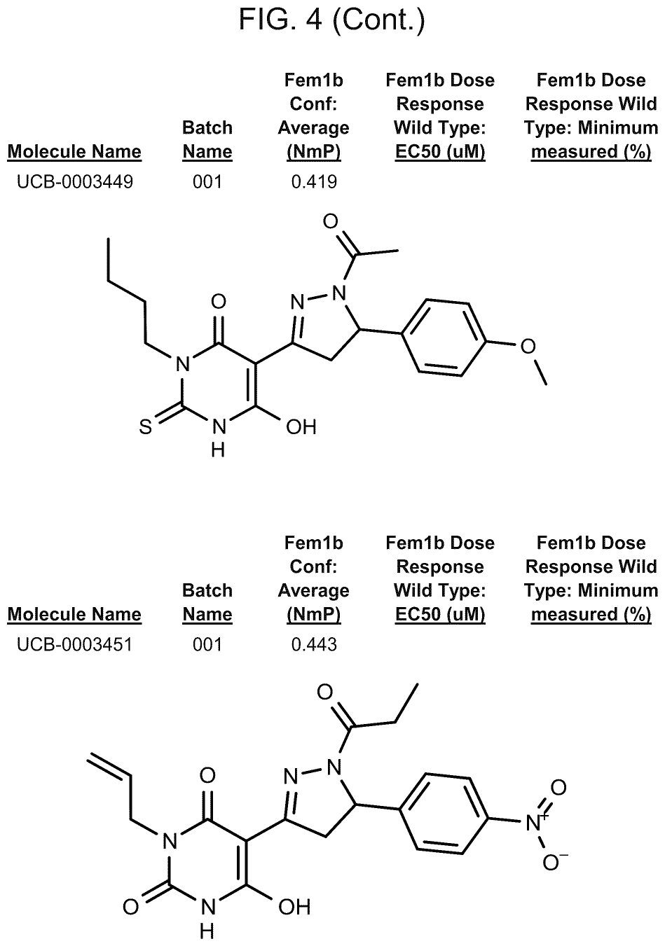

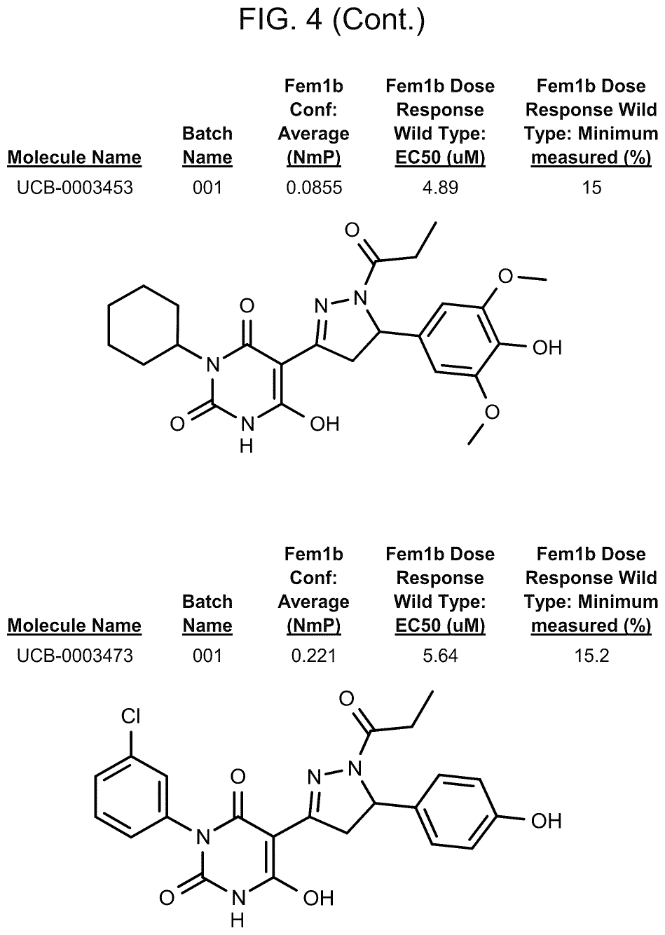

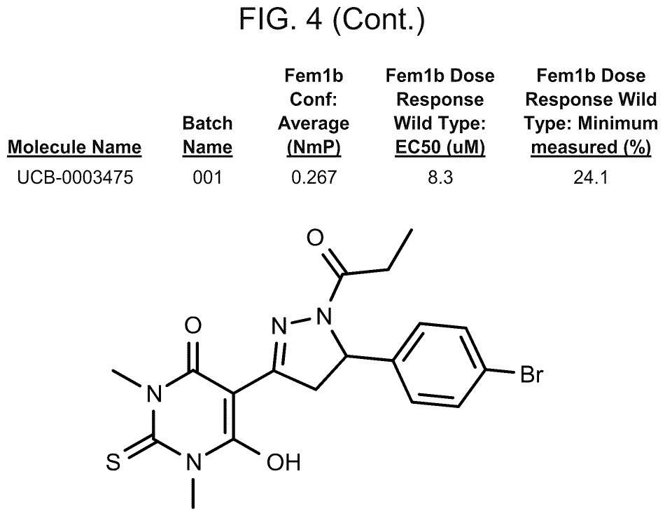

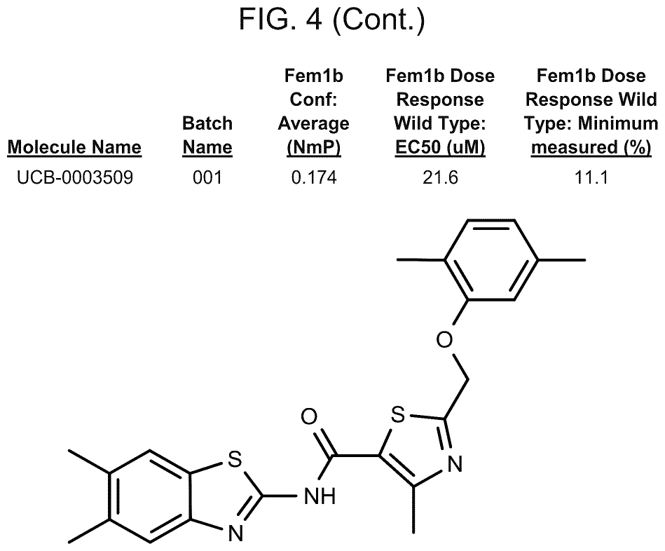

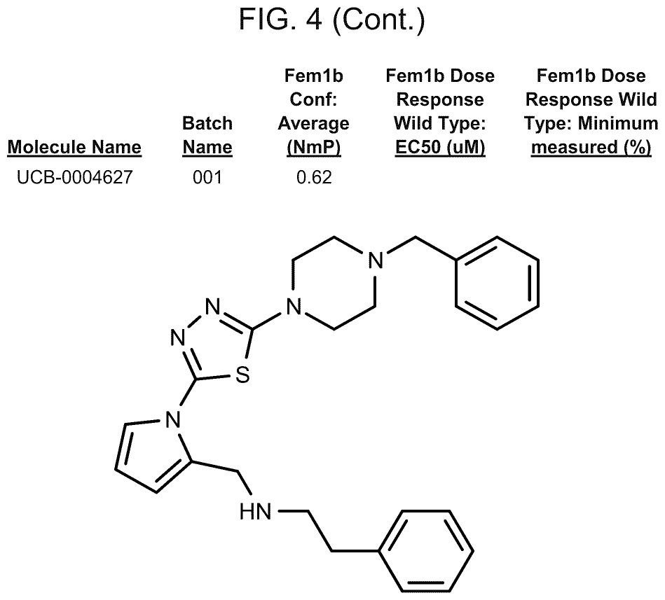

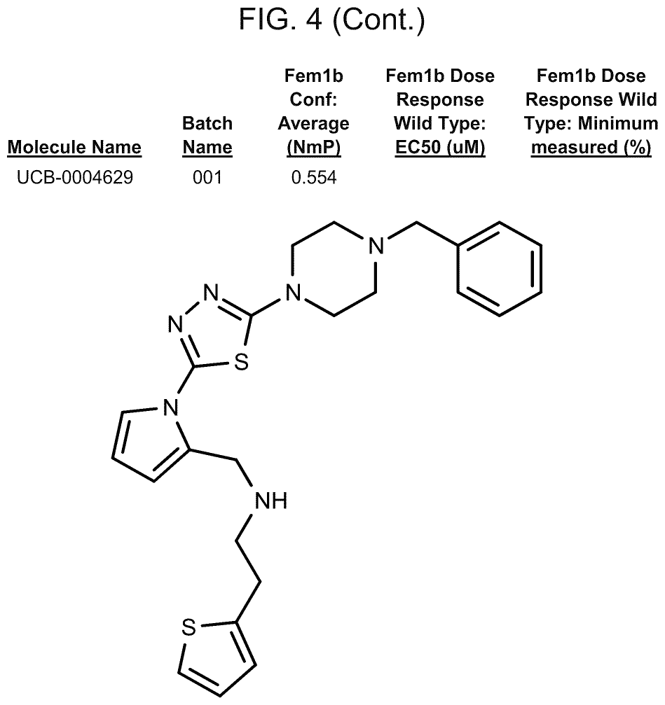

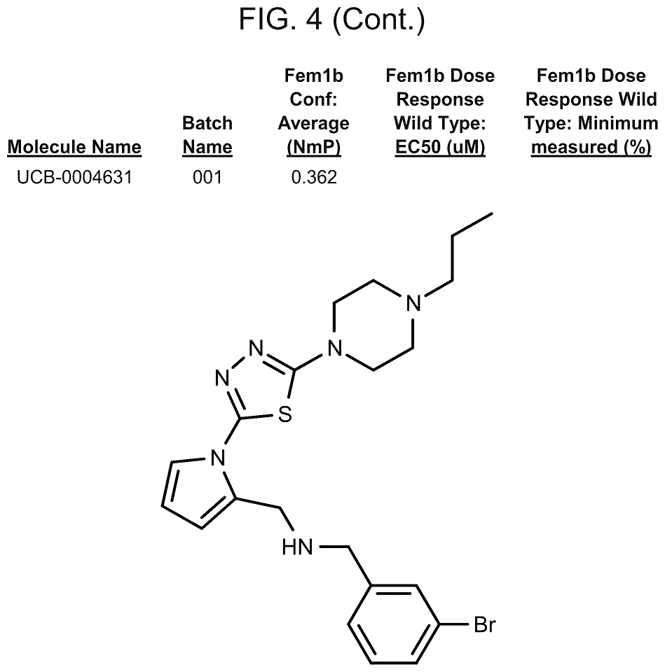

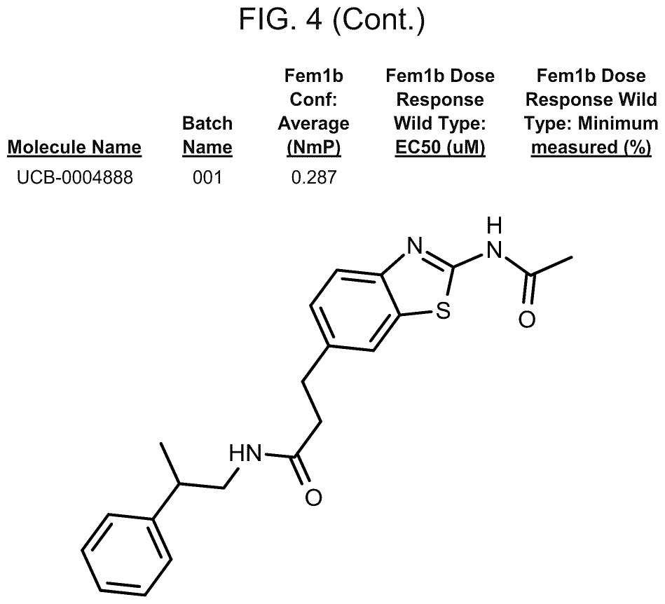

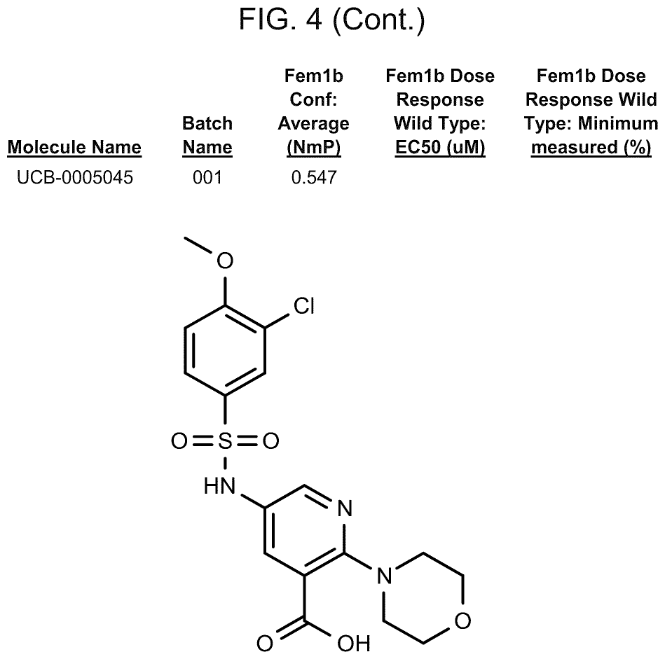

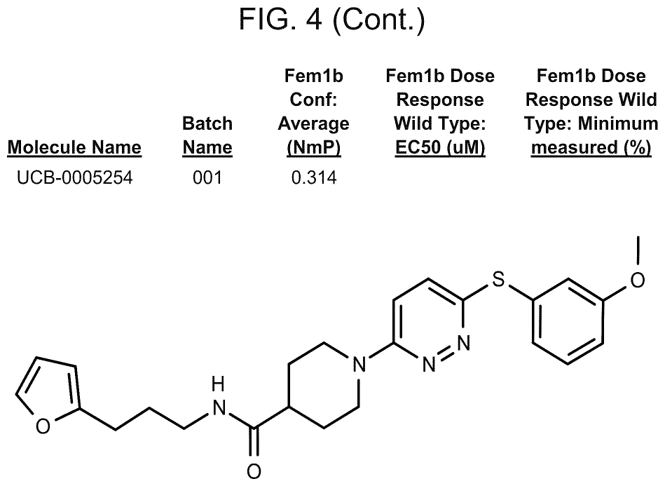

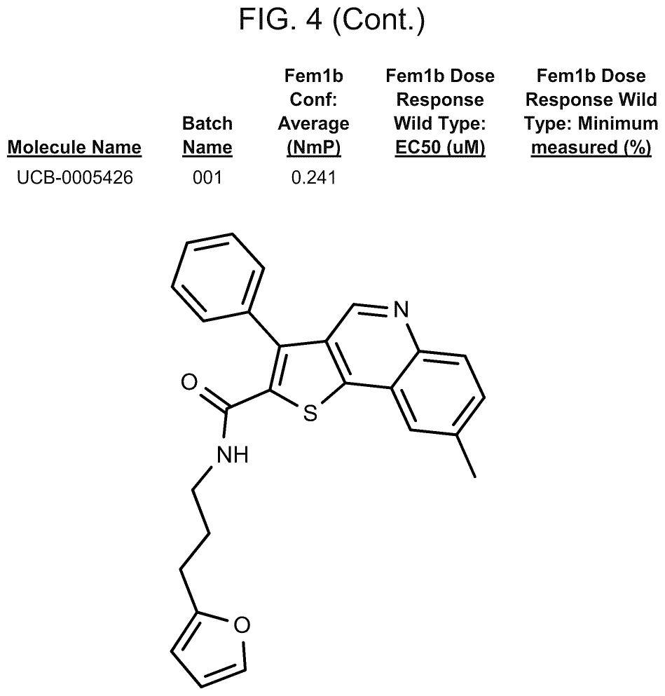

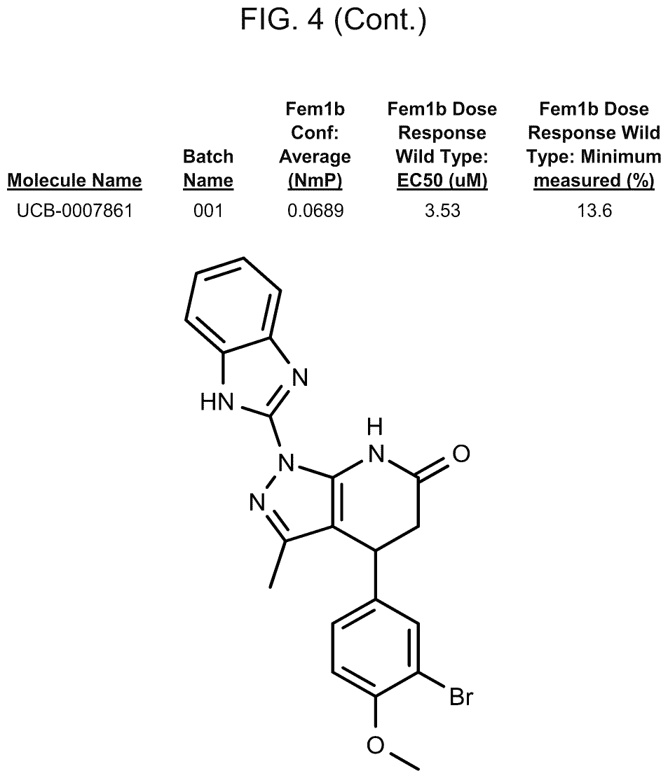

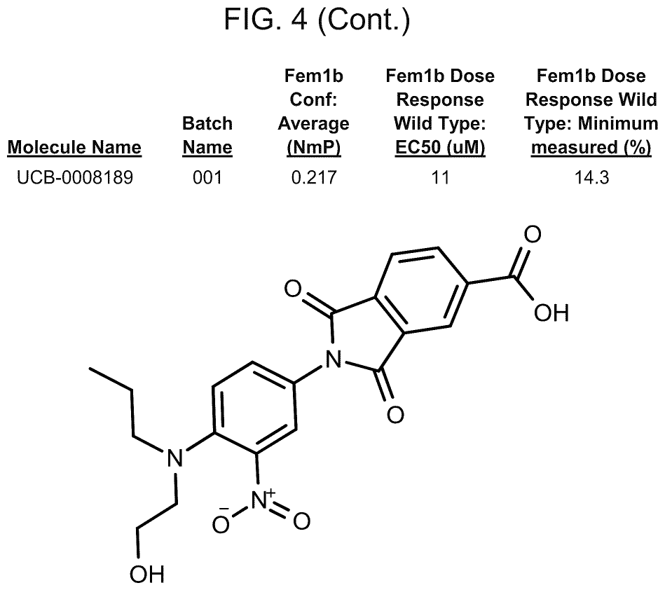

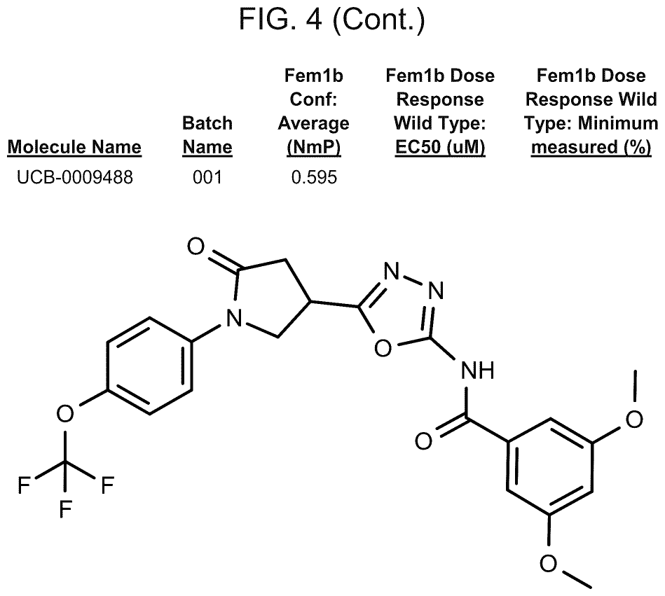

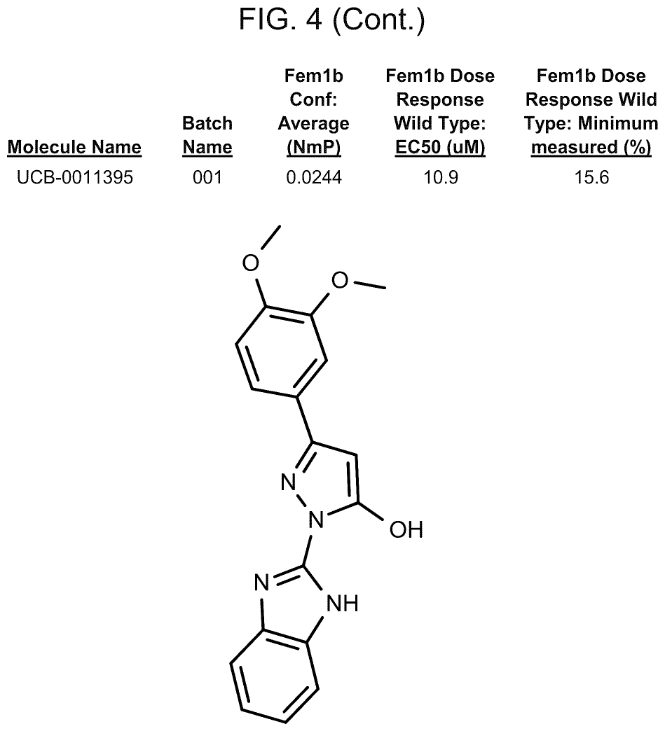

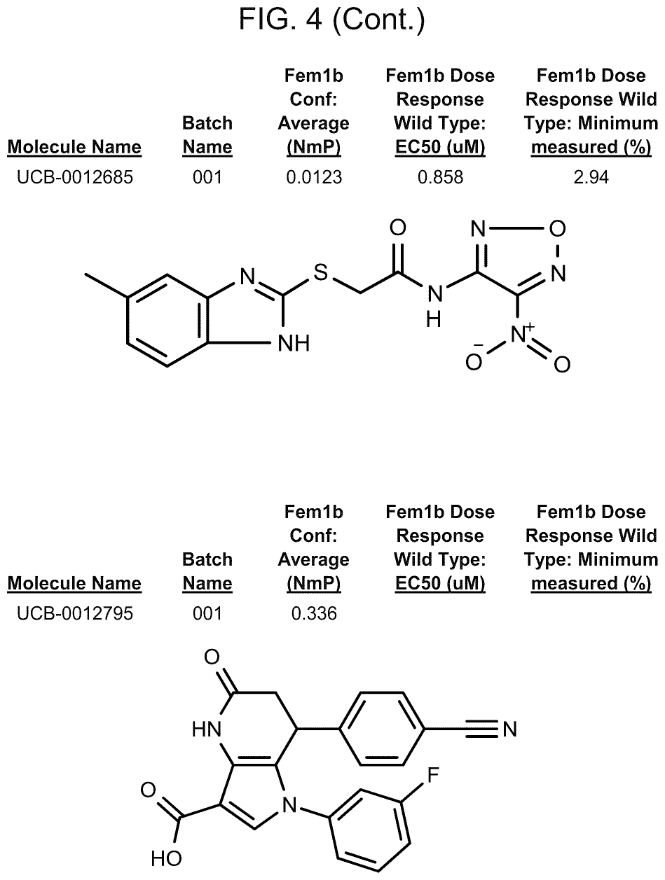

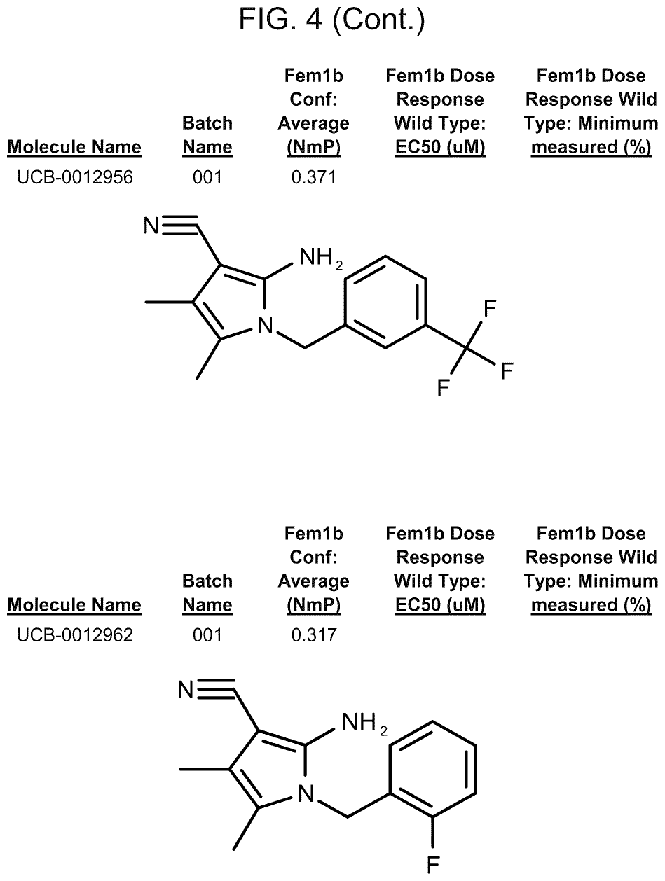

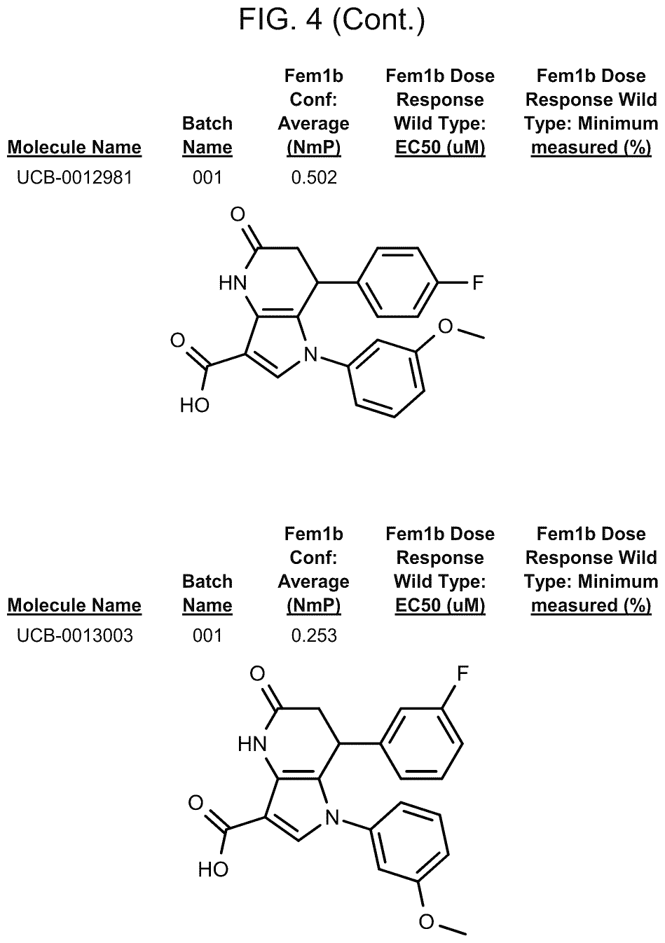

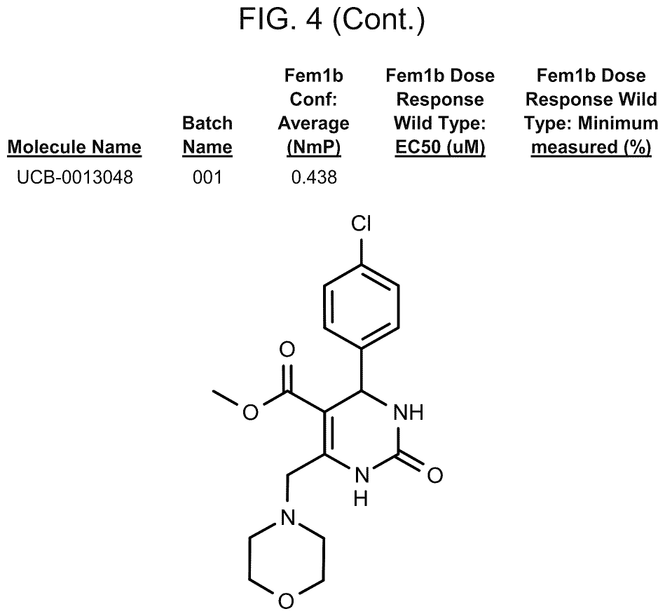

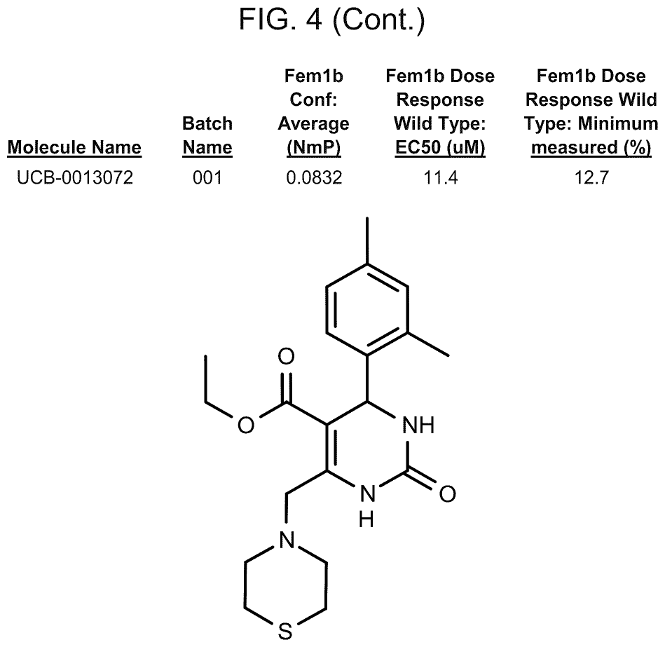

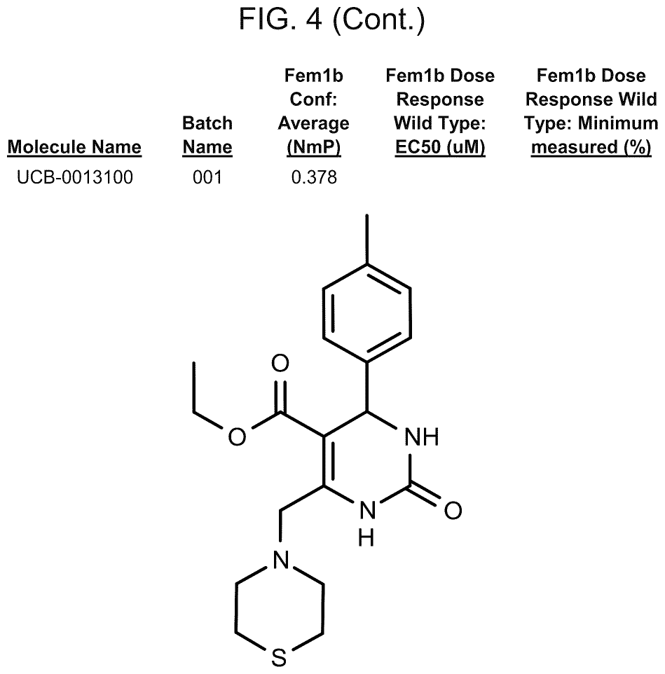

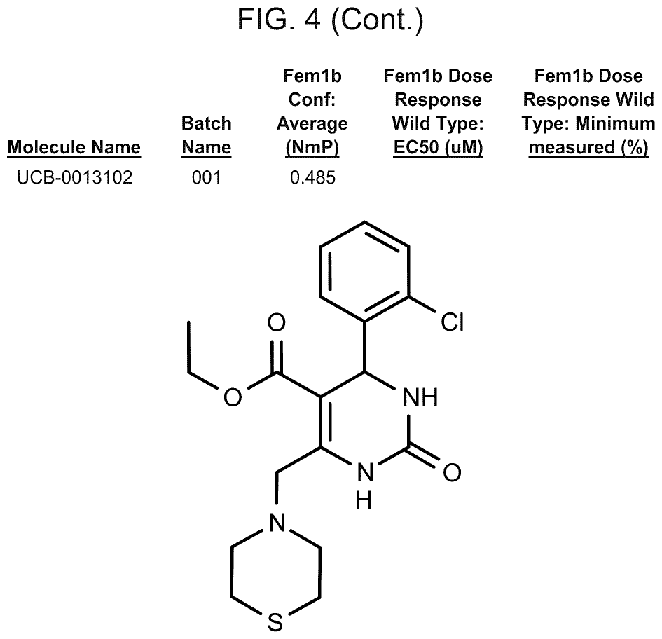

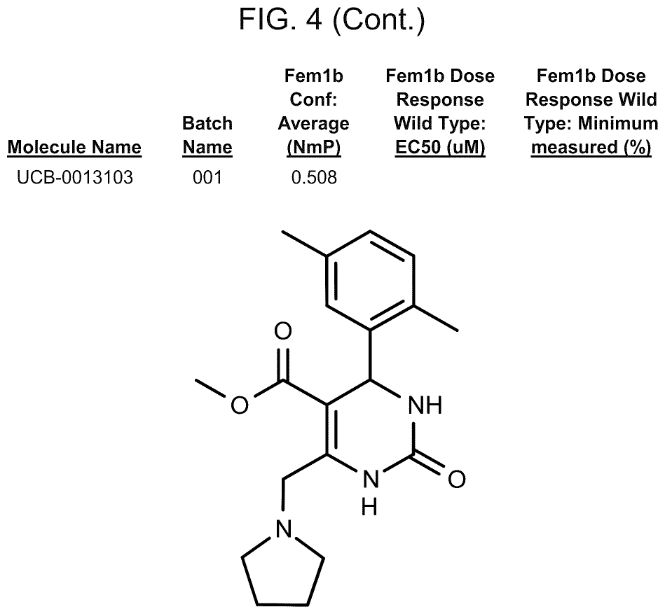

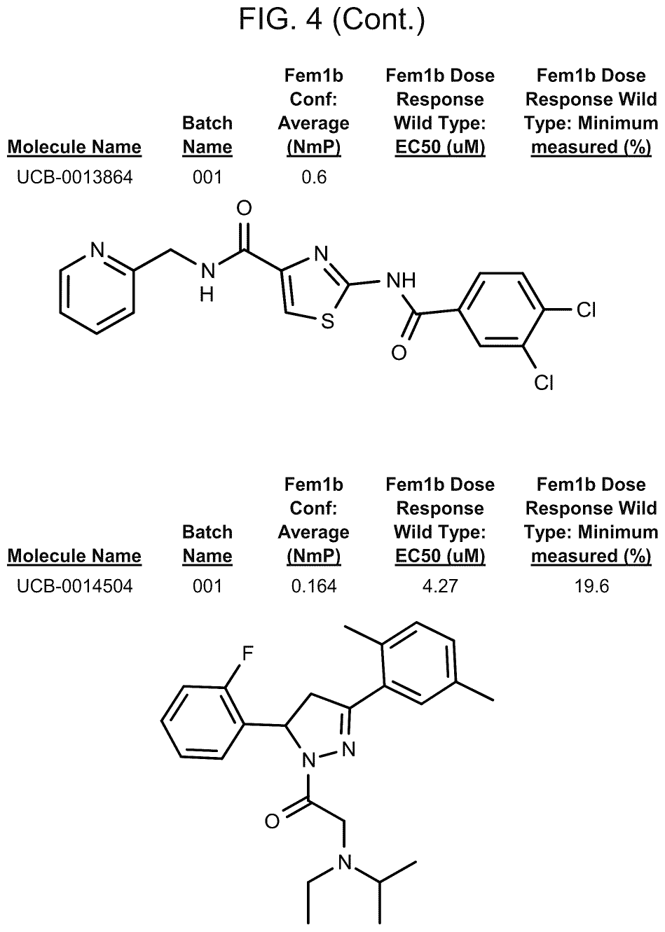

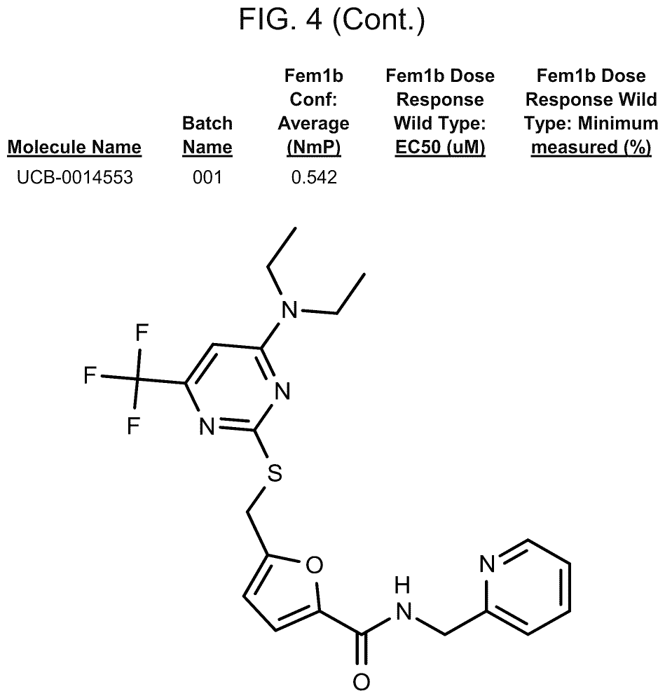

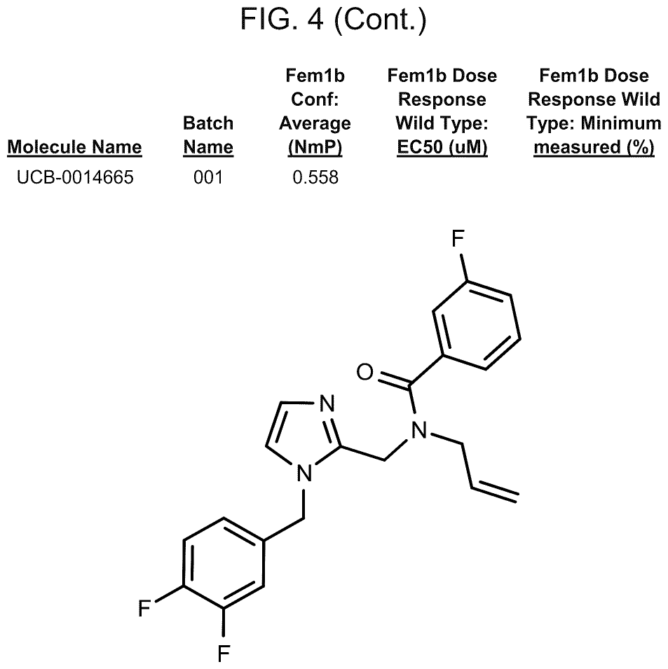

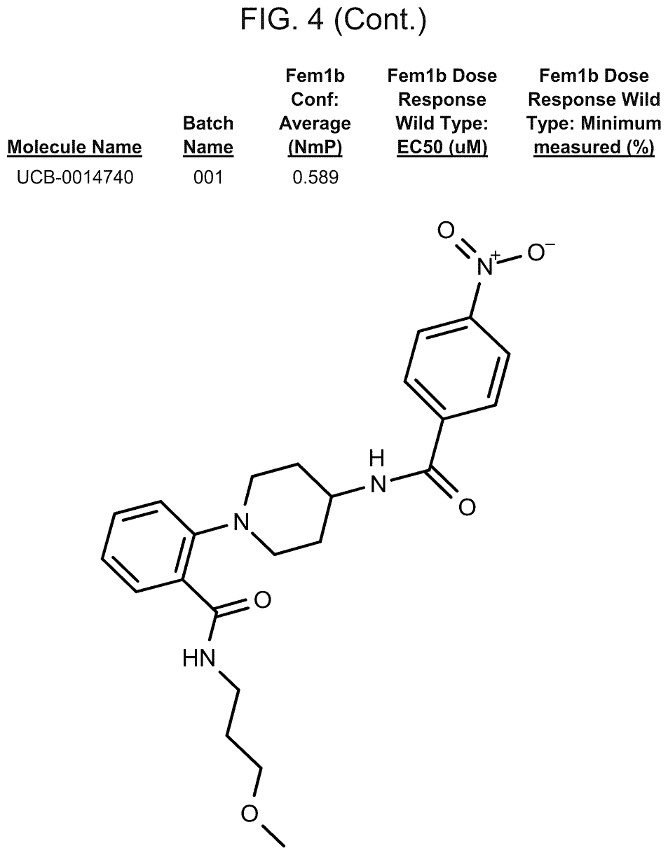

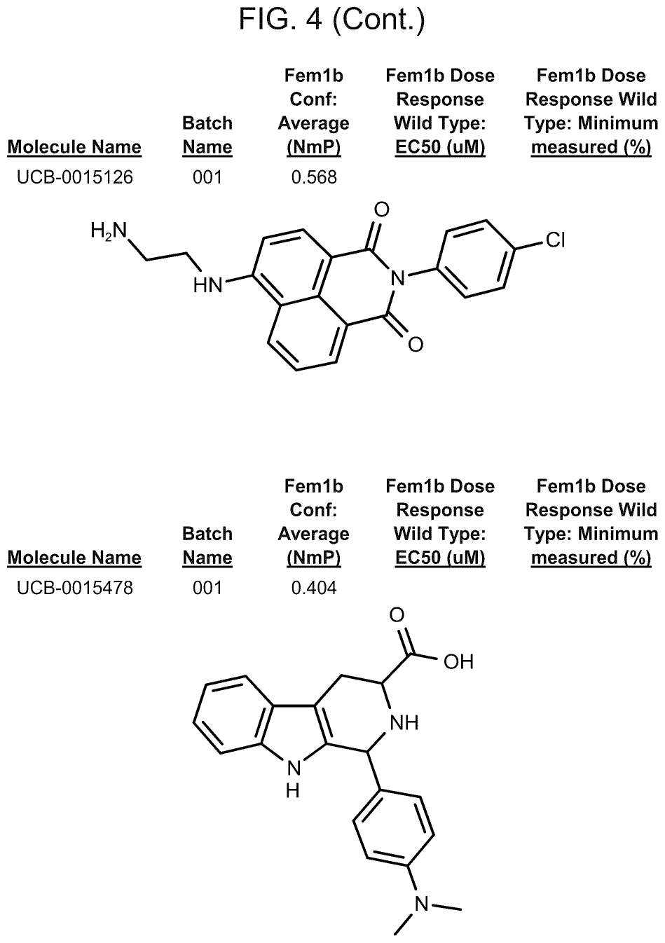

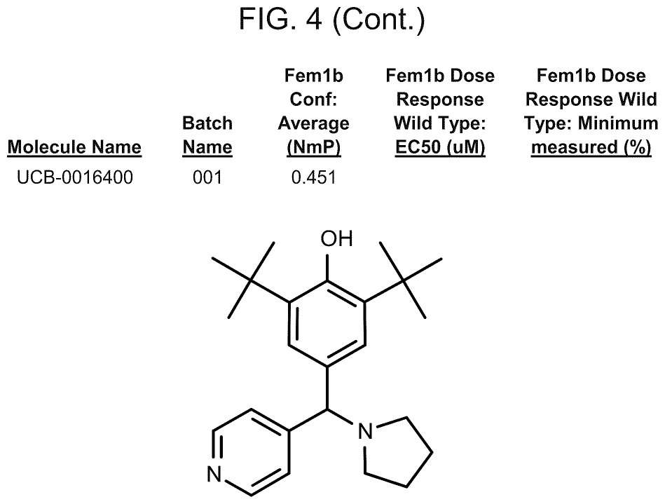

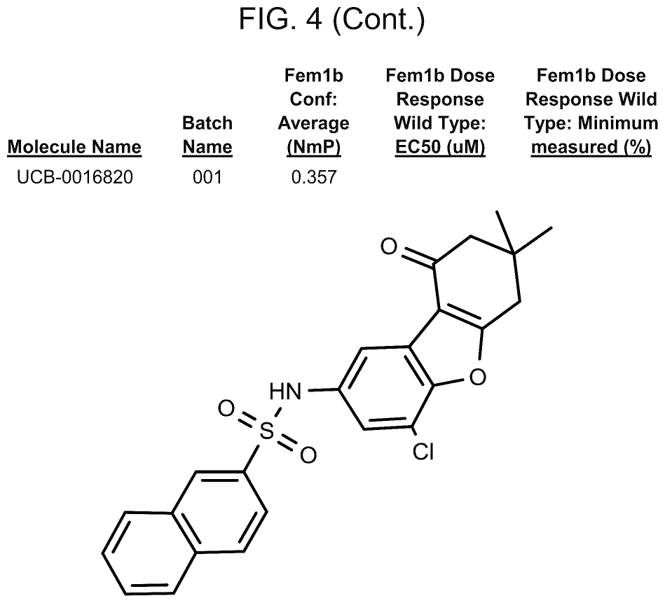

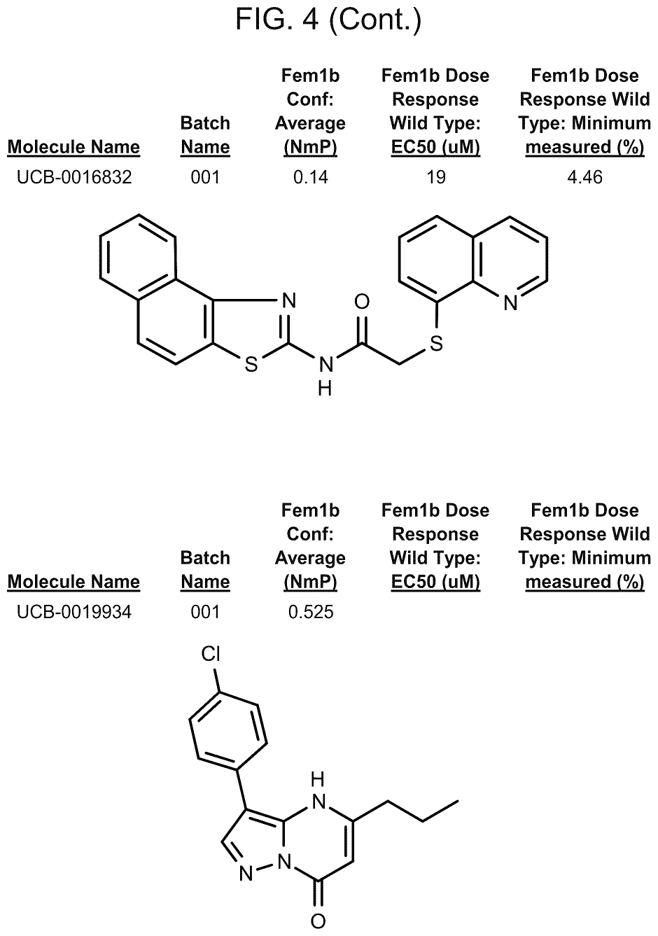

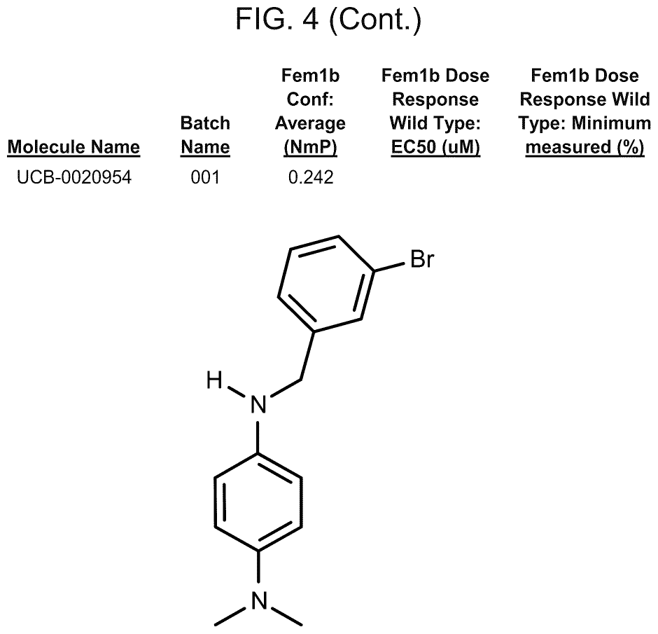

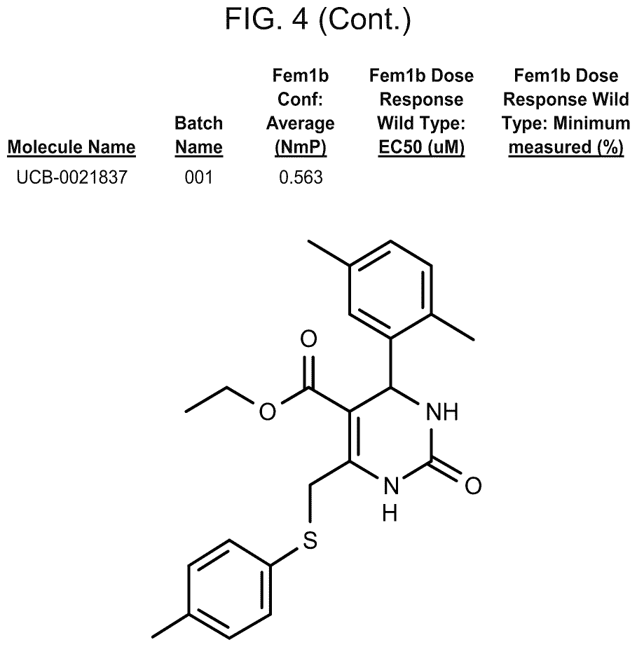

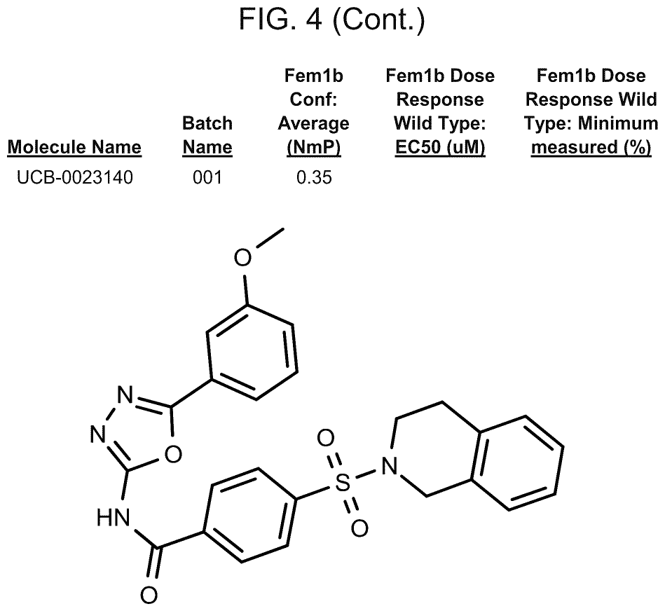

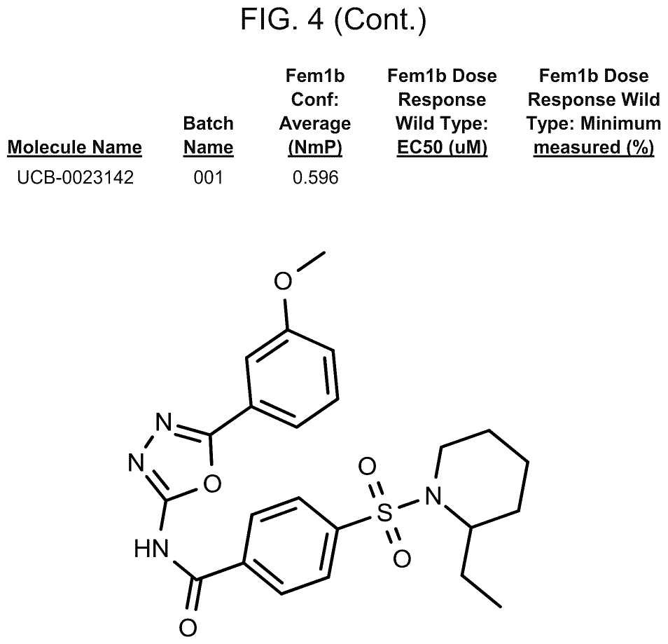

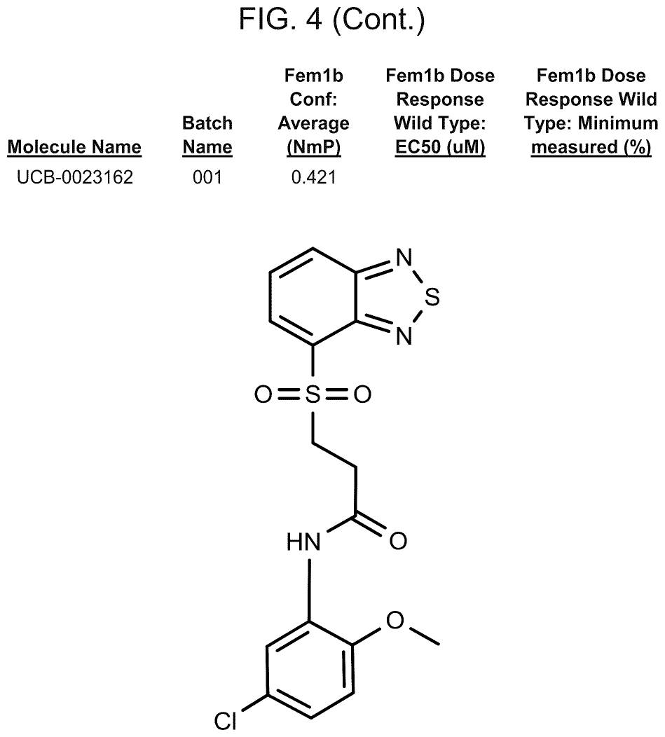

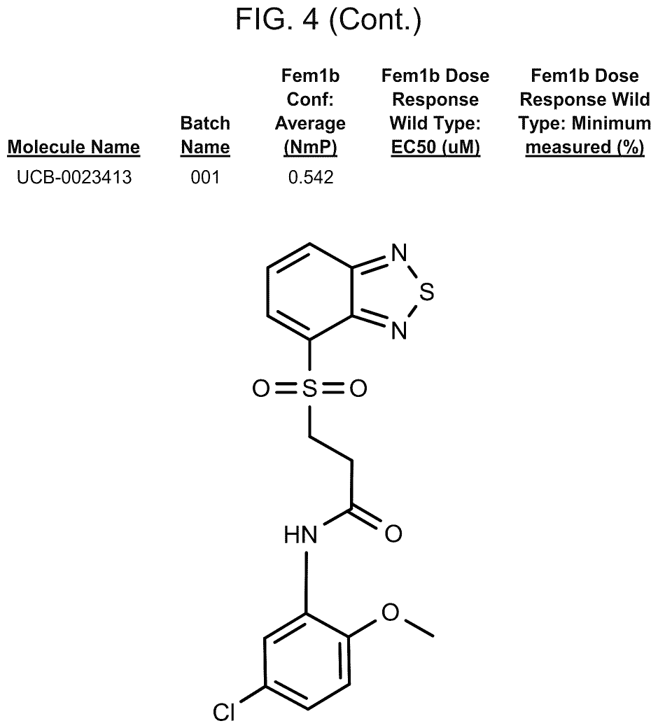

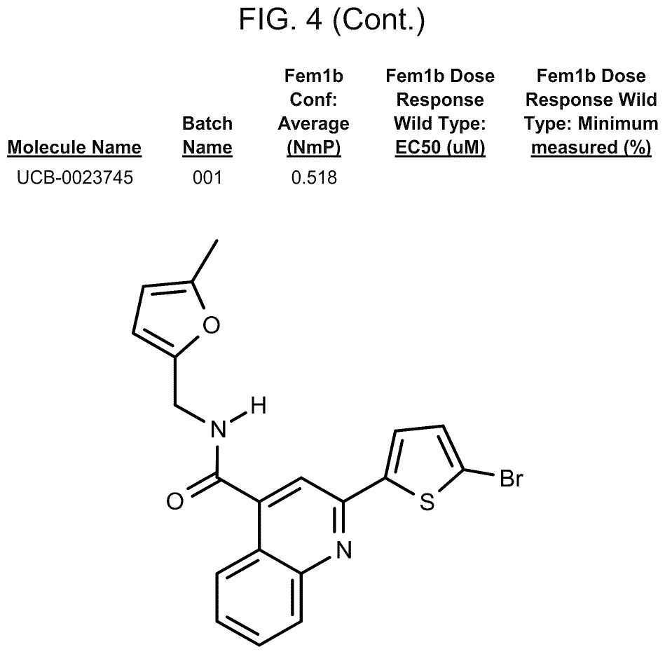

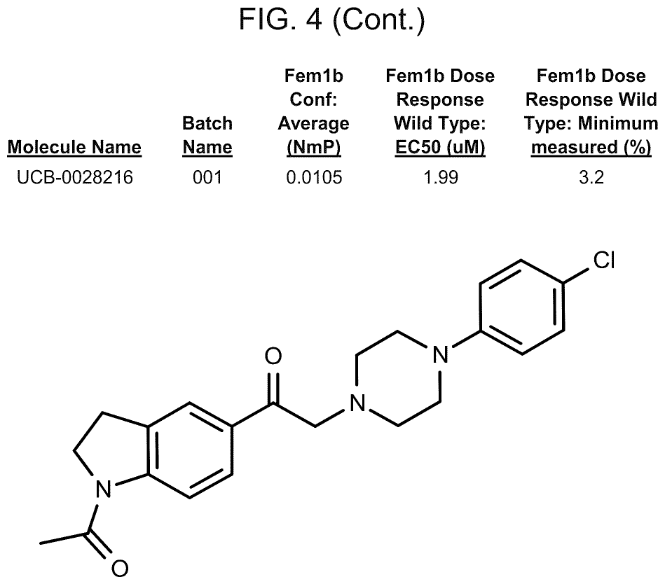

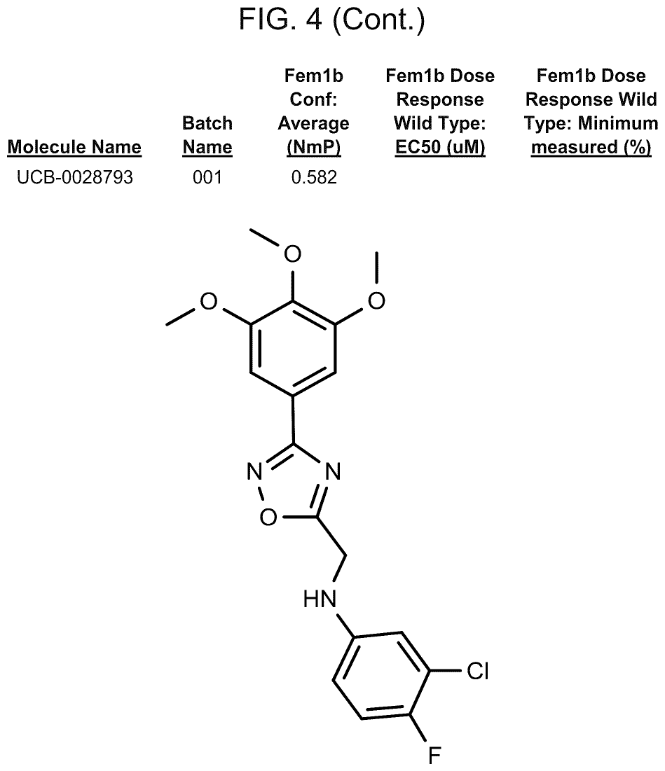

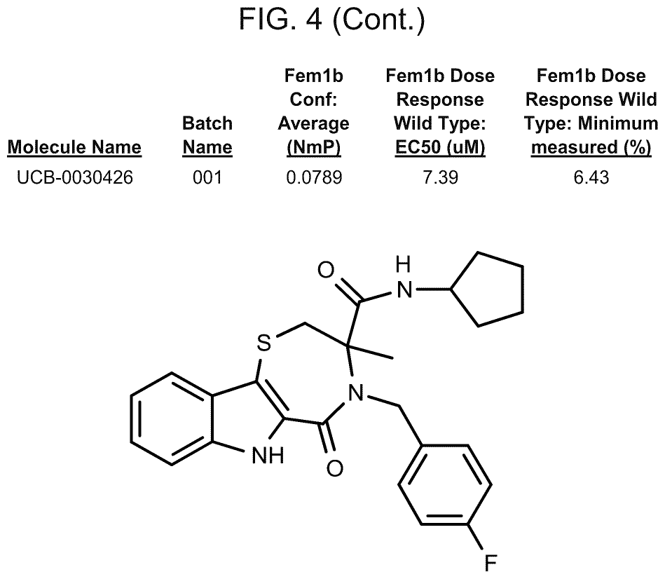

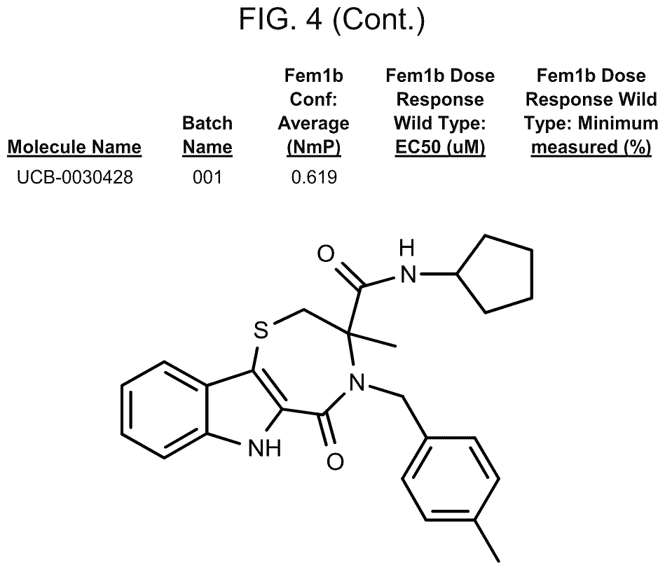

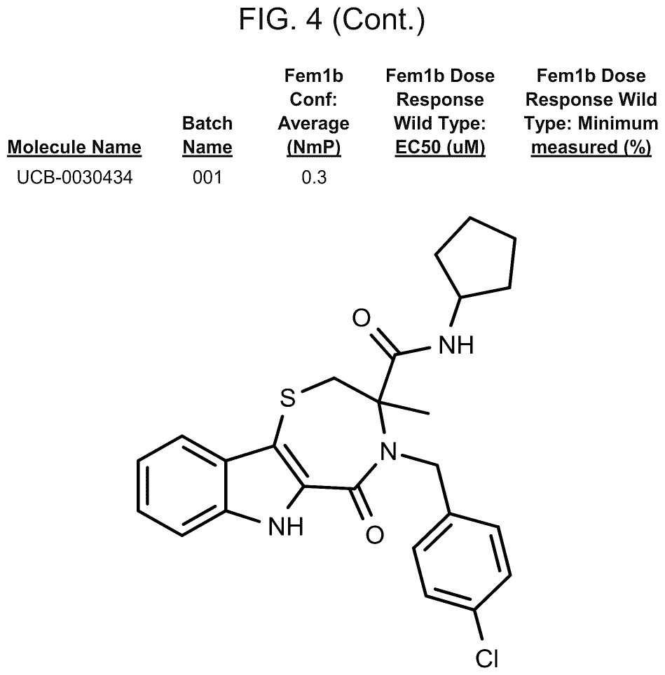

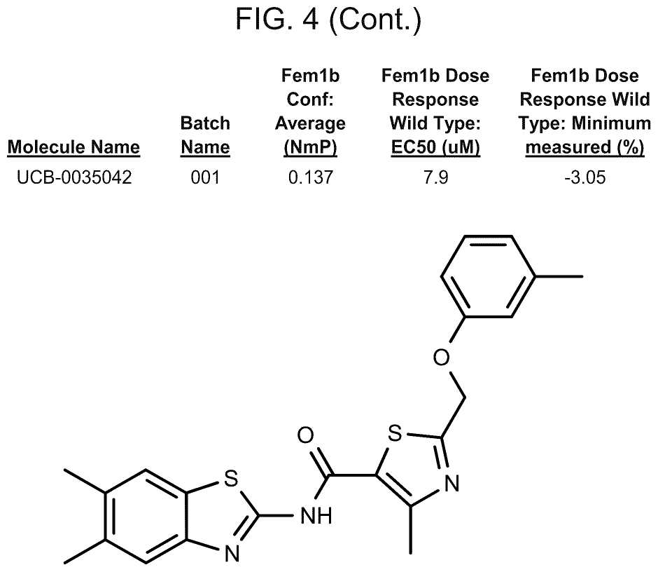

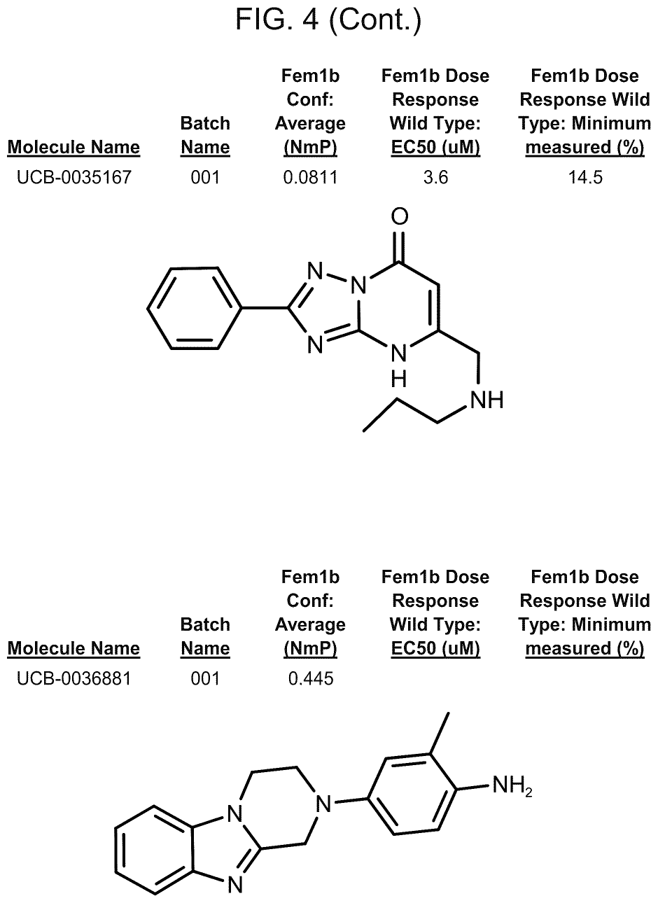

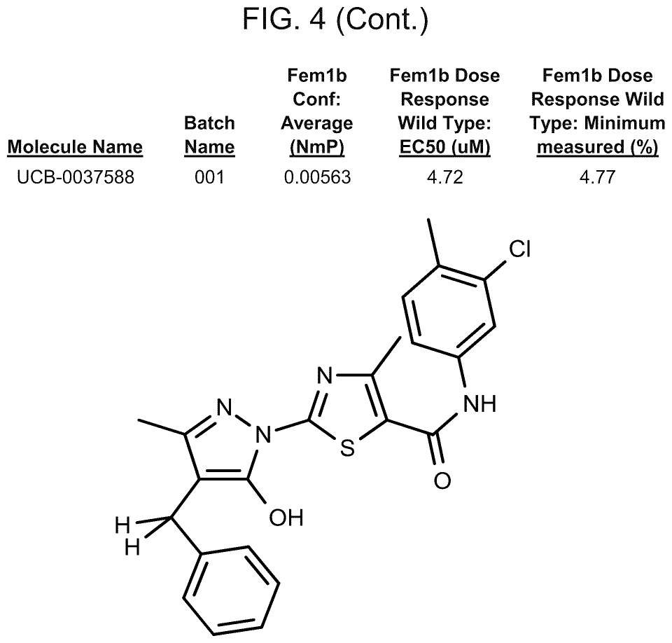

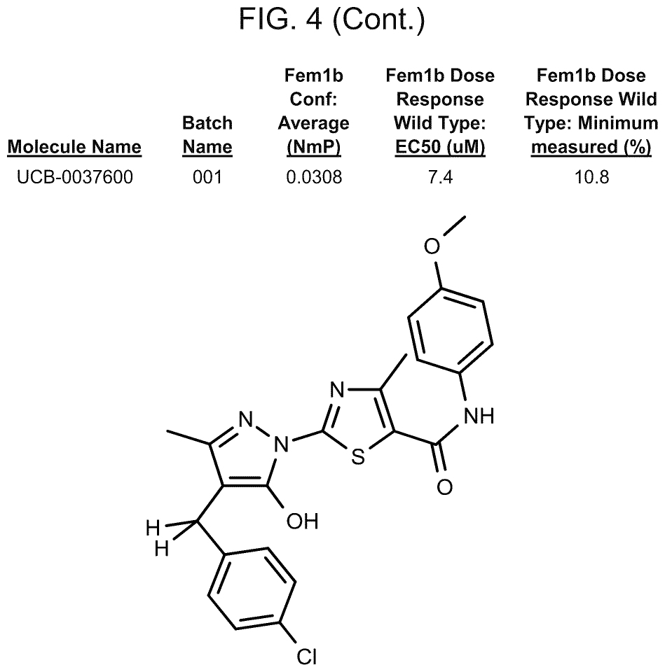

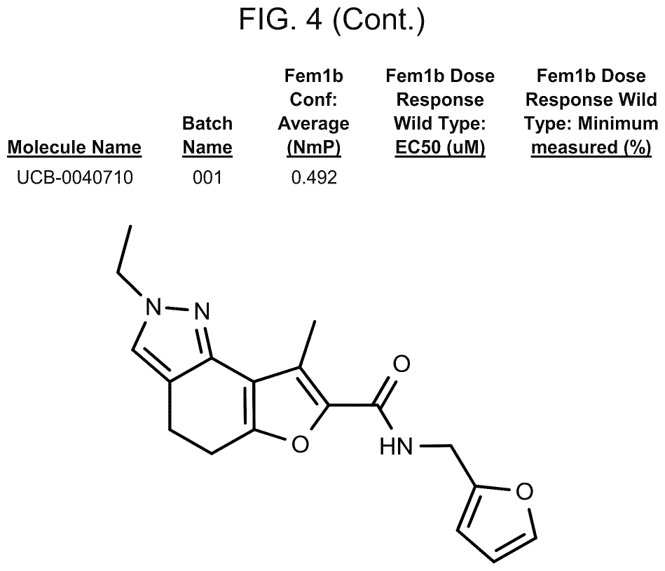

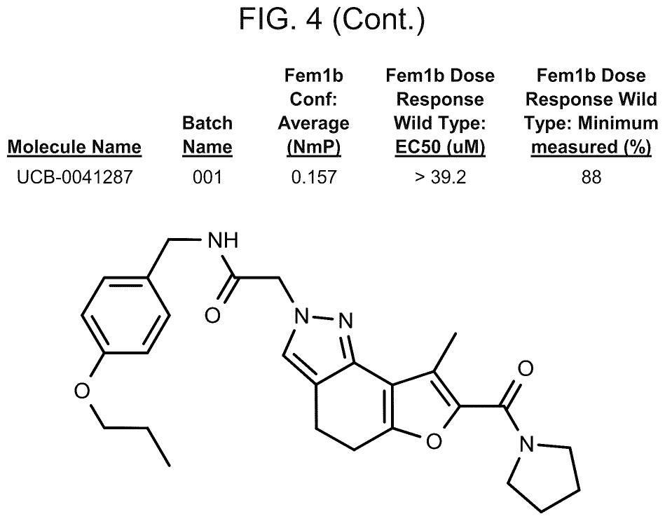

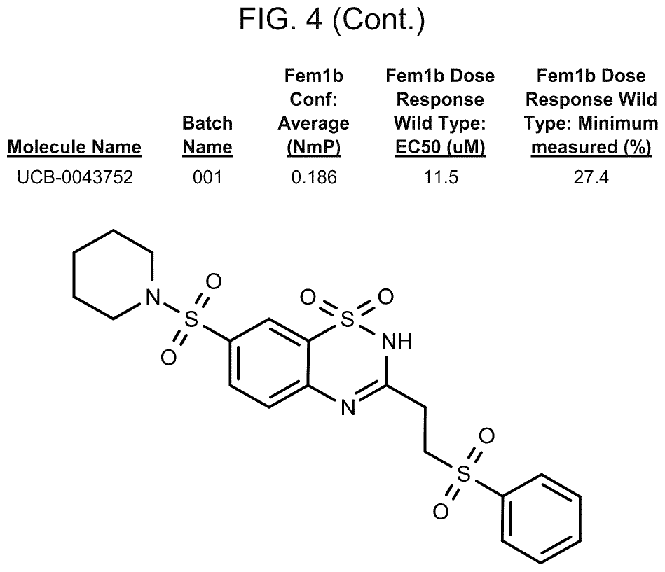

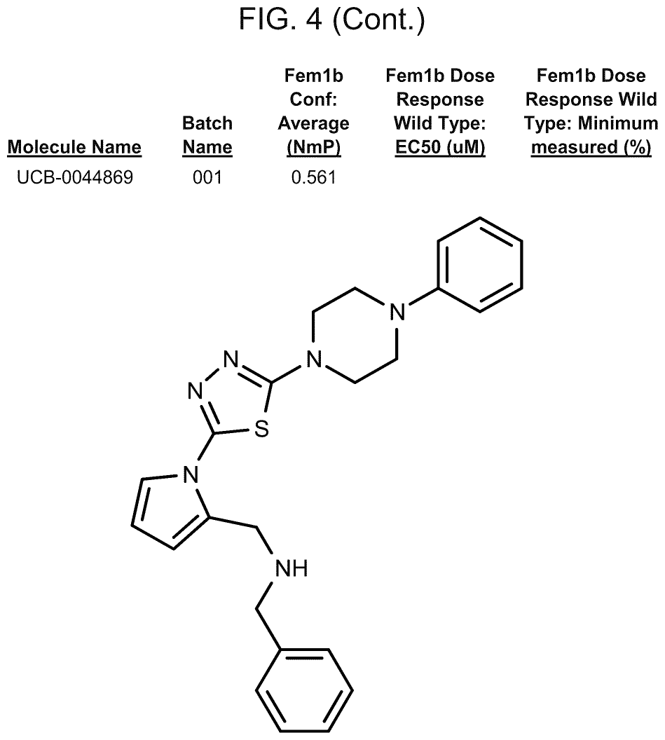

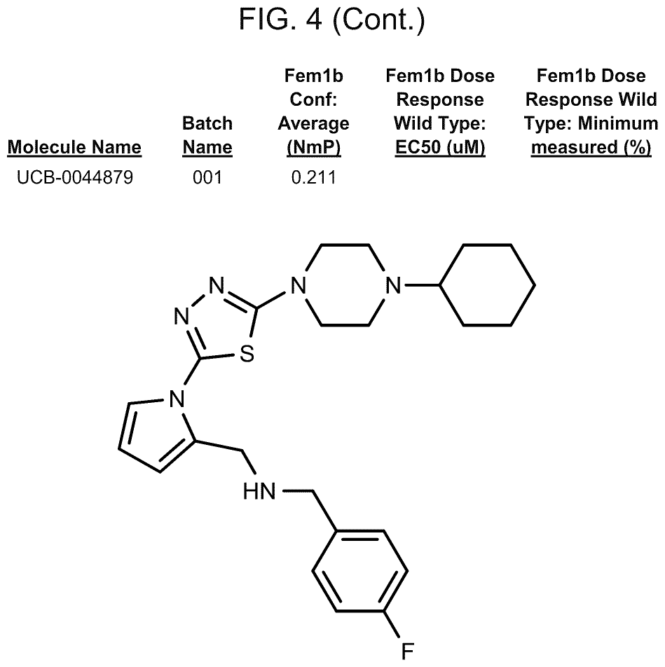

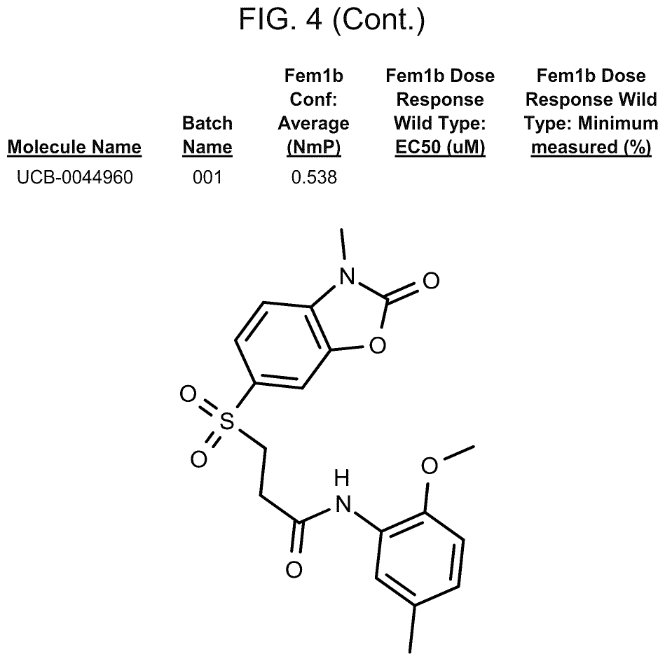

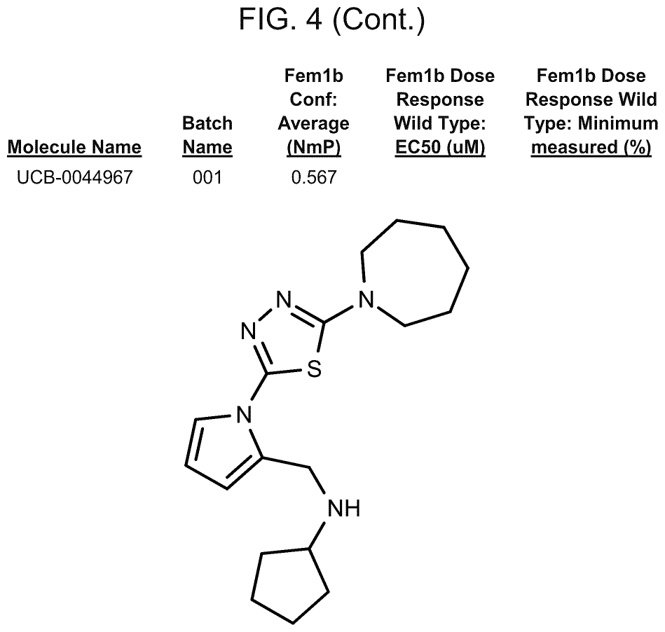

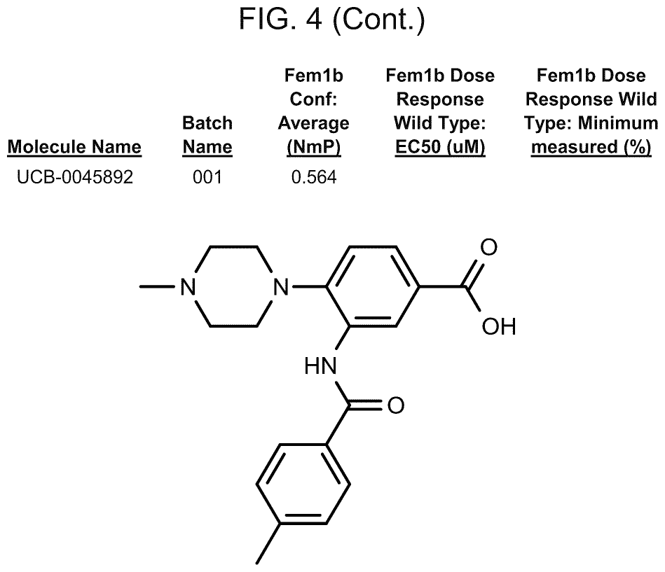

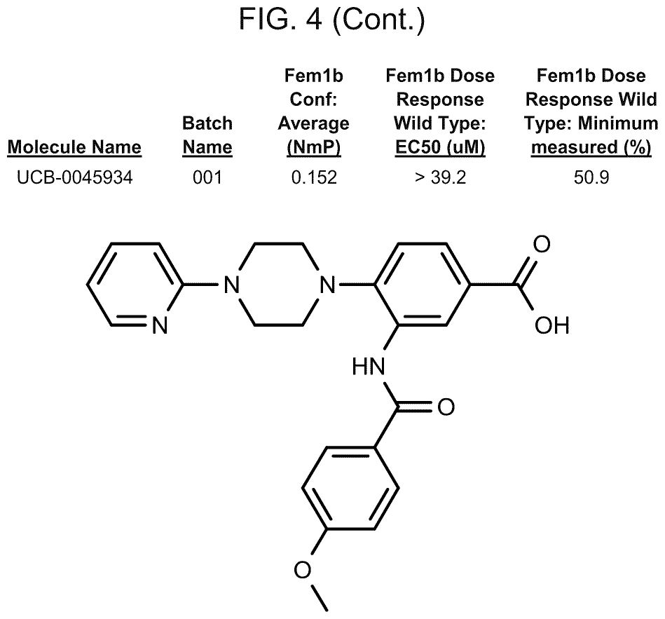

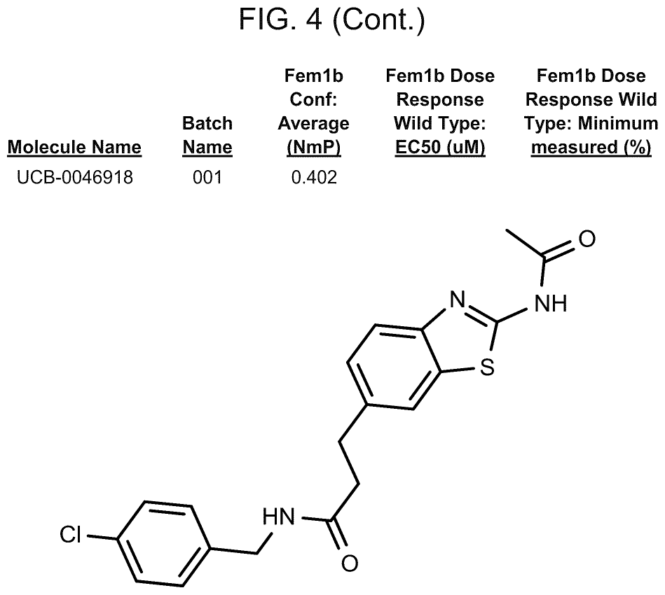

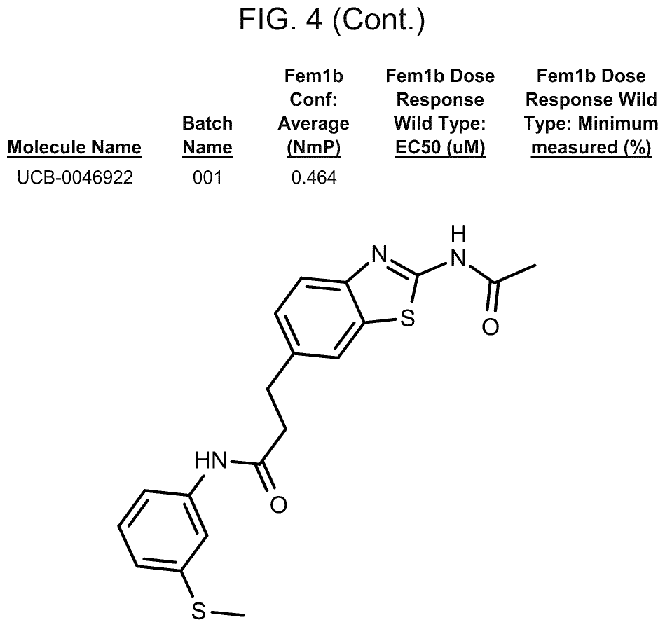

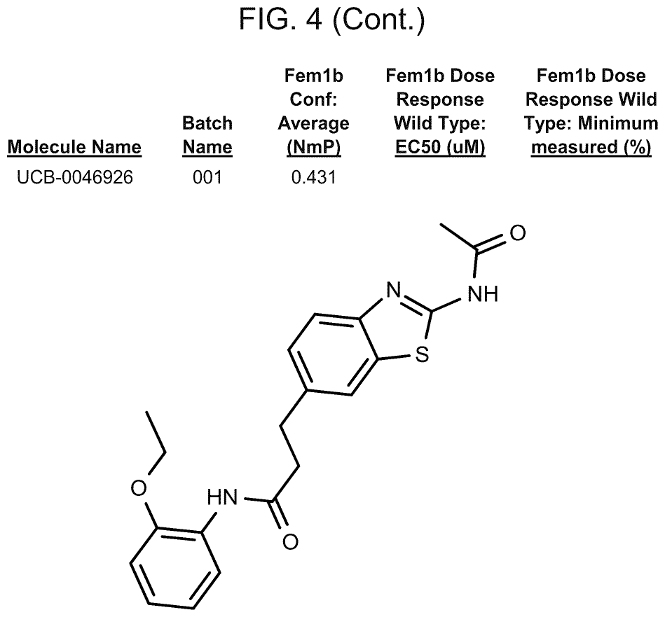

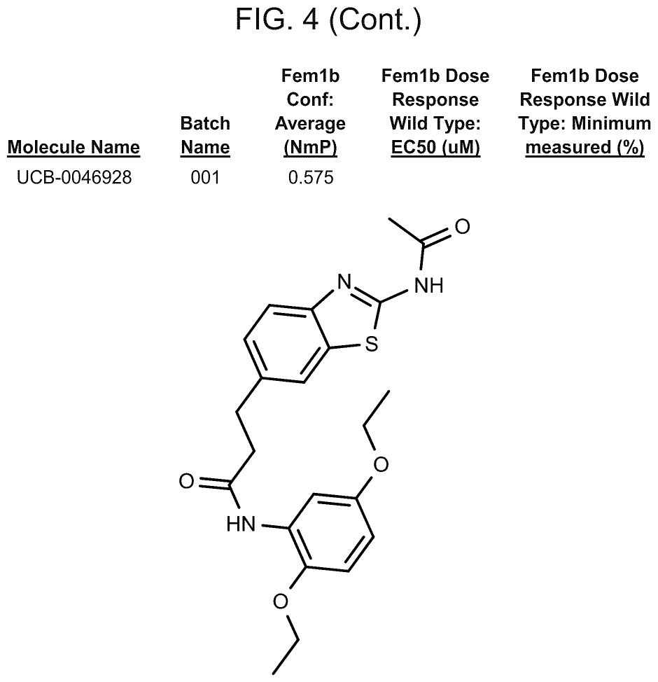

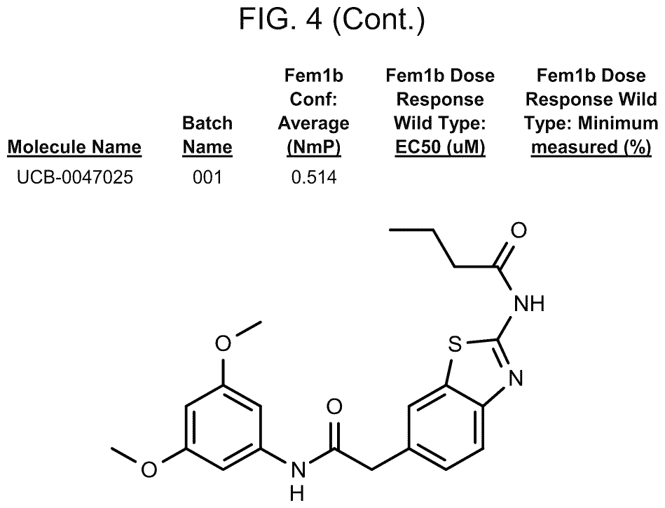

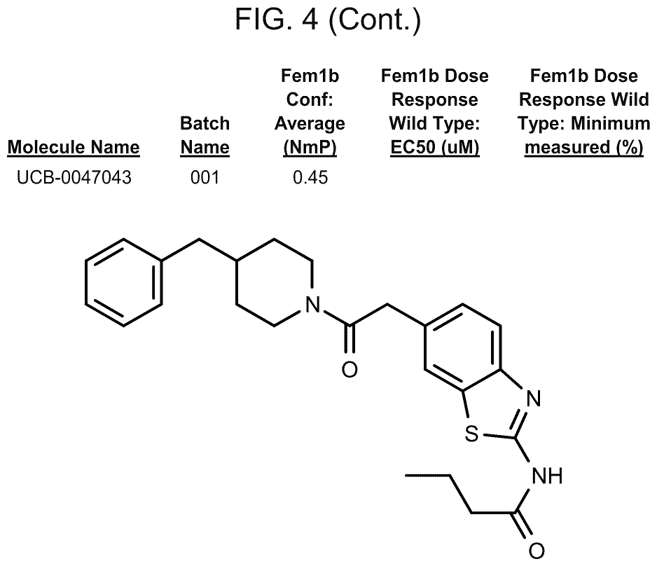

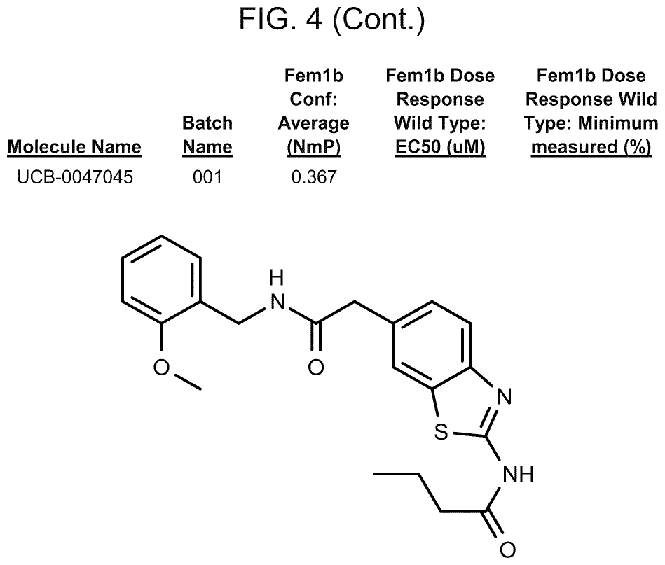

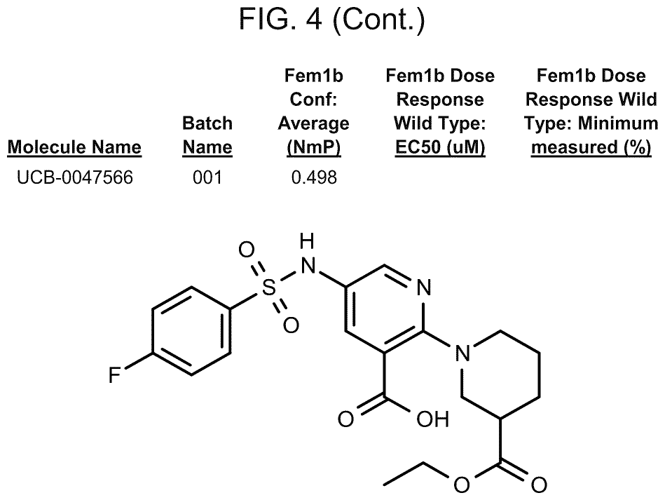

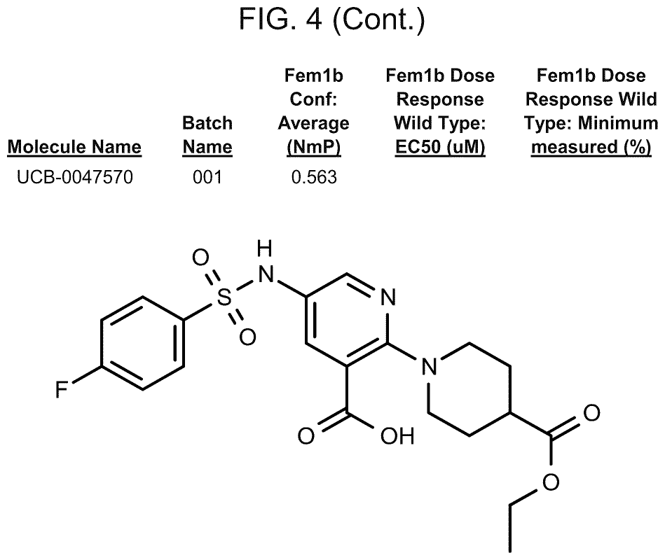

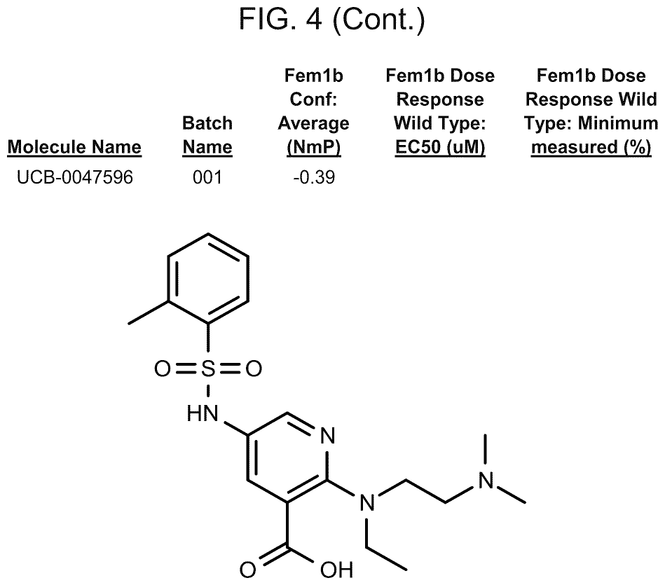

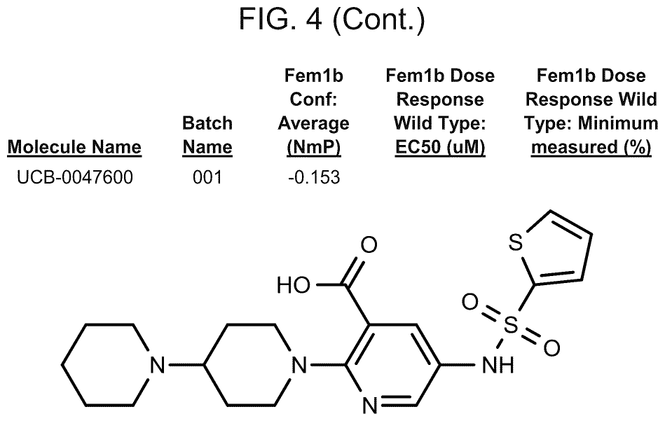

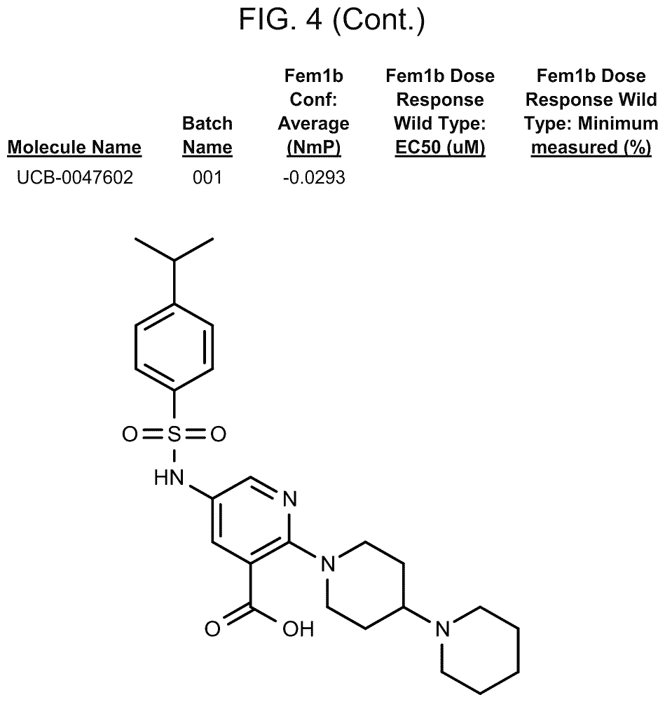

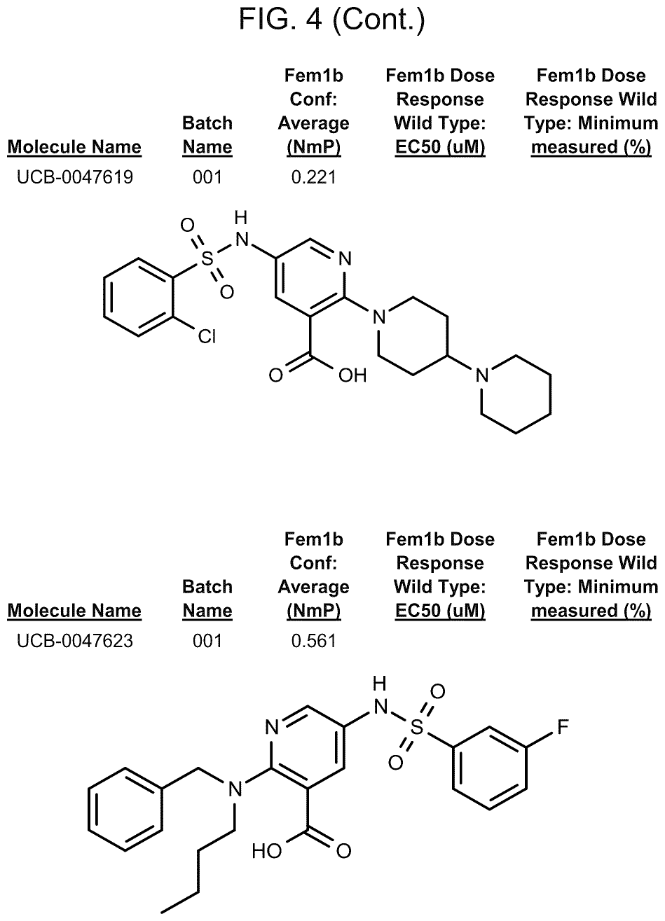

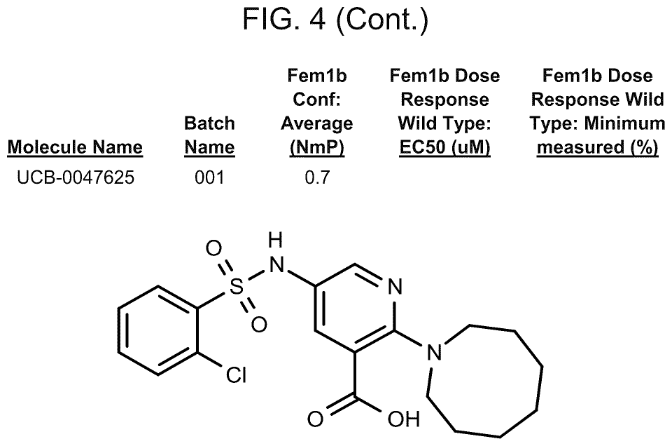

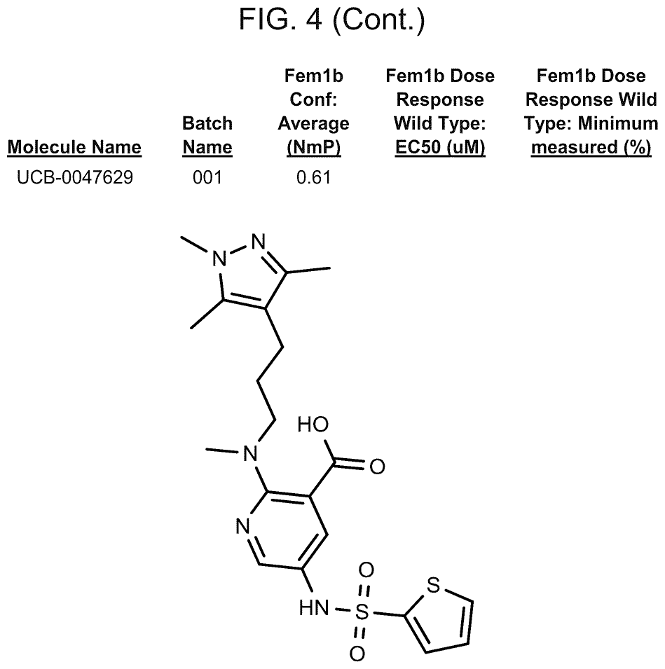

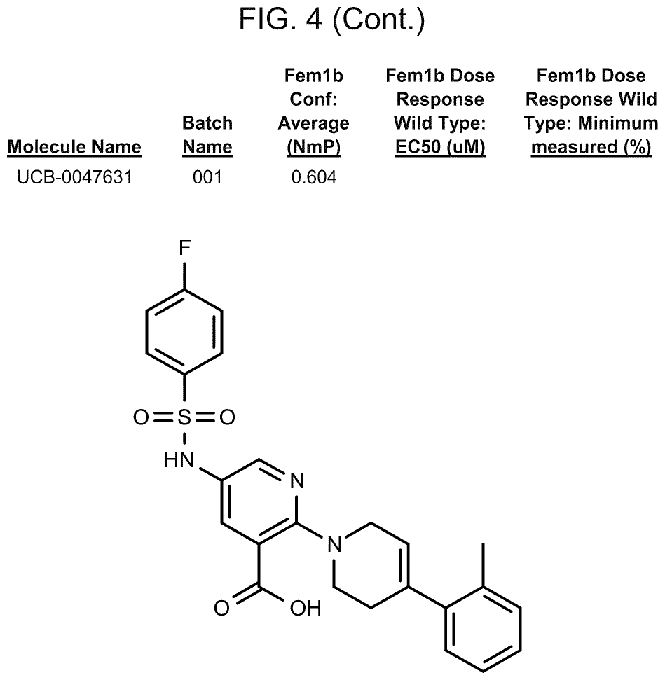

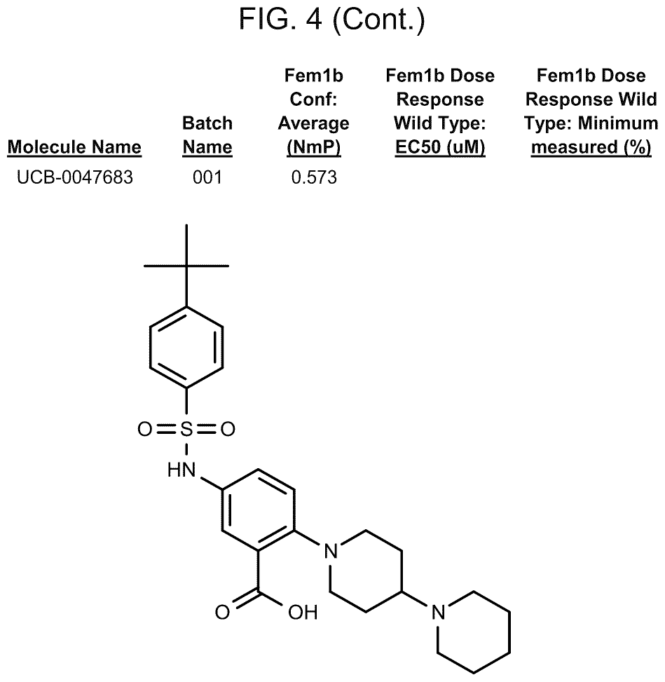

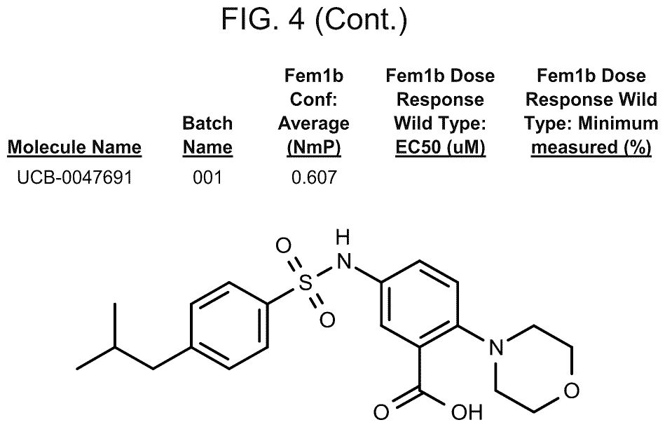

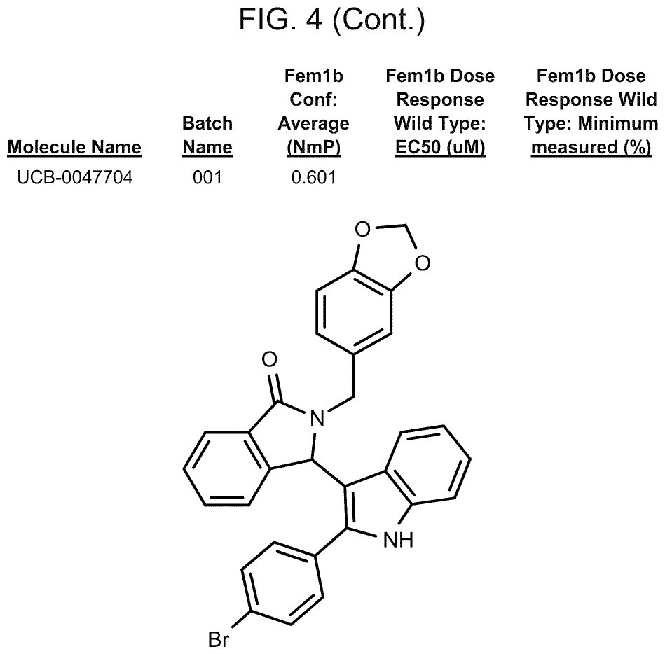

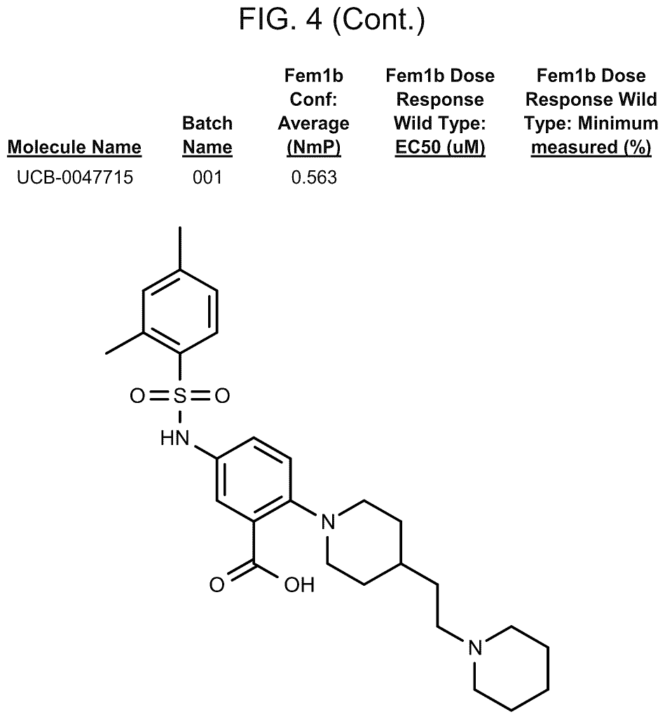

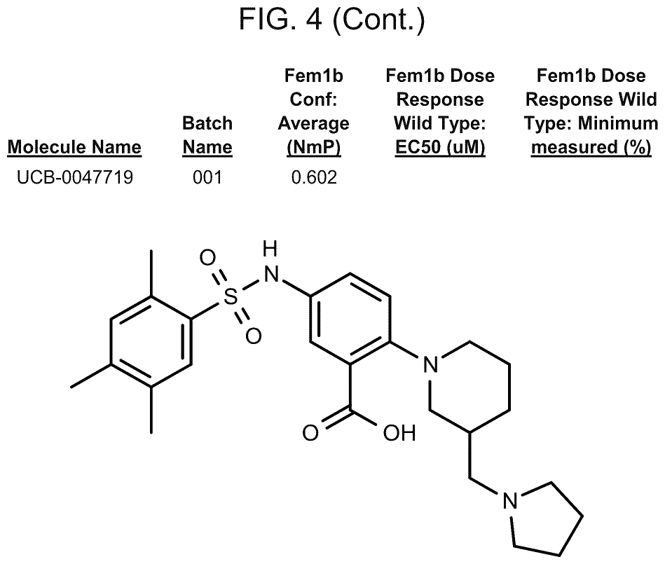

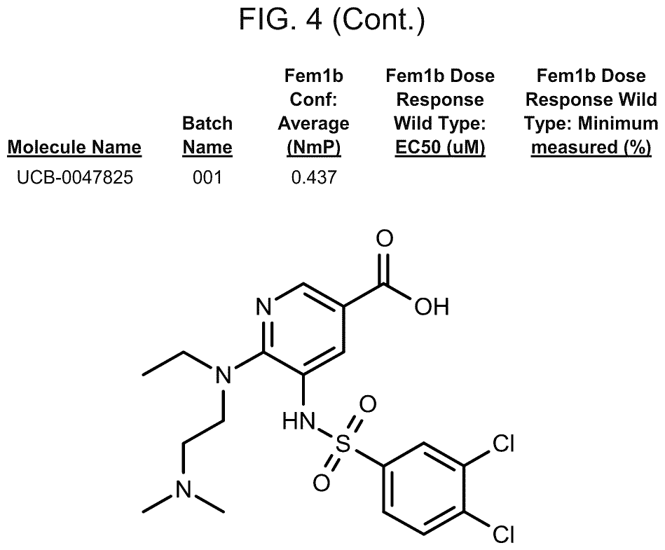

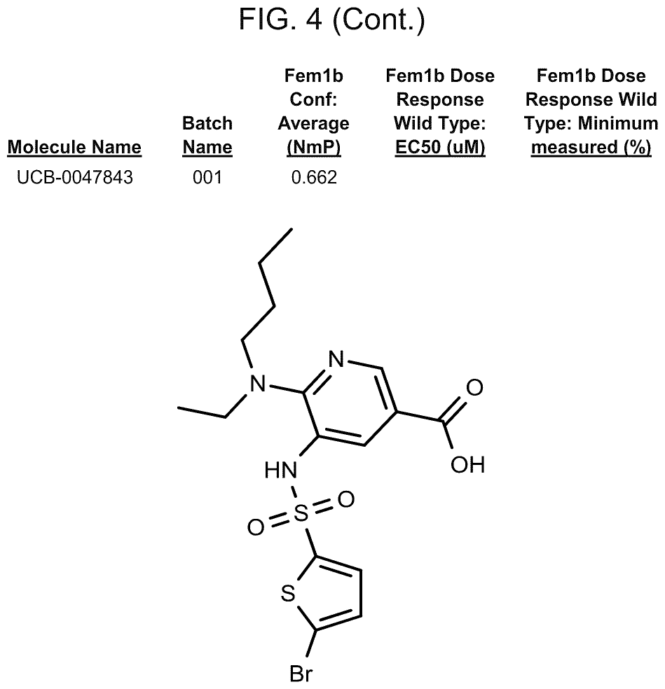

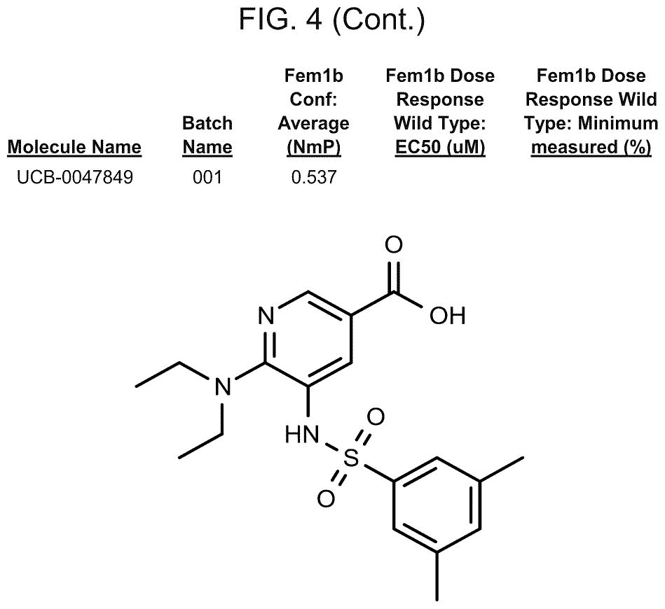

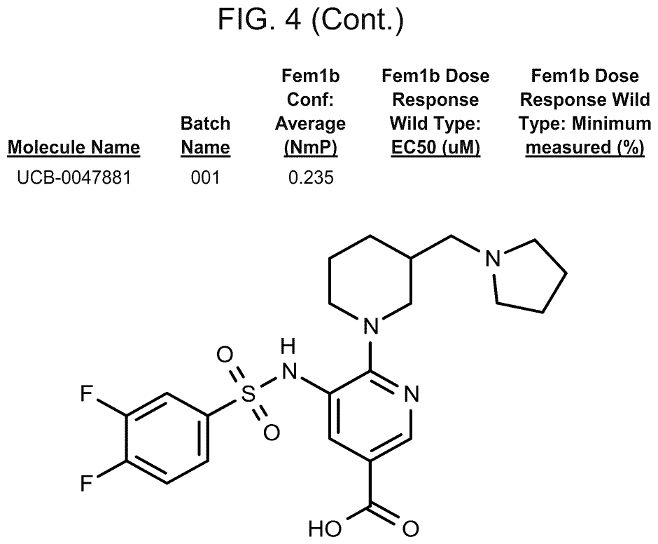

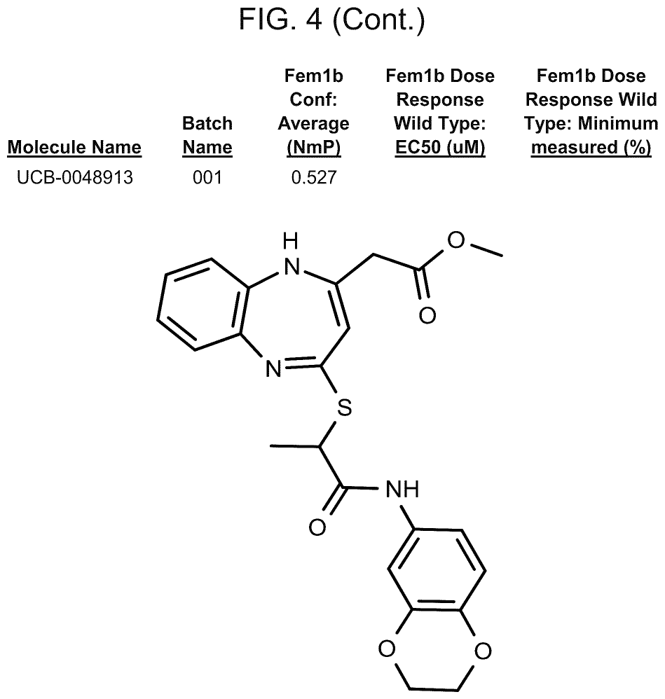

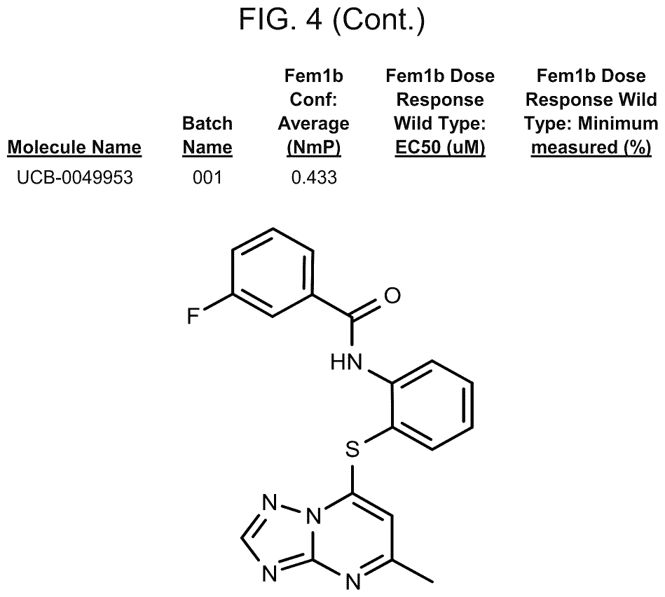

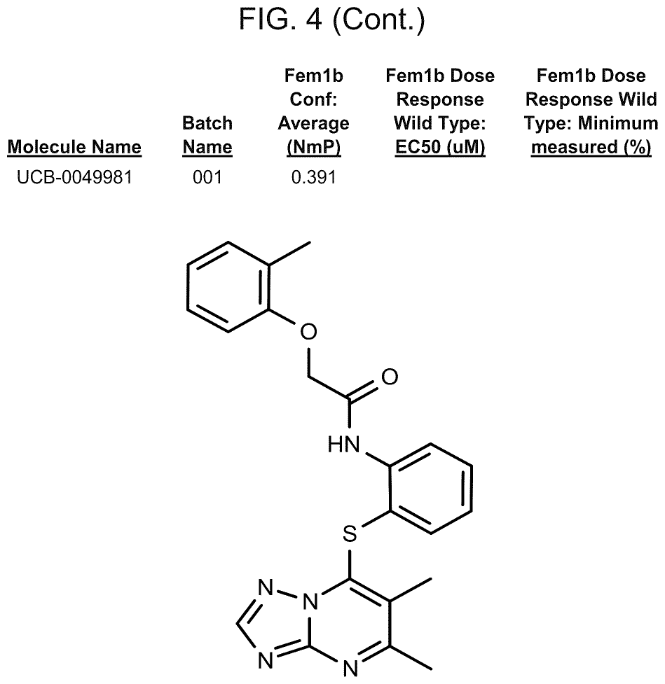

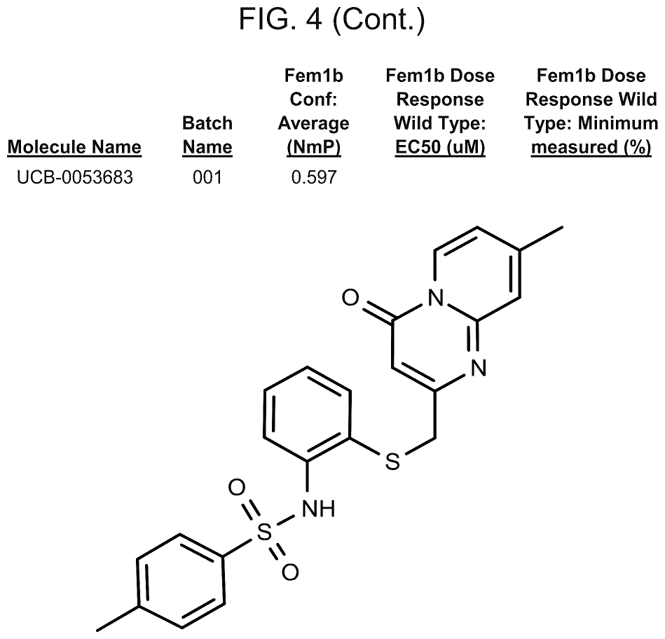

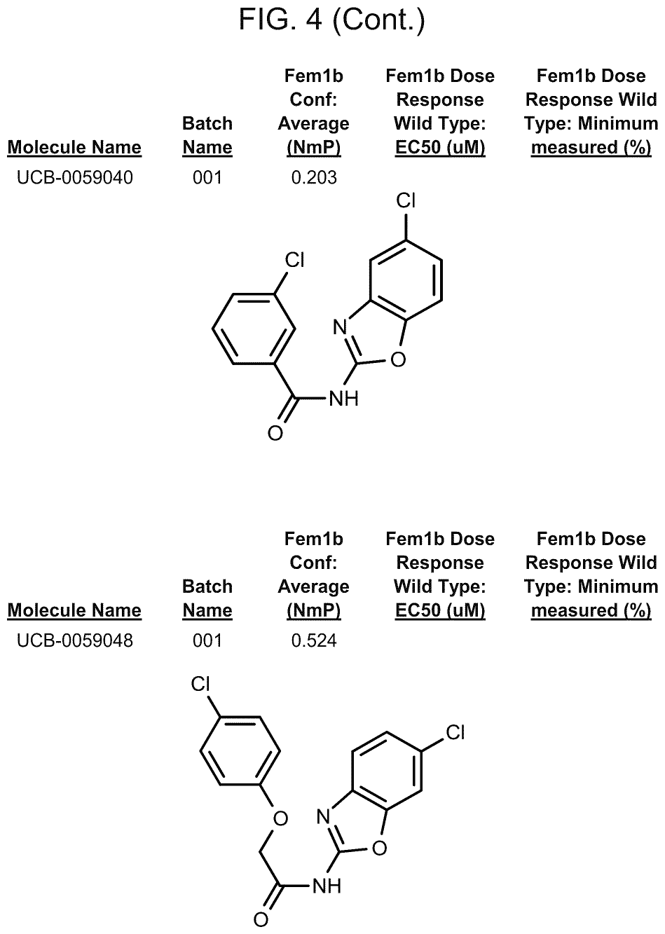

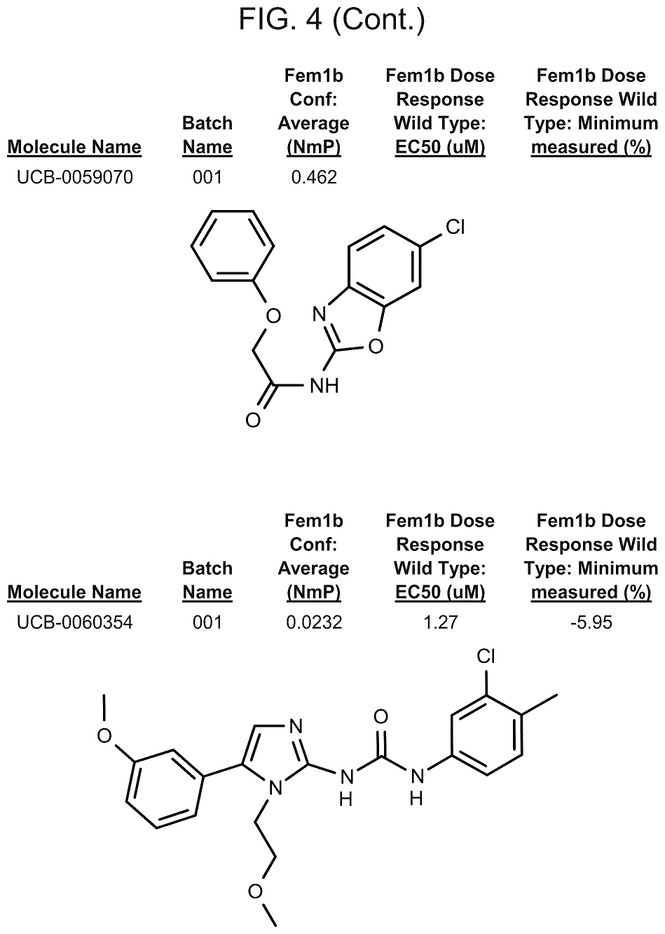

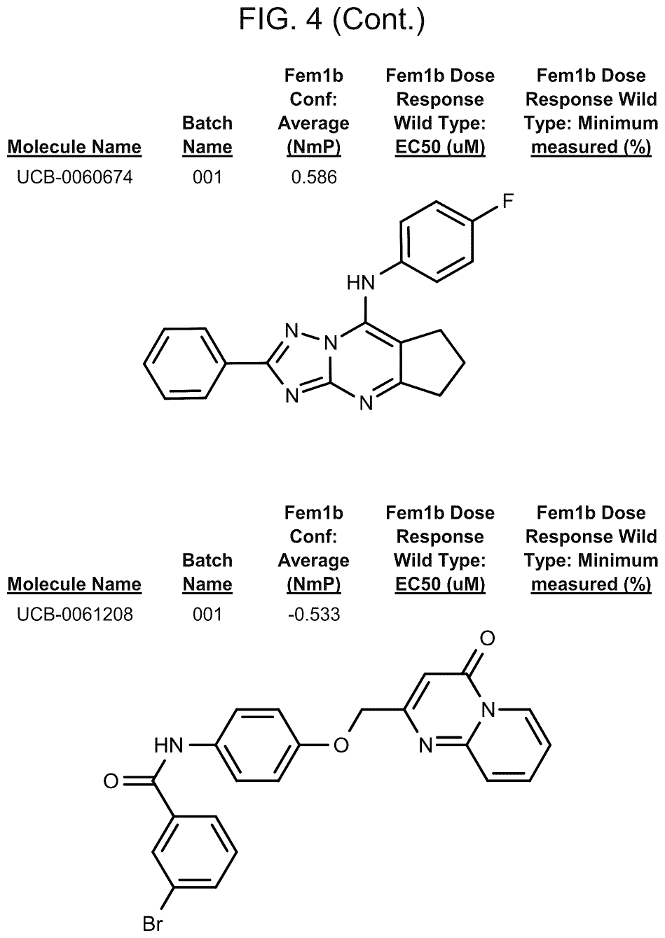

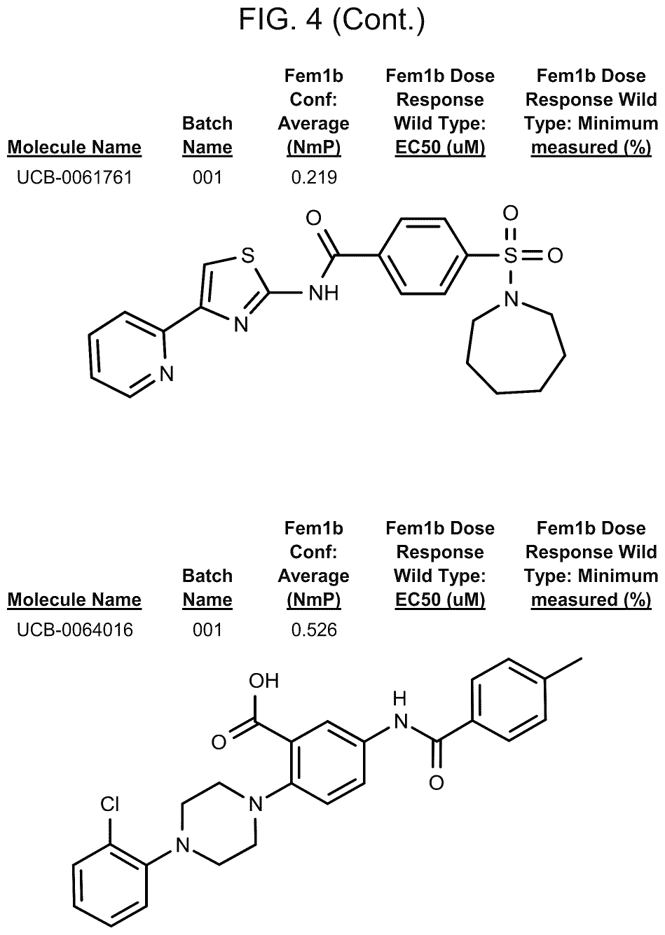

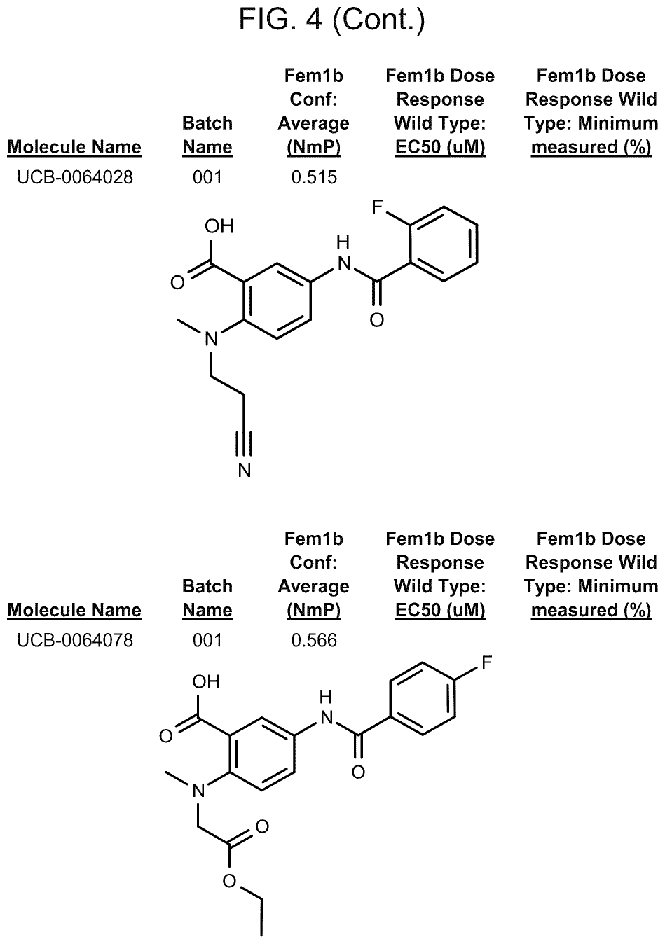

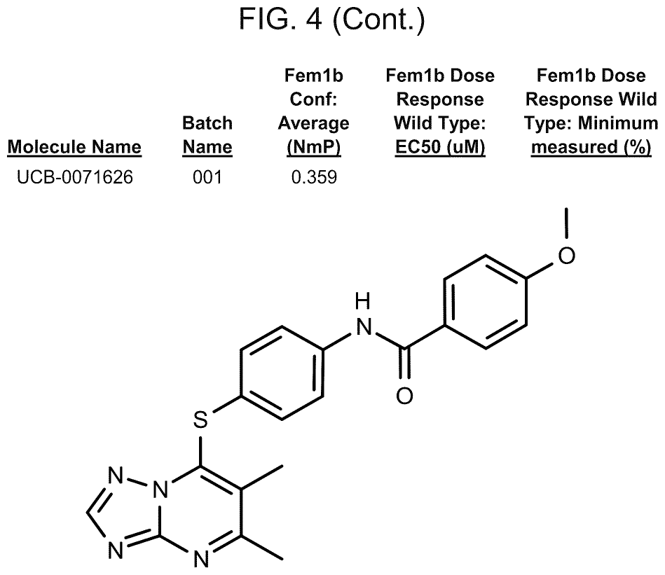

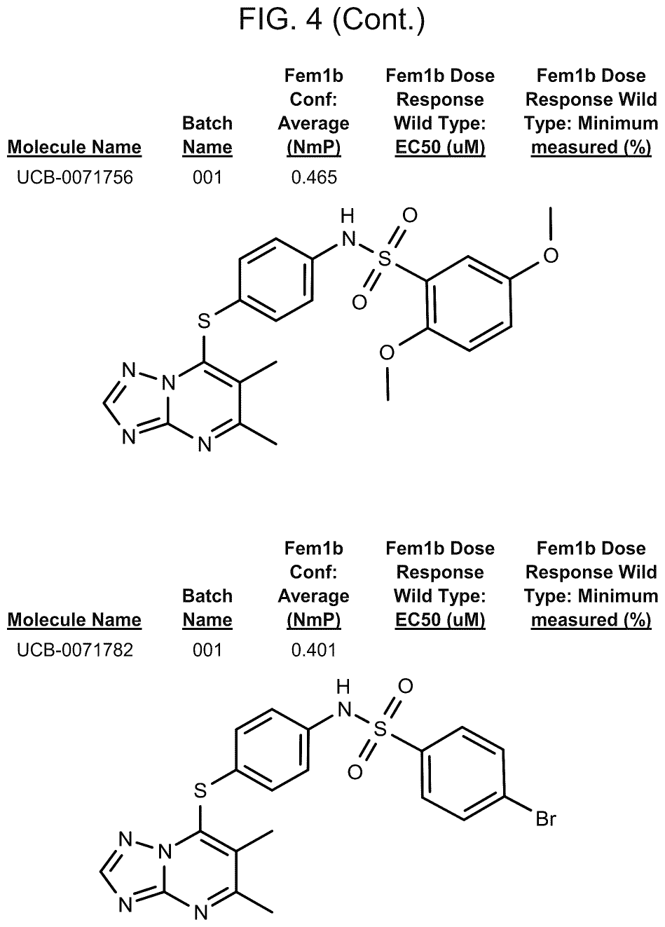

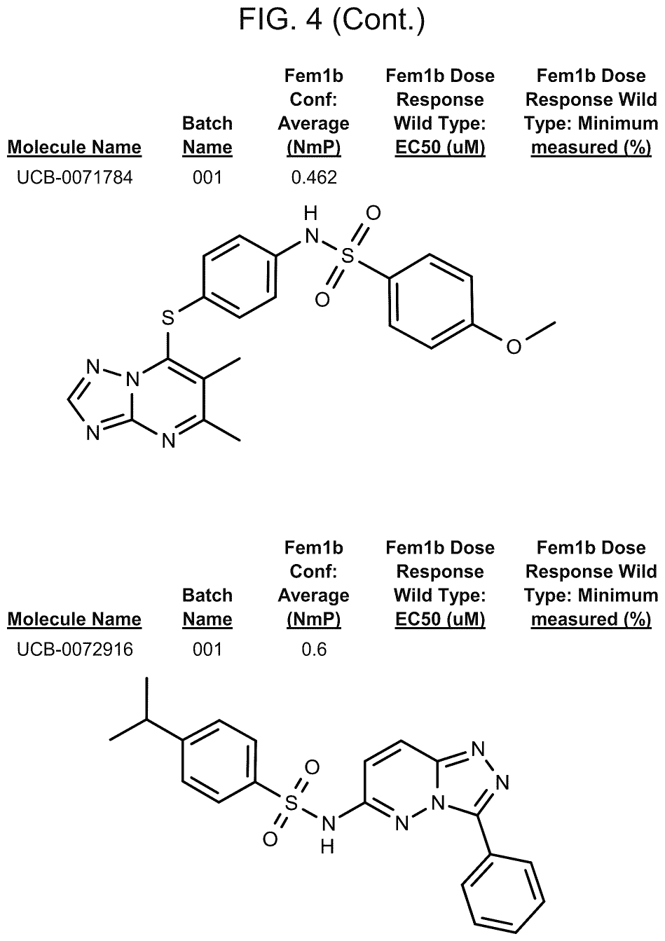

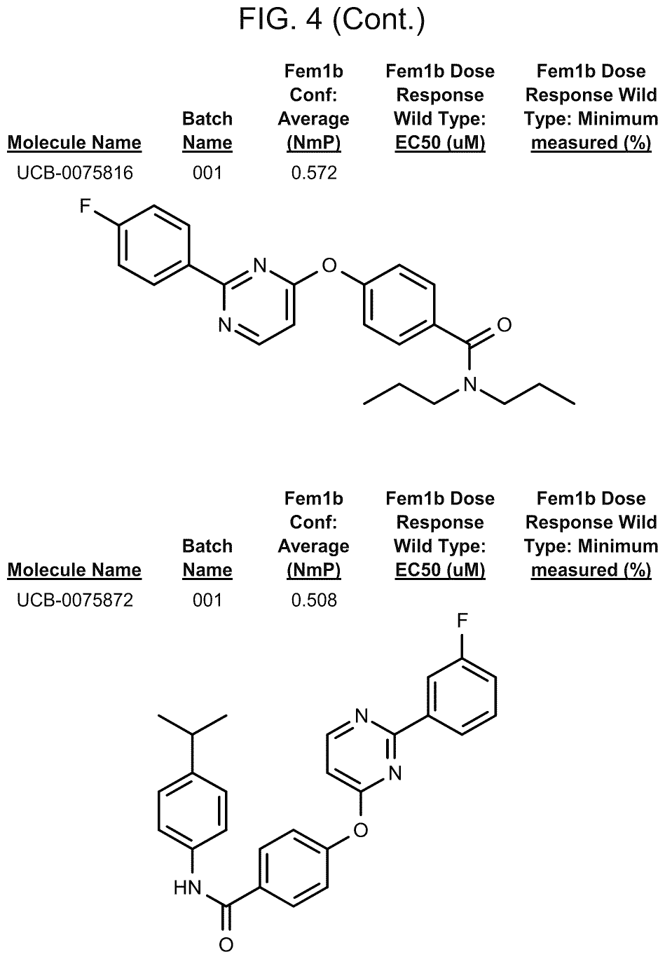

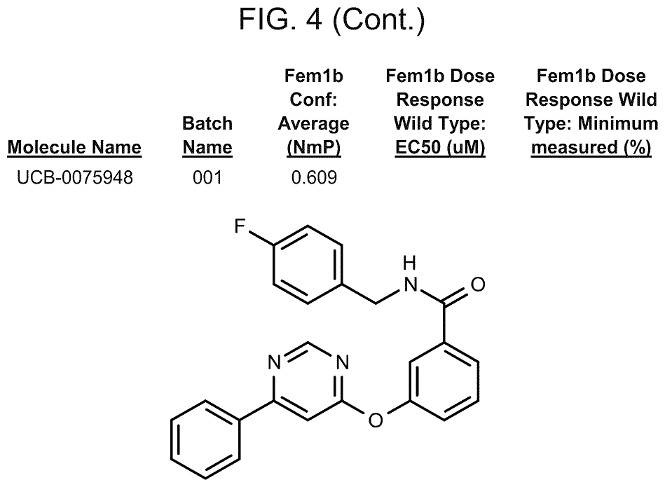

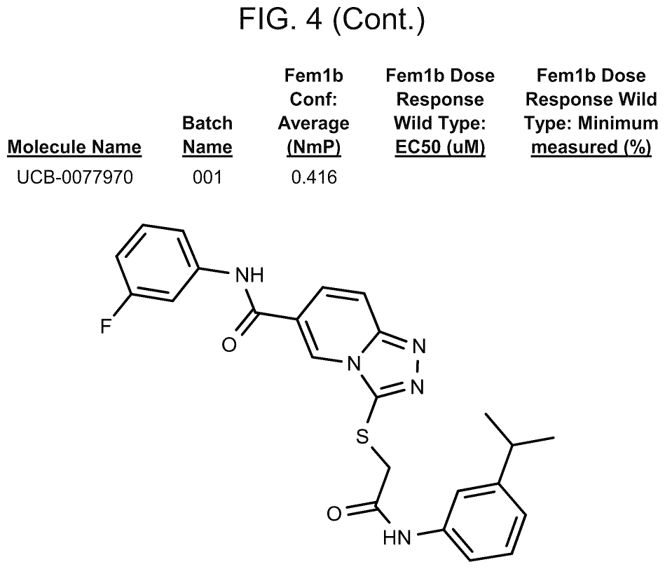

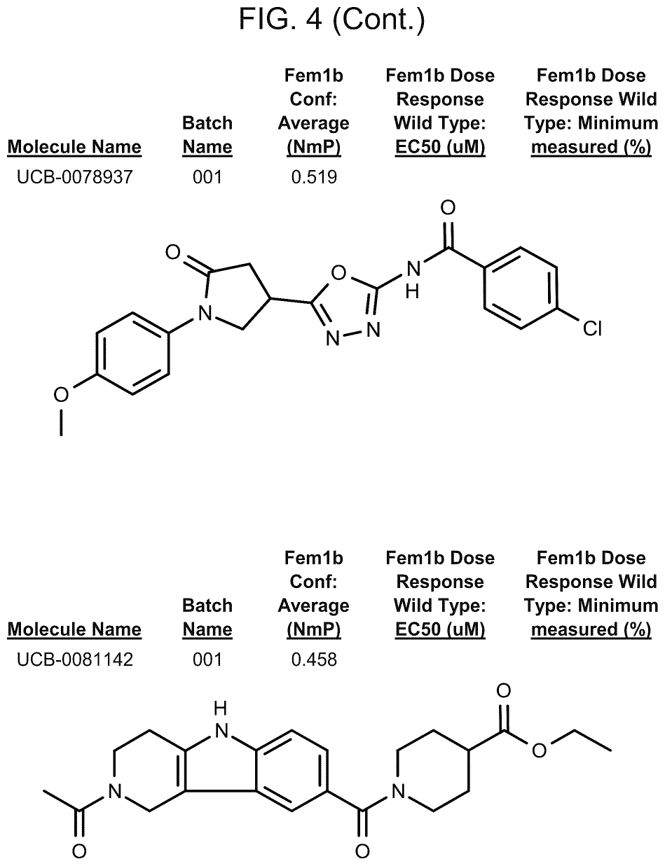

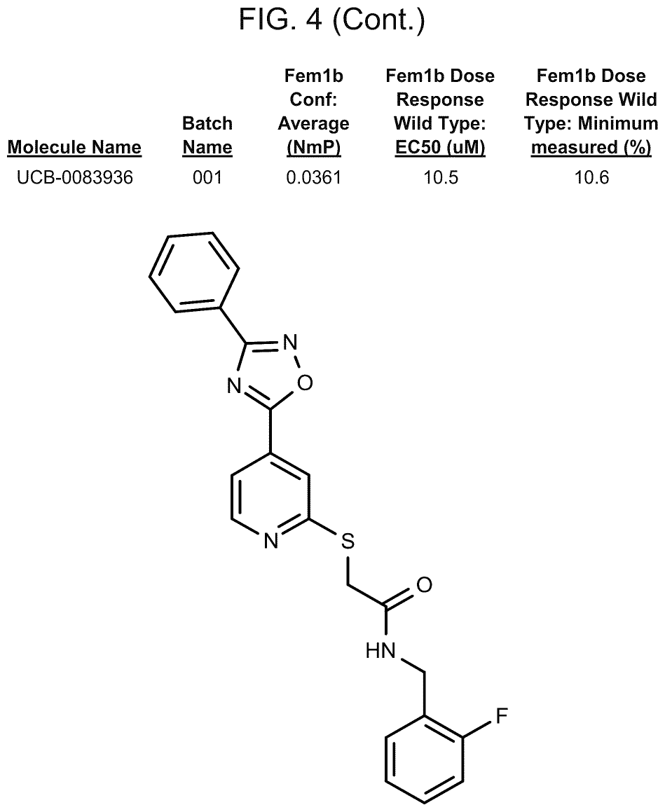

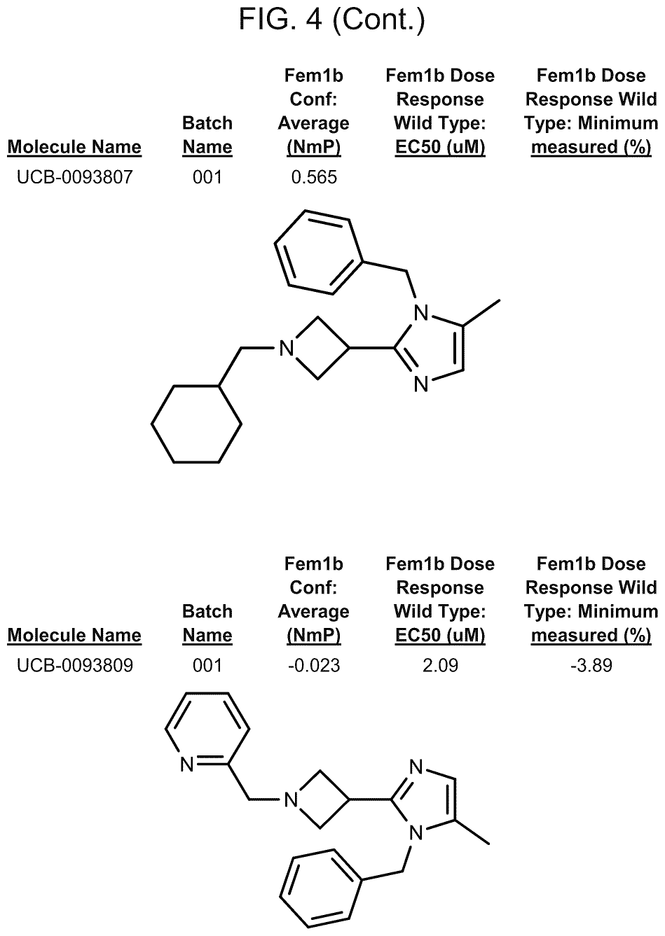

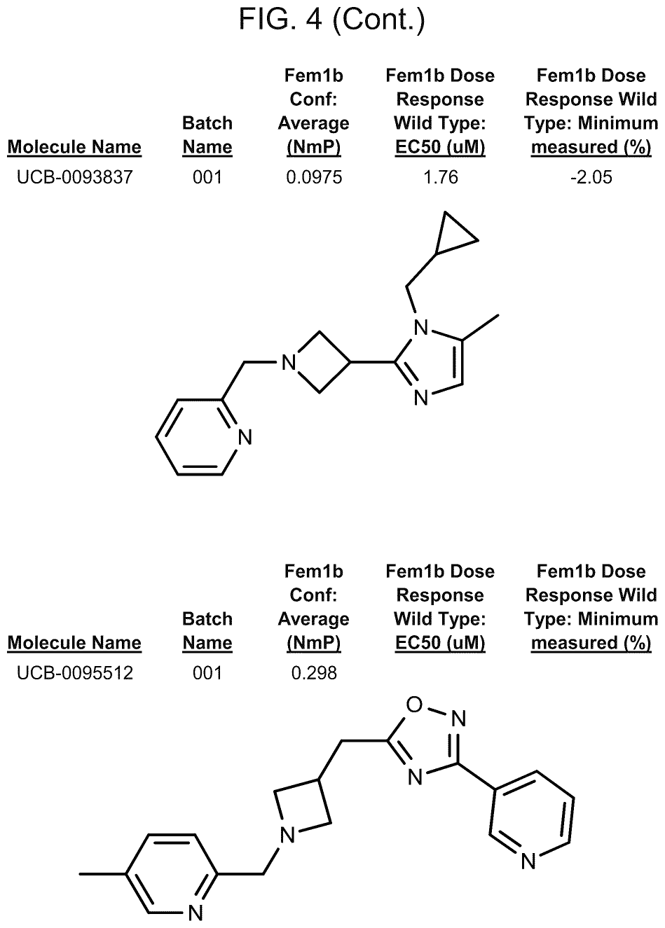

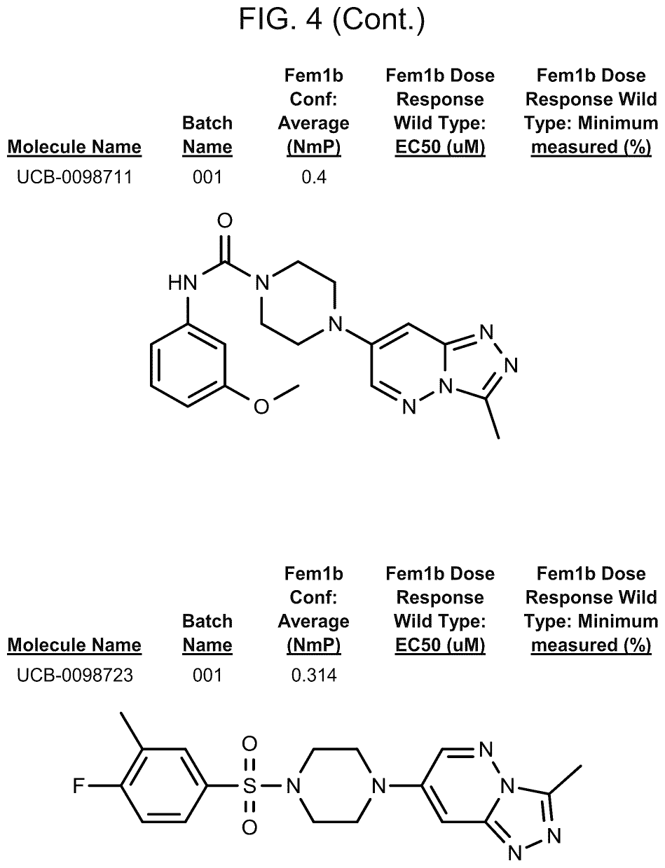

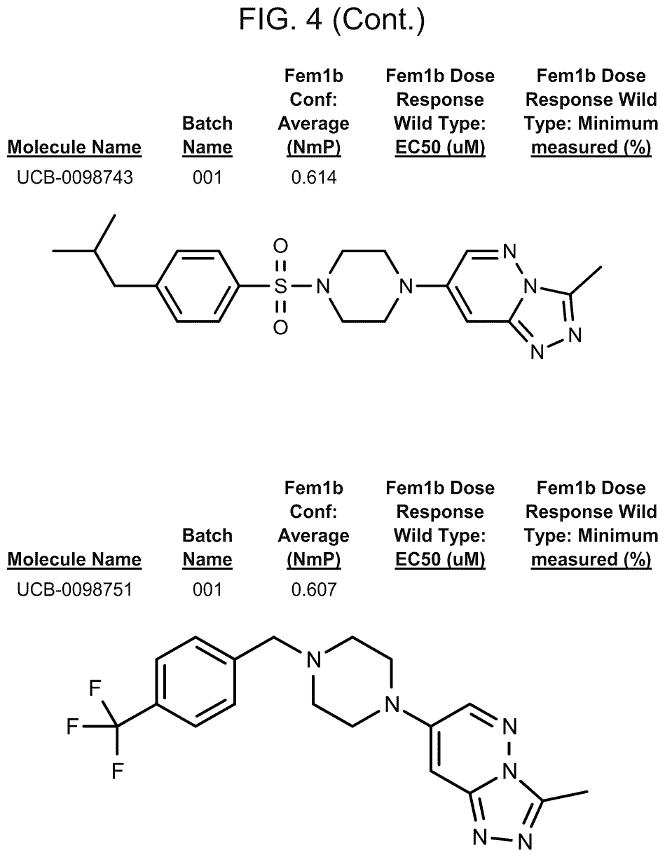

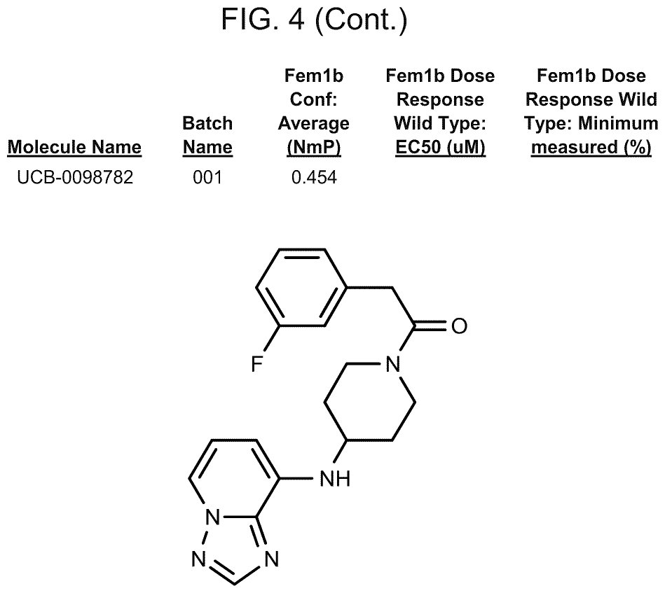

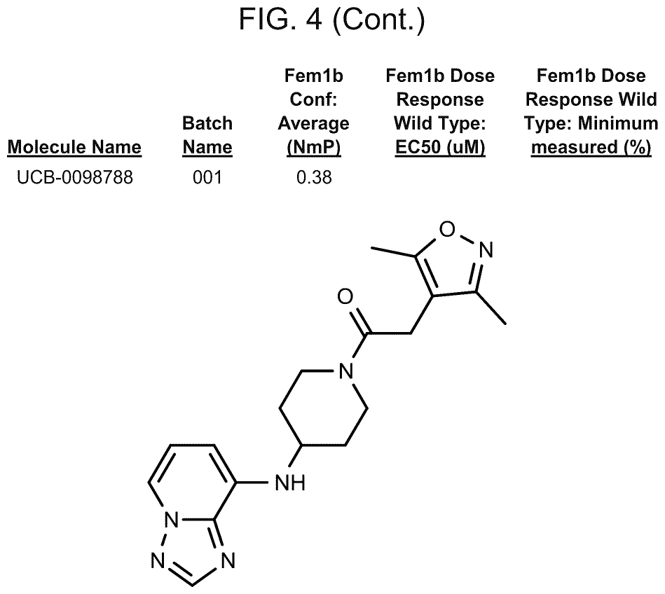

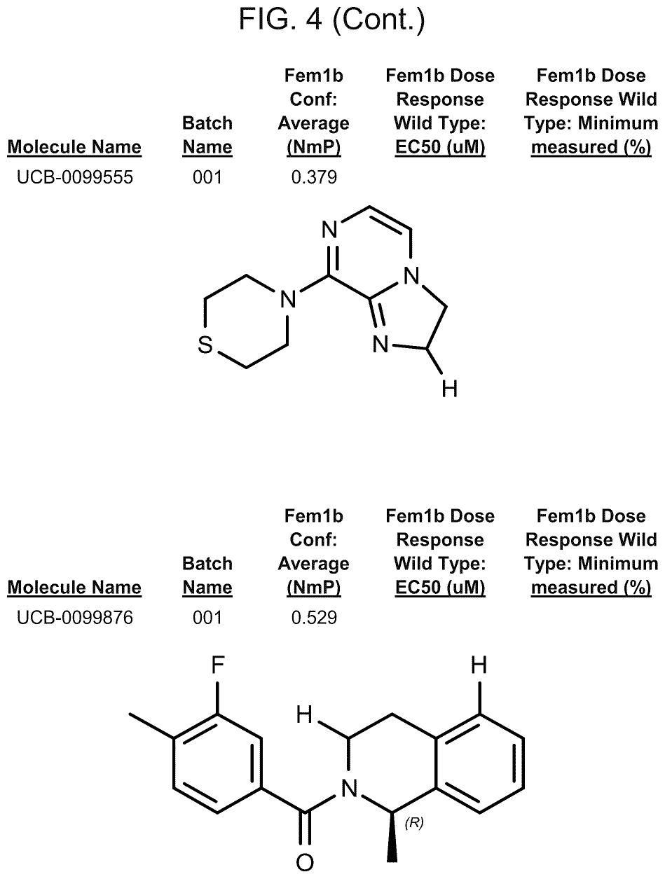

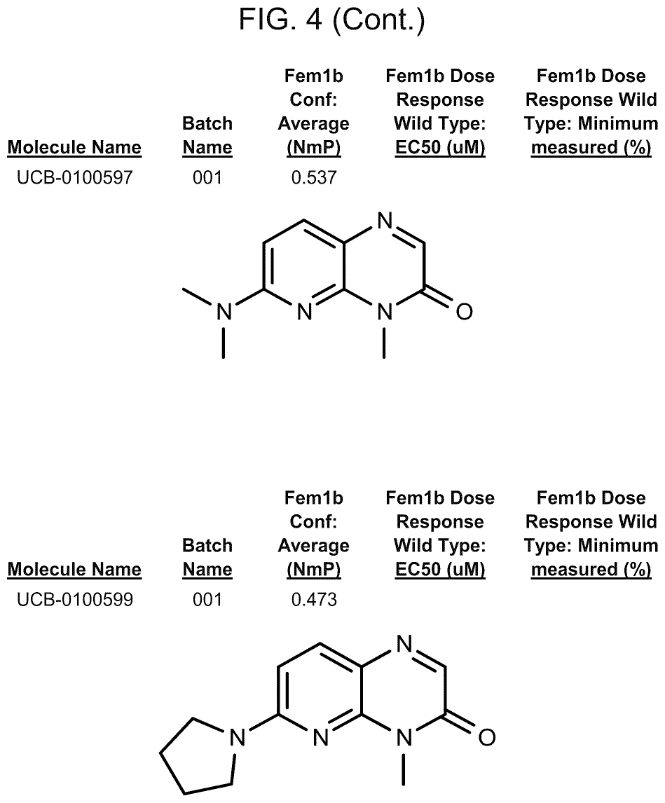

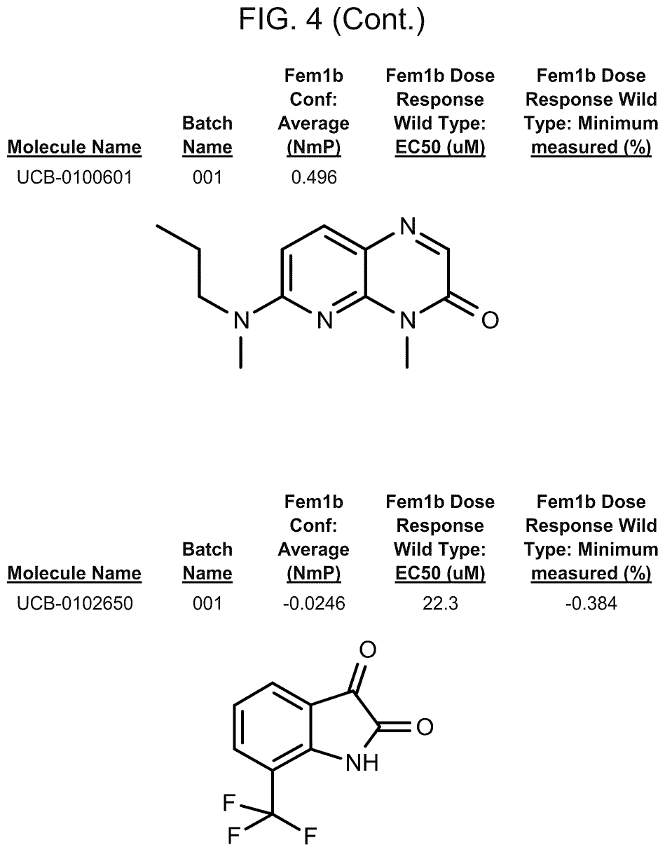

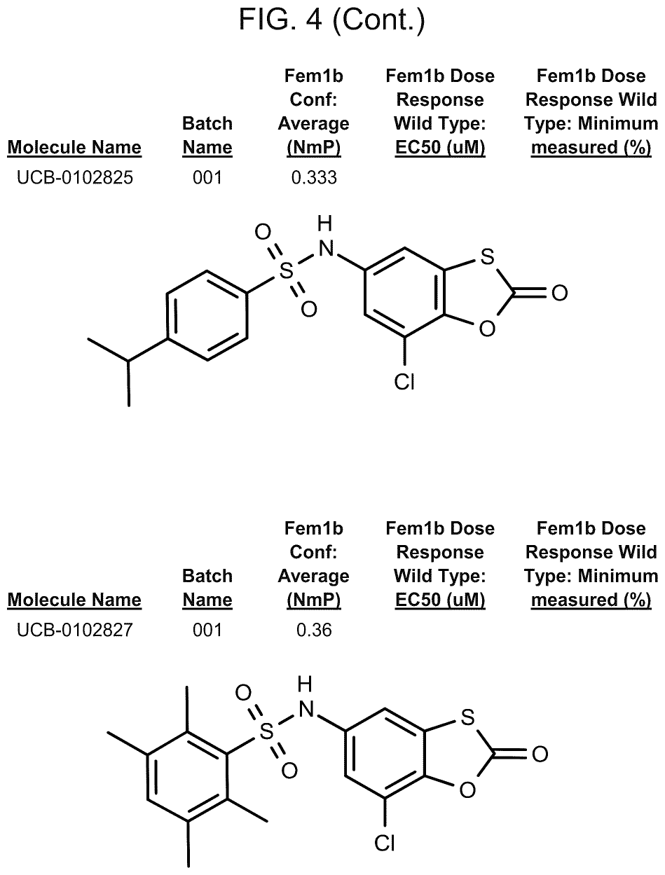

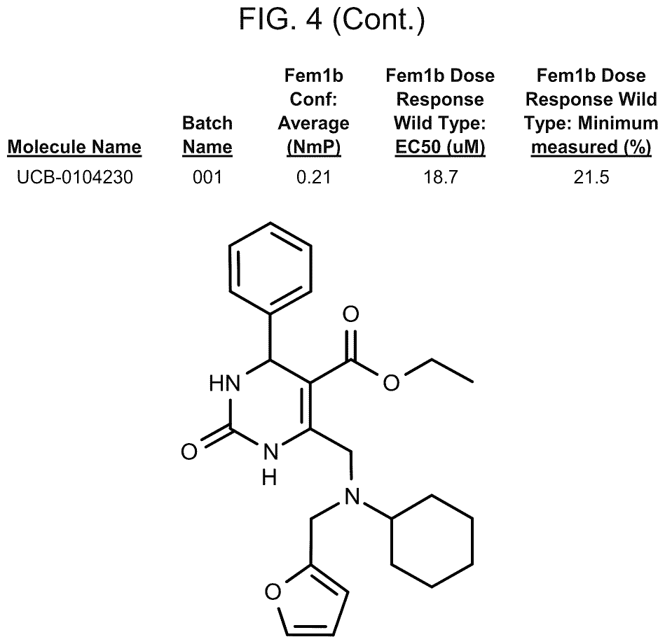

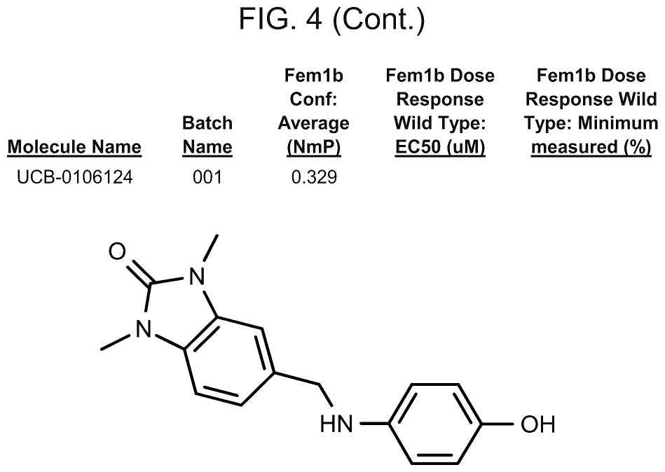

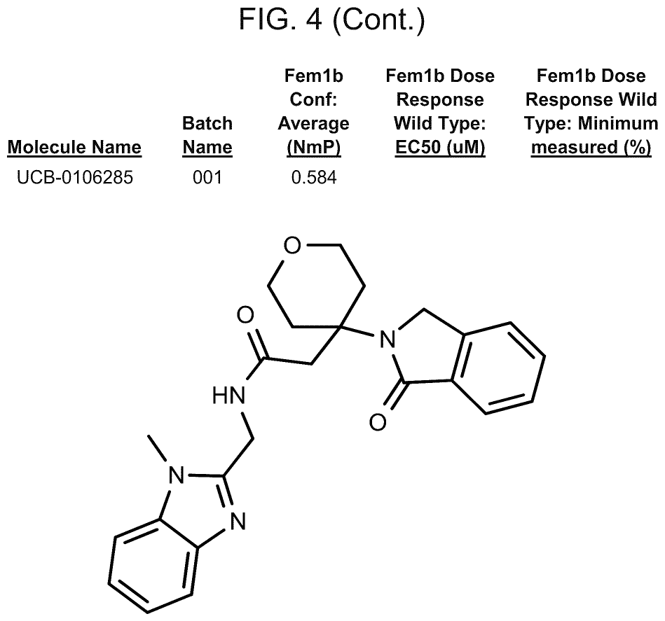

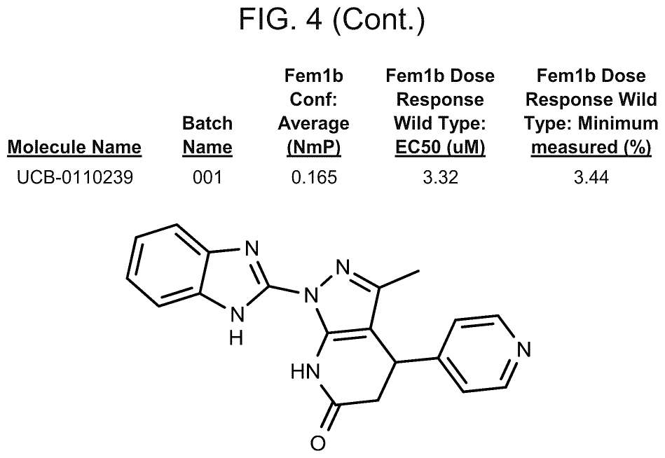

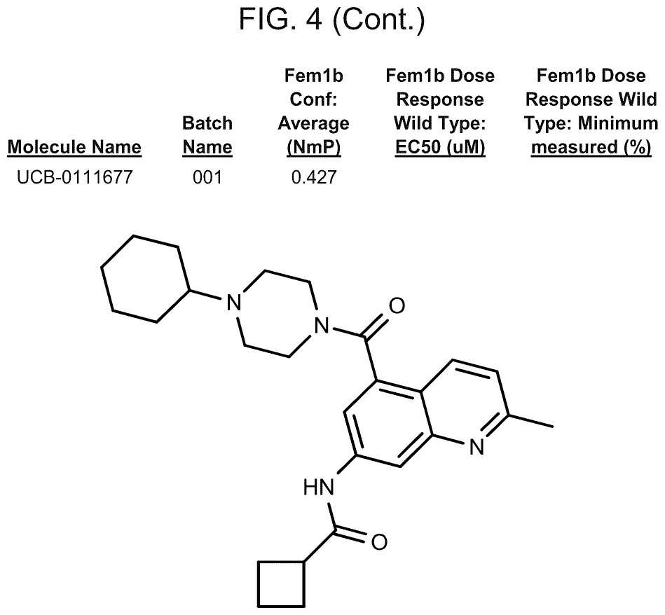

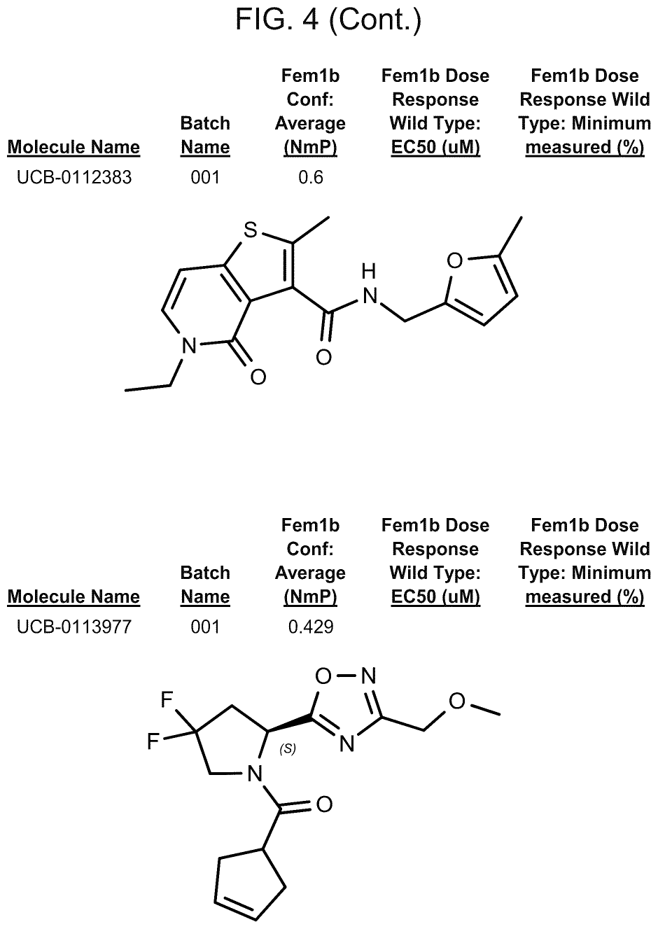

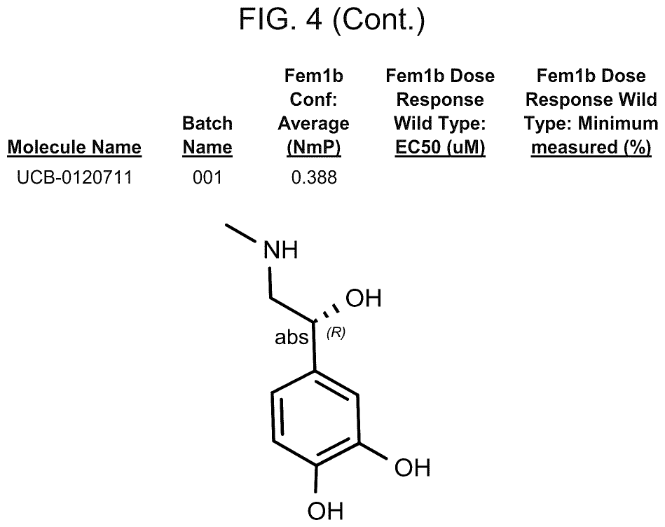

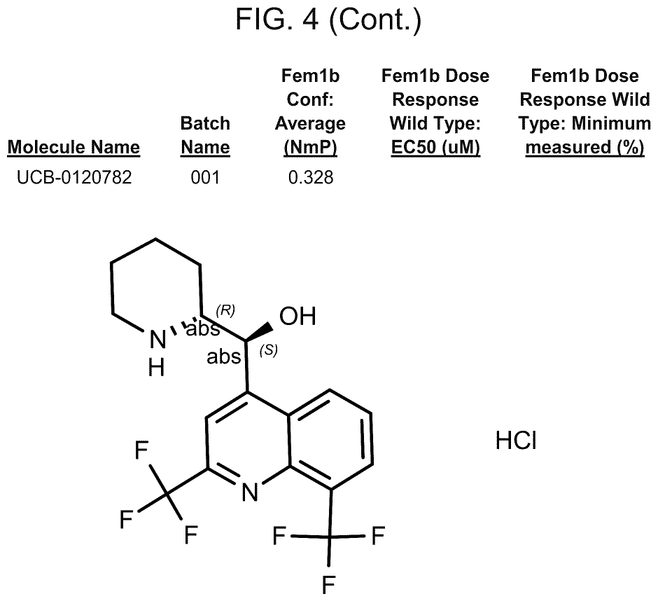

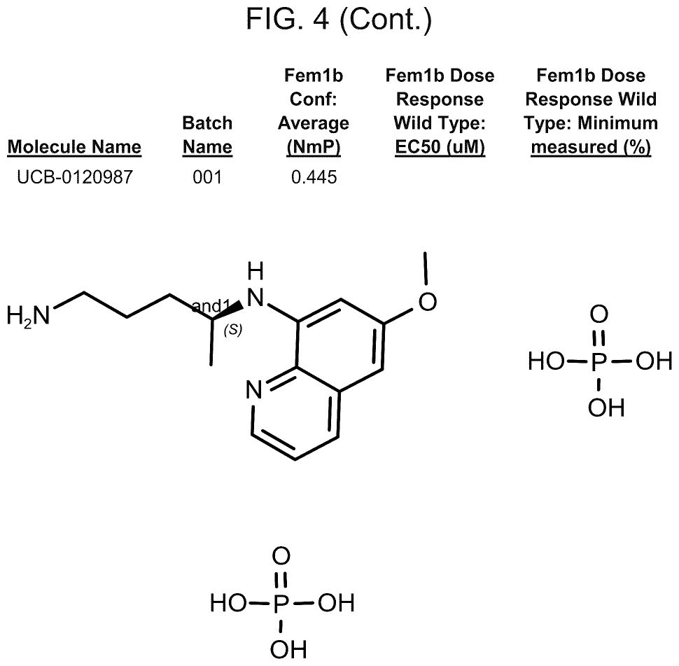

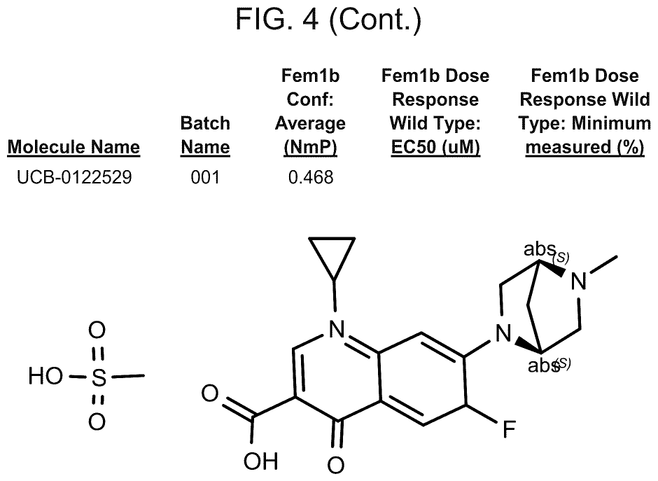

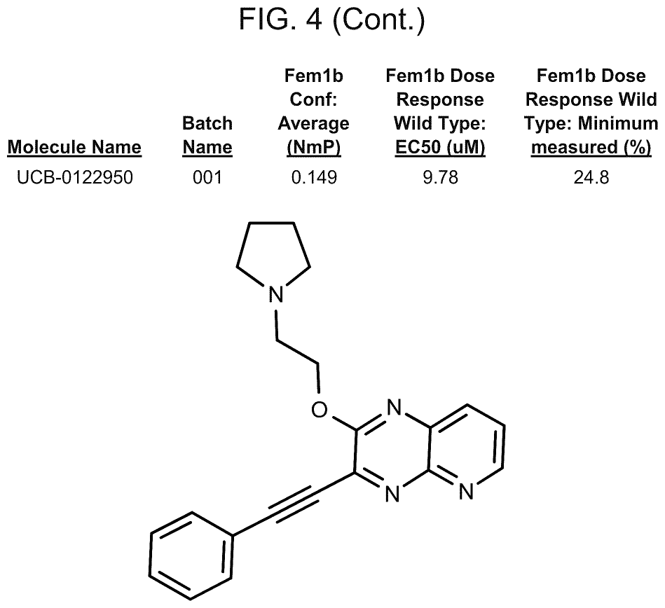

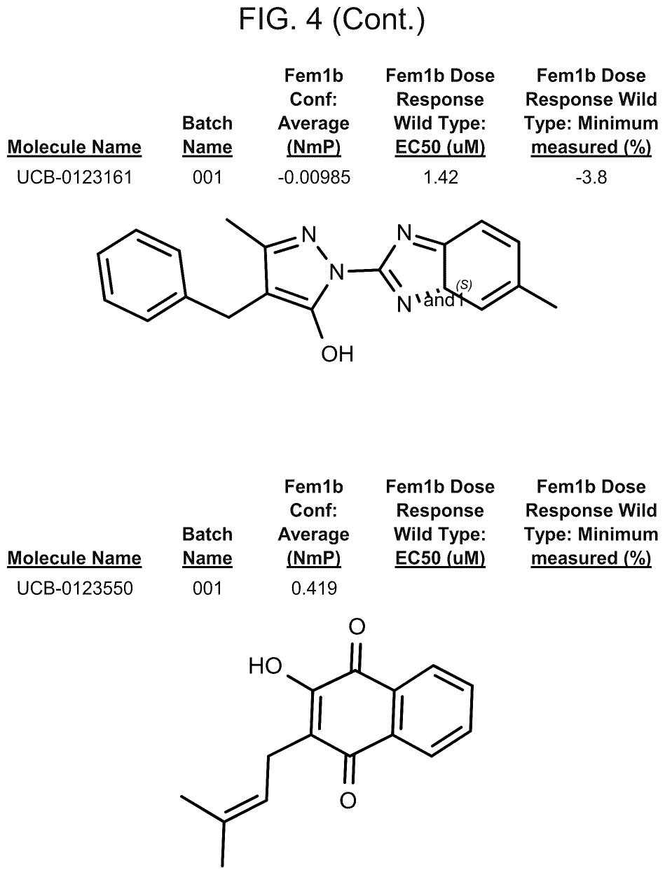

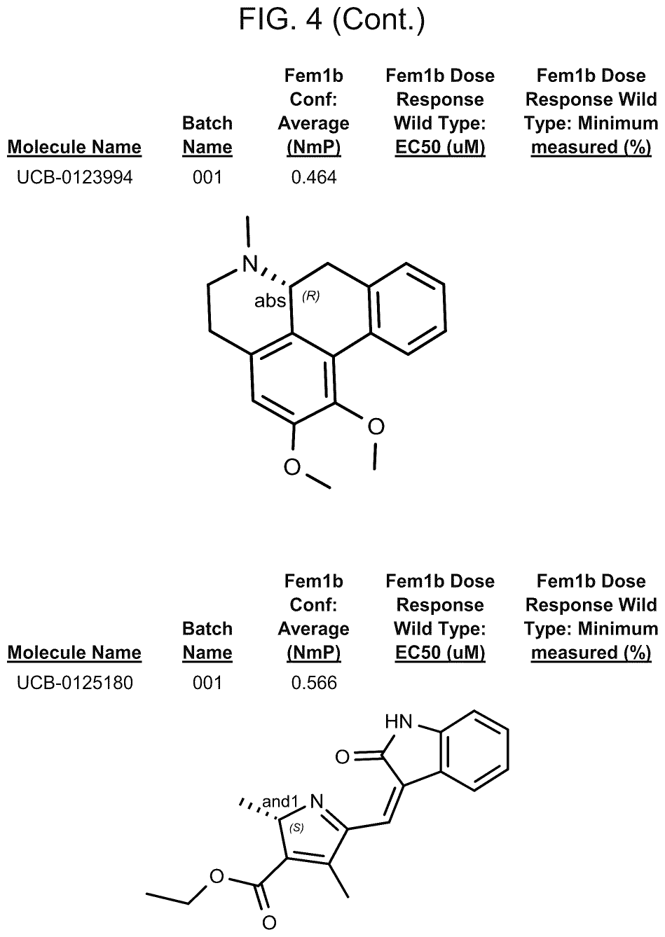

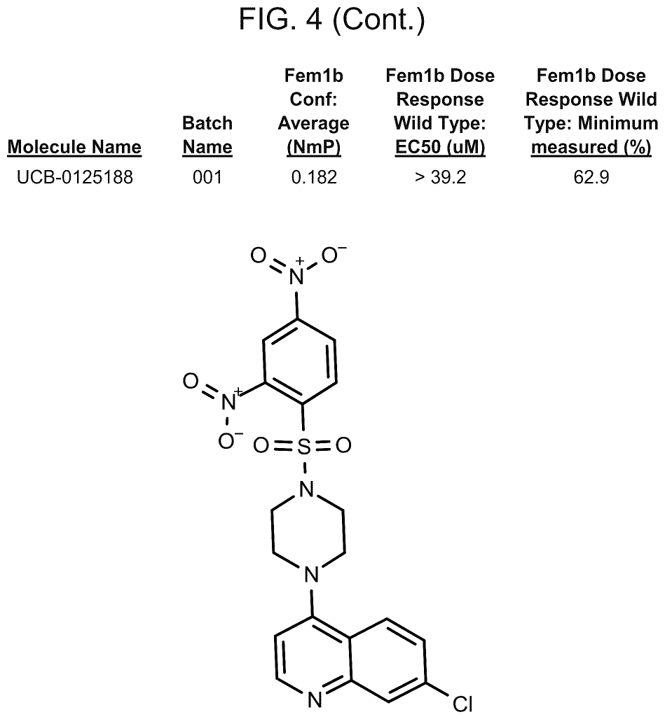

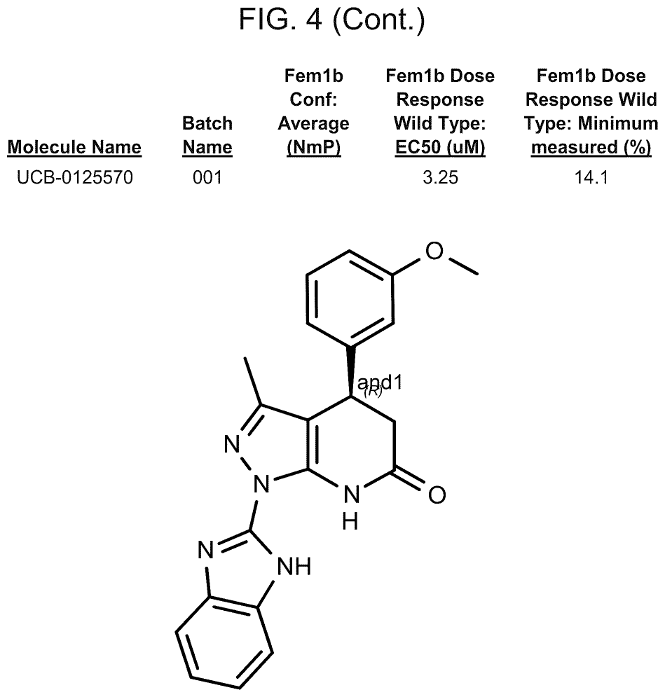

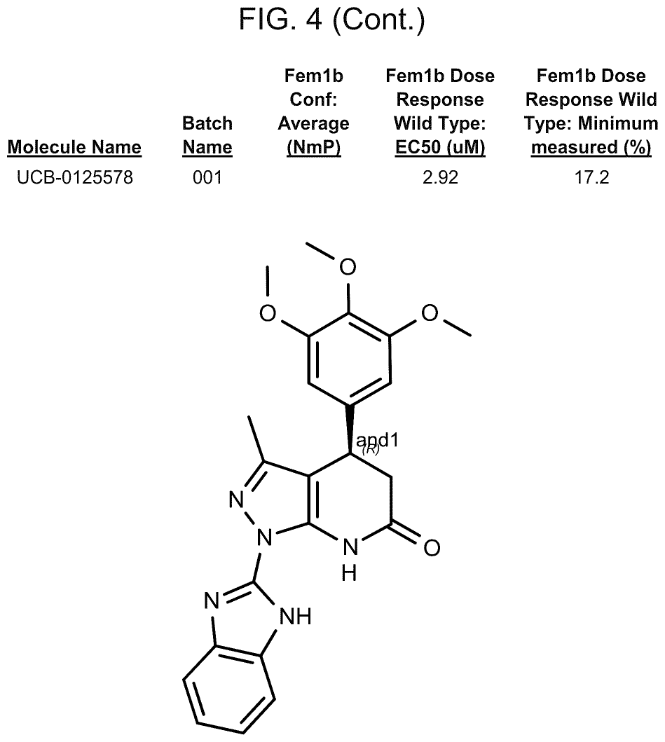

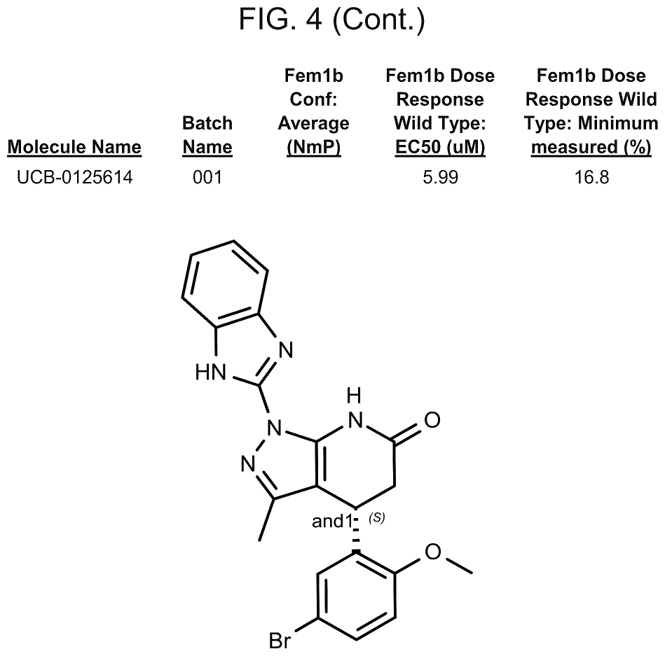

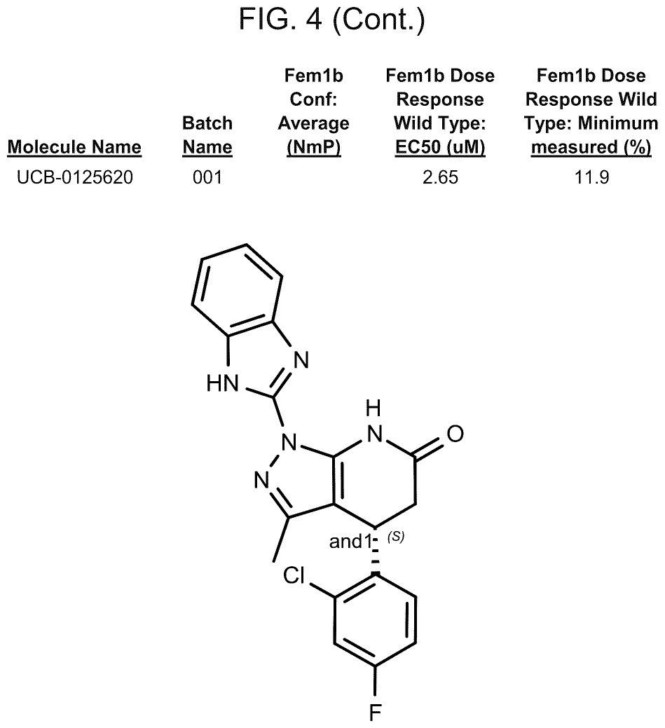

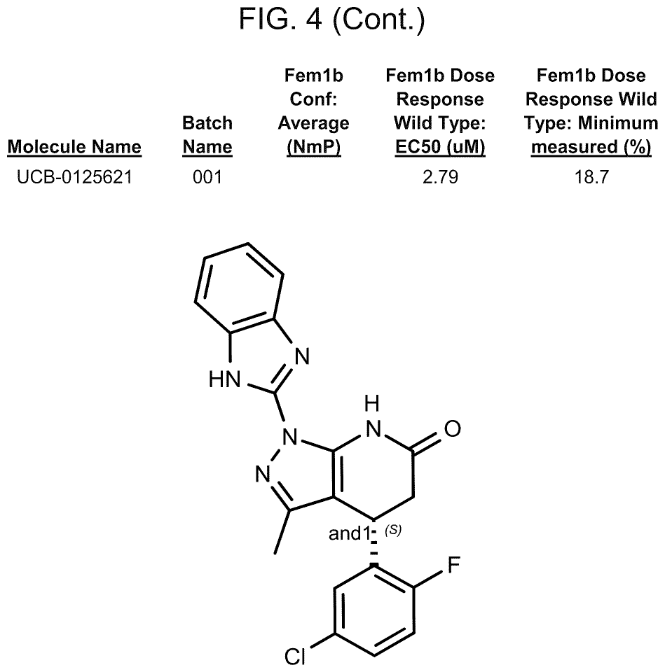

A “secondary treatment modality” refers to a secondary treatment (i.e. a second treatment following administration of a compound of formula I, II, III or IV, or a compound depicted in FIG. 1, FIG. 2, FIG. 3, or FIG. 4) that is directed to the disease or disorder that is being treated by a composition of the present disclosure. Secondary treatment modalities are often used to supplement a treatment that is currently in use in order to improve the outcomes of individuals in need thereof relative to any single treatment.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an E3 ligase in a cell” includes a plurality of such E3 ligases in a plurality of cells and reference to “the metabolic disorder” includes reference to one or more metabolic disorders and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides compositions and delivery devices comprising: i) a compound of formula I, II, III, or IV, or a compound depicted in FIG. 1, FIG. 2, FIG. 3, or FIG. 4; and ii) a pharmaceutical acceptable excipient. The present disclosure provides methods of inhibiting the activity of an E3 ligase. The present disclosure provides methods and compositions for the treatment of cancer. The present disclosure provides methods and compositions for the treatment of mitochondrial disorders. The present disclosure provides methods and compositions for the treatment of mental disorders. The present disclosure provides methods and compositions for the targeted degradation of target proteins using PROTAC.

### Compositions

The present disclosure provides compositions comprising: i) a compound of formula I, II, III, or IV; and ii) a pharmaceutical acceptable excipient. The present disclosure provides compositions comprising: i) a compound depicted in FIG. 1, FIG. 2, FIG. 3, or FIG. 4; and a pharmaceutical acceptable excipient. A compound of formula I, II, III, or IV binds to an E3 ligase non-covalently. In other words, the binding of a compound of formula I, II, III, or IV binds to an E3 ligase is non-covalent and is reversible. A compound depicted in any one FIG. 1, FIG. 2, FIG. 3, and FIG. 4 binds to an E3 ligase non-covalently. In other words, the binding of a compound depicted in any one of FIG. 1, FIG. 2, FIG. 3, and FIG. 4 to an E3 ligase is non-covalent and is reversible.

A composition of the present disclosure can comprise, in addition to a compound of formula I, II, III, or IV of the present disclosure, or a compound depicted in any of FIG. 1-FIG. 4, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like. In some cases, the composition does not include dimethyl sulfoxide (DMSO).

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. In some cases, the pharmaceutical acceptable excipient is not dimethyl sulfoxide (DMSO).

A pharmaceutical composition can comprise: i) a compound comprising formula I, II, III, or IV, or a compound depicted in any of FIG. 1-FIG. 4; and ii) a pharmaceutically acceptable excipient. In some cases, a subject pharmaceutical composition will be suitable for administration to a subject, e.g., will be sterile. For example, in some embodiments, a subject pharmaceutical composition will be suitable for administration to a human subject, e.g., where the composition is sterile and is free of detectable pyrogens and/or other toxins.

The protein compositions may comprise other components, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate, hydrochloride, sulfate salts, solvates (e.g., mixed ionic salts, water, organics), hydrates (e.g., water), and the like.

For example, compositions may include aqueous solution, powder form, granules, tablets, pills, suppositories, capsules, suspensions, sprays, and the like. The composition may be formulated according to the various routes of administration described below.

Suitable excipients (e.g., for oral formulations) include, e.g., croscarmellose sodium (disintegrant), microcrystalline cellulose (filler), magnesium stearate, (antiadherent, lubricant), povidone (disintegrant), sodium starch glycolate (disintegrant), silica, and colloidal anhydrous (glidant, lubricant).

Suitable excipients include, e.g., pyrazole-based heterocycle core excipient oral tablet formulation for cluster 47 compounds, croscarmellose sodium, magnesium stearate, microcrystalline cellulose, and sodium lauryl sulfate (sodium dodecyl sulfate; SDS).

Suitable excipients include, e.g., oxazole core with sulfone moiety excipient oral tablet formulation for cluster 75 compounds, microcrystalline cellulose, docusate sodium, magnesium stearate, povidone, and sodium starch glycolate.

Suitable excipients include, e.g., sulfone-based excipient oral tablet formulation for cluster 75 compounds, including: croscarmellose sodium, docusate sodium, microcrystalline cellulose, povidone, sodium benzoate, sodium starch glycolate, and stearic acid.

Suitable excipients include, e.g., sodium chloride, poly(ethylene glycol) (PEG), benzyl alcohol, sorbitol, saccharin sodium, sodium citrate, citric acid anhydrous, methyl parahydroxybenzoate, propyl parahydroxybenzoate, propylene glycol, maltodextrin, lactic acid, and glyceryl triacetate.

### Delivery Devices

The present disclosure provides a drug delivery device comprising a composition of the present disclosure. Suitable drug delivery devices include, without limitation, an injection device (e.g., syringe, pen injector, auto injector, large-volume device, pump, perfusion system, or other device configured for subcutaneous, intramuscular, or intravenous delivery; a skin patch (e.g., osmotic, chemical, micro-needle); an inhaler (e.g., nasal or pulmonary); an implantable device; and a feeding system for the gastro-intestinal tract.

### Methods of Inhibiting the Activity of an E3 Ligase

The present disclosure provides methods of inhibiting the activity of an E3 ligase. The methods comprise contacting the E3 ligase with a compound of the present disclosure. In some cases, the E3 ligase is present in a cell in vitro. In some cases, the E3 ligase is present in a cell, a tissue, an organ, or an extracellular fluid in an individual.

An E3 ligase is a complex of polypeptides. In some cases, the E3 ligase is a Cullin-RING ligase. In some cases, the E3 ligase is a CULE2 ligase.

In some cases, a compound of the present disclosure inhibits a FEM1B polypeptide. In some cases, the E3 ligase comprises a FEM1B polypeptide; for example, in some cases, the E3 ligase is composed of CUL2, EloB/C, RBX1, and FEM1B. FEM1B is a component of an E3 ligase, and may act as a substrate recognition subunit.

In some cases, the E3 ligase inhibitor is described the by Formula (I):

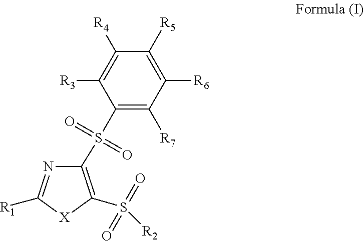

where: X is selected from an O, S or NH₂. In some cases, R₁ may be selected from an aryl, substituted aryl, arylalkyl, substituted arylalkyl, heteroaryl, substituted heteroaryl, heteroarylalkyl, or substituted heteroarylalkyl. In some cases, R₂ may be selected from an alkyl, substituted alkyl, heteroalkyl, substituted heteroalkyl, acyl, aminoacyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, or substituted heteroarylalkyl. In some cases, R₃, R₄, R₅, R₆, and R₇ are each individually selected from hydrogen, alkyl, substituted alkyl, alkoxy, halogen, nitro, amino, hydroxy, cyano, or thiol. In some cases, R₁ is selected from a thiophene, substituted thiophene, phenyl, or substituted phenyl. In some cases, R₁ is described by the following structure:

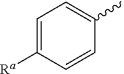

where Rₐ is selected from a hydrogen, hydroxyl, fluorine, chlorine, bromine, or iodine and [symbol] represents the R₁—C bond. In some cases, R₂ is selected from alkyl, substituted alkyl, alkylaryl, substituted alkylaryl, C1-C8 linear, C1-C8 branched alkyl, methyl, ethyl, n-propyl, isopropyl, n-butyl, isobutyl, pentyl, isopentyl, hexyl, heptyl, octyl, benzyl, substituted benzyl, fluorine, chlorine, bromine or iodine. In some cases, R5 is selected from a hydrogen, fluorine, chlorine, bromine, or iodine.

In some cases, the E3 ligase inhibitor is described the by Formula (II):

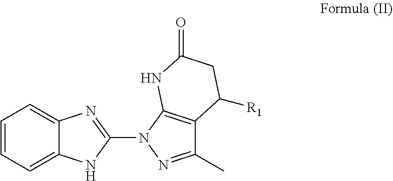

where: R₁ is selected from an aryl, substituted aryl, arylalkyl, substituted arylalkyl, heteroaryl, substituted heteroaryl, heteroarylalkyl, and substituted heteroarylalkyl. In some cases, R₁ may be selected from a phenyl, substituted phenyl, pyrazole, substituted pyrazole, N-ethyl-3-methyl pyrazole, N-methyl-5-methyl pyrazole, pyridine, or thiophene.

In some cases, the E3 ligase inhibitor is described the by Formula (III):

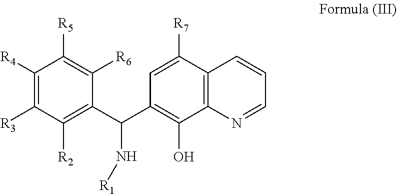

where R₁ is selected from a alkyl, substituted alkyl, acyl, acylalkyl, aryl, substituted aryl, heteroaryl or substituted heteroaryl. In some cases, R₂, R₃, R₄, R₅, R₆, and R₇ is selected from a hydrogen, alkyl, substituted alkyl, alkoxy, halogen, nitro, amino, hydroxy, cyano, or thiol. In some cases, R₁ is represented by the formula:

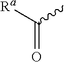

where Rₐ is selected from a C1-C8 alkyl, substituted C1-C8 alkyl, methyl, ethyl, n-propyl, isopropyl, n-butyl, isobutyl, pentyl, hexyl, heptyl, or octyl, and [symbol] represents the R₁—N bond. In some cases, C1-C8 alkyl substituted with an aryl, substituted aryl, heteroaryl, substituted heteroaryl, cycloalkyl, substituted cycloalkyl, heterocycloalkyl or substituted heterocycloalkyl. In some cases, Rₐ is a C2 alkyl substituted with a phenyl. In some cases, R₃ is selected from a hydroxyl, alkoxyl, methoxy, ethoxy, methyl, or C1-C8 alkyl. In some embodiments, R₄ is selected from a hydroxyl, alkoxyl, methoxy, ethoxy, methyl, or C1-C8 alkyl. In some cases, R₅ is selected from a hydroxy, alkoxy, methoxy, ethoxy, methyl, or C1-C8 alkyl.

In some cases, the E3 ligase inhibitor is described the by Formula (IV):

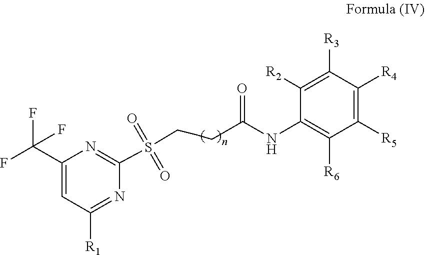

where R₁ is selected from an aryl, substituted aryl, heteroaryl, substituted heteroaryl, cycloalkyl, substituted cycloalkyl, heterocycloalkyl, substituted heterocycloalkyl, thiophene, substituted phenyl, o-methoxy phenyl. In some cases, R₁ is represented by the formula:

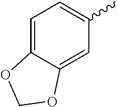

where [symbol] represents the R¹—C bond. In some cases, Formula (IV) comprises a hydroxyl in the R₂ position and a bromine in the R₅ position.

In some cases, the E3 ligase comprises an amino acid sequence having at least 75%, at least 80%, at least 85%, at least 90%, at least 95%, at least 98%, at least 99%, or 100%, amino acid sequence identity to amino acids 1-627 of the following amino acid sequence of the following human Fem1b amino acid sequence:

(SEQ ID NO: 1)
MEGLAGYVYKAASEGKVLTLAALLLNRSESDIRYLLGYVSQQGGQRSTP
LIIAARNGHAKVVRLLLEHYRVQTQQTGTVRFDGYVIDGATALWCAAGA
GHFEVVKLLVSHGANVNHTTVTNSTPLRAACFDGRLDIVKYLVENNANI
SIANKYDNTCLMIAAYKGHTDVVRYLLEQRADPNAKAHCGATALHFAAE
AGHIDIVKELIKWRAAIVVNGHGMTPLKVAAESCKADVVELLLSHADCD
RRSRIEALELLGASFANDRENYDIIKTYHYLYLAMLERFQDGDNILEKE
VLPPIHAYGNRTECRNPQELESIRQDRDALHMEGLIVRERILGADNIDV
SHPIIYRGAVYADNMEFEQCIKLWLHALHLRQKGNRNTHKDLLRFAQVF
SQMIHLNETVKAPDIECVLRCSVLEIEQSMNRVKNISDADVHNAMDNYE
CNLYTFLYLVCISTKTQCSEEDQCKINKQIYNLIHLDPRTREGFTLLHL
AVNSNTPVDDFHTNDVCSFPNALVTKLLLDCGAEVNAVDNEGNSALHII
VQYNRPISDFLTLHSIIISLVEAGAHTDMTNKQNKTPLDKSTTGVSEIL
LKTQMKMSLKCLAARAVRANDINYQDQIPRTLEEFVGFH.

In some cases, the E3 ligase comprises an amino acid sequence having at least 75%, at least 80%, at least 85%, at least 90%, at least 95%, at least 98%, at least 99%, or 100%, amino acid sequence identity to amino acids 1-627 of the following amino acid sequence of the following mouse Fem1b amino acid sequence:

(SEQ ID NO: 2)
MEGLAGYVYKAASEGKVLTLAALLLNRSESDIRYLLGYVSQQGGQRSTP
LIIAARNGHAKVVRLLLEHYRVQTQQTGTVRFDGYVIDGATALWCAAGA
GHFEVVKLLVSHGANVNHTTVTNSTPLRAACFDGRLDIVKYLVENNANI
SIANKYDNTCLMIAAYKGHTDVVRYLLEQRADPNAKAHCGATALHFAAE
AGHIDIVKELIKWRAAIVVNGHGMTPLKVAAESCKADVVELLLSHADCD
RRSRIEALELLGASFANDRENYDIMKTYHYLYLAMLERFQDGDNILEKE
VLPPIHAYGNRTECRNPQELEAIRQDRDALHMEGLIVRERILGADNIDV
SHPIIYRGAVYADNMEFEQCIKLWLHALHLRQKGNRNTHKDLLRFAQVF
SQMIHLNEAVKAPDIECVLRCSVLEIEQSMNRVKNISDADVHSAMDNYE
CNLYTFLYLVCISTKTQCSEEDQCRINKQIYNLIHLDPRTREGFSLLHL
AVNSNTPVDDFHTNDVCSFPNALVTKLLLDCGAEVNAVDNEGNSALHII
VQYNRPISDFLTLHSIIISLVEAGAHTDMTNKQNKTPLDKSTTGVSEIL
LKTQMKMSLKCLAARAVRANDINYQDQIPRTLEEFVGFH

A subject compound (e.g., a compound of any one of Formulas I, II, III, and IV; a compound depicted in any of FIG. 1-FIG. 4) can inhibit the activity an E3 ligase by at least about 10%, compared to the activity of the E3 ligase not contacted with the compound. In some cases, a subject compound can inhibit the activity of an E3 ligase by greater than 10% compared to the activity of the E3 ligase not contacted with the compound. For example, a subject compound may inhibit the activity of an E3 ligase by 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or 95-100%, compared to the activity of the E3 ligase not contacted with the compound.

In some cases, the E3 ligase is present in a cell in vitro. In some cases, the E3 ligase is present in a cell in vivo. In some cases, the cell is a cancer cell.

Whether a compound inhibits an E3 ligase can be determined using an assay such as those described in the Examples. For example, a peptide having the following amino acid sequence: RNKSSLLFKESEETRTPNCNCKYCSHPVLG (SEQ ID NO:3) and comprising a fluorescent label can be used as the substrate and contacted with the E3 ligase in the presence or absence of a compound being tested for inhibitory activity toward the E3 ligase. As another example, a degron reporter assay as described in the Examples can be used to determine whether a compound inhibits an E3 ligase (e.g., a FEM1B polypeptide). A reduction in the activity of the E3 ligase in the presence of the compound, compared to the activity of the E3 ligase in the absence of the compound, indicates that the compound inhibits E3 ligase activity.

### Treatment Methods

A compound of the present disclosure is useful for treating various diseases and disorders. The present disclosure provides a method of selectively inhibiting the activity of an E3 ligase in an individual, the method comprising administering to the individual an amount of a composition comprising: i) a compound of formula I, II, III, or IV, or a compound depicted in any of FIG. 1-FIG. 4; and ii) a pharmaceutical acceptable excipient. The compound of formula I, II, III, or IV, or the compound depicted in any of FIG. 1-FIG. 4 is administered in an amount effective to selectively inhibit the activity of an E3 ligase in an individual. In some cases, subject compound inhibits the activity of an E3 ligase by 10-15%, 15-20%, 20-25%, 25-30%, 30-35%, 35-40%, 40-45%, 45-50%, 50-55%, 55-60%, 60-65%, 65- 70%, 70-75%, 75-80%, 80-85%, 85-90%, 90-95%, or 95-100%, compared to the activity of the E3 ligase not contacted with the compound.

In some cases, a treatment method of the present disclosure comprises administering to an individual in need thereof a composition of the present disclosure. In some cases, a treatment method of the present disclosure comprises administering to an individual in need thereof a composition of the present disclosure in addition to a secondary treatment modality directed towards treating said condition.

### Methods of Treating Cancer

The present disclosure provides a method of treating cancer in an individual, the method comprising administering to the individual an effective amount of a composition of the present disclosure. In some cases, an “effective amount” of a composition is an amount that, when administered in one or more doses to an individual in need thereof, reduces the number of cancer cells in the individual. For example, in some cases, an “effective amount” of a composition of the present disclosure is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the composition, or in the absence of administration with the composition. In some cases, an “effective amount” of a composition of the present disclosure is an amount that, when administered in one or more doses to an individual in need thereof, reduces the number of cancer cells in the individual to undetectable levels.

In some cases, an “effective amount” of a composition of the present disclosure is an amount that, when administered in one or more doses to an individual in need thereof, reduces the tumor mass in the individual. For example, in some cases, an “effective amount” of a composition of the present disclosure is an amount that, when administered in one or more doses to an individual in need thereof, reduces the tumor mass in the individual by at least 10%, at least 15%, at least 20%, at least 25%, at least 30%, at least 40%, at least 50%, at least 60%, at least 70%, at least 80%, at least 90%, or at least 95%, compared to the tumor mass in the individual before administration of the composition, or in the absence of administration with the composition. In some cases, an “effective amount” of a composition of the present disclosure is an amount that, when administered in one or more doses to an individual in need thereof, increases survival time of the individual. For example, in some cases, an “effective amount” of a composition of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the composition of the present disclosure.

In some cases, a secondary treatment modality is used in addition to a composition of the present disclosure to treat cancer in an individual in need thereof. In some cases, the secondary treatment modality is selected from chemotherapy, hormone therapy, immune therapy, or target therapy. When chemotherapy is used in addition to administration of a composition of the present disclosure, numerous types of chemotherapy drugs may be used. Types of chemotherapy drugs that find use in a method of the present disclosure, include, without limitation, alkylating agents, antimetabolites, anthracyclines, topoisomerase inhibitors, plant alkaloids, corticosteroids, etc. Any suitable alkylating agents may be used in chemotherapy, including, without limitation, chlorambucil, cyclophosphamide, ifosfamide, melphalan, etc. Any suitable antimetabolites may be used in chemotherapy, including, without limitation, 5-fluorouracil (5-FU), 6-mercaptopurine (6-MP), cytarabine, capecitabine, fludarabine, gemcitabine, methotrexate, pemetrexed, pentostatin, thioguanine, etc. Any suitable anthracyclines may be used in chemotherapy, including, without limitation, doxorubicin, daunorubicin, idarubicin, epirubicin, etc. Any suitable topoisomerase inhibitors may be used in chemotherapy, including, without limitation, topotecan, irinotecan, etoposide, teniposide, etc. Any suitable plant alkaloids may be used in chemotherapy, including, without limitation, paclitaxel, docetaxel, vinblastine, vincristine, vinorelbine etc. Any suitable corticosteroid may be used in chemotherapy, including, without limitation, prednisone, methylprednisolone, dexamethasone, etc.

When hormone therapy is used in addition to administration of a composition of the present disclosure, numerous hormone therapy drugs may be used. Hormone therapy drugs that find use in a method of the present disclosure, include, without limitation, tamoxifen, letrozole, abiraterone, raloxifene, prednisone, etc.

When immune therapy is used in addition to administration of a composition of the present disclosure, numerous immune therapy drugs/monoclonal antibodies may be used. Immune therapy drugs/monoclonal antibodies that find use in a method of the present disclosure, include, without limitation, Alemtuzumab, Atezolizumab, Avelumab, Ipilimumab, Elotuzumab, Ofatumumab, Nivolumab, Pembrolizumab, Rituximab, Durvalumab, Magrolimab, interferons, interlukins, etc.

When targeted therapy is used in addition to administration of a composition of the present disclosure, numerous drugs/monoclonal antibodies may be used. Targeted therapy drugs/monoclonal antibodies that find use in a method of the present disclosure, include, without limitation, imatinib, dasatinib, nilotinib, erlotinib, gefitinib, osimertinib, olaparib, rucaparib, niraparib, talaparib, ruxolitinib, fedratinib, vemurafenib, dabrafenib, encorafenib, trametinib, cobimetinib, binimetinib, etc.

### Methods of Treating a Metabolic Disorder

The present disclosure provides methods for treating a metabolic disorder. The methods generally involve administering to an individual having a metabolic disorder an effective amount of a compound of the present disclosure.

Metabolic disorders that can be treated using a method of the present disclosure include, but are not limited to, obesity, type 1 diabetes mellitus (T1D), type II diabetes, non-alcoholic fatty liver disease (NAFLD), non-alcoholic steatohepatitis (NASH), and hepatic steatosis.

In some cases, an “effective amount” of a composition is an amount that, when administered in one or more doses to an individual in need thereof, reduces adverse symptoms in the individual. For example, in some cases, an “effective amount” of a composition of the present disclosure is an amount that, when administered in one or more doses to an individual in need thereof, reduces at least one adverse symptom of the metabolic disorder in the individual by at least 10%, at least 15%, at least 20%, at least 25%, at least 30%, at least 40%, at least 50%, at least 60%, at least 70%, at least 80%, at least 90%, or at least 95%, compared to the symptoms in the individual before administration of the composition, or in the absence of administration with the composition. In some cases, an “effective amount” of a composition of the present disclosure is an amount that, when administered in one or more doses to an individual in need thereof, reduces adverse symptoms in the individual to undetectable levels. For example, in some cases, an “effective amount” of a composition of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the composition of the present disclosure.

Subjects suitable for treatment using a method of the present disclosure include individuals who have been diagnosed with a metabolic disorder such as TID, type II diabetes, NAFLD, NASH, or hepatic steatosis. In some cases, the individual has a body mass index of greater than 30 kg/m².

In some cases, a secondary treatment modality is used in addition to a composition of the present disclosure to treat a metabolic disorder in an individual in need thereof. Secondary treatment modalities may be directed to the disease or disorder being treated. Secondary treatment modalities that find use in the present disclosure, include, without limitation, insulin, glucagon, a statin, a thiazolidinedione, sulfonylureas, glinides, metformin, starch blockers, incretin therapies, pramlintide, SGLT2 inhibitors, etc. When insulin is used as a secondary treatment modality, any suitable insulin may be used, including, without limitation, rapid-acting insulin, short-acting insulin, intermediate-acting insulin, long-acting insulin, etc. In some cases, the insulin administered is a combination of the insulins listed above. When a statin is used as a secondary treatment modality, any suitable statin may be used, including, without limitation, Atorvastatin, Cerivastatin, Fluvastatin, Lovastatin, Mevastatin, Pitavastatin, Pravastatin, Rosuvastatin, Simvastatin, etc. When a thialzolidinedione is used as a secondary treatment modality, any suitable thialzolidinedione may be used, including, without limitation, rosiglitazone, pioglitazone, etc. When a sulfonylureas are used as a secondary treatment modality, any suitable sulfonylurea may be used, including, without limitation, chlorpropamide, tolbutamide, glipizide, glyburide, glimepiride, etc. When a glinides are used as a secondary treatment modality, any suitable glinides may be used, including, without limitation, repaglinide, nateglinide, mitiglinide, etc. When starch blockers are used as a secondary treatment modality, any suitable starch blocker may be used, including, without limitation, acarbose, miglitol, etc. When incretin therapies are used as a secondary treatment modality, any suitable incretin therapy may be used, including, without limitation, sitagliptin, saxagliptin, linagliptin, exenatide, liraglutide, etc. When SGLT2 inhibitors are used as a secondary treatment modality, any suitable SGLT2 inhibitor may be used, including, without limitation, canagliflozin, dapagliflozin, and empagliflozin, etc.

### Methods of Treating a Mitochondrial Disorder

The present disclosure provides methods and compositions for the treatment of mitochondrial disorders. Mitochondrial disorders are often the result of misregulation of ATP generation and oxidative/reductive stress. Mitochondrial disorders can also be caused by mutations (acquired or inherited), in mitochondrial DNA (mtDNA), or in nuclear genes that code for mitochondrial components. They may also be the result of acquired mitochondrial dysfunction due to adverse effects of drugs, infections, or other environmental causes. Oxalate may enter cells where it is known to cause mitochondrial dysfunction.

In particular embodiments the methods are used in the treatment of a mitochondrial disorder, for example including but not limited to Mitochondrial myopathy, Diabetes mellitus and deafness (DAD), Leber's hereditary optic neuropathy, Leigh syndrome, Neuropathy, ataxia, retinitis, pigmentosa, and ptosis (NARP), Myoneurogenic gastrointestinal encephalopathy (MNGIE), Myoclonic epilepsy with ragged red fibers (MERRF), Mitochondrial encephalopathy, lactic acidosis and stroke-like episodes (MELAS), Mitochondrial DNA depletion syndrome, muscular dystrophy, Lou Gehrig's disease, type 1 diabetes, type 2 diabetes, non-alcoholic fatty liver disease (NASH), non-alcoholic steatohepatitis (NASH), hepatic steatosis, lung adenocarcinoma and cancer, etc. Other conditions and disorders that can be treated using a method of the present disclosure include obesity, chronic fatigue syndrome, aging, hypercholesteremia, heart disease, and cardiomyopathy.

### Methods of Treating Mental Disorders

The present disclosure provides a method of treating a mental disorder in an individual, the method comprising administering to the individual an effective amount of a composition of the present disclosure. In some cases, an “effective amount” of a composition is an amount that, when administered in one or more doses to an individual in need thereof, reduces adverse symptoms in the individual. For example, in some cases, an “effective amount” of a composition of the present disclosure is an amount that, when administered in one or more doses to an individual in need thereof, reduces adverse symptoms in the individual by at least 10%, at least 15%, at least 20%, at least 25%, at least 30%, at least 40%, at least 50%, at least 60%, at least 70%, at least 80%, at least 90%, or at least 95%, compared to the symptoms in the individual before administration of the composition, or in the absence of administration with the composition. In some cases, an “effective amount” of a composition of the present disclosure is an amount that, when administered in one or more doses to an individual in need thereof, reduces adverse symptoms in the individual to undetectable levels. For example, in some cases, an “effective amount” of a composition of the present disclosure is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the composition of the present disclosure.

In particular embodiments, the methods are used in the treatment of a mitochondrial disorder, for example including but not limited to Alzheimer's disease, syndromic intellectual disability, schizophrenia etc.

In some cases, a secondary treatment modality is used in addition to a composition of the present disclosure to treat a mental disorder in an individual in need thereof. Secondary treatment modalities may be directed to the disease or disorder being treated. Secondary treatment modalities that find use in the present disclosure, include, without limitation, a cholinesterase, memantine, psychotropic medications, etc. When a cholinesterase is used as a secondary treatment modality, any suitable cholinesterase may be used, including, without limitation, donepezil, galantamine, rivastigmine. When psychotropic medications are used as a secondary treatment modality, any suitable psychotropic medication may be used, including, without limitation, Risperidone, Carbamazepine, Sodium Valproate, Lamotrigine, Lithium Carbonate, Methylphenidate, Procyclidine, Aripiprazole, Asenapine, Brexpiprazole, Cariprazine, Clozapine, Iloperidone, Lurasidone, Olanzapine, Paliperidone, Quetiapine, Risperidone, Ziprasidone, Chlorpromazine, Fluphenazine, Haloperidol, Perphenazine, etc.

### Subjects Suitable for Treatment

Subjects suitable for treatment with a method of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment with a method of the present disclosure include individuals who have a metabolic disorder, including individuals who have been diagnosed as having a metabolic disorder, and individuals who have been treated for a metabolic disorder but who failed to respond to the treatment. Subjects suitable for treatment with a method of the present disclosure include individuals who have a mental disorder, including individuals who have been diagnosed as having a mental disorder, and individuals who have been treated for a mental disorder but who failed to respond to the treatment.

### Formulations

Suitable formulations are described above, where suitable formulations include a pharmaceutically acceptable excipient. In some cases, a suitable formulation comprises: a) a compound comprising formula I, II, III, or IV of the present disclosure, or a compound depicted in any of FIG. 1-FIG. 4; and b) a pharmaceutically acceptable excipient. Suitable pharmaceutically acceptable excipients are described above.

### Dosages

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. A composition of the present disclosure may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute. A composition of the present disclosure can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a composition of the present disclosure is from 0.01 g to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a multimeric polypeptide of the present disclosure is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

Those of skill will readily appreciate that dose levels can vary as a function of the specific composition of the present disclosure, the severity of the symptoms and the susceptibility of the subject to side effects. Preferred dosages for a given compound are readily determinable by those of skill in the art by a variety of means.

In some cases, multiple doses of a composition of the present disclosure are administered. The frequency of administration of a composition of the present disclosure can vary depending on any of a variety of factors, e.g., severity of the symptoms, etc. For example, in some cases, a composition of the present disclosure is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a composition of the present disclosure, e.g., the period of time over which a composition of the present disclosure can vary, depending on any of a variety of factors, e.g., patient response, etc. For example, a composition of the present disclosure can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Routes of Administration

An active agent (a composition or a compound of the present disclosure) is administered to an individual using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intracranial, subcutaneous, intradermal, topical application, intravenous, intraarterial, rectal, nasal, oral, and other enteral and parenteral routes of administration. Routes of administration may be combined, if desired, or adjusted depending upon the composition of the present disclosure and/or the desired effect. A composition of the present disclosure can be administered in a single dose or in multiple doses.

In some cases, a composition of the present disclosure is administered intravenously. In some cases, a composition of the present disclosure is administered intramuscularly. In some cases, a composition of the present disclosure is administered locally. In some cases, a composition of the present disclosure is administered intratumorally. In some cases, a composition of the present disclosure is administered peritumorally. In some cases, a composition of the present disclosure is administered intracranially. In some cases, a composition of the present disclosure is administered subcutaneously.

A composition of the present disclosure can be administered to a host using any available conventional methods and routes suitable for delivery of conventional drugs, including systemic or localized routes. In general, routes of administration contemplated for use in a method of the present disclosure include, but are not necessarily limited to, enteral, parenteral, and inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, peritumoral, and intravenous routes, i.e., any route of administration other than through the alimentary canal. Parenteral administration can be carried to effect systemic or local delivery of a composition of the present disclosure. Where systemic delivery is desired, administration typically involves invasive or systemically absorbed topical or mucosal administration of pharmaceutical preparations.

### Protac

The present disclosure provides a bifunctional compound for the targeted degradation of a protein of interest.

One type of small molecule applicable to the present disclosure is a degrader molecule. Proteolysis Targeting Chimera (PROTAC) technology employs small molecules that recruit target proteins for ubiquitination and removal by the proteasome (see, e.g., Bondeson and Crews, Targeted Protein Degradation by Small Molecules, Annu Rev Pharmacol Toxicol. 2017 Jan. 6; 57: 107-123; Lai et al., Modular PROTAC Design for the Degradation of Oncogenic BCR-ABL Angew Chem Int Ed Engl. 2016 Jan. 11; 55(2): 807-810; and Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481).

PROTACs are heterobifunctional small molecules with three chemical elements: a ligand binding to a target protein, a ligand binding to E3 ubiquitin ligase, and a linker for conjugating these two ligands. PROTAC is a chemical knockdown strategy that degrades the target protein through the ubiquitin-proteasome system. Different from the competitive- and occupancy-driven process of traditional inhibitors, PROTACs are catalytic in their mode of action, which can promote target protein degradation at low exposures.

A PROTAC molecule may also be referred to as a “bifunctional molecule.” In some cases, the present disclosure provides a bifunctional molecule comprising: i) a Fem1b E3 ubiquitin ligase binding moiety (ULM) (i.e., a ligand for an E3 ubquitin ligase or “ULM” group); and ii) a protein targeting moiety (PTM) (i.e., a protein/polypeptide targeting ligand or “PTM” group) connected directly or through a chemical linker wherein the ULM is a group according to the chemical structure of formula I, II, III, or IV or a compound depicted in any of FIG. 1-FIG. 4 and the PTM is a ligand that binds to a target polypeptide of targeted protein degradation.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A composition comprising:
a1) a compound of Formula (I):

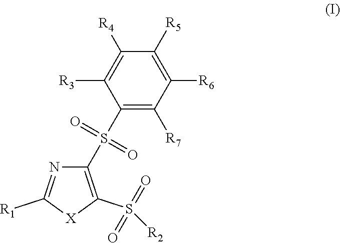

wherein:
X is O, S or NH;
R₁ is aryl, substituted aryl, arylalkyl, substituted arylalkyl, heteroaryl, substituted heteroaryl, heteroarylalkyl, and substituted heteroarylalkyl;
R₂ is alkyl, substituted alkyl, heteroalkyl, substituted heteroalkyl, acyl, aminoacyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R₃, R₄, R₅, R₆ and R₇ are each independently selected from hydrogen, alkyl, substituted alkyl, alkoxy, halogen, nitro, amino, hydroxy, cyano, thiol; and
b1) a pharmaceutically acceptable excipient; or
a2) a compound depicted in any one of FIG. 1-FIG. 4; and
b2) a pharmaceutically acceptable excipient.

Aspect 2. The composition of aspect 1, wherein R₁ is thiophene or substituted thiophene.

Aspect 3. The composition of aspect 1, wherein R₁ is phenyl or substituted phenyl.

Aspect 4. The composition of aspect 3, wherein R₁ is:

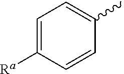

wherein R^{a} is hydrogen, hydroxyl or halogen and
wherein [symbol] represents the R¹—C bond.

Aspect 5. The composition of aspect 4, wherein R^{a} is hydrogen or a halogen selected from fluorine, chlorine, bromine or iodine.

Aspect 6. The composition of any one of aspects 1-5, wherein R₂ is alkyl, substituted alkyl, alkylaryl or substituted alkylaryl.

Aspect 7. The composition of aspect 6, wherein R₂ is a C1-C8 linear or C1-C8 branched alkyl.

Aspect 8. The composition of aspect 7, wherein R₂ is selected from methyl, ethyl, n-propyl, isopropyl, n-butyl, isobutyl, pentyl, isopentyl, hexyl, heptyl and octyl.

Aspect 9. The composition of aspect 6, wherein R₂ is benzyl or substituted benzyl.

Aspect 10. The composition of aspect 9, wherein R₂ is a halogen-substituted benzyl, wherein the halogen is selected from fluorine, chlorine, bromine or iodine.

Aspect 11. The composition of any one of aspects 1-5, wherein R₅ is hydrogen or a halogen selected from fluorine, chlorine, bromine or iodine.

Aspect 12. A composition comprising:
a compound of Formula (II):

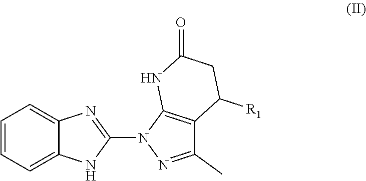

wherein R₁ is aryl, substituted aryl, arylalkyl, substituted arylalkyl, heteroaryl, substituted heteroaryl, heteroarylalkyl, and substituted heteroarylalkyl; and
a pharmaceutically acceptable excipient.

Aspect 13. The composition of aspect 12, wherein R₁ is phenyl or substituted phenyl.

Aspect 14. The composition of aspect 13, wherein R₁ is a halogen-substituted phenyl, wherein the halogen is selected from fluorine, chlorine, bromine or iodine.

Aspect 15. The composition of aspect 12, wherein R₁ is a pyrazole or substituted pyrazole.

Aspect 16. The composition of aspect 15, wherein R₁ is N-ethyl-3-methyl pyrazole.

Aspect 17. The composition of aspect 15, wherein R₁ is N-methyl-5-methyl pyrazole.

Aspect 18. The composition of aspect 12, wherein R₁ is a pyridine.

Aspect 19. The composition of aspect 12, wherein R₁ is a thiophene.

Aspect 20. A composition comprising:
a compound of Formula (III):

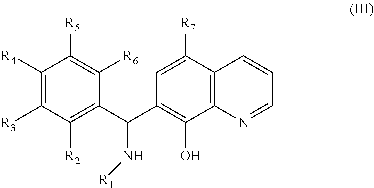

wherein:
R₁ is alkyl, substituted alkyl, acyl, acylalkyl, aryl, substituted aryl, heteroaryl and substituted heteroaryl;
R₂, R₃, R₄, R₅, R₆ and R₇ are each independently selected from hydrogen, alkyl, substituted alkyl, alkoxy, halogen, nitro, amino, hydroxy, cyano and thiol; and
a pharmaceutically acceptable excipient.

Aspect 21. The composition of aspect 20, wherein R₇ is hydrogen.

Aspect 22. The composition of aspect 20, wherein R₇ is nitro.

Aspect 23. The composition of any one of aspects 20-22, wherein R₁ is acyl.

Aspect 24. The composition of any aspect 23, wherein R₁ is,

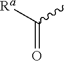

wherein R^{a} is a C1-C8 alkyl or substituted C1-C8 alkyl, and
wherein [symbol] represents the R¹—N bond.

Aspect 25. The composition of aspect 24, wherein R^{a} is selected from methyl, ethyl, n-propyl, isopropyl, n-butyl, isobutyl, pentyl, hexyl, heptyl and octyl.

Aspect 26. The composition of aspect 24, wherein R^{a} is a C1-C8 alkyl substituted with an aryl, substituted aryl, heteroaryl, substituted heteroaryl, cycloalkyl, substituted cycloalkyl, heterocycloalkyl and substituted heterocycloalkyl.

Aspect 27. The composition of aspect 26, wherein R^{a} is a C2 alkyl substituted with a phenyl.

Aspect 28. The composition of any one of aspects 20-27, wherein R₃ is hydroxy.

Aspect 29. The composition of any one of aspects 20-27, wherein R₃ is alkoxy.

Aspect 30. The composition of aspect 26, wherein R₃ is methoxy or ethoxy.

Aspect 31. The composition of any one of aspects 20-27, wherein R₃ is a C1-C8 alkyl.

Aspect 32. The composition of aspect 31, wherein R₃ is methyl.

Aspect 33. The composition of any one of aspects 20-32, wherein R₄ is hydroxy.

Aspect 34. The composition of any one of aspects 20-32, wherein R₄ is alkoxy.

Aspect 35. The composition of aspect 34, wherein R₄ is methoxy or ethoxy.

Aspect 36. The composition of any one of aspects 20-32, wherein R₄ is a C1-C8 alkyl.

Aspect 37. The composition of aspect 26, wherein R₄ is methyl.

Aspect 38. The composition of any one of aspects 20-37, wherein R₅ is hydroxy.

Aspect 39. The composition of any one of aspects 20-37, wherein R₅ is alkoxy.

Aspect 40. The composition of aspect 39, wherein R₅ is methoxy or ethoxy.

Aspect 41. The composition of any one of aspects 20-37, wherein R₅ is a C1-C8 alkyl.

Aspect 42. The composition of aspect 26, wherein R₅ is methyl.

Aspect 43. A composition comprising:
a compound of Formula (IV):

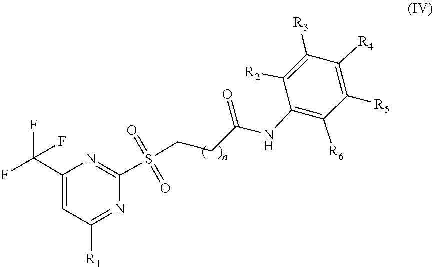

wherein:
R₁ is aryl, substituted aryl, heteroaryl, substituted heteroaryl, cycloalkyl, substituted cycloalkyl, heterocycloalkyl and substituted heterocycloalkyl;
R₂, R₃, R₄, R₅ and R₆ are each independently selected from hydrogen, alkyl, substituted alkyl, alkoxy, halogen, nitro, amino, hydroxy, cyano, thiol and thiolate.

a pharmaceutically acceptable excipient.

Aspect 44. The composition of aspect 43, wherein R₁ is thiophene.

Aspect 45. The composition of aspect 43, wherein R₁ is substituted phenyl.

Aspect 46. The composition of aspect 45, wherein R₁ is o-methoxy phenyl.

Aspect 47. The composition of aspect 45, wherein R₁ is:

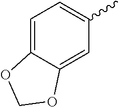

wherein [symbol] represents the R¹—C bond.

Aspect 48. The composition of aspect 43, wherein:
R₂ is hydroxyl; and
R₅ is bromine.

Aspect 49. The composition of aspect 43, wherein R₂ is methylthiolate.

Aspect 50. The composition of any one of aspects 1-49, wherein the pharmaceutically acceptable excipient is not dimethyl sulfoxide.

Aspect 51. The composition of any one of aspects 1-50, wherein the composition is sterile.

Aspect 52. A drug delivery device comprising the composition of any one of aspects 1-51.

Aspect 53. The drug delivery device of aspect 52, wherein the device comprises a syringe comprising the composition.

Aspect 54. The drug delivery device of aspect 52, wherein the device comprises a pump.

Aspect 55. A method of inhibiting the activity of an E3 ligase in a cell, the method comprising contacting the cell with the compound of Formula I, II, III, or IV, or a compound depicted in any one of FIG. 1-FIG. 4.

Aspect 56. The method of aspect 55, wherein the E3 ligase is Fem1b.

Aspect 57. The method of aspect 55 or aspect 56, wherein the cell is a cancer cell.

Aspect 58. A method of treating a cancer in an individual, the method comprising administering to the individual an effective amount of the composition any one of aspects 1-51.

Aspect 59. The method of aspect 58, wherein the cancer is lung adenocarcinoma.

Aspect 60. The method of aspect 58 or aspect 59, further comprising administering to the individual at least one additional cancer chemotherapeutic agent or anti-cancer polypeptide.

Aspect 61. The method of any one of aspects 58-60, wherein composition is administered orally, intraperitoneally, intramuscularly, or intravenously.

Aspect 62. A method of treating a metabolic disorder in an individual, the method comprising administering to the individual an effective amount of the composition of any one of aspects 1-51.

Aspect 63. The method of aspect 62, wherein the metabolic disorder is diabetes.

Aspect 64. The method of aspect 63, where the diabetes is type 2 diabetes.

Aspect 65. The method of aspect 62, wherein the metabolic disorder is a non-alcoholic fatty liver disease (NAFLD).

Aspect 66. The method of aspect 65, wherein the NAFLD comprises non-alcoholic steatohepatitis (NASH).

Aspect 67. The method of aspect 65, wherein the NAFLD comprises hepatic steatosis.

Aspect 68. The method of any one of aspects 62-67, wherein the individual has a body mass index greater than 30 kg/m².

Aspect 69. The method of any one of aspects 62-68, wherein the composition is administered orally, intraperitoneally, intramuscularly, or intravenously.

Aspect 70. The method of any one of aspects 62-69, further comprising administering to the individual at least one additional agent that treats a metabolic disorder.

Aspect 71. The method of aspect 70, wherein the at least one additional agent is selected from insulin, glucagon, a statin, and a thiazolidinedione.

Aspect 72. A method of inhibiting proliferation of a cancer cell, the method comprising contacting the cell with the compound of Formula I, II, III, or IV, or a compound depicted in any one of FIG. 1-FIG. 4.

Aspect 73. A method for treating a mental disorder in an individual, the method comprising administering to the individual an effective amount of the composition of any one of aspects 1-51.

Aspect 74. The method of aspect 73, wherein the mental disorder is syndromic intellectual disability.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Identifying Fem1b Inhibitors

### Chemical Library Composition

Food and Drug Administration (FDA)-approved and Bioactive chemical libraries containing 1,200 and 4,170 small molecule compounds, respectively, were stored at 10 mM in dimethyl sulfoxide (DMSO) in 384-well master plates (Targetmol, Wellesley Hills, MA) as well as 2 mM screening master plates. Diversity and Antibacterial chemical libraries containing 100,000 and 15,000 small molecule compounds, respectively, were purchased from (Chemdiv, San Diego, CA) with same storage conditions. Primary screening plates were generated by spotting 0.5 μl of 2 mM concentration screening master plates into 384 well non-binding plates (Greiner, 781900) using a Cybio Well Vario liquid handler (Analytik Jena, Jena, Germany) for a final concentration of 40 μM with a DMSO concentration of 2%.

Hits were cherry picked from 10 mM master plates and serial diluted with Tecan Freedom Evo 150 (Tecan Systems Inc, San Jose, CA)

### Fluorescence Polarization Screen

For the fluorescence polarization screens, mouse MBP-FEM1B and mutants (e.g., the R126A mutant of FEB1B) were purified as previously described (Manford et al. (2020) Cell 183:46) and the TAMRA-labeled folliculin-interacting protein 1 (FNIP1) peptide (5,6-TAMRA-RNKSSLLFKESEETRTPNCNCKYCSHPVLG; SEQ ID NO:3) was purchased from Koch Institute/MIT Biopolymers lab. The peptide was resuspended in DMSO to a 4 mM concentration and diluted to 200 μM stock concentration in binding buffer (40 mM HEPES 7.5, 150 mM NaCl, 0.2% NP40 substitute, and 100 μM TCEP (Tris(2-carboxyethyl)phosphine hydrochloride)) and aliquoted. The day of the screen, both MBP-FEM1B and the FNIP1 peptide were diluted into binding buffer to 250 nM and 100 nM respectively. 12.5 μl pf MBP-FEM1B was added to each well using a Cybio Well Vario liquid handler (Analytik Jena, Jena, Germany) and incubated for 1 hour at room temperature. After the incubation, 12.5 μl of the FNIP1 peptide was added to each well bringing the final concentration to 50 nM for the peptide, 125 nM for MBP-FEM1B, and 40 μM each compound.

Plates were mixed for 45 seconds at 2400 rpm using a QInstruments BioShake ELM3000 orbital shaker (QInstruments, Jena, Germany) and after 60 minutes of incubation plates were measured on a 2104 Envision plate reader (Perkin Elmer, Waltham, MA). Data was calculated from mP values (1000*(S−G*P)/(S+G*P), S=595s channel 2 and P=595p channel 1, G=1.1) with background subtraction calculated from a peptide only control plate. Each analysis group included a peptide and DMSO only plate, and FEM1B-FNIP1 peptide DMSO only plate containing a dose response of TPEN (N,N,N′,N′-Tetrakis(2-pyridylmethyl)ethylenediamine, Sigma-Aldrich, P4413) as a positive control (2 fold dilutions from 80 μM to −156 nM). Data was normalized to DMSO only controls on each plate.

### FNIP1 Degron Reporter Analysis

HEK293T cells were seeded at 300 k cells per well of a 6-well plate. The next day cells were transfected with 60 μl of a transfection mix consisting of 0.1 μg of the FEM1B mitochondrial reporter (FNIP1(562-591)-GFP-GlySer48 linker-OMP25(110-145)-IRES-mCherry) and 1.9 μg of pCS2+ in 300 μl Opti-MEM (Thermo Fisher, 31985-070) with 12 μg polyethyleneimine (PEI, Polysciences 23966-1). 12 hours post-transfection, 20 μM of indicated compound or DMSO was added. After 12 hours of treatment, cells were trypsinized, spun down, resuspended in DMEM+10% fetal bovine serum (FBS) and analyzed on Fortessa X20. Data was processed using FlowJo.

### Results of Primary Screen

The results of the primary screen are depicted in FIG. 1.

### Secondary Screen

A FNIP1 degron was cloned in frame with a mitochondrial targeting sequence and GFP and is expressed together with an mCherry fluorescent protein (“mito-reporter”). Both GFP and mCherry were measured by fluorescence activated cell sorting (FACS). The results of the secondary screen are depicted in FIG. 2A-20.

The ratio between the degron-GFP and mCherry signals indicates the stability of the GFP fusion dependent on FEM1B-dependent degradation (if the distribution is at low GFP/mCherry ratios, i.e. to the left in the graphs, then the degron is degraded; if the ratio is higher, i.e. shifted to the right in the graph as seen with active compounds, then the degron is stabilized).

### Additional Compounds

Additional compounds identified in the screen are shown in FIG. 3 and FIG. 4.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising:
a1) a compound of Formula (I): 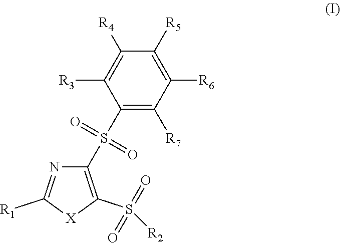
wherein:
X is O, S or NH;
R₁ is aryl, substituted aryl, arylalkyl, substituted arylalkyl, heteroaryl, substituted heteroaryl, heteroarylalkyl, and substituted heteroarylalkyl;
R₂ is alkyl, substituted alkyl, heteroalkyl, substituted heteroalkyl, acyl, aminoacyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
R₃, R₄, R₅, R₆ and R₇ are each independently selected from hydrogen, alkyl, substituted alkyl, alkoxy, halogen, nitro, amino, hydroxy, cyano, thiol; and
b1) a pharmaceutically acceptable excipient; or
a2) a compound depicted in any one of FIG. 1-4; and
b2) a pharmaceutically acceptable excipient.

2. The composition of claim 1, wherein R₁ is thiophene or substituted thiophene.

3. The composition of claim 1, wherein R₁ is phenyl or substituted phenyl.

4. The composition of claim 3, wherein R₁ is: 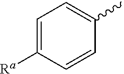
wherein R^{a} is hydrogen, hydroxyl or halogen and
wherein [symbol] represents the R¹—C bond.

5. The composition of claim 4, wherein R^{a} is hydrogen or a halogen selected from fluorine, chlorine, bromine or iodine.

6. The composition of any one of claims 1-5, wherein R₂ is alkyl, substituted alkyl, alkylaryl or substituted alkylaryl.

7. The composition of claim 6, wherein R₂ is a C1-C8 linear or C1-C8 branched alkyl.

8. The composition of claim 7, wherein R₂ is selected from methyl, ethyl, n-propyl, isopropyl, n-butyl, isobutyl, pentyl, isopentyl, hexyl, heptyl and octyl.

9. The composition of claim 6, wherein R₂ is benzyl or substituted benzyl.

10. The composition of claim 9, wherein R₂ is a halogen-substituted benzyl, wherein the halogen is selected from fluorine, chlorine, bromine or iodine.

11. The composition of any one of claims 1-5, wherein R₅ is hydrogen or a halogen selected from fluorine, chlorine, bromine or iodine.

12. A composition comprising:
a compound of Formula (II): 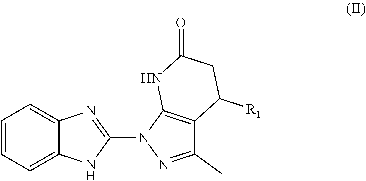
wherein R₁ is aryl, substituted aryl, arylalkyl, substituted arylalkyl, heteroaryl, substituted heteroaryl, heteroarylalkyl, and substituted heteroarylalkyl; and
a pharmaceutically acceptable excipient.

13. The composition of claim 12, wherein R₁ is phenyl or substituted phenyl.

14. The composition of claim 13, wherein R₁ is a halogen-substituted phenyl, wherein the halogen is selected from fluorine, chlorine, bromine or iodine.

15. The composition of claim 12, wherein R₁ is a pyrazole or substituted pyrazole.

16. The composition of claim 15, wherein R₁ is N-ethyl-3-methyl pyrazole.

17. The composition of claim 15, wherein R₁ is N-methyl-5-methyl pyrazole

18. The composition of claim 12, wherein R₁ is a pyridine.

19. The composition of claim 12, wherein R₁ is a thiophene.

20. A composition comprising:
a compound of Formula (III): 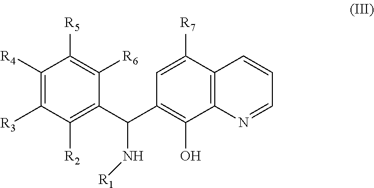
wherein:
R₁ is alkyl, substituted alkyl, acyl, acylalkyl, aryl, substituted aryl, heteroaryl and substituted heteroaryl;
R₂, R₃, R₄, R₅, R₆ and R₇ are each independently selected from hydrogen, alkyl, substituted alkyl, alkoxy, halogen, nitro, amino, hydroxy, cyano and thiol; and
a pharmaceutically acceptable excipient.

21. The composition of claim 20, wherein R₇ is hydrogen.

22. The composition of claim 20, wherein R₇ is nitro.

23. The composition of any one of claims 20-22, wherein R₁ is acyl.

24. The composition of any claim 23, wherein R₁ is, 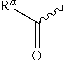
wherein R^{a} is a C1-C8 alkyl or substituted C1-C8 alkyl, and
wherein [symbol] represents the R¹—N bond.

25. The composition of claim 24, wherein R^{a} is selected from methyl, ethyl, n-propyl, isopropyl, n-butyl, isobutyl, pentyl, hexyl, heptyl and octyl.

26. The composition of claim 24, wherein R^{a} is a C1-C8 alkyl substituted with an aryl, substituted aryl, heteroaryl, substituted heteroaryl, cycloalkyl, substituted cycloalkyl, heterocycloalkyl and substituted heterocycloalkyl.

27. The composition of claim 26, wherein R^{a} is a C2 alkyl substituted with a phenyl.

28. The composition of any one of claims 20-27, wherein R₃ is hydroxy.

29. The composition of any one of claims 20-27, wherein R₃ is alkoxy.

30. The composition of claim 26, wherein R₃ is methoxy or ethoxy.

31. The composition of any one of claims 20-27, wherein R₃ is a C1-C8 alkyl.

32. The composition of claim 31, wherein R₃ is methyl.

33. The composition of any one of claims 20-32, wherein R₄ is hydroxy.

34. The composition of any one of claims 20-32, wherein R₄ is alkoxy.

35. The composition of claim 34, wherein R₄ is methoxy or ethoxy.

36. The composition of any one of claims 20-32, wherein R₄ is a C1-C8 alkyl.

37. The composition of claim 26, wherein R₄ is methyl.

38. The composition of any one of claims 20-37, wherein R₅ is hydroxy.

39. The composition of any one of claims 20-37, wherein R₅ is alkoxy.

40. The composition of claim 39, wherein R₅ is methoxy or ethoxy.

41. The composition of any one of claims 20-37, wherein R₅ is a C1-C8 alkyl.

42. The composition of claim 26, wherein R₅ is methyl.

43. A composition comprising:
a compound of Formula (IV): 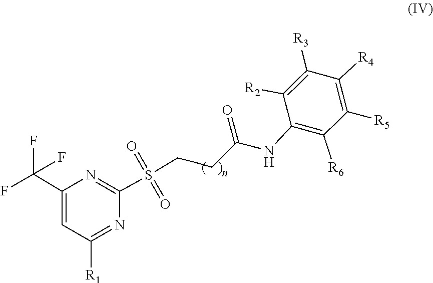
wherein:
R₁ is aryl, substituted aryl, heteroaryl, substituted heteroaryl, cycloalkyl, substituted cycloalkyl, heterocycloalkyl and substituted heterocycloalkyl;
R₂, R₃, R₄, R₅ and R₆ are each independently selected from hydrogen, alkyl, substituted alkyl, alkoxy, halogen, nitro, amino, hydroxy, cyano, thiol and thiolate.
a pharmaceutically acceptable excipient.

44. The composition of claim 43, wherein R₁ is thiophene.

45. The composition of claim 43, wherein R₁ is substituted phenyl.

46. The composition of claim 45, wherein R₁ is o-methoxy phenyl.

47. The composition of claim 45, wherein R₁ is: 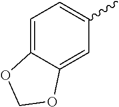
wherein [symbol] represents the R¹—C bond.

48. The composition of claim 43, wherein:
R₂ is hydroxyl; and
R₅ is bromine.

49. The composition of claim 43, wherein R₂ is methylthiolate.

50. The composition of any one of claims 1-49, wherein the pharmaceutically acceptable excipient is not dimethyl sulfoxide.

51. The composition of any one of claims 1-50, wherein the composition is sterile.

52. A drug delivery device comprising the composition of any one of claims 1-51.

53. The drug delivery device of claim 52, wherein the device comprises a syringe comprising the composition.

54. The drug delivery device of claim 52, wherein the device comprises a pump.

55. A method of inhibiting the activity of an E3 ligase in a cell, the method comprising contacting the cell with the compound of Formula I, II, III, or IV, or a compound depicted in any one of FIG. 1-FIG. 4.

56. The method of claim 55, wherein the E3 ligase is Fem1b.

57. The method of claim 55 or claim 56, wherein the cell is a cancer cell.

58. A method of treating a cancer in an individual, the method comprising administering to the individual an effective amount of the composition any one of claims 1-51.

59. The method of claim 58, wherein the cancer is lung adenocarcinoma.

60. The method of claim 58 or claim 59, further comprising administering to the individual at least one additional cancer chemotherapeutic agent or anti-cancer polypeptide.

61. The method of any one of claims 58-60, wherein composition is administered orally, intraperitoneally, intramuscularly, or intravenously.

62. A method of treating a metabolic disorder in an individual, the method comprising administering to the individual an effective amount of the composition of any one of claims 1-51.

63. The method of claim 62, wherein the metabolic disorder is diabetes.

64. The method of claim 63, where the diabetes is type 2 diabetes.

65. The method of claim 62, wherein the metabolic disorder is a non-alcoholic fatty liver disease (NAFLD).

66. The method of claim 65, wherein the NAFLD comprises non-alcoholic steatohepatitis (NASH).

67. The method of claim 65, wherein the NAFLD comprises hepatic steatosis.

68. The method of any one of claims 62-67, wherein the individual has a body mass index greater than 30 kg/m².

69. The method of any one of claims 62-68, wherein the composition is administered orally, intraperitoneally, intramuscularly, or intravenously.

70. The method of any one of claims 62-69, further comprising administering to the individual at least one additional agent that treats a metabolic disorder.

71. The method of claim 70, wherein the at least one additional agent is selected from insulin, glucagon, a statin, and a thiazolidinedione.

72. A method of inhibiting proliferation of a cancer cell, the method comprising contacting the cell with the compound of Formula I, II, III, or IV, or a compound depicted in any one of FIG. 1-FIG. 4.

73. A method for treating a mental disorder in an individual, the method comprising administering to the individual an effective amount of the composition of any one of claims 1-51.

74. The method of claim 73, wherein the mental disorder is syndromic intellectual disability.


SEQUENCE LISTING (3 sequences)

SEQ ID NO: 1 - protein, 627 aa, Homo sapiens
MEGLAGYVYKAASEGKVLTLAALLLNRSESDIRYLLGYVSQQGGQRSTPLIIAARNGHAKVVRLLLEHYRVQTQQTGTVRFDGYVIDGATALWCAAGAGHFEVVKLLVSHGANVNHTTVTNSTPLRAACFDGRLDIVKYLVENNANISIANKYDNTCLMIAAYKGHTDVVRYLLEQRADPNAKAHCGATALHFAAEAGHIDIVKELIKWRAAIVVNGHGMTPLKVAAESCKADVVELLLSHADCDRRSRIEALELLGASFANDRENYDIIKTYHYLYLAMLERFQDGDNILEKEVLPPIHAYGNRTECRNPQELESIRQDRDALHMEGLIVRERILGADNIDVSHPIIYRGAVYADNMEFEQCIKLWLHALHLRQKGNRNTHKDLLRFAQVFSQMIHLNETVKAPDIECVLRCSVLEIEQSMNRVKNISDADVHNAMDNYECNLYTFLYLVCISTKTQCSEEDQCKINKQIYNLIHLDPRTREGFTLLHLAVNSNTPVDDFHTNDVCSFPNALVTKLLLDCGAEVNAVDNEGNSALHIIVQYNRPISDFLTLHSIIISLVEAGAHTDMTNKQNKTPLDKSTTGVSEILLKTQMKMSLKCLAARAVRANDINYQDQIPRTLEEFVGFH

SEQ ID NO: 2 - protein, 627 aa, Mus musculus
MEGLAGYVYKAASEGKVLTLAALLLNRSESDIRYLLGYVSQQGGQRSTPLIIAARNGHAKVVRLLLEHYRVQTQQTGTVRFDGYVIDGATALWCAAGAGHFEVVKLLVSHGANVNHTTVTNSTPLRAACFDGRLDIVKYLVENNANISIANKYDNTCLMIAAYKGHTDVVRYLLEQRADPNAKAHCGATALHFAAEAGHIDIVKELIKWRAAIVVNGHGMTPLKVAAESCKADVVELLLSHADCDRRSRIEALELLGASFANDRENYDIMKTYHYLYLAMLERFQDGDNILEKEVLPPIHAYGNRTECRNPQELEAIRQDRDALHMEGLIVRERILGADNIDVSHPIIYRGAVYADNMEFEQCIKLWLHALHLRQKGNRNTHKDLLRFAQVFSQMIHLNEAVKAPDIECVLRCSVLEIEQSMNRVKNISDADVHSAMDNYECNLYTFLYLVCISTKTQCSEEDQCRINKQIYNLIHLDPRTREGFSLLHLAVNSNTPVDDFHTNDVCSFPNALVTKLLLDCGAEVNAVDNEGNSALHIIVQYNRPISDFLTLHSIIISLVEAGAHTDMTNKQNKTPLDKSTTGVSEILLKTQMKMSLKCLAARAVRANDINYQDQIPRTLEEFVGFH

SEQ ID NO: 3 - protein, 30 aa, Artificial sequence
  TAMRA-labeled folliculin-interacting protein 1 (FNIP1) peptide
RNKSSLLFKESEETRTPNCNCKYCSHPVLG